(19) United States
(12) Patent Application Publication
(10) Pub. No.: US 2016/0130340 A1
(43) Pub. Date: May 12, 2016

(54) DOSING REGIMENS USING ANTI-IL-6 ANTIBODIES FOR THE TREATMENT OF RHEUMATOID AND PSORIATIC ARTHRITIS

(71) Applicant: ALDERBIO HOLDINGS LLC, LAS VEGAS, NV (US)

(72) Inventor: Jeffrey T.L. Smith, Bellevue, WA (US)

(21) Appl. No.: 14/876,376

(22) Filed: Oct. 6, 2015

Related U.S. Application Data
(63) Continuation-in-part of application No. PCT/US2014/059543, filed on Oct. 7, 2014.
(60) Provisional application No. 62/060,797, filed on Oct. 7, 2014.
(60) Provisional application No. 61/887,666, filed on Oct. 7, 2013.

(51) Int. Cl. C07K 16/24; A61K 39/395; A61K 9/00; A61K 31/519

(52) U.S. Cl. CPC C07K 16/248; A61K 31/519; A61K 39/3955; A61K 9/0019; C07K 2317/56; C07K 2317/565; C07K 2317/567; C07K 2317/76; C07K 2317/94; C07K 2317/92; A61K 2039/505

(57) ABSTRACT

The present invention relates to therapeutic methods of using an antibody, or antigen-binding fragment thereof, which selectively binds IL-6 for the treatment or prevention of psoriatic arthritis or rheumatoid arthritis and for managing the side effects and symptoms of psoriatic or rheumatoid arthritis and therapeutic compositions for use therein comprising an antibody, or antigen-binding fragment thereof, which selectively binds IL-6 for the treatment or prevention of psoriatic or rheumatoid arthritis. The invention further relates to low dosing therapeutic regimens for treating inflammatory IL-6 associated diseases, i.e., characterized by elevated 11-6 levels such as psoriatic arthritis or rheumatoid arthritis that provided for reduced adverse side effects and improved safety. Also the invention further relates to compositions for use in low dosing therapeutic regimens for treating inflammatory IL-6 associated diseases, i.e., diseases characterized by elevated IL-6 levels such as psoriatic arthritis or rheumatoid arthritis, wherein such compositions may be administered by self-injection or by a caregiver using an autoinjector pen and a syringe containing the low dosage of anti-IL-6 antibody, e.g., 1, 5, 10, 15, 20 or 25 mg.

### RELATED APPLICATIONS

This application claims the benefit of priority to U.S. Provisional Ser. No. 62/060,797, filed on Oct. 7, 2014 and PCT Application entitled “ANTI-IL-6 ANTIBODIES FOR THE TREATMENT OF PSORIATIC ARTHRITIS” PCT/US2014/059543 filed on Oct. 7, 2014 which in turn claims priority to U.S. Provisional application No. 61/887,666 filed Oct. 7, 2013, the entire content of which are hereby incorporated herein by reference in their entirety as though fully set forth herein.

This application includes as part of its disclosure a biological sequence listing text file which is being submitted via EFS-Web. Said biological sequence listing is contained in the file named “43272o3005” having a size of 362,137 bytes that was created Oct. 6, 2015, which is hereby incorporated by reference in its entirety.

### FIELD OF THE SUBJECT TECHNOLOGY

Anti-IL-6 antibodies and antigen-binding fragments thereof are used to reduce C-reactive protein (“CRP levels”) and inflammation and in methods and compositions for the treatment and prevention of psoriatic arthritis (PsA).

### BACKGROUND

### Interleukin-6 (IL-6)

Interleukin-6 (“IL-6”) is a multifunctional cytokine involved in numerous biological processes such as the regulation of the acute inflammatory response, the modulation of specific immune responses including B- and T-cell differentiation, bone metabolism, thrombopoiesis, epidermal proliferation, menses, neuronal cell differentiation, neuroprotection, aging, cancer, and the inflammatory reaction occurring in Alzheimer's disease. See Papassotiropoulos, et al. (2001) Neurobiology of Aging 22: 863-871.

IL-6 is a pleiotropic pro-inflammatory cytokine, which regulates the acute phase response and the transition from the innate to the adaptive immune response. IL-6 increases hepatic synthesis of proteins that are involved in the ‘acute phase response’ leading to symptoms such as fever, chills, and fatigue. It stimulates B cell differentiation and secretion of antibodies and prevents apoptosis of activated B cells. IL-6 activates and induces proliferation of T cells and in the presence of IL-2, induces differentiation of mature and immature CD8 T cells into cytotoxic T cells. IL-6 is also involved in the differentiation of Th17 cells and IL-17 production and inhibits regulatory T cells (Treg) differentiation. IL-6 also activates osteoclasts, synoviocytes, neutrophils, and other hematopoietic cells. Park, et al. (2007) Bulletin of the NYU Hospital for Joint Diseases 65 (suppl 1): S4-10; Guerre, et al. (1989) J Clin Invest. 83(2): 585-92; Houssiau, et al. (1988) Arthritis Rheum. 31(6): 784-8; Nishimotor, et al. (2006) Nat Clin Pract Rheumatol. 2(11): 619-26; Kishimoto (1989) Blood 74(1): 1-10; and Van Snick (1990) Annu Rev Immunol. 8: 253-78.

IL-6 is a member of a family of cytokines that promote cellular responses through a receptor complex consisting of at least one subunit of the signal-transducing glycoprotein gp130 and the IL-6 receptor (“IL-6R”) (also known as gp80). The IL-6R may also be present in a soluble form (“sIL-6R”). IL-6 binds to IL-6R, which then dimerizes the signal-transducing receptor gp130. See Jones (2005) Immunology 175: 3463-3468.

In humans, the gene encoding IL-6 is organized in five exons and four introns, and maps to the short arm of chromosome 7 at 7p21. Translation of IL-6 RNA and post-translational processing result in the formation of a 21 to 28 kDa protein with 184 amino acids in its mature form. See Papassotiropoulos, et al. (2001) Neurobiology of Aging 22:863-871.

The function of IL-6 is not restricted to the immune response as it acts in hematopoiesis, thrombopoiesis, osteoclast formation, elicitation of hepatic acute phase response resulting in the elevation of C-reactive protein (CRP) and serum amyloid A (SAA) protein. It is known to be a growth factor for epidermal keratinocytes, renal mesangial cells, myeloma and plasmacytoma cells. Grossman, et al. (1989) Prot Natl Acad Sci. 86(16): 6367-6371; Horii, et al. (1989) J Immunol. 143(12): 3949-3955; and Kawano, et al. (1988) Nature 332: 83-85. IL-6 is produced by a wide range of cell types including monocytes/macrophages, fibroblasts, epidermal keratinocytes, vascular endothelial cells, renal messangial cells, glial cells, condrocytes, T and B-cells and some tumor cells. Akira, et al. (1990) FASEB J. 4(11): 2860-2867. Except for tumor cells that constitutively produce IL-6, normal cells do not express IL-6 unless appropriately stimulated.

Elevated IL-6 levels have been observed in many types of cancer, including breast cancer, leukemia, ovarian cancer, prostate cancer, pancreatic cancer, lymphoma, lung cancer, renal cell carcinoma, colorectal cancer, and multiple myeloma (e.g., Chopra et al. (2004) MJAFI 60:45-49; Songur et al. (2004) Tumori 90:196-200; Blay et al. (1992) Cancer Research 52: 3317-3322; Nikiteas et al. (2005) World J. Gasterenterol. 11:1639-1643; reviewed in Heikkila et al. (2008) Eur J Cancer 44:937-945). Clinical studies (reviewed in Trikha et al. (2003) Clinical Cancer Research 9: 4653-4665) have shown some improvement in patient outcomes due to administration of various anti-IL-6 antibodies, particularly in those cancers in which IL-6 plays a direct role promoting cancer cell proliferation or survival.

As noted above, IL-6 stimulates the hepatic acute phase response, resulting in increased production of CRP and elevated serum CRP levels. For this reason, C-reactive protein (CRP) has been reported to comprise a surrogate marker of IL-6 activity. Thus, elevated IL-6 activity can be detected through measurement of serum CRP. Conversely, effective suppression of IL-6 activity, e.g., through administration of a neutralizing anti-IL-6 antibody, can be detected by the resulting decrease in serum CRP levels. Although no diagnostic blood tests available for psoriatic arthritis, elevated CRP levels and erythrocyte sedimentation rate are known markers of inflammation and may reflect the severity of inflammation in the joints experienced in psoraitic arthritis. DermNet NZ by the New Zealand Dermatological Society Incorporated (2011).

### Rheumatoid Arthritis

Rheumatoid arthritis (RA) is one of the most common forms of chronic inflammatory arthritis, affecting approximately 1% of the population worldwide. Women are 2 to 3 times more likely to develop the disease compared to men, with a peak incidence between the fourth and sixth decades of life. While RA is recognized clinically because of the severe inflammation affecting the synovial joints, it is also a systemic disease with frequent extra-articular manifestations. The natural history of RA is characterized by joint destruction, impaired physical function, and poor health-related quality of life.

In general, treatment options for RA patients range from agents that provide symptomatic relief (such as analgesics, non-steroidal anti-inflammatory drugs [NSAIDs], and corticosteroids) to disease modifying agents that affect long-term structural damage. The current approach to RA treatment involves early intervention and progressive changes of therapy to improve signs and symptoms, and to prevent long term structural damage. Patients who are early in their disease course most commonly initiate treatment with one or more conventional synthetic DMARDs (eg. MTX). If there is an inadequate response (IR) to signs and symptoms or physical function with conventional synthetic DMARDs, these patients are often candidates for biologic therapy, most commonly anti tumor necrosis factor (anti-TNF) treatments. If patients do not attain adequate efficacy goals with anti-TNF agents, they are treated by switching to an alternative biologic therapy either within the anti-TNF class or with a different mechanism of action (e.g. co-stimulation blockade, anti-B-cell therapy, or anti-interleukin-6 [anti-IL-6] therapy).

Despite ongoing research and therapeutic advances that have led to significant improvement in patient health, RA remains a disease with considerable unmet medical need. The biological DMARD therapies now common in clinical practice perform reasonably well with respect to ACR criteria for 20% improvement (ACR20) response. However, their ability to achieve higher levels of efficacy is quite limited. For example, fewer than half of adult patients with moderate to severe active RA, who have had an inadequate response to MTX, achieve ACR criteria for 50% improvement (ACR50) response, and only approximately 20% of patients achieve ACR criteria for 70% improvement (ACR70) in recent trials of biologic therapies. More importantly, few patients achieve sustained levels of low disease activity or clinical remission. Data from clinical trials suggest that 5-20% of patients on conventional synthetic DMARD or biologic monotherapy, and 20-30% of patients on combination DMARD/biologic therapy achieve low disease activity or remission.

Low rates of low disease activity are of concern because the attainment of clinical remission is associated with less long term structural damage and physical disability. Recent treatment guidelines from an international task force have highlighted this need and recommended that control of disease activity is the therapeutic objective in RA, and have recommended a treat-to-target approach. As a result, there is a considerable need for new therapies that can help greater numbers of patients achieve low disease activity and clinical remission.

As disclosed infra, Clazakizumab, is a humanized monoclonal antibody that binds to the IL-6 cytokine which has demonstrated to be efficacious in RA with an acceptable safety profile and has shown numerically higher low disease activity and clinical remission rates as compared to adalimumab in a Phase 2b dose ranging study.

### Psoriatic Arthritis

Psoriatic arthritis (PsA) is a chronic inflammatory arthritis that occurs in individuals with psoriasis. It is estimated that about 1-3% of the general population and approximately 4.5 million patients in the United States have psoriasis. Between 10 and 30% of the psoriatic patients develop arthritis. PsA is a chronic inflammatory disease. The pathogenesis of PsA is not fully understood. Both genetic predisposition and environmental triggers are implicated in the deregulation of immune functions involved in PsA. A number of inflammatory cytokines including Interferon γ (INF γ), Tumor Necrosis Factor α (TNF α), IL-6, IL-8, IL-12, IL-17, and IL-18 are involved in the pathogenesis of psoriasis and PsA. Spadaro, et al. (1996) Clinical and Experimental Rheumatology 14: 413-416; Neuner, et al. (1991) The Journal of Investigative Dermatology 97(1): 27-33; Arican, et al. (2005) Mediators of Inflammation 5: 273-279; and Goodman, et al. (2009) The Journal of Immunology.

Psoriatic arthritis, a seronegative spondyloarthropathy is a complex disease involving peripheral and axial joints, periarticular structures (e.g., enthesitis and other soft tissues, resulting in dactylitis) as well as the skin and nails. Mease (2006) Bulletin of the NYU Hospital for Joint Diseases. 65(1-2): 25-31. Without appropriate management, the number of joints affected by PsA and the severity of joint damage increase over time, which can lead to marked restrictions of the daily activities and to substantially compromised quality of life. Evidence has shown that accelerated atherosclerosis, obesity, metabolic syndrome and cardiovascular disease are associated with active PsA. Other co-morbidities such as pulmonary fibrosis, uveitis, and, less commonly, aorta and aortic valve inflammation also contribute to complexity of PsA. Mease (2005) Expert Opin. Biol. Ther. 5(11): 1491-1504; Mease (2006) Bulletin of the NYU Hospital for Joint Diseases. 65(1-2): 25-31; and Weger (2010) British Journal of Pharmacology. 160: 810-820.

Treatment options are limited for PsA. Saad, et al. (2008) J Rheumatol. 35(5): 883-890; Kavanuah, et al. (2006) J Rheumatol. 33(7): 1417-142; Gavin, (2010) The Hong Kong Medical Diary. 15(5): 26-27; and Nash (2006) J Rheumatol. 33(7): 1431-1424. Responses to the traditional disease-modifying anti-rheumatic drugs (DMARDs) have been suboptimal. Kavanuah, et al. (2006) J Rheumatol. 33(7): 1417-1421 and Soriana, et al. (2006) J Rheumatol. 33(7): 1422-1430. Anti-tumor necrosis factor (TNF) therapies are efficacious for both skin and joint diseases but approximately 40% of patients treated with anti-TNF agents do not show at least minimal improvement and a large portion of patients do not achieve substantial relief. Mease (2006) Bulletin of the NYU Hospital for Joint Diseases. 65(1-2): 25-31; Weger (2010) British Journal of Pharmacology. 160: 810-820; Mease, et al. (2000) The Lancet 356: 385-390. Mease, et al. (2004) Arthritis and Rheum. 50(7): 2264-2272; Genovese, et al. (2007) J Rheumatol. 34(5): 1040-1050; Mease, et al. (2005) Arthritis and Rheum. 52(10: 3279-3289; Mease (2007) Therapeutics and Clinical Risk Management 3(1): 133-148; Antoni, et al. (2005) Arthritis and Rheum. 52(4): 1227-1236; Antoni, et al. (2008) J Rheumatol. 35(5): 869-876; and Antoni, et al. (2005) Ann Rhem Dis. 64: 1150-1157. For example, several effective rheumatoid arthritis therapies do not provide the relief in PsA, leaving anti-TNF agents as the only class of approved biologic therapy for PsA. Several agents under development for PsA had good efficacy for psoriatic skin lesions but with less optimal joint efficacy. Mease (2006) Bulletin of the NYU Hospital for Joint Diseases. 65(1-2): 25-31. Weger (2010) British Journal of Pharmacology. 160: 810-820. Gottlieb, et al. (2009) The Lancet 373: 633-640.

More severe arthritis has been treated with drugs called disease-modifying antirheumatic drugs (DMARDs), such as Leflunomide, Methotrexate, and Sulfasalazine. New medications that block an inflammatory protein called tumor necrosis factor (TNF) are becoming the treatment of choice for psoriatic arthritis including Adalimumab (Humira), Etanercept (Enbrel), Golimumab (Simponi), Infliximab (Remicade). Occasionally, very painful joints may be injected with steroid medications. A.D.A.M. Medical Encyclopedia (Jun. 29, 2011). However, many patients do not experience relief from psoriatic arthritis with DMARDs or non-steroidal anti-inflammatory drugs (NSAIDs). Therefore, there is still significant unmet need in PsA for therapies that provide higher levels of efficacy in a greater proportion of patients for both joints and skin along with the additional attributes of durability of effect over time, low immunogenicity, a subcutaneous dosing regimen that may allow for less frequent administration, and a risk benefit profile that remains acceptable.

Data suggest that IL-6 plays an important role in the pathogenesis that leads to the joint inflammation as well as the characteristic skin lesions associated with psoriasis arthritis. Spadaro, et al. (1996) Clinical and Experimental Rheumatology 14: 413-416. Neuner, et al. (1991) The Journal of Investigative Dermatology 97(1): 27-33. Arican, et al. (2005) Mediators of Inflammation 5: 273-279. Goodman, et al. (2009) The Journal of Immunology. Grossman, et al. (1989) Medical Sciences, Proc. Natl. Acad. Sci. USA. 86: 6367-6371. Circulating IL-6 levels were significantly higher in PsA patients and correlated highly with ESR and CRP. IL-6 levels also strongly correlated with disease activities such as the number of painful and swollen joints, physician's assessments as well as psoriasis area and severity index (PASI). Spadaro, et al. (1996) Clinical and Experimental Rheumatology 14: 413-416. The histopathology of psoriatic skin lesions is characterized by epidermal hyperplasia and inflammation. Studies have shown IL-6 mRNA and protein levels are elevated in psoriatic plaques. IL-6 was also shown to stimulate keratinocyte proliferation in vitro and contribute to the plaque formation. IL-6 signaling in psoriasis was also shown to prevent immune suppression by regulatory T cells. Neuner, et al. (1991) The Journal of Investigative Dermatology 97(1): 27-33. Arican, et al. (2005) Mediators of Inflammation 5: 273-279. Goodman, et al. (2009) The Journal of Immunology. Grossman, et al. (1989) Medical Sciences, Proc. Natl. Acad. Sci. USA. 86: 6367-6371. Alenius, et al. (2009) Clinical and Experimental Rheumatology 27: 120-123. Therefore, blocking IL-6 may provide therapeutic benefits in PsA.

Therefore, there is a strong need in the art for improved methods of treating and preventing psoriatic arthritis.

### SUMMARY

The present technology provides compositions comprising humanized monoclonal antibodies that selectively bind IL-6 and methods of treating psoriatic arthritis. In one embodiment, anti-IL-6 antibodies (e.g., ALD518 antibodies) are used in methods for the treatment of psoriatic arthritis. In this embodiment of the subject technology anti-IL-6 antibody or antibody fragment are administered prophylactically to patients at significant risk of developing psoriatic arthritis. The subject technology also provides for humanized monoclonal anti-IL-6 antibodies which are used in the treatment of psoriatic arthritis. The present subject technology further includes the prevention or treatment of inflammatory conditions by administration of anti-IL-6 antibodies according to the subject technology.

The subject technology provides a method of treating or preventing psoriatic arthritis comprising administration of a composition comprising an effective amount of an Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, Ab21, Ab22, Ab23, Ab24, Ab25, Ab26, Ab27, Ab28, Ab29, Ab30, Ab31, Ab32, Ab33, Ab34, Ab35, or Ab36 antibody, or an antigen-binding fragment thereof, to a subject in need thereof, wherein the antibody, or antigen-binding fragment thereof, specifically binds to IL-6.

The subject technology also provides a method of treating psoriatic arthritis comprising administration of a composition comprising an effective amount of an Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, Ab21, Ab22, Ab23, Ab24, Ab25, Ab26, Ab27, Ab28, Ab29, Ab30, Ab31, Ab32, Ab33, Ab34, Ab35, or Ab36 antibody, or an antigen-binding fragment thereof, to a subject in need thereof, wherein the antibody, or antigen-binding fragment thereof, specifically binds to IL-6.

The subject technology further provides a method of preventing psoriatic arthritis comprising administration of a composition comprising an effective amount of an Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, Ab21, Ab22, Ab23, Ab24, Ab25, Ab26, Ab27, Ab28, Ab29, Ab30, Ab31, Ab32, Ab33, Ab34, Ab35, or Ab36 antibody, or an antigen-binding fragment thereof, to a subject in need thereof, wherein the antibody, or antigen-binding fragment thereof, specifically binds to IL-6.

The subject technology provides a composition for the treatment or prevention of psoriatic arthritis comprising an effective amount of an Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, Ab21, Ab22, Ab23, Ab24, Ab25, Ab26, Ab27, Ab28, Ab29, Ab30, Ab31, Ab32, Ab33, Ab34, Ab35, or Ab36 antibody, or an antigen-binding fragment thereof, to a subject in need thereof, wherein the antibody, or antigen-binding fragment thereof, specifically binds to IL-6.

The subject technology also provides a composition for the treatment of psoriatic arthritis comprising an effective amount of an Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, Ab21, Ab22, Ab23, Ab24, Ab25, Ab26, Ab27, Ab28, Ab29, Ab30, Ab31, Ab32, Ab33, Ab34, Ab35, or Ab36 antibody, or an antigen-binding fragment thereof, to a subject in need thereof, wherein the antibody, or antigen-binding fragment thereof, specifically binds to IL-6.

The subject technology further provides a composition for the prevention of psoriatic arthritis comprising an effective amount of an Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, Ab21, Ab22, Ab23, Ab24, Ab25, Ab26, Ab27, Ab28, Ab29, Ab30, Ab31, Ab32, Ab33, Ab34, Ab35, or Ab36 antibody, or an antigen-binding fragment thereof, to a subject in need thereof, wherein the antibody, or antigen-binding fragment thereof, specifically binds to IL-6.

The subject technology provides a composition comprising an effective amount of an Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, Ab21, Ab22, Ab23, Ab24, Ab25, Ab26, Ab27, Ab28, Ab29, Ab30, Ab31, Ab32, Ab33, Ab34, Ab35, or Ab36 antibody, or an antigen-binding fragment thereof, to a subject in need thereof, wherein the antibody, or antigen-binding fragment thereof, specifically binds to IL-6.

The subject technology also provides for a pharmaceutical composition comprising an effective amount of an Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, Ab21, Ab22, Ab23, Ab24, Ab25, Ab26, Ab27, Ab28, Ab29, Ab30, Ab31, Ab32, Ab33, Ab34, Ab35, or Ab36 antibody, or an antigen-binding fragment thereof, to a subject in need thereof, wherein the antibody, or antigen-binding fragment thereof, specifically binds to IL-6.

The subject technology provides for the use of a composition comprising an effective amount of an Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, Ab21, Ab22, Ab23, Ab24, Ab25, Ab26, Ab27, Ab28, Ab29, Ab30, Ab31, Ab32, Ab33, Ab34, Ab35, or Ab36 antibody, or an antigen-binding fragment thereof, to a subject in need thereof, wherein the antibody, or antigen-binding fragment thereof, specifically binds to IL-6, for the manufacture of a medicament for the treatment or prevention of psoriatic arthritis. In a further embodiment, said composition may be formulated for subcutaneous administration.

The subject technology also provides for the use of a composition comprising an effective amount of an Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, Ab21, Ab22, Ab23, Ab24, Ab25, Ab26, Ab27, Ab28, Ab29, Ab30, Ab31, Ab32, Ab33, Ab34, Ab35, or Ab36 antibody, or an antigen-binding fragment thereof, to a subject in need thereof, wherein the antibody, or antigen-binding fragment thereof, specifically binds to IL-6, for the manufacture of a medicament for the treatment of psoriatic arthritis. In a further embodiment, said composition may be formulated for subcutaneous administration.

The subject technology provides for the use of a composition comprising an effective amount of an Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, Ab21, Ab22, Ab23, Ab24, Ab25, Ab26, Ab27, Ab28, Ab29, Ab30, Ab31, Ab32, Ab33, Ab34, Ab35, or Ab36 antibody, or an antigen-binding fragment thereof, to a subject in need thereof, wherein the antibody, or antigen-binding fragment thereof, specifically binds to IL-6, for the manufacture of a medicament for the prevention of psoriatic arthritis. In a further embodiment, said composition may be formulated for subcutaneous administration.

In one embodiment, the antibody includes at least one light chain amino acid sequence with at least about 50% identity to an amino acid sequence selected from the group consisting of SEQ ID NOs: 2, 20, 21, 37, 53, 69, 85, 101, 119, 122, 138, 154, 170, 186, 202, 218, 234, 250, 266, 282, 298, 314, 330, 346, 362, 378, 394, 410, 426, 442, 458, 474, 490, 506, 522, 538, 554, 570, 647, 648, 649, 650, 651, 655, 660, 666, 667, 671, 675, 679, 683, 687, 693, 699, 702, 706, and 709. In another embodiment, the antibody may comprise at least one light chain of nucleic acid sequences with at least 50% identity to a nucleic acid sequence selected from the group consisting of SEQ ID NOs: 10, 29, 45, 61, 77, 93, 109, 130, 146, 162, 178, 194, 210, 226, 242, 258, 274, 290, 306, 322, 338, 354, 370, 386, 402, 418, 434, 450, 466, 482, 498, 514, 530, 546, 562, 578, 662, 669, 673, 677, 681, 685, 689, 698, 701, 705, 720, 721, 722, and 723, wherein said nucleic acid sequence encodes said light chain.

In one embodiment, the antibody includes at least one heavy chain amino acid sequence with at least about 50% identity to an amino acid sequence selected from the group consisting of SEQ ID NOs: 3, 18, 19, 22, 38, 54, 70, 86, 102, 117, 118, 123, 139, 155, 171, 187, 203, 219, 235, 251, 267, 283, 299, 315, 331, 347, 363, 379, 395, 411, 427, 443, 459, 475, 491, 507, 523, 539, 555, 571, 652, 653, 654, 655, 656, 657, 658, 661, 664, 665, 668, 672, 676, 680, 684, 688, 691, 692, 704, and 708. In another embodiment, the antibody includes at least one heavy chain nucleic acid sequences with at least 50% identity to a nucleic acid sequence selected from the group consisting of SEQ ID NOs: 11, 30, 46, 62, 78, 94, 110, 131, 147, 163, 179, 195, 211, 227, 243, 259, 275, 291, 307, 323, 339, 355, 371, 387, 403, 419, 435, 451, 467, 483, 499, 515, 531, 547, 563, 579, 663, 670, 674, 678, 682, 686, 690, 700, 703, 707, 724, and 725, wherein said nucleic acid sequence encodes said heavy chain.

In one embodiment, the antibody includes at least one CDR amino acid sequence with at least about 50% identity to an amino acid sequence selected from the group consisting of SEQ ID NOs: 4, 7, 23, 26, 39, 42, 55, 58, 71, 74, 87, 90, 103, 106, 124, 127, 140, 143, 156, 159, 172, 175, 188, 191, 204, 207, 220, 223, 236, 239, 252, 255, 268, 271, 284, 287, 300, 303, 316, 319, 332, 335, 348, 351, 364, 367, 380, 383, 396, 399, 412, 415, 428, 431, 444, 447, 460, 463, 476, 479, 492, 495, 508, 511, 524, 527, 540, 543, 556, 559, 572, 575, 710, 711, 712, 716, 5, 8, 24, 27, 40, 43, 56, 59, 72, 75, 88, 91, 104, 107, 120, 121, 125, 128, 141, 144, 157, 160, 173, 176, 189, 192, 205, 208, 221, 224, 237, 240, 253, 256, 269, 272, 285, 288, 301, 304, 317, 320, 333, 336, 349, 352, 365, 368, 381, 384, 397, 400, 413, 416, 429, 432, 445, 448, 461, 464, 477, 480, 493, 496, 509, 512, 525, 528, 541, 544, 557, 560, 573, 576, 659, 713, 714, 715, 717, 718, 6, 9, 25, 28, 41, 44, 57, 60, 73, 76, 89, 92, 105, 108, 126, 129, 142, 145, 158, 161, 174, 177, 190, 193, 206, 209, 222, 225, 238, 241, 254, 257, 270, 273, 286, 289, 302, 305, 318, 321, 334, 337, 350, 353, 366, 369, 382, 385, 398, 401, 414, 417, 430, 433, 446, 449, 462, 465, 478, 481, 494, 497, 510, 513, 526, 529, 542, 545, 558, 561, 574, and 577. In one embodiment, the antibody includes at least one CDR nucleic acid sequences with at least 50% identity to a nucleic acid sequence selected from the group consisting of SEQ ID NOs: 12, 15, 31, 34, 47, 50, 63, 66, 79, 82, 95, 98, 111, 114, 132, 135, 148, 151, 164, 167, 180, 183, 196, 199, 212, 215, 228, 231, 244, 247, 260, 263, 276, 279, 292, 295, 308, 311, 324, 327, 340, 343, 356, 359, 372, 375, 388, 391, 404, 407, 420, 423, 436, 439, 452, 455, 468, 471, 484, 487, 500, 503, 516, 519, 532, 535, 548, 551, 564, 567, 580, 583, 694, 13, 16, 32, 35, 48, 51, 64, 67, 80, 83, 96, 99, 112, 115, 133, 136, 149, 152, 165, 168, 181, 184, 197, 200, 213, 216, 229, 232, 245, 248, 261, 264, 277, 280, 293, 296, 309, 312, 325, 328, 341, 344, 357, 360, 373, 376, 389, 392, 405, 408, 421, 424, 437, 440, 453, 456, 469, 472, 485, 488, 501, 504, 517, 520, 533, 536, 549, 552, 565, 568, 581, 584, 696, 14, 17, 33, 36, 49, 52, 65, 68, 81, 84, 97, 100, 113, 116, 134, 137, 150, 153, 166, 169, 182, 185, 198, 201, 214, 217, 230, 233, 246, 249, 262, 265, 278, 281, 294, 297, 310, 313, 326, 329, 342, 345, 358, 361, 374, 377, 390, 393, 406, 409, 422, 425, 438, 441, 454, 457, 470, 473, 486, 489, 502, 505, 518, 521, 534, 537, 550, 553, 566, 569, 582, 585, 695, and 697, wherein said nucleic acid sequence encodes said CDR sequence.

In another embodiment, the antibody or antigen-binding fragment thereof includes at least one light chain CDR amino acid sequence with at least about 50% identity to an amino acid sequence selected from the group consisting of SEQ ID NOs: 4, 23, 39, 55, 71, 74, 87, 103, 124, 140, 156, 172, 188, 204, 220, 236, 252, 268, 284, 300, 316, 332, 348, 364, 380, 396, 412, 428, 444, 460, 476, 492, 508, 524, 540, 556, 572, 710, 711, 712, 5, 6, 24, 40, 56, 72, 88, 104, 125, 141, 157, 173, 189, 205, 221, 237, 253, 269, 285, 301, 317, 333, 349, 365, 381, 397, 413, 429, 445, 461, 477, 493, 509, 525, 541, 557, 573, 713, 714, 715, 718, 25, 41, 57, 73, 89, 105, 126, 142, 158, 174, 190, 206, 222, 238, 254, 270, 286, 302, 318, 334, 350, 366, 382, 398, 414, 430, 446, 462, 478, 494, 510, 526, 542, 558, and 574. In another embodiment, the antibody or antigen-binding fragment thereof includes at least one light chain CDR1 amino acid sequence with at least about 50% identity to an amino acid sequence selected from the group consisting of SEQ ID NOs: 4, 23, 39, 55, 71, 74, 87, 103, 124, 140, 156, 172, 188, 204, 220, 236, 252, 268, 284, 300, 316, 332, 348, 364, 380, 396, 412, 428, 444, 460, 476, 492, 508, 524, 540, 556, 572, 710, 711, and 712. In another embodiment, the antibody or antigen-binding fragment thereof includes at least one light chain CDR2 amino acid sequence with at least about 50% identity to an amino acid sequence selected from the group consisting of SEQ ID NOs: 5, 24, 40, 56, 72, 88, 104, 125, 141, 157, 173, 189, 205, 221, 237, 253, 269, 285, 301, 317, 333, 349, 365, 381, 397, 413, 429, 445, 461,7 477, 493, 509, 525, 541, 557, 573, 713, 714, 715, and 718. In another embodiment, the antibody or antigen-binding fragment thereof includes at least one light chain CDR3 amino acid sequence with at least about 50% identity to an amino acid sequence selected from the group consisting of SEQ ID NOs: 6, 25, 41, 57, 73, 89, 105, 126, 142, 158, 174, 190, 206, 222, 238, 254, 270, 286, 302, 318, 334, 350, 366, 382, 398, 414, 430, 446, 462, 478, 494, 510, 526, 542, 558, and 574. In another embodiment, the antibody or antigen-binding fragment thereof includes at least two light chain CDR polypeptides. In another embodiment, the antibody or antigen-binding fragment thereof may comprise three light chain CDR polypeptides.

In another embodiment, the antibody or antigen-binding fragment thereof includes at least one heavy chain CDR amino acid sequence with at least about 50% identity to an amino acid sequence selected from the group consisting of SEQ ID NOs: 7, 26, 42, 58, 74, 90, 106, 127, 143, 159, 175, 191, 207, 223, 239, 255, 271, 287, 303, 319, 335, 351, 367, 383, 399, 415, 431, 447, 463, 479, 495, 511, 527, 543, 559, 575, 716, 8, 27, 43, 59, 75, 91, 107, 120, 121, 128, 144, 160, 176, 192, 208, 224, 240, 256, 272, 288, 304, 320, 336, 352, 368, 384, 400, 416, 432, 448, 464, 480, 496, 512, 528, 544, 560, 576, 659, 717, 718, 9, 28, 44, 60, 76, 92, 108, 129, 145, 161, 177, 193, 209, 225, 241, 257, 273, 289, 305, 321, 337, 353, 369, 385, 401, 417, 433, 449, 465, 481, 497, 513, 529, 545, 561, and 577. In a further embodiment, the antibody or antigen-binding fragment thereof includes at least one heavy chain CDR1 amino acid sequence with at least about 50% identity to an amino acid sequence selected from the group consisting of SEQ ID NOs: 7, 26, 42, 58, 74, 90, 106, 127, 143, 159, 175, 191, 207, 223, 239, 255, 271, 287, 303, 319, 335, 351, 367, 383, 399, 415, 431, 447, 463, 479, 495, 511, 527, 543, 559, 575, and 716. In a further embodiment, the antibody or antigen-binding fragment thereof includes at least one heavy chain CDR2 amino acid sequence with at least about 50% identity to an amino acid sequence selected from the group consisting of SEQ ID NOs: 8, 27, 43, 59, 75, 91, 107, 120, 121, 128, 144, 160, 176, 192, 208, 224, 240, 256, 272, 288, 304, 320, 336, 352, 368, 384, 400, 416, 432, 448, 464, 480, 496, 512, 528, 544, 560, 576, 659, 717, and 718. In a further embodiment, the antibody or antigen-binding fragment thereof includes at least one heavy chain CDR3 amino acid sequence with at least about 50% identity to an amino acid sequence selected from the group consisting of SEQ ID NOs: 9, 28, 44, 60, 76, 92, 108, 129, 145, 161, 177, 193, 209, 225, 241, 257, 273, 289, 305, 321, 337, 353, 369, 385, 401, 417, 433, 449, 465, 481, 497, 513, 529, 545, 561, and 577. In a further embodiment, the antibody or antigen-binding fragment thereof includes at least two heavy chain CDR amino acid sequences. In a further embodiment, the antibody or antigen-binding fragment thereof includes three heavy chain CDR amino acid sequences.

In one embodiment, the light chain of said antibody is selected from the amino acid sequences of light chains listed in TABLE 1. In one embodiment, the light chain of said antibody is selected from the amino acid sequences of heavy chains listed in TABLE 1. In one embodiment, at least one CDR of said antibody is selected from the amino acid sequences of CDRs listed in TABLE 1. In another embodiment, the light chain has at least 80% identity to an amino acid sequence listed in TABLE 1. In another embodiment, the light chain has at least 90% identity to an amino acid sequence listed in TABLE 1. In another embodiment, the light chain includes an amino acid sequence listed in TABLE 1. In further embodiment, the heavy chain has at least 80% identity to an amino acid sequence listed in TABLE 1. In further embodiment, the heavy chain has at least 90% identity to an amino acid sequence listed in TABLE 1. In further embodiment, the heavy chain includes an amino acid sequence listed in TABLE 1. In a still further embodiment, the CDR sequence of the antibody has at least 80% identity to an amino acid sequence listed in TABLE 1. In a still further embodiment, the CDR sequence of the antibody has at least 90% identity to an amino acid sequence listed in TABLE 1. In a still further embodiment, the CDR sequence of the antibody includes an amino acid sequence listed in TABLE 1.

In one embodiment, the antibody or antigen-binding fragment thereof includes at least one of the CDRs contained in the V_{H} polypeptide sequences comprising: SEQ ID NO: 3, 18, 19, 22, 38, 54, 70, 86, 102, 117, 118, 123, 139, 155, 171, 187, 203, 219, 235, 251, 267, 283, 299, 315, 331, 347, 363, 379, 395, 411, 427, 443, 459, 475, 491, 507, 523, 539, 555, 571, 652, 656, 657, 658, 661, 664, 665, 668, 672, 676, 680, 684, 688, 691, 692, 704, or 708 and/or at least one of the CDRs contained in the V_{L} polypeptide sequence consisting of: 2, 20, 21, 37, 53, 69, 85, 101, 119, 122, 138, 154, 170, 186, 202, 218, 234, 250, 266, 282, 298, 314, 330, 346, 362, 378, 394, 410, 426, 442, 458, 474, 490, 506, 522, 538, 554, 570, 647, 651, 660, 666, 667, 671, 675, 679, 683, 687, 693, 699, 702, 706, or 709.

In one embodiment, the antibody is an Ab1 antibody. In one embodiment, the antibody includes a light chain comprising the amino acid sequence of SEQ ID NO: 2, 20, 647, 648, 649, 650, 651, 660, 666, 699, 702, 706, or 709. In one embodiment, the antibody includes a humanized light chain comprising the amino acid sequence of SEQ ID NO: 648, 649, and 650. In one embodiment, the antibody includes at least one light chain CDR comprising the amino acid sequence selected from the group consisting of SEQ ID NO: 4, 5, 6, 710, 711, 712, 713, 714, and 715. In one embodiment, the antibody includes at least one humanized light chain CDR comprising the amino acid sequence selected from the group consisting of SEQ ID NO: 710, 711, 712, 713, 714, and 715. In another embodiment, the antibody includes a heavy chain comprising the amino acid sequence of SEQ ID NO: 3, 18, 19, 652, 653, 654, 655, 656, 657, 658, 661, 664, 665, 704, 708. In another embodiment, the antibody includes a humanized heavy chain comprising the amino acid sequence of SEQ ID NO: 653, 654, and 655. In another embodiment, the antibody includes at least one heavy chain CDR comprising the amino acid sequence selected from the group consisting of SEQ ID NO: 7, 9, 74, 716, 8, 120, 659, 717, and 718. In another embodiment, the antibody includes at least one humanized heavy chain CDR comprising the amino acid sequence selected from the group consisting of SEQ ID NO: 74, 716, 717, and 718.

In one embodiment, the antibody or antigen-binding fragment thereof includes a Fab, Fab′, F(ab′)₂, Fv, scFv, IgNAR, SMIP, camelbody, or nanobody. In one embodiment, the antibody or antigen-binding fragment thereof may have an in vivo half-life of at least about 30 days in a healthy human subject. In one embodiment, the antibody or antigen-binding fragment thereof may have a binding affinity (Kd) for IL-6 of less than about 50 picomolar, or a rate of dissociation (K_{off}) from IL-6 of less than or equal to 10⁻⁴ S⁻¹. In one embodiment, the antibody or antigen-binding fragment thereof may specifically binds to the same linear or conformational epitope(s) and/or competes for binding to the same linear or conformational epitope(s) on an intact human IL-6 polypeptide or fragment thereof as an anti-IL-6 antibody comprising the polypeptides of SEQ ID NO: 702 and SEQ ID NO: 704 or the polypeptides of SEQ ID NO: 2 and SEQ ID NO: 3. In one embodiment, the binding to the same linear or conformational epitope(s) and/or competition for binding to the same linear or conformational epitope(s) on an intact human IL-6 polypeptide or fragment thereof is ascertained by epitopic mapping using overlapping linear peptide fragments which span the full length of the native human IL-6 polypeptide and includes at least one residues comprised in IL-6 fragments selected from those respectively encompassing amino acid residues 37-51, amino acid residues 70-84, amino acid residues 169-183, amino acid residues 31-45 and/or amino acid residues 58-72 of SEQ ID NO: 1.

In one embodiment, the antibody, or antigen-binding fragment thereof, may be aglycosylated. In one embodiment, the antibody, or antigen-binding fragment thereof, contains an Fc region that has been modified to alter effector function, half-life, proteolysis, and/or glycosylation. In one embodiment, the antibody, or antigen-binding fragment thereof, is a human, humanized, single chain, or chimeric antibody. In one embodiment, the antibody, or antigen-binding fragment thereof, includes a Fab, Fab′, F(ab′)₂, Fv, or scFv. In one embodiment, the antibody, or antigen-binding fragment thereof, further comprises a human F_{c}. In another embodiment, the F_{c} is derived from IgG1, IgG2, IgG3, IgG4, IgG5, IgG6, IgG7, IgG8, IgG9, IgG10, IgG11, IgG12, IgG13, IgG14, IgG15, IgG16, IgG17, IgG18, or IgG19.

In one embodiment, the composition includes at least about 25, 80, 100, 160, 200, or 320 mg. In one embodiment, the effective amount is between about 0.1 and 100 mg/kg of body weight of the subject. In one embodiment, the subject is administered at least 1, 2, 3, 4, 5, 7, 8, 9 or 10 doses. In one embodiment, composition is administered every 4 weeks. In one embodiment, the subject is administered 25 mg every 4 weeks. In one embodiment, the subject is administered 80 mg every 4 weeks. In one embodiment, the subject is administered 100 mg every 4 weeks. In one embodiment, the subject is administered 160 mg every 4 weeks. In one embodiment, the subject is administered 200 mg every 4 weeks. In one embodiment, the subject is administered 320 mg every 4 weeks. In another embodiment, the composition is administered every 4 weeks for at least 16 weeks. In another embodiment, the composition is administered every 4 weeks for at least 24 weeks.

In one embodiment, the patient to whom the methods and compositions of the subject technology are applied may have an elevated C-reactive protein (“CRP”). In one embodiment, the patient may have an elevated IL-6 serum level. In one embodiment, the patient may have an elevated IL-6 level in the joints. In one embodiment, the patient may have had an inadequate response to non-steroidal anti-inflammatory drugs (NSAIDs). In one embodiment, the patient may have had an inadequate response to non-biologic Disease Modifying Anti-Rheumatic Drugs (DMARDs).

In one embodiment, the antibody, or antigen-binding fragment thereof, inhibits at least one activity associated with IL-6. In another embodiment, the at least one activity associated with IL-6 is an in vitro activity comprising stimulation of proliferation of T1165 cells; binding of IL-6 to IL-6R; activation (dimerization) of the gp130 signal-transducing glycoprotein; formation of IL-6/IL-6R/130 multimers; stimulation of haptoglobin production by HepG2 cells modified to express human IL-6 receptor; or any combination thereof. In one embodiment, prior to administration of the antibody, or antigen-binding fragment thereof, the subject has exhibited or is at risk for developing at least one of the following symptoms: elevated serum C-reactive protein (“CRP”); elevated erythrocyte sedimentation rate; or a combination thereof.

In one embodiment, the antibody or antigen-binding fragment is directly or indirectly coupled to a detectable label, cytotoxic agent, therapeutic agent, or an immunosuppressive agent. In one embodiment, the detectable label includes a fluorescent dye, bioluminescent material, radioactive material, chemiluminescent moietie, streptavidin, avidin, biotin, radioactive material, enzyme, substrate, horseradish peroxidase, acetylcholinesterase, alkaline phosphatase, β-galactosidase, luciferase, rhodamine, fluorescein, fluorescein isothiocyanate, umbelliferone, dichlorotriazinylamine, phycoerythrin, dansyl chloride, luminol, luciferin, aequorin, Iodine 125 (¹²⁵I), Carbon 14 (¹⁴C), Sulfur 35 (³⁵S), Tritium (³H), Phosphorus 32 (³²P), or any combination thereof.

In one embodiment, the antibody or antigen-binding fragment is co-administered with another therapeutic agent selected from the group consisting of analgesics, antibiotics, anti-cachexia agents, anti-coagulants, anti-cytokine agents, antiemetic agents, anti-fatigue agent, anti-fever agent, anti-inflammatory agents, anti-nausea agents, antipyretics, antiviral agents, anti-weakness agent, chemotherapy agents, cytokine antagonist, cytokines, cytotoxic agents, gene therapy agents, growth factor, IL-6 antagonists, immunosuppressive agents, statins, and any combination thereof. In one embodiment, the cytokine antagonist is an antagonist of a factor comprising tumor necrosis factor-alpha, interferon gamma, interleukin 1 alpha, interleukin 1 beta, interleukin 6, or any combination thereof. In one embodiment, the cytokine antagonist is an antagonist of TNF-α, IL-1α, IL-1β, IL-2, IL-4, IL-6, IL-10, IL-12, IL-13, IL-18, IFN-α, IFN-γ, BAFF, CXCL13, IP-10, leukemia-inhibitory factor, or a combination thereof. In one embodiment, the growth factor is VEGF, EPO, EGF, HRG, Hepatocyte Growth Factor (HGF), Hepcidin, or any combination thereof. In one embodiment, the IL-6 antagonist includes an anti-IL-6 antibodies or antigen-binding fragments thereof, antisense nucleic acids, polypeptides, small molecules, or any combination thereof.

In another embodiment, the antisense nucleic acid includes at least approximately 10 nucleotides of a sequence encoding IL-6, IL-6 receptor alpha, gp130, p38 MAP kinase, JAK1, JAK2, JAK3, or SYK. In another embodiment, the antisense nucleic acid includes DNA, RNA, peptide nucleic acid, locked nucleic acid, morpholino (phosphorodiamidate morpholino oligo), glycerol nucleic acid, threose nucleic acid, or any combination thereof. In another embodiment, the IL-6 antagonist polypeptide includes a fragment of a polypeptide having a sequence selected from the group consisting IL-6, IL-6 receptor alpha, gp130, p38 MAP kinase, JAK1, JAK2, JAK3, and SYK. In another embodiment, the fragment is at least about 40 amino acids in length. In another embodiment, the IL-6 antagonist includes a soluble IL-6, IL-6 receptor alpha, gp130, p38 MAP kinase, JAK1, JAK2, JAK3, SYK, or any combination thereof. In another embodiment, the IL-6 antagonist may be coupled to a half-life increasing moiety.

In one embodiment, the antibody or antigen-binding fragment thereof is administered to the subject in the form of at least one nucleic acids that encode the antibody. In one embodiment, the light chain of said antibody or antigen-binding fragment thereof is encoded by at least one of the following nucleic acid sequences of SEQ ID NOs: 10, 29, 45, 61, 77, 93, 109, 130, 146, 162, 178, 194, 210, 226, 242, 258, 274, 290, 306, 322, 338, 354, 370, 386, 402, 418, 434, 450, 466, 482, 498, 514, 530, 546, 562, 578, 662, 669, 673, 677, 681, 685, 689, 698, 701, 705, 720, 721, 722, or 723. In another embodiment, the heavy chain of said antibody or antigen-binding fragment thereof is encoded by at least one of the following nucleic acid sequences of SEQ ID NOs: 11, 30, 46, 62, 78, 94, 110, 131, 147, 163, 179, 195, 211, 227, 243, 259, 275, 291, 307, 323, 339, 355, 371, 387, 403, 419, 435, 451, 467, 483, 499, 515, 531, 547, 563, 579, 663, 670, 674, 678, 682, 686, 690, 700, 703, 707, 724, or 725. In another embodiment, at least one of the CDRs of said antibody or antigen-binding fragment thereof is encoded by at least one of the following nucleic acid sequences of SEQ ID NOs: 12, 15, 31, 34, 47, 50, 63, 66, 79, 82, 95, 98, 111, 114, 132, 135, 148, 151, 164, 167, 180, 183, 196, 199, 212, 215, 228, 231, 244, 247, 260, 263, 276, 279, 292, 295, 308, 311, 324, 327, 340, 343, 356, 359, 372, 375, 388, 391, 404, 407, 420, 423, 436, 439, 452, 455, 468, 471, 484, 487, 500, 503, 516, 519, 532, 535, 548, 551, 564, 567, 580, 583, 694, 13, 16, 32, 35, 48, 51, 64, 67, 80, 83, 96, 99, 112, 115, 133, 136, 149, 152, 165, 168, 181, 184, 197, 200, 213, 216, 229, 232, 245, 248, 261, 264, 277, 280, 293, 296, 309, 312, 325, 328, 341, 344, 357, 360, 373, 376, 389, 392, 405, 408, 421, 424, 437, 440, 453, 456, 469, 472, 485, 488, 501, 504, 517, 520, 533, 536, 549, 552, 565, 568, 581, 584, 696, 14, 17, 33, 36, 49, 52, 65, 68, 81, 84, 97, 100, 113, 116, 134, 137, 150, 153, 166, 169, 182, 185, 198, 201, 214, 217, 230, 233, 246, 249, 262, 265, 278, 281, 294, 297, 310, 313, 326, 329, 342, 345, 358, 361, 374, 377, 390, 393, 406, 409, 422, 425, 438, 441, 454, 457, 470, 473, 486, 489, 502, 505, 518, 521, 534, 537, 550, 553, 566, 569, 582, 585, 695, or 697. In another embodiment, at least one nucleic acids includes the heavy and light chain polynucleotide sequences of SEQ ID NO: 723 and SEQ ID NO: 700; SEQ ID NO: 701 and SEQ ID NO: 703; SEQ ID NO: 705 and SEQ ID NO: 707; SEQ ID NO: 720 and SEQ ID NO: 724; and SEQ ID NO: 10 and SEQ ID NO: 11.

In one embodiment, the composition is administered subcutaneously. In another embodiment, the composition is a pharmaceutical composition. In a further embodiment, the composition is formulated for subcutaneous administration.

In one embodiment, the antibody or antigen-binding fragment thereof is asialated. In one embodiment, the antibody or antigen-binding fragment thereof is humanized. In one embodiment, the antibody or antigen-binding fragment thereof has a half-life of at least about 30 days. In one embodiment, the antibody or antigen-binding fragment thereof includes the humanized variable light sequence of amino acid sequence of SEQ ID NO: 20. In one embodiment, the antibody or antigen-binding fragment thereof includes humanized variable heavy sequence of amino acid sequence of SEQ ID NO: 19. In another embodiment, the antibody or antigen-binding fragment thereof includes at least one light chain CDRs as set forth in the amino acid sequence of SEQ ID NOs: 4, 5, or 6. In another embodiment, the antibody or antigen-binding fragment thereof includes at least one heavy chain CDRs as set forth in the amino acid sequence of SEQ ID NOs: 7, 120, 8, or 9. In further embodiment, the antibody or antigen-binding fragment thereof is an asialated, humanized anti-IL-6 monoclonal antibody with a half-life of ˜30 days comprising the humanized variable light and heavy sequences as set forth in SEQ ID NO: 20 and 19 or 702 and 704, respectively.

One embodiment encompasses specific humanized antibodies and fragments and variants thereof for treatment or prevention of psoriatic arthritis capable of binding to IL-6 and/or the IL-6/IL-6R complex. These antibodies bind to soluble IL-6 or cell surface expressed IL-6. Also, these antibodies inhibit the formation or the biological effects of at least one of IL-6, IL-6/IL-6R complexes, IL-6/IL-6R/gp130 complexes and/or multimers of IL-6/IL-6R/gp130. The present subject technology relates to novel therapies and therapeutic protocols using anti-IL-6 antibodies, preferably those described herein.

In a preferred embodiment this is effected by the administration of the antibodies described herein, comprising the sequences of the V_{H}, V_{L} and CDR polypeptides described in Table 1, or humanized or chimeric or single chain versions thereof containing at least one of the CDRs of the exemplified anti-IL-6 antibody sequences and the polynucleotides encoding them. Preferably these antibodies will be aglycosylated. In more specific embodiments of the subject technology these antibodies will block gp130 activation and/or possess binding affinities (Kds) less than 50 picomolar and/or K_{off} values less than or equal to 10⁻⁴ S⁻¹.

The subject technology also contemplates methods of making said humanized anti-IL-6 or anti-IL-6/IL-6R complex antibodies and binding fragments and variants thereof. In one embodiment, binding fragments include, but are not limited to, Fab, Fab′, F(ab′)₂, Fv and scFv fragments.

In one embodiment, the anti-IL-6 antibodies block the effects of IL-6. In another embodiment, the anti-IL-6 antibody is a humanized monoclonal antibody that binds to free human IL-6 and soluble IL-6R/IL-6 complex with an affinity of at least about 4 pM. In another embodiment, the anti-IL-6 antibody, has a serum half-life about at least 30 days. In another embodiment, the anti-IL-6 antibody is based on a consensus human IgG1 kappa framework that had asparagines modified to alanine to eliminate N-glycosylation sites.

In another embodiment, the antibodies and humanized versions are derived from rabbit immune cells (B lymphocytes) and are selected based on their homology (sequence identity) to human germ line sequences. These antibodies may require minimal or no sequence modifications, thereby facilitating retention of functional properties after humanization. In exemplary embodiments, the humanized antibodies include human frameworks which are highly homologous (possess high level of sequence identity) to that of a parent (e.g. rabbit) antibody.

In an embodiment of the subject technology, the anti-IL-6 antibody or antibody fragment or variant thereof specifically bind to the same linear or conformational epitopes on an intact IL-6 polypeptide or fragment thereof which may include at least fragments selected from those encompassing amino acid residues 37-51, amino acid residues 70-84, amino acid residues 169-183, amino acid residues 31-45 and/or amino acid residues 58-72.

In a preferred exemplary embodiment, the anti-IL-6 antibody comprises at least one of the CDRs in listed in Table 1. In a more preferred embodiment the anti-IL-6 antibody comprises the variable heavy and light chain sequences in SEQ ID NO: 657 and SEQ ID NO: 709, or variants thereof.

In a preferred embodiment the humanized anti-IL-6 antibody comprises the variable heavy and variable light chain sequences respectively set forth in SEQ ID NO: 657 and SEQ ID NO: 709, respectively, and preferably further comprising the heavy chain and light chain constant regions respectively set forth in SEQ ID NO: 588 and SEQ ID NO: 586, and variants thereof comprising at least one amino acid substitutions or deletions that do not substantially affect IL-6 binding and/or desired effector function. This embodiment also contemplates polynucleotides comprising, or alternatively consisting of, at least one of the nucleic acids encoding the variable heavy chain (SEQ ID NO: 700) and variable light chain (SEQ ID NO: 723) sequences and the constant region heavy chain (SEQ ID NO: 589) and constant region light chain (SEQ ID NO: 587) sequences. This embodiment further contemplates nucleic acids encoding variants comprising at least one amino acid substitutions or deletions to the variable heavy and variable light chain sequences respectively set forth in SEQ ID NO: 657 and SEQ ID NO: 709 and the heavy chain and light chain constant regions respectively set forth in SEQ ID NO: 588 and SEQ ID NO: 586, that do not substantially affect IL-6 binding and/or desired effector function.

In an embodiment of the subject technology, the anti-IL-6 antibody or antibody fragment or variant thereof is aglycosylated or substantially aglycosylated, e.g., as a result of one or more modifications in the Fc region of the antibody.

In an embodiment of the subject technology, the anti-IL-6 antibody or antibody fragment or variant thereof contain an Fc region that has been modified to alter effector function, half-life, proteolysis, and/or glycosylation. Preferably the Fc region is modified to eliminate glycosylation.

In an embodiment of the subject technology, the anti-IL-6 antibody or antibody fragment or variant thereof is a human, humanized, single chain or chimeric antibody.

In an embodiment of the subject technology, the anti-IL-6 antibody or antibody fragment or variant thereof is a humanized antibody derived from a rabbit (parent) anti-IL-6 antibody.

In an embodiment of the subject technology, the framework regions (FRs) in the variable light region and the variable heavy regions of said anti-IL-6 antibody or antibody fragment or variant thereof respectively is human FRs which are unmodified or which have been modified by the substitution of at most 2 or 3 human FR residues in the variable light or heavy chain region with the corresponding FR residues of the parent rabbit antibody, and the human FRs have been derived from human variable heavy and light chain antibody sequences which have been selected from a library of human germline antibody sequences based on their high level of homology to the corresponding rabbit variable heavy or light chain regions relative to other human germline antibody sequences contained in the library. As disclosed in detail infra in a preferred embodiment the antibody will comprise human FRs which are selected based on their high level of homology (degree of sequence identity) to that of the parent antibody that is humanized.

In one embodiment of the subject technology, the anti-IL-6 antibody or antibody fragment or variant thereof includes a heavy chain polypeptide sequence comprising: SEQ ID NO: 3, 18, 19, 652, 656, 657, 658, 661, 664, 665, 704, or 708; and may further comprise a V_{L} polypeptide sequence comprising: SEQ ID NO: 2, 20, 647, 651, 660, 666, 699, 702, 706, or 709 or a variant thereof wherein at least one of the framework residues (FR residues) in said V_{H} or V_{L} polypeptide may have been substituted with another amino acid residue resulting in an anti-IL-6 antibody or antibody fragment or variant thereof that specifically binds human IL-6, or includes a polypeptide wherein the CDRs therein are incorporated into a human framework homologous to said sequence. Preferably the variable heavy and light sequences comprise those in SEQ ID NO: 657 and 709, respectively.

In an embodiment of the subject technology, at least one of said FR residues may be substituted with an amino acid present at the corresponding site in a parent rabbit anti-IL-6 antibody from which the complementarity determining regions (CDRs) contained in said V_{H} or V_{L} polypeptides have been derived or by a conservative amino acid substitution.

In an embodiment of the subject technology, said anti-IL-6 antibody, or antibody fragment or variant thereof, is humanized.

In an embodiment of the subject technology, said anti-IL-6 antibody, or antibody fragment or variant thereof, is chimeric.

In an embodiment of the subject technology, said anti-IL-6 antibody, or antibody fragment or variant thereof, further includes a human Fc, e.g., an Fc region comprised of the variable heavy and light chain constant regions set forth in SEQ ID NO: 704 and 702.

In an embodiment of the subject technology, said human Fc may be derived from IgG1, IgG2, IgG3, IgG4, IgG5, IgG6, IgG7, IgG8, IgG9, IgG10, IgG11, IgG12, IgG13, IgG14, IgG15, IgG16, IgG17, IgG18 or IgG19.

In an embodiment of the subject technology, the anti-IL-6 antibody or antibody fragment or variant thereof includes a polypeptide having at least about 90% sequence homology to at least one of the polypeptide sequences of SEQ ID NO: 3, 18, 19, 652, 656, 657, 658, 661, 664, 665, 704, 708, 2, 20, 647, 651, 660, 666, 699, 702, 706, or 709.

In an embodiment of the subject technology, the anti-IL-6 antibody or antibody fragment or variant thereof has an elimination half-life of at least about 30 days.

The subject technology also contemplates the administration of conjugates of anti-IL-6 antibodies and humanized, chimeric or single chain versions thereof and other binding fragments and variants thereof conjugated to at least one functional or detectable moieties.

In an embodiment of the subject technology, the anti-IL-6 antibody or antibody fragment or variant thereof is directly or indirectly attached to a detectable label or therapeutic agent.

In one embodiment, the IL-6 antagonist is an antisense nucleic acid. In another embodiment of the subject technology, the IL-6 antagonist is an antisense nucleic acid, for example comprising at least approximately 10 nucleotides of a sequence encoding IL-6, IL-6 receptor alpha, gp130, p38 MAP kinase, JAK1, JAK2, JAK3, or SYK. In a further embodiment of the subject technology, the antisense nucleic acid includes DNA, RNA, peptide nucleic acid, locked nucleic acid, morpholino (phosphorodiamidate morpholino oligo), glycerol nucleic acid, threose nucleic acid, or any combination thereof.

In one embodiment, the IL-6 antagonist includes Actemra® (Tocilizumab), Remicade®, Zenapax® (daclizumab), or any combination thereof.

In one embodiment, the IL-6 antagonist includes a polypeptide having a sequence comprising a fragment of IL-6, IL-6 receptor alpha, gp130, p38 MAP kinase, JAK1, JAK2, JAK3, SYK, or any combination thereof, such as a fragment or full-length polypeptide that is at least 40 amino acids in length. In another embodiment of the subject technology, the IL-6 antagonist includes a soluble IL-6, IL-6 receptor alpha, gp130, p38 MAP kinase, JAK1, JAK2, JAK3, SYK, or any combination thereof.

In an embodiment of the subject technology, the IL-6 antagonist is coupled to a half-life increasing moiety.

In another aspect the subject technology provides novel pharmaceutical compositions and their use in novel combination therapies and comprising administration of an anti-IL-6 antibody, such as any one of Ab1-Ab36 antibodies described in Table 1 or a fragment or variant thereof, and at least one other therapeutic compound such as an anti-cytokine agent.

In an embodiment of the subject technology, the IL-6 antagonist may target IL-6, IL-6 receptor alpha, gp130, p38 MAP kinase, JAK1, JAK2, JAK3, SYK, or any combination thereof. In one embodiment, the IL-6 antagonist includes an antibody, an antibody fragment, a peptide, a glycoalkoid, an antisense nucleic acid, a ribozyme, a retinoid, an avemir, a small molecule, or any combination thereof. In one embodiment, the IL-6 antagonist includes an anti-IL-6R, anti-gp130, anti-p38 MAP kinase, anti-JAK1, anti-JAK2, anti-JAK3, or anti-SYK antibody or antibody fragment. In an embodiment of the subject technology, the antagonist includes an anti-IL-6 antibody (e.g., any one of Ab1-Ab36 antibodies described in Table 1) or antibody fragment or variant thereof.

The present subject technology also pertains to methods of improving survivability or quality of life of a patient having or at risk of developing psoriatic arthritis comprising administering to the patient an anti-IL-6 antibody (e.g., ALD518 antibody) or antibody fragment or variant thereof, whereby the patient's C-reactive protein (“CRP”) level is lowered.

In one embodiment of the subject technology, the anti-IL-6 antibody or antibody fragment or variant thereof is administered to the patient with a frequency at most once per period of approximately 4, 8, 12, 16, 20, or 24 weeks.

In an embodiment of the subject technology, the patient's quality of life is improved.

This subject technology relates to novel anti-IL-6 antibodies, novel therapies and therapeutic protocols utilizing anti-IL-6 antibodies, and pharmaceutical formulations containing anti-IL-6 antibodies. In preferred embodiments, an anti-IL-6 antibody is any one of Ab1-Ab36 antibodies described in Table 1, which includes rabbit or humanized forms thereof, as well as heavy chains, light chains, fragments, variants, and CDRs thereof, or an antibody or antibody fragment that specifically binds to the same linear or conformational epitope(s) on an intact human IL-6 polypeptide fragment thereof as Ab1. The subject application pertains in particular to preferred formulations and therapeutic uses of an exemplary humanized antibody referred to herein as any one of Ab1-Ab36 antibodies described in Table 1 and variants thereof. In preferred embodiments, the anti-IL-6 antibody has an in vivo half-life of at least about 30 days, has an in vivo effect of lowering C-reactive protein, possesses a binding affinity (Kd) for IL-6 of less than about 50 picomolar, and/or has a rate of dissociation (K_{off}) from IL-6 of less than or equal to 10⁻⁴ S⁻¹.

In one aspect, this subject technology pertains to methods of improving survivability or quality of life of a patient in need thereof, comprising administering to a patient with or at risk of developing psoriatic arthritis as a result of disease or a therapeutic regimen comprising the administration of an anti-IL-6 antibody, such as any one of Ab1-Ab36 antibodies described in Table 1 antibody or a fragment or variant thereof (e.g., Ab1).

In this embodiment, anti-IL-6 antibodies, or antigen-binding fragments thereof is administered at effective doses to less inflammation, pain, and loss of mobility experienced from psoriatic arthritis, e.g., dosages ranging from about 25-500 mg, preferably at least about 25, 80, 100, 120, 160, 200, 240, or 320 mg dosages. In an embodiment, the effective dosage ranges between about 25 to 160 mg/4 weeks, per person, delivered to a subject in need thereof by a subcutaneous injection.

Another embodiment relates to methods of improving survivability or quality of life of a patient diagnosed with psoriatic arthritis, comprising administering to the patient an anti-IL-6 antibody or antigen-binding fragment or variant thereof, whereby the patient's serum C-reactive protein (“CRP”) level is stabilized and preferably reduced, and monitoring the patient to assess the reduction in the patient's serum CRP level. In an embodiment, the patient has an elevated C-reactive protein (CRP) level prior to treatment. In an embodiment, the patient may have an elevated serum CRP level prior to treatment.

In an embodiment of the subject technology, the patient's serum CRP level remains decreased for an entire period intervening two consecutive anti-IL-6 antibody administrations.

In one embodiment, the patient may have been diagnosed psoriatic arthritis.

In one embodiment, the antibody, or antigen-binding fragment thereof, is engineered, e.g., produced by genetic engineering methods such as having been expressed from a recombinant cell. In another embodiment, the cell may be selected from a mammalian, yeast, bacterial, and insect cell. In another embodiment, the cell may be a yeast cell. In another embodiment, the cell is a diploidal yeast cell. In another embodiment, the yeast cell is a Pichia yeast. In another embodiment, the anti-IL-6 antibody is produced in a yeast based (Pichia pastoris) expression system using conventional fermentation processes and downstream purification. In one embodiment, the antibodies and antibody fragments described herein is expressed in yeast cells. In one embodiment, the mating competent yeast is a member of the Saccharomycetaceae family, which includes the genera Arxiozyma; Ascobotryozyma; Citeromyces; Debaryomyces; Dekkera; Eremothecium; Issatchenkia; Kazachstania; Kluyveromyces; Kodamaea; Lodderomyces; Pachysolen; Pichia; Saccharomyces; Saturnispora; Tetrapisispora; Torulaspora; Williopsis; and Zygosaccharomyces. Other types of yeast potentially useful in the subject technology include Yarrowia, Rhodosporidium, Candida, Hansenula, Filobasium, Filobasidellla, Sporidiobolus, Bullera, Leucosporidium, and Filobasidella. In a preferred embodiment, the mating competent yeast may a member of the genus Pichia. In a further preferred embodiment, the mating competent yeast of the genus Pichia is one of the following species: Pichia pastoris, Pichia methanolica, and Hansenula polymorphs (Pichia angusta). In a particularly preferred embodiment, the mating competent yeast of the genus Pichia may the species Pichia pastoris.

In exemplary embodiments the invention comprises or consists of a method for treating or preventing psoriatic or rheumatoid arthritis, or managing one or more of the symptoms of psoriatic or rheumatoid arthritis comprising administration of a composition comprising an effective amount of an anti-IL-6 antibody or antibody fragment thereof to a subject in need thereof, wherein the anti-IL-6 antibody or antibody fragment thereof comprises a variable light (V_{L}) chain polypeptide comprising a CDR1 sequence of SEQ ID NO:4, a CDR2 sequence of SEQ ID NO:5, and a CDR3 sequence of SEQ ID NO:6, and a variable heavy (V_{H}) chain polypeptide comprising a CDR1 sequence of SEQ ID NO:7, a CDR2 sequence of SEQ ID NOs:8 or 120, and a CDR3 sequence of SEQ ID NO:9.

In another exemplary embodiments the invention comprises or consists of a method for treating or preventing psoriatic or rheumatoid arthritis or managing one or more of the symptoms of psoriatic arthritis, comprising administration of a composition comprising an effective amount of an anti-IL-6 antibody or antibody fragment thereof to a subject in need thereof, wherein the anti-IL-6 antibody or antibody fragment thereof comprises a variable light (V_{L}) chain polypeptide comprising the amino acid sequence in SEQ ID NO:20, 702 or 709, and a variable heavy (V_{H}) chain polypeptide comprising the amino acid sequence in SEQ ID NO:18, 19, 657 or 704.

In another exemplary embodiments the invention comprises or consists of a method for treating or preventing psoriatic or rheumatoid arthritis or managing one or more of the symptoms of psoriatic arthritis of claim 1, comprising administration of a composition comprising an effective amount of an anti-IL-6 antibody or antibody fragment thereof to a subject in need thereof, wherein the anti-IL-6 antibody or antibody fragment thereof comprises a variable light (V_{L}) chain polypeptide comprising the amino acid sequence in SEQ ID NO:20 or 709, and a variable heavy (V_{H}) chain polypeptide comprising the amino acid sequence in SEQ ID NO:18, 19, or 657.

In yet another exemplary embodiments the invention comprises or consists of a method for treating or preventing psoriatic or rheumatoid arthritis or managing one or more of the symptoms of psoriatic arthritis, comprising administration of a composition comprising an effective amount of an anti-IL-6 antibody or antibody fragment thereof to a subject in need thereof, wherein the anti-IL-6 antibody or antibody fragment thereof comprises a light chain polypeptide comprising the polypeptide having the amino acid sequence in SEQ ID NO:702 and a heavy chain comprising the polypeptide having the amino acid sequence of SEQ ID NO:704.

In any of the foregoing embodiments said antibody fragment may e.g., be a Fab fragment, a Fab′ fragment, a F(ab′)2 fragment, an scFv, a camelbody, a nanobody, a MetMab like monovalent agent, or an IgNAR (single-chain antibodies derived from sharks).

In yet another exemplary embodiments the invention comprises or consists of a method for treating or preventing psoriatic arthritis or managing one or more of the symptoms of psoriatic arthritis, comprising administration of a composition comprising an anti-IL-6 antibody or antibody fragment thereof comprises a V_{L} chain polypeptide at least 80% identical to the amino acid sequence of SEQ ID NO:709, and/or a V_{H} chain polypeptide at least 80% identical to the amino acid sequence of SEQ ID NO:657.

In yet another exemplary embodiments the invention comprises or consists of a method for treating or preventing psoriatic or rheumatoid arthritis or managing one or more of the symptoms of psoriatic or rheumatoid arthritis, comprising administration of a composition comprising an anti-IL-6 antibody or antibody fragment thereof which comprises a V_{L} chain polypeptide at least 95% identical to the amino acid sequence of SEQ ID NO:709, and/or a V_{H} chain polypeptide at least 95% identical to the amino acid sequence of SEQ ID NO:657.

In yet another exemplary embodiments the invention comprises or consists of a method for treating or preventing psoriatic or rheumatoid arthritis or managing one or more of the symptoms of psoriatic arthritis, comprising administration of a composition comprising an anti-IL-6 antibody or antibody fragment thereof which comprises a V_{L} chain polypeptide at least 95% identical to the amino acid sequence of SEQ ID NO:709, and/or a V_{H} chain polypeptide at least 95% identical to the amino acid sequence of SEQ ID NO:657.

In yet another exemplary embodiments the invention comprises or consists of a method for treating or preventing psoriatic or rheumatoid arthritis or managing one or more of the symptoms of psoriatic or rheumatoid arthritis, comprising administration of a composition comprising an anti-IL-6 antibody or antibody fragment thereof which comprises a V_{L} chain polypeptide identical to the amino acid sequence of SEQ ID NO:709, and/or a V_{H} chain polypeptide identical to the amino acid sequence of SEQ ID NO:657.

In yet another exemplary embodiments the invention comprises or consists of a method for treating or preventing psoriatic arthritis or managing one or more of the symptoms of psoriatic arthritis, comprising administration of a composition comprising an anti-IL-6 antibody or antibody fragment thereof which comprises a light chain polypeptide at least 90, 95 or 99% identical to the amino acid sequence of SEQ ID NO:702, and/or a heavy chain polypeptide at least 90, 95 or 99% identical to the amino acid sequence of SEQ ID NO:704.

In any of the foregoing exemplary embodiments, said anti-IL-6 antibody or antibody fragment thereof is aglycosylated.

In any of the foregoing exemplary embodiments said antibody or antibody fragment comprises an Fc region that has been modified to alter effector function, half-life, proteolysis, and/or glycosylation.

In any of the foregoing exemplary embodiments wherein said antibody or antibody fragment comprises a human Fc derived from IgG1, IgG2, IgG3, or IgG4.

In any of the foregoing exemplary embodiments said antibody or antibody fragment thereof is a human, humanized, single chain or chimeric antibody.

In any of the foregoing exemplary embodiments said antibody or antibody fragment specifically binds to human cell surface expressed IL-6 and/or to circulating soluble IL-6 molecules in vivo.

In any of the foregoing exemplary embodiments said antibody or antibody fragment specifically binds to IL-6 expressed on or by human cells in the subject.

In any of the foregoing exemplary embodiments said antibody or antibody fragment has an in vivo half-life of at least about 30 days in a healthy human subject.

In any of the foregoing exemplary embodiments said antibody or antibody fragment has a binding affinity (Kd) for IL-6 of less than about 50 picomolar, or a rate of dissociation (K_{off}) from IL-6 of less than or equal to 10⁻⁴ S⁻¹.

In any of the foregoing exemplary embodiments said antibody or antibody fragment specifically binds to the same linear or conformational epitope(s) and/or competes for binding to the same linear or conformational epitope(s) on an intact human IL-6 polypeptide or fragment thereof as an anti-IL-6 antibody comprising the polypeptides of SEQ ID NO: 702 and SEQ ID NO: 704.

In any of the foregoing exemplary embodiments said antibody or antibody fragment contains an Fc region that has been modified to alter effector function, half-life, proteolysis, and/or glycosylation.

In any of the foregoing exemplary embodiments a single dosage effective amount of aid anti-IL-6 antibody or antibody fragment comprises at least or consists of 1, 5, 10, 15, 20, 25, 50, 60, 80, 100, 120, 160, 200, or 320 mg of said anti-IL-6 antibody or antibody fragment.

In any of the foregoing exemplary embodiments a dosage effective amount of said anti-IL-6 antibody or antibody fragment is between about 0.1 and 100 mg/kg of body weight of the subject.

In any of the foregoing exemplary embodiments said anti-IL-6 antibody or fragment thereof is administered at least 1, 2, 3, 4, or 5 doses.

In any of the foregoing exemplary embodiments said anti-IL-6 antibody or fragment composition is administered in a dosage regimen that comprises or consists of administering said anti-IL-6 antibody or antibody fragment every 4 weeks or every month.

In any of the foregoing exemplary embodiments said anti-IL-6 antibody or fragment composition is administered in a dosage regimen that comprises or consists of administering 1, 5, 10, 15, 20 or 25 mg of said anti-IL-6 antibody or antibody fragment every 4 weeks or every month.

In any of the foregoing exemplary embodiments said anti-IL-6 antibody or fragment composition is administered in a dosage regimen that comprises or consists of administering 25 mg of said anti-IL-6 antibody or antibody fragment every 4 weeks or every month.

In any of the foregoing exemplary embodiments said anti-IL-6 antibody or fragment composition is administered in a dosage regimen that comprises or consists of administering 25 mg of said anti-IL-6 antibody or antibody fragment every 4 weeks.

In any of the foregoing exemplary embodiments said anti-IL-6 antibody or fragment composition is administered in a dosage regimen is effected every 4 weeks or monthly for 8, 12, 16, 20, 24, 28, 32, 36 weeks or more or for 2, 3, 4, 5, 6 or more months.

In any of the foregoing exemplary embodiments said anti-IL-6 antibody or fragment composition is administered in a dosage regimen wherein said subject is treated by a dosage regimen that comprises or consists of administering 50, 60 or 75 mg of said anti-IL-6 antibody or antibody fragment every 4 weeks or every month.

In any of the foregoing exemplary embodiments said anti-IL-6 antibody or fragment composition is administered in a dosage regimen The method of any of the foregoing claims, wherein said subject is treated by a dosage regimen that comprises or consists of administering 80, 100 or 120 mg of said anti-IL-6 antibody or antibody fragment every 4 weeks or every month.

In any of the foregoing exemplary embodiments said anti-IL-6 antibody or fragment composition is administered in a dosage regimen wherein said subject is treated by a dosage regimen that comprises or consists of administering 160 mg of said anti-IL-6 antibody or antibody fragment every 4 weeks or every month.

In any of the foregoing exemplary embodiments said anti-IL-6 antibody or fragment composition is administered in a dosage regimen that comprises or consists of administering 200 mg of said anti-IL-6 antibody or antibody fragment every 4 weeks or every month.

In any of the foregoing exemplary embodiments said anti-IL-6 antibody or fragment composition is administered in a dosage regimen wherein said subject is subject is treated by a dosage regimen that comprises or consists of administering said anti-IL-6 antibody or antibody fragment every 4 weeks or every month for at least 16 weeks or 4 months.

In any of the foregoing exemplary embodiments said anti-IL-6 antibody or fragment composition is administered in a dosage regimen wherein said subject is subject is treated by a dosage regimen that comprises or consists of administering said anti-IL-6 antibody or antibody fragment every 4 weeks or every month for at least 20 or 24 weeks or at least 5 or 6 months.

In any of the foregoing exemplary embodiments the treated patient or subject has elevated C-reactive protein (“CRP”).

In any of the foregoing exemplary embodiments the treated patient or subject has elevated IL-6 serum level.

In any of the foregoing exemplary embodiments the treated patient or subject has elevated IL-6 level in the joints.

In any of the foregoing exemplary embodiments the treated patient or subject has had an inadequate response to non-steroidal anti-inflammatory drugs (NSAIDs).

In any of the foregoing exemplary embodiments the treated patient or subject has had an inadequate response to non-biologic Disease Modifying Anti-Rheumatic Drugs (DMARDs).

In any of the foregoing exemplary embodiments said antibody or antibody fragment thereof inhibits with at least one activity associated with IL-6.

In any of the foregoing exemplary embodiments the antibody or antibody fragment inhibits at least one of the at least one activity associated with IL-6 is an in vitro activity comprising stimulation of proliferation of T1165 cells; binding of IL-6 to IL-6R; activation (dimerization) of the gp130 signal-transducing glycoprotein; formation of IL-6/IL-6R/gp130 multimers; stimulation of haptoglobin production by HepG2 cells modified to express human IL-6 receptor; or any combination thereof.

In any of the foregoing exemplary embodiments the treated patient or subject prior to administration of the antibody, or antigen-binding fragment thereof, the subject has exhibited or is at risk for developing at least one of the following symptoms: elevated serum C-reactive protein (“CRP”); elevated erythrocyte sedimentation rate; or a combination thereof.

In any of the foregoing exemplary embodiments the antibody or antibody fragment thereof is directly or indirectly coupled to a detectable label, cytotoxic agent, therapeutic agent, or an immunosuppressive agent.

In any of the foregoing exemplary embodiments the antibody or antibody fragment thereof is directly or indirectly coupled to a detectable label comprising fluorescent dyes, bioluminescent materials, radioactive materials, chemiluminescent moieties, streptavidin, avidin, biotin, radioactive materials, enzymes, substrates, horseradish peroxidase, acetylcholinesterase, alkaline phosphatase, □-galactosidase, luciferase, rhodamine, fluorescein, fluorescein isothiocyanate, umbelliferone, dichlorotriazinylamine, phycoerythrin, dansyl chloride, luminol, luciferin, aequorin, Iodine 125 (1251), Carbon 14 (14C), Sulfur 35 (35S), Tritium (3H), Phosphorus 32 (32P), or any combination thereof.

In any of the foregoing exemplary embodiments the antibody or antibody fragment thereof is co-administered with another therapeutic agent selected from the group consisting of analgesics, antibiotics, anti-cachexia agents, anti-coagulants, anti-cytokine agents, antiemetic agents, anti-fatigue agent, anti-fever agent, anti-inflammatory agents, anti-nausea agents, antipyretics, antiviral agents, anti-weakness agent, chemotherapy agents, cytokine antagonist, cytokines, cytotoxic agents, gene therapy agents, growth factor, IL-6 antagonists, immunosuppressive agents, statins, or any combination thereof.

In any of the foregoing exemplary embodiments the antibody or antibody fragment is co-administered with an agonist of a factor comprising tumor necrosis factor-alpha, interferon gamma, interleukin 1 alpha, interleukin 1 beta, interleukin 6, or any combination thereof.

In any of the foregoing exemplary embodiments the antibody or antibody fragment is co-administered with a cytokine antagonist which is an antagonist of TNF-α, IL-1β, IL-1α, IL-2, IL-4, IL-6, IL-10, IL-12, IL-13, IL-18, IFN-α, IFN-β, IFN-γ, BAFF, CXCL13, IP-10, leukemia-inhibitory factor, or a combination thereof.

In any of the foregoing exemplary embodiments the antibody or antibody fragment is co-administered with an antagonist of VEGF, EPO, EGF, HRG, Hepatocyte Growth Factor (HGF), Hepcidin, or any combination thereof.

In any of the foregoing exemplary embodiments the antibody or antibody fragment is co-administered with other anti-IL-6 antibodies or antigen-binding fragments thereof, antisense nucleic acids, polypeptides, small molecules, or any combination thereof.

In any of the foregoing exemplary embodiments the antibody or antibody fragment is co-administered with an antisense nucleic acid comprises at least approximately 10 nucleotides of a sequence encoding IL-6, IL-6 receptor alpha, gp130, p38 MAP kinase, JAK1, JAK2, JAK3, or SYK.

In any of the foregoing exemplary embodiments the antibody or antibody fragment is co-administered with an IL-6 antagonist polypeptide that comprises a fragment of a polypeptide having a sequence selected from the group consisting IL-6, IL-6 receptor alpha, gp130, p38 MAP kinase, JAK1, JAK2, JAK3, and SYK.

In any of the foregoing exemplary embodiments the antibody or antibody fragment is co-administered with an IL-6 antagonist comprising a soluble IL-6, IL-6 receptor alpha, gp130, p38 MAP kinase, JAK1, JAK2, JAK3, SYK, or any combination thereof, e.g., one coupled to a half-life increasing moiety.

In any of the foregoing exemplary embodiments the antibody or antibody fragment is administered to the subject in the form of at least one nucleic acid that encodes said antibody or antibody fragment thereof.

In any of the foregoing exemplary embodiments the antibody or antibody fragment is in a pharmaceutical composition comprising a pharmaceutical excipient.

In another exemplary embodiment the invention is directed to a dosage composition, or syringe or injector pen containing a single dosage of an anti-IL-6 antibody or antibody fragment which is for use in treating or preventing psoriatic arthritis according to any of the foregoing claims, and wherein said anti-IL-6 antibody or antibody fragment comprises or consists of CDRs, variable heavy or light polypeptides or light and heavy polypeptides having the amino acid sequences as set forth in any of the foregoing claims, wherein the single dosage of said anti-IL-6 antibody or antibody fragment contained in said composition or syringe or injector pen containing same comprises at most or consists of 1, 5, 10, 15, 20, or 25 mg of said anti-IL-6 antibody or antibody fragment.

In another exemplary embodiment the invention is directed to a single dosage composition, or syringe or injector pen comprising an anti-IL-6 antibody or fragment, which is for administration every 4 weeks or monthly for treating or managing the symptoms of psoriatic arthritis.

In another exemplary embodiment the invention is directed to a single dosage composition, syringe or injector pen of claim which contains an antibody dosage comprising or consisting of 25 mg of said anti- an anti-IL-6 antibody or antibody fragment according to the invention.

In another exemplary embodiment the invention is directed to a single dosage composition, syringe or injector pen of claim which contains an antibody dosage comprising or consisting of 25 mg of said anti- an anti-IL-6 antibody or antibody fragment according to the invention, wherein the antibody or antibody fragment is comprised in an aqueous or 0.9% saline solution.

In another exemplary embodiment the invention is directed to a therapeutic regimen for treating or preventing psoriatic arthritis or managing the side effects of psoriatic arthritis in a subject in need thereof, wherein the therapeutic regimen comprises or consists of administering a single dosage of an anti-IL-6 antibody or antibody fragment every 4 weeks or monthly using a syringe or injector pen which single dosage comprises at most or consists of 1, 5, 10, 15, 20, or 25 mg of—an anti-IL-6 antibody or antibody fragment comprising or consisting of any of the anti-IL-6 antibody sequences set forth herein, preferably an anti-IL-6 antibody or antibody fragment that comprises the VL polypeptide of SEQ ID NO:20 or 709 and a V_{H} polypeptides having the amino acid sequence of SEQ ID NO:18, 19 or 657, or that comprises the VL polypeptide of SEQ ID NO:709 and a V_{H} polypeptides having the amino acid sequence of SEQ ID NO:657 or which comprises a light chain polypeptide having the amino acid sequence of SEQ ID NO:702 and a heavy chain polypeptide having the amino acid sequence of SEQ ID NO:704.

In another exemplary embodiment the invention is directed methods or regimens as above-described wherein the treated subject or caregiver subcutaneously administers the single dosage every 4 weeks or monthly.

In another exemplary embodiment the invention is directed to methods or regimens as above-described wherein the treated subject or caregiver subcutaneously administers a single dosage of anti-IL-6 antibody according to the invention every 4 weeks or monthly by use of an injector pen.

In another exemplary embodiment the invention is directed to methods or regimens as above-described which further include the administration of a DMARD, a corticosteroid.

In another exemplary embodiment the invention is directed to methods or regimens as above-described which further include the administration of methotrexate.

In another exemplary embodiment the invention is directed to methods or regimens as above-described wherein the treated subject has developed a resistance or tolerance to methotrexate.

In another exemplary embodiment the invention is directed to methods or regimens as above-described wherein the treated subject has previously received another anti-IL-6 antagonist or an anti-TNF biologic.

In another exemplary embodiment the invention is directed to methods or regimens as above-described wherein the treated subject has previously received Humira®, Remicade®, or Actmera®.

In another exemplary embodiment the invention is directed to methods or regimens as above-described wherein the treated subject is treated for at least 12 weeks or 3 months.

In another exemplary embodiment the invention is directed to methods or regimens as above-described wherein the treated subject is treated for at least 16 weeks or 4 months.

In another exemplary embodiment the invention is directed to methods or regimens as above-described wherein the treated subject is treated for at least 20 weeks or 5 months.

In another exemplary embodiment the invention is directed to methods or regimens as above-described wherein the treated subject is treated for at least 24 weeks or 6 months.

In another exemplary embodiment the invention is directed to methods or regimens as above-described wherein the treated subject is treated for more than at least 24 weeks or 6 months.

In another exemplary embodiment the invention is directed to methods or regimens as above-described wherein the treated subject exhibits prolonged disease remission after said antibody treatment.

### BRIEF DESCRIPTION OF THE DRAWINGS

FIG. 1 shows alignments of variable light and variable heavy sequences between a rabbit antibody variable light and variable heavy sequences and homologous human sequences and the humanized sequences. Framework regions are identified FR1-FR4. Complementarity determining regions are identified as CDR1-CDR3. Amino acid residues are numbered as shown. The initial rabbit sequences are called RbtV_{L} and RbtV_{H} for the variable light and variable heavy sequences respectively. Three of the most similar human germline antibody sequences, spanning from Framework 1 through to the end of Framework 3, are aligned below the rabbit sequences. The human sequence that is considered the most similar to the rabbit sequence is shown first. In this example those most similar sequences are L12A for the light chain and 3-64-04 for the heavy chain. Human CDR3 sequences are not shown. The closest human Framework 4 sequence is aligned below the rabbit Framework 4 sequence. The vertical dashes indicate a residue where the rabbit residue is identical with at least one of the human residues at the same position. The bold residues indicate that the human residue at that position is identical to the rabbit residue at the same position. The final humanized sequences are called V_{L}h and V_{H}h for the variable light and variable heavy sequences respectively. The underlined residues indicate that the residue is the same as the rabbit residue at that position but different than the human residues at that position in the three aligned human sequences.

FIGS. 2 and 3 show alignments between a rabbit antibody light and variable heavy sequences and homologous human sequences and the humanized sequences. Framework regions are identified as FR1-FR4. Complementarity determining regions are identified as CDR1-CDR3.

FIGS. 4A-B and 5A-B show alignments between light and variable heavy sequences, respectively, of different forms of Ab1. Framework regions are identified as FR1-FR4. Complementarity determining regions are identified as CDR1-CDR3. Sequence differences within the CDR regions highlighted.

FIG. 6 provides a pharmacokinetic profile of antibody Ab1 in cynomolgus monkey. Plasma levels of antibody Ab1 were quantitated through antigen capture ELISA. This protein displays a half-life of between 12 and 17 days consistent with other full length humanized antibodies.

FIG. 7A-D provides binding data for antibodies Ab4, Ab3, Ab8 and Ab2, respectively. FIG. 7E provides binding data for antibodies Ab1, Ab6 and Ab7.

FIG. 8 shows the mean plasma concentration of Ab1 resulting from a single administration of Ab1 to patients with advanced cancer.

FIG. 9A demonstrates suppression of serum CRP levels in healthy individuals.

FIG. 9B demonstrates suppression of serum CRP levels in advanced cancer patients.

FIG. 10 shows the mean CRP values for each dosage concentrations (placebo, 80 mg, 160 mg, and 320 mg) of the Ab1 monoclonal antibody.

FIG. 11 shows the change in median values of CRP from each dosage concentration group corresponding to FIG. 10.

FIG. 12 shows a reduction in serum CRP levels in patients with various cancers after dosing at 80, 160 or 320 mg for 12 weeks.

FIG. 13 shows the effect of subcutaneous and intravenous administration of ALD518 through week 12 after antibody dosing at 50 or 100 mg.

FIG. 14 shows the effect of subcutaneous and intravenous administration of ALD518 through week 12 after antibody dosing at 50 or 100 mg.

FIG. 15 shows plasma CRP level concentrations after subcutaneous or intravenous dosing of humanized Ab1.

FIG. 16 shows the study design used in Example 21 for safety and efficacy studies of Ab1, also known as MBS-945429 or clazakizumab. Single asterisk (*) indicates that rescue was allowed during Period II during the Long Term (Open-Label) Extension until all ongoing subjects were switched to the final dose of 2 mg. Double asterisks (**) indicate that during Long Term (Open Label) Extension, subjects continued the doses from Period II until a final dose was selected based on the Week 24 final analysis. The selected dose was 25 mg after the Week 24 analysis and was used for subjects remaining in the study for the rest of the long term extension.

FIG. 17 shows a bar plot of ACR20 response by treatment by clazakizumab at day 113 (Week 16) for all randomized and treated subjects.

FIG. 18 shows the ACR20 response rate to clazakizumab at the scheduled time point during the double-blind period (periods I and II) in all randomized and treated subjects. As shown, beginning as early as Day 29 (Week 4) and increasing through Day 169 (Week 24), subjects in the 3 clazakizumab groups achieved a numerically higher ACR20 response rate compared with the placebo group. At Day 113 (Week 16) the differences from placebo in ACR 20 response rates were numerically higher in the 25 mg and 100 mg clazakizumab groups compared with the difference from placebo and the difference from placebo with the 200 mg clazakizumab group (as noted previously). ACR20 response rates continued to improve over time and the difference from placebo in the 25 mg and 100 mg clazakizumab groups was again numerically higher compared with the difference from placebo in the 200 mg clazakizumab group at Day 141 (Week 20) and Day 169 (Week 24).

FIG. 19 shows ACR50 response rate to clazakizumab at the scheduled time point during the double blind (periods I and II) for all randomized and treated subjections. As shown, beginning as early as Day 29 (Week 4) and continuing through Day 169 (Week 24), subjects in all 3 clazakizumab dose groups achieved a numerically higher ACR50 response rate compared with the placebo group. At Day 113 (Week 16) the difference from placebo was numerically higher in the 25 mg and 100 mg clazakizumab groups compared with the 200 mg clazakizumab group. A similar trend was noted at Day 169 (Week 24) with numerically higher differences from placebo noted in the 25 mg and 100 mg clazakizumab groups compared to the 200 mg clazakizumab group.

FIG. 20 shows ACR70 response rate to clazakizumab at the scheduled time point during the double blind (periods I and II) for all randomized and treated subjections. As shown, beginning as early as Day 57 (Week 8) and continuing through Day 169 (Week 24), subjects in all 3 clazakizumab groups achieved a higher ACR70 response rate compared with the placebo group. At Day 113 (Week 16) the difference from placebo was numerically higher in the 25 mg and 100 mg clazakizumab groups compared with the difference from placebo for the 200 mg clazakizumab group. A similar trend was noted at Day 169 (Week 24) with numerically higher differences from placebo noted in the 25 mg and 100 mg clazakizumab groups compared to the 200 mg clazakizumab group.

FIG. 21 shows the median percent improvement in tender joint count after clazakizumab administration and during the double-blind period (periods I and II) for all randomized and treated subjects). As shown, beginning at Day 8 and continuing through Day 169 (Week 24), a numerically greater improvement in the median change from baseline tender joint count was shown for at least 1 dose of clazakizumab compared with the placebo group. Mean change from baseline results also showed numerically greater improvement in the tender joint count for all 3 clazakizumab groups compared with the placebo group at Day 113 (Week 16) and Day 169 (Week 24).

FIG. 22 shows the median percent improvement in swollen joint count after clazakizumab administration and during the double-blind period (periods I and II) for all randomized and treated subjects. As shown, beginning at Day 8 and continuing through Day 169 (Week 24), the median change from baseline swollen joint count showed numerically greater improvement in all 3 clazakizumab groups compared with the placebo group. Mean change from baseline results for swollen joint counts also showed numerically greater improvement in the 3 clazakizumab groups compared with the placebo group at Day 113 (Week 16) and Day 169 (Week 24).

FIG. 23 shows the response mean values of total IL-6 biomarker over time by treatment with clazakizumab during the double-blind period (periods I and II) in all pharmacodynamic analysis subjects.

FIG. 24 shows the mean values of free IL-6 biomarker over time by treatment with clazakizumab during the double-blind period (periods I and II) in all pharmacodynamic analysis subjects.

FIG. 25 shows that clazakizumab when used at doses of 25 mg, 100 mg, and 200 mg/month with background MTX, 100 mg and 200 mg monotherapy) demonstrated efficacy over placebo. In addition, each of the clazakizumab+MTX doses was associated with more patients achieving stringent measures of response than with adalimumab+MTX. Overall, there was not a strong dose response relationship at the doses tested on ACR20, ACR50, ACR70, DAS28-CRP<2.6, CDAI<2.8, or SDAI<3.3. Within the range of Cmins achieved with the 25 mg and higher, the relationship between Cmins and the probability of achieving an ACR response was relatively flat.

FIG. 26 examines clazakizumab IL-6/IL-6 soluble receptor complex inhibition data by ACR20 response. The data indicates that ACR20 responders had higher levels of inhibition at Week 12 than non-responders with the 25 mg dose in combination with MTX.

### DETAILED DESCRIPTION

### Definitions

It is to be understood that this subject technology is not limited to the particular methodology, protocols, cell lines, animal species or genera, and reagents described, as such may vary. It is also to be understood that the terminology used herein is for the purpose of describing particular embodiments only, and is not intended to limit the scope of the present subject technology which will be limited only by the appended claims.

As used herein the singular forms “a”, “and”, and “the” include plural referents unless the context clearly dictates otherwise. Thus, for example, reference to “a cell” includes a plurality of such cells and reference to “the protein” includes reference to at least one proteins and equivalents thereof known to those skilled in the art, and so forth. All technical and scientific terms used herein have the same meaning as commonly understood to one of ordinary skill in the art to which this subject technology belongs unless clearly indicated otherwise.

Amplification as used herein refers broadly to the amplification of polynucleotide sequences is the in vitro production of multiple copies of a particular nucleic acid sequence. The amplified sequence is usually in the form of DNA. A variety of techniques for carrying out such amplification are known in the art. See, e.g., Van Brunt (1990) Bio/Technol. 8(4): 291-294. Polymerase chain reaction or PCR is a prototype of nucleic acid amplification, and use of PCR herein should be considered exemplary of other suitable amplification techniques.

Engineered, as used herein with an antibody, refers to a non-naturally occurring antibody produced by recombinant or genetic engineering methodologies known in the art or described herein.

Antibody, as used herein, refers broadly to any polypeptide chain-containing molecular structure with a specific shape that fits to and recognizes an epitope, where at least one non-covalent binding interactions stabilize the complex between the molecular structure and the epitope. The archetypal antibody molecule is the immunoglobulin, and all types of immunoglobulins, IgG, IgM, IgA, IgE, IgD, from all sources, e.g., human, rodent, rabbit, cow, sheep, pig, dog, chicken, are considered to be “antibodies.” Antibodies include but are not limited to chimeric antibodies, human antibodies and other non-human mammalian antibodies, humanized antibodies, single chain antibodies (scFvs), camelbodies, nanobodies, IgNAR (single-chain antibodies derived from sharks), small-modular immunopharmaceuticals (SMIPs), and antibody fragments (e.g., Fabs, Fab′, F(ab′)₂.) Numerous antibody coding sequences have been described; and others may be raised by methods well-known in the art. See Streltsov, et al. (2005) Protein Sci. 14(11): 2901-9; Greenberg, et al. (1995) Nature 374(6518): 168-173; Nuttall, et al. (2001) Mol Immunol. 38(4): 313-26; Hamers-Casterman, et al. (1993) Nature 363(6428): 446-8; Gill, et al. (2006) Curr Opin Biotechnol. 17(6): 653-8.

Antigen-binding fragment, as used herein, refers broadly to a fragment of an antibody which recognizes an antigen (e.g., paratopes.) The antigen-binding fragment may comprise a paratope that may be a small region (e.g., 15-22 amino acids) of the antibody's Fv region and may contain parts of the antibody's heavy and light chains. See Goldsby, et al. Antigens (Chapter 3) Immunology p Ed.) New York: W.H. Freeman and Company, pages 57-75.

C-Reactive Protein (CRP), as used herein, refers broadly to a 224 amino acid protein found in the blood that rise in response to inflammation (e.g., GenBank Protein Accession No. NP_000558 and SEQ ID NO: 726). CRP also encompasses any pre-pro, pro- and mature forms of this CRP amino acid sequence, as well as mutants and variants including allelic variants of this sequence. CRP levels, e.g. in the serum, liver, or elsewhere in the body, can be readily measured using routine methods and commercially available reagents, e.g. ELISA, antibody test strip, immunoturbidimetry, rapid immunodiffusion, visual agglutination, Western blot, Northern blot As mentioned above CRP levels may in addition be measured in patients having or at risk of developing thrombosis according to the subject technology.

Coding sequence, as used herein refers broadly to an in-frame sequence of codons that (in view of the genetic code) correspond to or encode a protein or peptide sequence. Two coding sequences correspond to each other if the sequences or their complementary sequences encode the same amino acid sequences. A coding sequence in association with appropriate regulatory sequences may be transcribed and translated into a polypeptide. A polyadenylation signal and transcription termination sequence will usually be located 3′ to the coding sequence. A “promoter sequence” is a DNA regulatory region capable of binding RNA polymerase in a cell and initiating transcription of a downstream (3′ direction) coding sequence. Promoter sequences typically contain additional sites for binding of regulatory molecules (e.g., transcription factors) which affect the transcription of the coding sequence. A coding sequence is “under the control” of the promoter sequence or “operatively linked” to the promoter when RNA polymerase binds the promoter sequence in a cell and transcribes the coding sequence into mRNA, which is then in turn translated into the protein encoded by the coding sequence. A polynucleotide sequence “corresponds” to a polypeptide sequence if translation of the polynucleotide sequence in accordance with the genetic code yields the polypeptide sequence (i.e., the polynucleotide sequence “encodes” the polypeptide sequence), one polynucleotide sequence “corresponds” to another polynucleotide sequence if the two sequences encode the same polypeptide sequence.

Complementarity determining region, hypervariable region, or CDR, as used herein refer broadly to at least one of the hyper-variable or complementarity determining regions (CDRs) found in the variable regions of light or heavy chains of an antibody (See Kabat, E. A. et al. (1987) Sequences of Proteins of Immunological Interest, National Institutes of Health, Bethesda, Md.). These expressions include the hypervariable regions as defined by Kabat et al. (“Sequences of Proteins of Immunological Interest,” Kabat E., et al. (1983) US Dept. of Health and Human Services) or the hypervariable loops in 3-dimensional structures of antibodies. Chothia and Leska (1987) J Mol. Biol. 196: 901-917. The CDRs in each chain are held in close proximity by framework regions and, with the CDRs from the other chain, contribute to the formation of the antigen binding site. Within the CDRs there are select amino acids that have been described as the selectivity determining regions (SDRs) which represent the critical contact residues used by the CDR in the antibody-antigen interaction (Kashmiri (2005) Methods 36:25-34). CDRs for exemplary anti-IL-6 antibodies are provided herein.

Disease or condition, as used herein, refers broadly to a disease or condition that a patient has been diagnosed with or is suspected of having, particularly a disease or condition associated with elevated IL-6. A disease or condition encompasses, without limitation thereto, psoriatic arthritis, as well as idiopathic conditions characterized by symptoms that include elevated IL-6.

Effective amount, as used herein, refers broadly to an amount of an active ingredient that is effective to relieve or reduce to some extent at least one of the symptoms of the disease in need of treatment, or to retard initiation of clinical markers or symptoms of a disease in need of prevention, when the compound is administered. Thus, an effective amount refers to an amount of the active ingredient which exhibit effects such as (i) reversing the rate of progress of a disease; (ii) inhibiting to some extent further progress of the disease; and/or, (iii) relieving to some extent (or, preferably, eliminating) at least one symptoms associated with the disease. The effective amount may be empirically determined by experimenting with the compounds concerned in known in vivo and in vitro model systems for a disease in need of treatment. The context in which the phrase “effective amount” is used may indicate a particular desired effect. For example, “an amount of an anti-IL-6 antibody effective to prevent or treat a hypercoagulable state” and similar phrases refer to an amount of anti-IL-6 antibody that, when administered to a subject, will cause a measurable improvement in the subject's coagulation profile, or prevent, slow, delay, or arrest, a worsening of the coagulation profile for which the subject is at risk. Similarly, “an amount of an anti-IL-6 antibody effective to reduce serum CRP levels” and similar phrases refer to an amount of anti-IL-6 antibody that, when administered to a subject, will cause a measurable decrease in serum CRP levels, or prevent, slow, delay, or arrest, an increase in serum CRP levels for which the subject is at risk. Similarly, “an amount of an anti-IL-6 antibody effective to increase serum albumin levels” and similar phrases refer to an amount of anti-IL-6 antibody that, when administered to a subject, will cause a measurable increase in serum albumin levels, or prevent, slow, delay, or arrest, a decrease in serum albumin levels for which the subject is at risk. Similarly, “an amount of an anti-IL-6 antibody effective to reduce weakness” and similar phrases refer to an amount of anti-IL-6 antibody that, when administered to a subject, will cause a measurable decrease in weakness as determined by the hand grip strength test. Similarly, “an amount of an anti-IL-6 antibody effective to increase weight” and similar phrases refer to an amount of anti-IL-6 antibody that, when administered to a subject, will cause a measurable increase in a patient's weight. An effective amount will vary according to the weight, sex, age and medical history of the individual, as well as the severity of the patient's condition(s), the type of disease(s), mode of administration, and the like. An effective amount may be readily determined using routine experimentation, e.g., by titration (administration of increasing dosages until an effective dosage is found) and/or by reference to amounts that were effective for prior patients. Generally, the anti-IL-6 antibodies of the present subject technology will be administered in dosages ranging between about 0.1 mg/kg and about 20 mg/kg of the patient's body-weight.

Expression Vector, as used herein, refers broadly to a DNA vectors contain elements that facilitate manipulation for the expression of a foreign protein within the target host cell. Conveniently, manipulation of sequences and production of DNA for transformation is first performed in a bacterial host, e.g. E. coli, and usually vectors will include sequences to facilitate such manipulations, including a bacterial origin of replication and appropriate bacterial selection marker. Selection markers encode proteins necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, (b) complement auxotrophic deficiencies, or (c) supply critical nutrients not available from complex media. Exemplary vectors and methods for transformation of yeast are described, for example, in Burke, D., Dawson, D., & Stearns, T. (2000). Methods in yeast genetics: a Cold Spring Harbor Laboratory course manual. Plainview, N.Y.: Cold Spring Harbor Laboratory Press.

Folding, as used herein, refers broadly to the three-dimensional structure of polypeptides and proteins, where interactions between amino acid residues act to stabilize the structure. While non-covalent interactions are important in determining structure, usually the proteins of interest will have intra- and/or intermolecular covalent disulfide bonds formed by two cysteine residues. For naturally occurring proteins and polypeptides or derivatives and variants thereof, the proper folding is typically the arrangement that results in optimal biological activity, and can conveniently be monitored by assays for activity, e.g. ligand binding, enzymatic activity.

Framework region or FR, as used herein refers broadly to at least one of the framework regions within the variable regions of the light and heavy chains of an antibody. See Kabat, et al. (1987) Sequences of Proteins of Immunological Interest, National Institutes of Health, Bethesda, Md. These expressions include those amino acid sequence regions interposed between the CDRs within the variable regions of the light and heavy chains of an antibody. As mentioned in the preferred embodiments, the FRs may comprise human FRs highly homologous to the parent antibody (e.g., rabbit antibody).

gp130 (also called Interleukin-6 receptor subunit beta), as used herein, refers broadly to a transmembrane protein that forms one subunit of type I cytokine receptors in the IL-6 receptor family (e.g., 918 precursor amino acid sequence available as Swiss-Prot Protein Accession No. P40189 and SEQ ID NO: 728). gp130 also encompasses any pre-pro, pro- and mature forms of this amino acid sequence, such as the mature form encoded by amino acids 23 through 918 of the sequence shown, as well as mutants and variants including allelic variants of this sequence.

Heterologous region or domain of a DNA construct, as used herein, refers broadly to an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous region is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

Homology, as used herein, refers broadly to a degree of similarity between a nucleic acid sequence and a reference nucleic acid sequence or between a polypeptide sequence and a reference polypeptide sequence. Homology may be partial or complete. Complete homology indicates that the nucleic acid or amino acid sequences are identical. A partially homologous nucleic acid or amino acid sequence is one that is not identical to the reference nucleic acid or amino acid sequence. The degree of homology can be determined by sequence comparison. The term “sequence identity” may be used interchangeably with “homology.”

Host cell, as used herein, refers broadly to a cell that contains an expression vector and supports the replication or expression of the expression vector. Host cells may be prokaryotic cells such as E. coli, or eukaryotic cells such as yeast, insect (e.g., SF9), amphibian, or mammalian cells such as CHO, HeLa, HEK-293 (e.g., cultured cells, explants, and cells in vivo.)

Isolated, as used herein, refers broadly to material removed from its original environment in which it naturally occurs, and thus is altered by the hand of man from its natural environment. Isolated material may be, for example, exogenous nucleic acid included in a vector system, exogenous nucleic acid contained within a host cell, or any material which has been removed from its original environment and thus altered by the hand of man (e.g., “isolated antibody”).

Improved, as used herein, refers broadly to any beneficial change resulting from a treatment. A beneficial change is any way in which a patient's condition is better than it would have been in the absence of the treatment. “Improved” includes prevention of an undesired condition, slowing the rate at which a condition worsens, delaying the development of an undesired condition, and restoration to an essentially normal condition. For example, improvement in psoriatic arthritis encompasses any decrease in pain, swelling, joint stiffness, or inflammation, and/or an increase in joint mobility.

IL-6 antagonist, as used herein, refers broadly to any composition that prevents, inhibits, or lessens the effect(s) of IL-6 signaling. Generally, such antagonists may reduce the levels or activity of IL-6, IL-6 receptor alpha, gp130, or a molecule involved in IL-6 signal transduction, or may reduce the levels or activity complexes between the foregoing (e.g., reducing the activity of an IL-6/IL-6 receptor complex). Antagonists include antisense nucleic acids, including DNA, RNA, or a nucleic acid analogue such as a peptide nucleic acid, locked nucleic acid, morpholino (phosphorodiamidate morpholino oligo), glycerol nucleic acid, or threose nucleic acid. See Heasman (2002) Dev Biol. 243(2): 209-14; Hannon and Rossi (2004) Nature 431(7006):371-8; Paul, et al. (2002) Nat Biotechnol. 20(5):505-8; Zhang, et al. (2005) J Am Chem Soc. 127(12):4174-5; Wahlestedt, et al. (2000) Proc Natl Acad Sci USA. 97(10):5633-8; Hanvey, et al. (1992) Science 258 (5087):1481-5; Braasch, et al. (2002) Biochemistry 41(14): 4503-10; Schoning, et al. (2000) Science 290(5495): 1347-51. In addition IL-6 antagonists specifically include peptides that block IL-6 signaling such as those described in any of U.S. Pat. Nos. 5,210,075; 6,172,042; 6,599,875; 6,841,533; and 6,838,433. Also, IL-6 antagonists according to the subject technology may include p38 MAP kinase inhibitors such as those reported in U.S. Patent Application No. 2007/0010529 given this kinase's role in cytokine production and more particularly IL-6 production. Further, IL-6 antagonists according to the subject technology include the glycoalkaloid compounds reported in U.S. Patent Application Publication No. 2005/0090453 as well as other IL-6 antagonist compounds isolatable using the IL-6 antagonist screening assays reported therein. Other IL-6 antagonists include antibodies, such as anti-IL-6 antibodies, anti-IL-6 receptor alpha antibodies, anti-gp130 antibodies, and anti-p38 MAP kinase antibodies including (but not limited to) the anti-IL-6 antibodies disclosed herein, Actemra® (Tocilizumab), Remicade®, Zenapax® (daclizumab), or any combination thereof. Other IL-6 antagonists include portions or fragments of molecules involved in IL-6 signaling, such as IL-6, IL-6 receptor alpha, and gp130, which may be native, mutant, or variant sequence, and may optionally be coupled to other moieties (such as half-life-increasing moieties, e.g. an Fc domain). For example, an IL-6 antagonist may be a soluble IL-6 receptor or fragment, a soluble IL-6 receptor:Fc fusion protein, a small molecule inhibitor of IL-6, an anti-IL-6 receptor antibody or antibody fragment or variant thereof, antisense nucleic acid. Other IL-6 antagonists include avemirs, such as C326 (Silverman, et al. (2005) Nat Biotechnol. 23(12): 1556-61) and small molecules, such as synthetic retinoid AM80 (tamibarotene) (Takeda, et al. (2006) Arterioscler Thromb Vasc Biol. 26(5): 1177-83). Such IL-6 antagonists may be administered by any means known in the art, including contacting a subject with nucleic acids which encode or cause to be expressed any of the foregoing polypeptides or antisense sequences.

Interleukin-6 (IL-6), as used herein, refers broadly to interleukin-6 (IL-6) encompasses not only the following 212 amino acid sequence available as GenBank Protein Accession No. NP_000591 (e.g., SEQ ID NO: 1), but also any pre-pro, pro- and mature forms of this IL-6 amino acid sequence, as well as mutants and variants including allelic variants of this sequence.

Interleukin-6 receptor (IL-6R) (IL-6 receptor alpha (IL-6RA) [CD126], as used herein, refers broadly to 468 amino acid protein that binds IL-6, a potent pleiotropic cytokine that regulates cell growth and differentiation and also plays an important role in immune response (e.g., Swiss-Prot Protein Accession No. P08887 and SEQ ID NO: 727). IL-6R also includes any pre-pro, pro- and mature forms of this amino acid sequence, as well as mutants and variants including allelic variants of this sequence.

Mammal, as used herein, refers broadly to any and all warm-blooded vertebrate animals of the class Mammalia, including humans, characterized by a covering of hair on the skin and, in the female, milk-producing mammary glands for nourishing the young. Examples of mammals include but are not limited to alpacas, armadillos, capybaras, cats, camels, chimpanzees, chinchillas, cattle, dogs, goats, gorillas, hamsters, horses, humans, lemurs, llamas, mice, non-human primates, pigs, rats, sheep, shrews, squirrels, and tapirs. Mammals include but are not limited to bovine, canine, equine, feline, murine, ovine, porcine, primate, and rodent species. Mammal also includes any and all those listed on the Mammal Species of the World maintained by the National Museum of Natural History, Smithsonian Institution in Washington DC.

Nucleic acid or nucleic acid sequence, as used herein, refers broadly to a deoxy-ribonucleotide or ribonucleotide oligonucleotide in either single- or double-stranded form. The term encompasses nucleic acids, i.e., oligonucleotides, containing known analogs of natural nucleotides. The term also encompasses nucleic-acid-like structures with synthetic backbones. Unless otherwise indicated, a particular nucleic acid sequence also implicitly encompasses conservatively modified variants thereof (e.g., degenerate codon substitutions) and complementary sequences, as well as the sequence explicitly indicated. The term nucleic acid is used interchangeably with gene, cDNA, mRNA, oligonucleotide, and polynucleotide.

Operatively linked, as used herein, refers broadly to when two DNA fragments are joined such that the amino acid sequences encoded by the two DNA fragments remain in-frame.

Paratope, as used herein, refers broadly to the part of an antibody which recognizes an antigen (e.g., the antigen-binding site of an antibody.) Paratopes may be a small region (e.g., 15-22 amino acids) of the antibody's Fv region and may contain parts of the antibody's heavy and light chains. See Goldsby, et al. Antigens (Chapter 3) Immunology (5^{th} Ed.) New York: W.H. Freeman and Company, pages 57-75.

Patient, as used herein, refers broadly to any animal who is in need of treatment either to alleviate a disease state or to prevent the occurrence or reoccurrence of a disease state. Also, “Patient” as used herein, refers broadly to any animal who has risk factors, a history of disease, susceptibility, symptoms, signs, was previously diagnosed, is at risk for, or is a member of a patient population for a disease. The patient may be a clinical patient such as a human or a veterinary patient such as a companion, domesticated, livestock, exotic, or zoo animal. The term “subject” may be used interchangeably with the term “patient”.

Polyploid yeast that stably expresses or expresses a desired secreted heterologous polypeptide for prolonged time, as used herein, refers broadly to a yeast culture that secretes said polypeptide for at least several days to a week, more preferably at least a month, still more preferably at least about 1-6 months, and even more preferably for more than a year at threshold expression levels, typically at least about 10-25 mg/liter and preferably substantially greater.

Polyploidal yeast culture that secretes desired amounts of recombinant polypeptide, as used herein, refers broadly to cultures that stably or for prolonged periods secrete at least about 10-25 mg/liter of heterologous polypeptide, more preferably at least about 50-500 mg/liter, and most preferably at least about 500-1000 mg/liter or more.

Prolonged improvement in coagulation profile, as used herein, refers broadly to a measurable improvement in the subject's coagulation profile relative to the initial coagulation profile (i.e. the coagulation profile at a time before treatment begins) that is detectable within about a week from when treatment begins (e.g. administration of an IL-6 antagonist such as Ab1) and remains improved for a prolonged duration, e.g., at least about 14 days, at least about 21 days, at least about 28 days, at least about 35 days, at least about 40 days, at least about 50 days, at least about 60 days, at least about 70 days, at least about 11 weeks, or at least about 12 weeks from when the treatment begins.

Prolonged reduction in serum CRP, as used herein, refers broadly to a measurable decrease in serum CRP level relative to the initial serum CRP level (i.e. the serum CRP level at a time before treatment begins) that is detectable within about a week from when a treatment begins (e.g. administration of an anti-IL-6 antibody) and remains below the initial serum CRP level for an prolonged duration, e.g. at least about 14 days, at least about 21 days, at least about 28 days, at least about 35 days, at least about 40 days, at least about 50 days, at least about 60 days, at least about 70 days, at least about 11 weeks, or at least about 12 weeks from when the treatment begins.

Promoter, as used herein, refers broadly to an array of nucleic acid sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation.

Prophylactically effective amount, as used herein, refers broadly to the amount of a compound that, when administered to a patient for prophylaxis of a disease or prevention of the reoccurrence of a disease, is sufficient to effect such prophylaxis for the disease or reoccurrence. The prophylactically effective amount may be an amount effective to prevent the incidence of signs and/or symptoms. The “prophylactically effective amount” may vary depending on the disease and its severity and the age, weight, medical history, predisposition to conditions, preexisting conditions, of the patient to be treated.

Prophylaxis, as used herein, refers broadly to a course of therapy where signs and/or symptoms are not present in the patient, are in remission, or were previously present in a patient. Prophylaxis includes preventing disease occurring subsequent to treatment of a disease in a patient. Further, prevention includes treating patients who may potentially develop the disease, especially patients who are susceptible to the disease (e.g., members of a patent population, those with risk factors, or at risk for developing the disease).

Recombinant as used herein, refers broadly with reference to a product, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

Selectable Marker, as used herein, refers broadly to a selectable marker is a gene or gene fragment that confers a growth phenotype (physical growth characteristic) on a cell receiving that gene as, for example through a transformation event. The selectable marker allows that cell to survive and grow in a selective growth medium under conditions in which cells that do not receive that selectable marker gene cannot grow. Selectable marker genes generally fall into several types, including positive selectable marker genes such as a gene that confers on a cell resistance to an antibiotic or other drug, temperature when two ts mutants are crossed or a ts mutant is transformed; negative selectable marker genes such as a biosynthetic gene that confers on a cell the ability to grow in a medium without a specific nutrient needed by all cells that do not have that biosynthetic gene, or a mutagenized biosynthetic gene that confers on a cell inability to grow by cells that do not have the wild type gene; and the like. Suitable markers include but are not limited to ZEOMYCIN® (zeocin), neomycin, G418, LYS3, MET1, MET3a, ADE1, ADE3, and URA3.

Specifically (or selectively) binds to an antibody or “specifically (or selectively) immunoreactive with,” or “specifically interacts or binds,” as used herein, refers broadly to a protein or peptide (or other epitope), refers, in some embodiments, to a binding reaction that is determinative of the presence of the protein in a heterogeneous population of proteins and other biologics. For example, under designated immunoassay conditions, the specified antibodies bind to a particular protein at least two times greater than the background (non-specific signal) and do not substantially bind in a significant amount to other proteins present in the sample. Typically a specific or selective reaction will be at least twice background signal or noise and more typically more than about 10 to 100 times background.

Signs of disease, as used herein, refers broadly to any abnormality indicative of disease, discoverable on examination of the patient; an objective indication of disease, in contrast to a symptom, which is a subjective indication of disease.

Solid support, support, and substrate, as used herein, refers broadly to any material that provides a solid or semi-solid structure with which another material can be attached including but not limited to smooth supports (e.g., metal, glass, plastic, silicon, and ceramic surfaces) as well as textured and porous materials.

Subjects as used herein, refers broadly to anyone suitable to be treated according to the present subject technology include, but are not limited to, avian and mammalian subjects, and are preferably mammalian. Mammals of the present subject technology include, but are not limited to, canines, felines, bovines, caprines, equines, ovines, porcines, rodents (e.g., rats and mice), lagomorphs, primates, humans. Any mammalian subject in need of being treated according to the present subject technology is suitable. Human subjects of both genders and at any stage of development (i.e., neonate, infant, juvenile, adolescent, adult) can be treated according to the present subject technology. The present subject technology may also be carried out on animal subjects, particularly mammalian subjects such as mice, rats, dogs, cats, cattle, goats, sheep, and horses for veterinary purposes, and for drug screening and drug development purposes. “Subjects” is used interchangeably with “patients.”

Mating competent yeast species, as used herein refers broadly encompass any diploid or tetraploid yeast which can be grown in culture. Such species of yeast may exist in a haploid, diploid, or tetraploid form. The cells of a given ploidy may, under appropriate conditions, proliferate for indefinite number of generations in that form. Diploid cells can also sporulate to form haploid cells. Sequential mating can result in tetraploid strains through further mating or fusion of diploid strains. In the present subject technology the diploid or polyploidal yeast cells are preferably produced by mating or spheroplast fusion.

Haploid Yeast Cell, as used herein, refers broadly to a cell having a single copy of each gene of its normal genomic (chromosomal) complement.

Polyploid Yeast Cell, as used herein, refers broadly to a cell having more than one copy of its normal genomic (chromosomal) complement.

Diploid Yeast Cell, as used herein, refers broadly to a cell having two copies (alleles) of essentially every gene of its normal genomic complement, typically formed by the process of fusion (mating) of two haploid cells.

Tetraploid Yeast Cell, as used herein, refers broadly to a cell having four copies (alleles) of essentially every gene of its normal genomic complement, typically formed by the process of fusion (mating) of two haploid cells. Tetraploids may carry two, three, four, or more different expression cassettes. Such tetraploids might be obtained in S. cerevisiae by selective mating homozygotic heterothallic a/a and alpha/alpha diploids and in Pichia by sequential mating of haploids to obtain auxotrophic diploids. For example, a [met his] haploid can be mated with [ade his] haploid to obtain diploid [his]; and a [met arg] haploid can be mated with [ade arg] haploid to obtain diploid [arg]; then the diploid [his] x diploid [arg] to obtain a tetraploid prototroph. It will be understood by those of skill in the art that reference to the benefits and uses of diploid cells may also apply to tetraploid cells.

Yeast Mating, as used herein, refers broadly to a process by which two haploid yeast cells naturally fuse to form one diploid yeast cell.

Meiosis, as used herein, refers broadly to a process by which a diploid yeast cell undergoes reductive division to form four haploid spore products. Each spore may then germinate and form a haploid vegetatively growing cell line.

Variable region or VR as used herein refers broadly to the domains within each pair of light and heavy chains in an antibody that are involved directly in binding the antibody to the antigen. Each heavy chain has at one end a variable domain (V_{H}) followed by a number of constant domains. Each light chain has a variable domain (V_{L}) at one end and a constant domain at its other end; the constant domain of the light chain is aligned with the first constant domain of the heavy chain, and the light chain variable domain is aligned with the variable domain of the heavy chain.

Variants, as used herein refers broadly to single-chain antibodies, dimers, multimers, sequence variants, and domain substitution variants. Single-chain antibodies such as SMIPs, shark antibodies, nanobodies (e.g., Camelidiae antibodies). Sequence variants can be specified by percentage identity (similarity, sequence homology) e.g., 99%, 95%, 90%, 85%, 80%, 70%, 60%, or by numbers of permitted conservative or non-conservative substitutions. Domain substitution variants include replacement of a domain of one protein with a similar domain of a related protein. A similar domain may be identified by similarity of sequence, structure (actual or predicted), or function. For example, domain substitution variants include the substitution of at least one CDRs and/or framework regions.

The techniques and procedures are generally performed according to conventional methods well known in the art and as described in various general and more specific references that are cited and discussed throughout the present specification. See, e.g., Sambrook, et al. (2001) Molec. Cloning: Lab. Manual[3^{rd} Ed] Cold Spring Harbor Laboratory Press. Standard techniques may be used for recombinant DNA, oligonucleotide synthesis, and tissue culture, and transformation (e.g., electroporation, lipofection). Enzymatic reactions and purification techniques may be performed according to manufacturer's specifications or as commonly accomplished in the art or as described herein. The nomenclatures utilized in connection with, and the laboratory procedures and techniques of, analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, and delivery, and treatment of patients.

### Anti-IL-6 Antibodies for Treating Psoriatic Arthritis

The present technology also relates to compositions, methods, and uses of anti-IL-6 antibodies and/or antigen-binding fragments thereof according to the subject technology for treating, preventing, or alleviating the onset of psoriatic arthritis.

The subject therapy may comprise administering the antibody prior or concurrent to development of the symptoms of psoriatic arthritis. Particularly this may be used in patients who have shown signs of inadequate response to Non-Steroidal Anti-Inflammatory Drugs (NSAIDs) and/or non-biologic Disease Modifying Anti-Rheumatic Drugs (DMARDs). Non-biologic DMARDs include, but are not limited to: Mycophenolate mofetil (CellCept®), calcineurin inhibitors (e.g., cyclosporine, sirolimus, everolimus), oral retinoids, azathioprine, fumeric acid esters, D-penicillamine, and cyclophosphamide. Non-Steroidal Anti-Inflammatory Drugs (NSAIDs) including but are not limited to Salicylates (e.g., Aspirin (acetylsalicylic acid), Diflunisal, Salsalate); Propionic acid derivatives (e.g., Ibuprofen, Naproxen, Fenoprofen, Ketoprofen, Flurbiprofen, Oxaprozin, Loxoprofen); Acetic acid derivatives (e.g., Indomethacin, Sulindac, Etodolac, Ketorolac, Diclofenac, Nabumetone); Enolic acid (Oxicam) derivatives (e.g., Piroxicam, Meloxicam, Tenoxicam, Droxicam, Lornoxicam, Isoxicam); Fenamic acid derivatives (Fenamates) (e.g., Mefenamic acid, Meclofenamic acid, Flufenamic acid, Tolfenamic acid); Selective COX-2 inhibitors (Coxibs) (e.g., Celecoxib, Rofecoxib, Valdecoxib, Parecoxib, Lumiracoxib, Etoricoxib, Firocoxib), Sulphonanilides (e.g., Nimesulide), and Licofelone.

The subject technology provides for method of treating psoriatic arthritis comprising administration of a composition comprising an effective amount of an Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, Ab21, Ab22, Ab23, Ab24, Ab25, Ab26, Ab27, Ab28, Ab29, Ab30, Ab31, Ab32, Ab33, Ab34, Ab35, or Ab36 antibody, or an antigen-binding fragment thereof, to a subject in need thereof, wherein the antibody, or antigen-binding fragment thereof, specifically binds to IL-6.

The subject technology also provides for method of preventing psoriatic arthritis comprising administration of a composition comprising an effective amount of an Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, Ab21, Ab22, Ab23, Ab24, Ab25, Ab26, Ab27, Ab28, Ab29, Ab30, Ab31, Ab32, Ab33, Ab34, Ab35, or Ab36 antibody, or an antigen-binding fragment thereof, to a subject in need thereof, wherein the antibody, or antigen-binding fragment thereof, specifically binds to IL-6.

In either the methods of treatment or prevention, the antibody, or antigen-binding fragment thereof, is aglycosylated. Further, the antibody, or antigen-binding fragment thereof, may contain an Fc region that has been modified to alter effector function, half-life, proteolysis, and/or glycosylation. Additionally, the antibody, or antigen-binding fragment thereof, is a human, humanized, single chain, or chimeric antibody.

Further, the method of treating or preventing psoriatic arthritis may comprise administering a composition comprises at least about 25, 80, 100, 160, 200, or 320 mg of an Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, Ab21, Ab22, Ab23, Ab24, Ab25, Ab26, Ab27, Ab28, Ab29, Ab30, Ab31, Ab32, Ab33, Ab34, Ab35, or Ab36 antibody, or an antigen-binding fragment thereof. The method of treating or preventing psoriatic arthritis may comprise administering a composition comprises at least about 25, 80, 100, 160, 200, or 320 mg of an Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, Ab21, Ab22, Ab23, Ab24, Ab25, Ab26, Ab27, Ab28, Ab29, Ab30, Ab31, Ab32, Ab33, Ab34, Ab35, or Ab36 antibody, or an antigen-binding fragment thereof, subcutaneously every 4 weeks for at least 8, 16, 20, or 24 weeks.

In an embodiment of the subject technology, IL-6 antagonists such as Ab1 described herein are useful for ameliorating or reducing the symptoms of, or treating, or preventing, psoriatic arthritis. The IL-6 antagonists described herein (e.g., Ab1-Ab36) is administered in a therapeutically effective amount to patients in need of treatment of psoriatic arthritis in the form of a pharmaceutical composition formulated for the treatment of psoriatic arthritis.

The dosing regimen is based on pharmacokinetic and pharmacodynamic data from previous studies. For example, in the advanced cancer clinical trial, single IV doses of 80, 160, and 320 mg ALD518 decreased CRP levels to normal or near normal for 12 weeks. In the rheumatoid arthritis clinical trial, 2 IV doses of 80, 160, and 320 mg ALD518 given 8 weeks apart decreased CRP levels to normal or nearly normal for 16 weeks. However, in the NSCLC clinical trial, CRP levels were decreased 2 weeks after the first of 3 IV doses of 80, 160, and 320 mg of ALD518, 8 weeks apart, but increased prior to the second dose. In addition, the elimination half-life, which is based on free ALD518, was 28 days in the normal subject, advanced cancer, but was reduced to 21 days in the NSCLC clinical trial. ALD518* is an asialated, humanized anti-IL-6 monoclonal antibody with a half-life of ˜30 days containing the humanized variable heavy and light sequences set forth in SEQ ID NO: 19 and 20. Pharmacokinetic (PK) modeling of data from the NSCLC clinical trial indicates that doses of ALD518 80 mg administered once every 3 weeks would not result in trough ALD518 concentrations high enough to fully suppress CRP. Since CRP levels in the published data in head and neck cancer studies can be as high as those seen in subjects with NSCLC, the doses may be 160 mg and 320 mg of humanized monoclonal antibody that selectively binds IL-6 administered every 4 weeks. See, e.g., Gallo, et al. (1992) Br J Cancer 65:479-80; Duffy, et al. (2008) Cancer 113:750-7. Examplary ALD518 plasma concentration effective to inhibit CRP may be at least about 15 μg/mL.

ALD518 is an exemplary humanized anti-IL-6 monoclonal antibody. ALD518 may be supplied as a pH 6.0 frozen injection in single-use vials (e.g., 80, 160, or 320 mg) for intravenous administration. In the 80 mg dose, exemplary non-active excipients include but are not limited to 25 mM histidine and 250 mM sorbitol. In the 160 mg formulation, exemplary non-active excipients include but are not limited to 25 mM histidine, 250 mM sorbitol, and 0.015% polysorbate 80. Compositions comprising humanized monoclonal antibodies that selectively bind IL-6 (e.g., ALD518) may be sterile, preservative-free frozen liquid injection in depyrogenated sterile vials, which are stoppered and sealed containing approximately 80 mg (e.g., 7.6 mL in a 10 mLvial) or approximately 160 mg (e.g., 4 mL in a 5 mL vial). For example, one dose of ALD518 (e.g., 160 mg or 320 mg) in 250 mL 0.9% saline may be administered IV over a period of at least about one hour (±15 minutes) on the morning of RT Day 1 and RT Treatment Week 4.

In one embodiment of the subject technology, IL-6 antagonists described herein (e.g., Ab1) are useful for ameliorating or reducing the symptoms of, or treating, or preventing psoriatic arthritis.

In another embodiment of the subject technology, IL-6 antagonists described herein are administered to a patient in combination with another active agent. For example, an IL-6 antagonist such as Ab1 may be co-administered with at least one chemotherapy agents, such as VEGF antagonists, EGFR antagonists, platins, taxols, irinotecan, 5-fluorouracil, gemcytabine, leucovorine, steroids, cyclophosphamide, melphalan, vinca alkaloids (e.g., vinblastine, vincristine, vindesine and vinorelbine), mustines, tyrosine kinase inhibitors, radiotherapy, sex hormone antagonists, selective androgen receptor modulators, selective estrogen receptor modulators, PDGF antagonists, TNF antagonists, IL-1 antagonists, interleukins (e.g. IL-12 or IL-2), IL-12R antagonists, Erbitux® (cetuximab), Avastin® (bevacizumab), Pertuzumab, anti-CD20 antibodies, Rituxan® (rituximab), ocrelizumab, ofatumumab, DXL625, Herceptin® (trastuzumab), or any combination thereof.

### Anti-IL-6 Antibodies and Binding Fragments Thereof

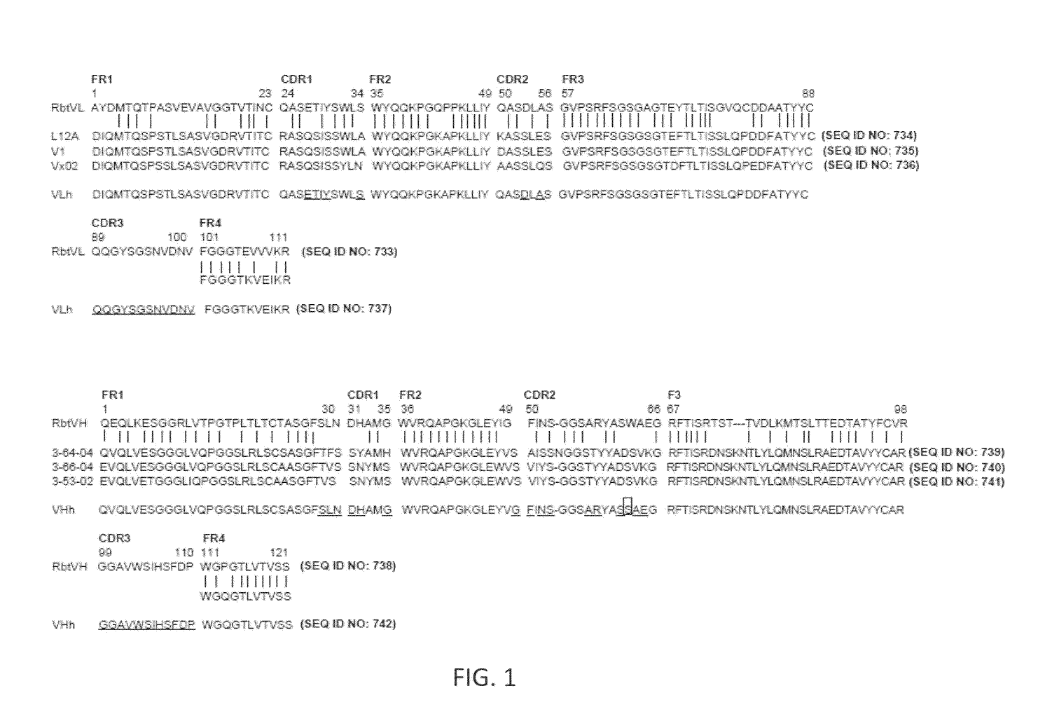

The subject technology includes antibodies having binding specificity to IL-6 and possessing a variable light chain sequence comprising the sequence set forth in the polypeptide sequence of SEQ ID NO: 2 or SEQ ID NO: 709 and humanized versions and variants thereof including those set forth in FIGS. 1 and 8-11, and those identified in Table 1.

Antibodies consist of two identical light polypeptide chains of molecular weight approximately 23,000 daltons (the “light chain”), and two identical heavy chains of molecular weight 53,000-70,000 (the “heavy chain”). The four chains are joined by disulfide bonds in a “Y” configuration wherein the light chains bracket the heavy chains starting at the mouth of the “Y” configuration. The “branch” portion of the “Y” configuration is designated the Fab region; the stem portion of the “Y” configuration is designated the Fc region. The amino acid sequence orientation runs from the N-terminal end at the top of the “Y” configuration to the C-terminal end at the bottom of each chain. The N-terminal end possesses the variable region having specificity for the antigen that elicited it, and is approximately 100 amino acids in length, there being slight variations between light and heavy chain and from antibody to antibody.

The variable region is linked in each chain to a constant region that extends the remaining length of the chain and that within a particular class of antibody does not vary with the specificity of the antibody (i.e., the antigen eliciting it). There are five known major classes of constant regions that determine the class of the immunoglobulin molecule (IgG, IgM, IgA, IgD, and IgE corresponding to γ, μ, α, δ, and ε (gamma, mu, alpha, delta, or epsilon) heavy chain constant regions). The constant region or class determines subsequent effector function of the antibody, including activation of complement (Kabat, E. A. (1976) Structural Concepts in Immunology and Immunochemistry, 2nd Ed., pp. 413-436, Holt, Rinehart, Winston), and other cellular responses (Andrews, et al. (1980) Clinical Immunobiology pp 1-18, W. B. Sanders; Kohl, et al. (1983) Immunology 48: 187); while the variable region determines the antigen with which it will react. Light chains are classified as either κ (kappa) or λ (lambda). Each heavy chain class can be paired with either kappa or lambda light chain. The light and heavy chains are covalently bonded to each other, and the “tail” portions of the two heavy chains are bonded to each other by covalent disulfide linkages when the immunoglobulins are generated either by hybridomas or by B cells.

For example, antibodies or antigen binding fragments or variants thereof may be produced by genetic engineering. In this technique, as with other methods, antibody-producing cells are sensitized to the desired antigen or immunogen. The messenger RNA isolated from antibody producing cells is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library is constructed by combining the heavy chain gene library with the light chain gene library. This results in a library of clones which co-express a heavy and light chain (resembling the Fab fragment or antigen binding fragment of an antibody molecule). The vectors that carry these genes are co-transfected into a host cell. When antibody gene synthesis is induced in the transfected host, the heavy and light chain proteins self-assemble to produce active antibodies that can be detected by screening with the antigen or immunogen.

Antibody coding sequences of interest include those encoded by native sequences, as well as nucleic acids that, by virtue of the degeneracy of the genetic code, are not identical in sequence to the disclosed nucleic acids, and variants thereof. Variant polypeptides can include amino acid (aa) substitutions, additions or deletions. The amino acid substitutions can be conservative amino acid substitutions or substitutions to eliminate non-essential amino acids, such as to alter a glycosylation site, or to minimize misfolding by substitution or deletion of at least one cysteine residues that are not necessary for function. Variants can be designed so as to retain or have enhanced biological activity of a particular region of the protein (e.g., a functional domain, catalytic amino acid residues). Variants also include fragments of the polypeptides disclosed herein, particularly biologically active fragments and/or fragments corresponding to functional domains. Techniques for in vitro mutagenesis of cloned genes are known. Also included in the subject technology are polypeptides that have been modified using ordinary molecular biological techniques so as to improve their resistance to proteolytic degradation or to optimize solubility properties or to render them more suitable as a therapeutic agent.

Chimeric antibodies may be made by recombinant means by combining the variable light and heavy chain regions (V_{L} and V_{H}), obtained from antibody producing cells of one species with the constant light and heavy chain regions from another. Typically chimeric antibodies utilize rodent or rabbit variable regions and human constant regions, in order to produce an antibody with predominantly human domains. The production of such chimeric antibodies is well known in the art, and may be achieved by standard means (as described, e.g., in U.S. Pat. No. 5,624,659, incorporated herein by reference in its entirety). It is further contemplated that the human constant regions of chimeric antibodies of the subject technology may be selected from IgG1, IgG2, IgG3, IgG4, IgG5, IgG6, IgG7, IgG8, IgG9, IgG10, IgG11, IgG12, IgG13, IgG14, IgG15, IgG16, IgG17, IgG18 or IgG19 constant regions.

Humanized antibodies are engineered to contain even more human-like immunoglobulin domains, and incorporate only the complementarity-determining regions of the animal-derived antibody. This is accomplished by carefully examining the sequence of the hyper-variable loops of the variable regions of the monoclonal antibody, and fitting them to the structure of the human antibody chains. Although facially complex, the process is straightforward in practice. See, e.g., U.S. Pat. No. 6,187,287. In a preferred embodiment, humanization may be effected as disclosed in detail infra. This scheme grafts CDRs onto human FRs highly homologous to the parent antibody that is being humanized.

Immunoglobulins and fragments thereof may be modified post-translationally, e.g. to add effector moieties such as chemical linkers, detectable moieties, such as fluorescent dyes, enzymes, toxins, substrates, bioluminescent materials, radioactive materials, chemiluminescent moieties and the like, or specific binding moieties, such as streptavidin, avidin, or biotin, and the like may be utilized in the methods and compositions of the present subject technology. Examples of additional effector molecules are provided infra.

The subject technology also includes antibodies having binding specificity to IL-6 and possessing a variable heavy chain sequence comprising the sequence set forth in the polypeptide sequences of SEQ ID NO: 3 and SEQ ID NO: 657 and humanized versions and variants thereof including those set forth in FIGS. 1 and 8-11, and those identified in Table 1.

The subject technology further includes antibodies having binding specificity to IL-6 and possessing a variable heavy chain sequence which is a modified version of SEQ ID NO: 3 wherein the tryptophan residue in CDR2 is changed to a serine as set forth in the polypeptide sequence of SEQ ID NO: 658 and humanized versions and variants thereof including those set forth in FIGS. 1 and 8-11, and those identified in Table 1.

The subject technology further contemplates antibodies comprising at least one of the polypeptide sequences of SEQ ID NO: 4; SEQ ID NO: 5; and SEQ ID NO: 6 which correspond to the complementarity-determining regions (CDRs, or hypervariable regions) of the variable light chain sequence of SEQ ID NOs: 2 or 709, and/or at least one of the polypeptide sequences of SEQ ID NO: 7; SEQ ID NO: 8 or 120; and SEQ ID NO: 9 which correspond to the complementarity-determining regions (CDRs, or hypervariable regions) of the variable heavy chain sequence of SEQ ID NOs: 3 or 19 or 657, or combinations of these polypeptide sequences. In another embodiment of the subject technology, the antibodies of the subject technology include combinations of the CDRs and the variable heavy and light chain sequences set forth above.

In another embodiment, the subject technology contemplates other antibodies, such as for example chimeric antibodies, comprising at least one of the polypeptide sequences of SEQ ID NO: 4; SEQ ID NO: 5; and SEQ ID NO: 6 which correspond to the complementarity-determining regions (CDRs, or hypervariable regions) of the variable light chain sequence of SEQ ID NOs: 2 or 709, and/or at least one of the polypeptide sequences of SEQ ID NO: 7; SEQ ID NO: 8 or 120; and SEQ ID NO: 9 which correspond to the complementarity-determining regions (CDRs, or hypervariable regions) of the variable heavy chain sequence of SEQ ID NOs: 3 or 19 or 657, or combinations of these polypeptide sequences. In another embodiment of the subject technology, the antibodies of the subject technology include combinations of the CDRs and humanized versions of the variable heavy and light chain sequences set forth above.

The subject technology also contemplates fragments of the antibody having binding specificity to IL-6. In one embodiment of the subject technology, antibody fragments of the subject technology comprise, or alternatively consist of, humanized versions of the polypeptide sequence of SEQ ID NO: 2, 20, 647, 651, 660, 666, 699, 702, 706, or 709. In another embodiment of the subject technology, antibody fragments of the subject technology comprise, or alternatively consist of, humanized versions of the polypeptide sequence of SEQ ID NO: 3, 18, 19, 652, 656, 657, 658, 661, 664, 665, 704, or 708.

In a further embodiment of the subject technology, fragments of the antibody having binding specificity to IL-6 comprise, or alternatively consist of, at least one of the polypeptide sequences of SEQ ID NO: 4; SEQ ID NO: 5; and SEQ ID NO: 6 which correspond to the complementarity-determining regions (CDRs, or hypervariable regions) of the variable light chain sequence of SEQ ID NO: 2 or SEQ ID NO: 709.

In a further embodiment of the subject technology, fragments of the antibody having binding specificity to IL-6 comprise, or alternatively consist of, at least one of the polypeptide sequences of SEQ ID NO: 7; SEQ ID NO: 8 or SEQ ID NO: 120; and SEQ ID NO: 9 which correspond to the complementarity-determining regions (CDRs, or hypervariable regions) of the variable heavy chain sequence of SEQ ID NO: 3 or 657 or 19.

The subject technology also contemplates antibody fragments which include at least one of the antibody fragments described herein. In one embodiment of the subject technology, fragments of the antibodies having binding specificity to IL-6 comprise, or alternatively consist of, one, two, three or more, including all of the following antibody fragments: the variable light chain region of SEQ ID NO: 2; the variable heavy chain region of SEQ ID NO: 3; the complementarity-determining regions (SEQ ID NO: 4; SEQ ID NO: 5; and SEQ ID NO: 6) of the variable light chain region of SEQ ID NOs: 2 or 709; and the complementarity-determining regions (SEQ ID NO: 7; SEQ ID NO: 8 or SEQ ID NO: 120; and SEQ ID NO: 9) of the variable heavy chain region of SEQ ID NOs: 3 or 657 or 19.

The subject technology also contemplates variants wherein either of the heavy chain polypeptide sequences of SEQ ID NO: 18 or SEQ ID NO: 19 is substituted for the heavy chain polypeptide sequence of SEQ ID NOs: 3 or 657; the light chain polypeptide sequence of SEQ ID NO: 20 is substituted for the light chain polypeptide sequence of SEQ ID NO: 2 or SEQ ID NO: 709; and the heavy chain CDR sequence of SEQ ID NO: 120 is substituted for the heavy chain CDR sequence of SEQ ID NO: 8.

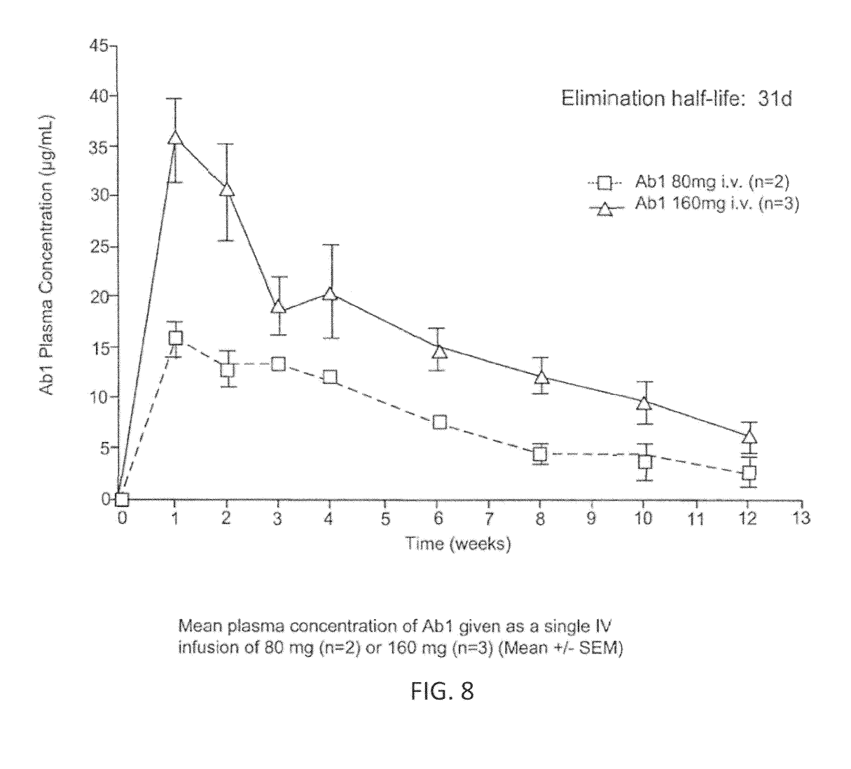

In a preferred embodiment of the subject technology, the anti-IL-6 antibody is Ab1, comprising SEQ ID NO: 2 and SEQ ID NO: 3, or more particularly an antibody comprising SEQ ID NO: 657 and SEQ ID NO: 709 (which are respectively encoded by the nucleic acid sequences in SEQ ID NO: 700 and SEQ ID NO: 723) or one comprised of the alternative SEQ ID NOs set forth in the preceding paragraph, and having at least one of the biological activities set forth herein. In a preferred embodiment the anti-IL-6 antibody will comprise a humanized sequence as shown in FIGS. 8-11.

Sequences of anti-IL-6 antibodies of the present subject technology are shown in Table 1. Exemplary sequence variants other alternative forms of the heavy and light chains of Ab1 through Ab7 are shown. The antibodies of the present subject technology encompass additional sequence variants, including conservative substitutions, substitution of at least one CDR sequences and/or FR sequences.

Exemplary Ab1 embodiments include an antibody comprising a variant of the light chain and/or heavy chain. Exemplary variants of the light chain of Ab1 include the sequence of any of the Ab1 light chains shown (i.e., any of SEQ ID NO: 2, 20, 647, 651, 660, 666, 699, 702, 706, or 709) wherein the entire CDR1 sequence is replaced or wherein at least one residues in the CDR1 sequence is substituted by the residue in the corresponding position of any of the other light chain CDR1 sequences set forth (i.e., any of SEQ ID NO: 23, 39, 55, 71, 87, 103, 124, 140, 156, 172, 188, 204, 220, 236, 252, 268, 284, 300, 316, 332, 348, 364, 380, 396, 412, 428, 444, 460, 476, 492, 508, 524, 540, 556, or 572); and/or wherein the entire CDR2 sequence is replaced or wherein at least one residues in the CDR2 sequence is substituted by the residue in the corresponding position of any of the other light chain CDR2 sequences set forth (i.e., any of SEQ ID NOs: 24, 40, 56, 72, 88, 104, 125, 141, 157, 173, 189, 205, 221, 237, 253, 269, 285, 301, 317, 333, 349, 365, 381, 397, 413, 429, 445, 461, 477, 493, 509, 525, 541, 557, or 573); and/or wherein the entire CDR3 sequence is replaced or wherein at least one residues in the CDR3 sequence is substituted by the residue in the corresponding position of any of the other light chain CDR3 sequences set forth (i.e., any of SEQ ID NOs: 25, 41, 57, 73, 89, 105, 126, 142, 158, 174, 190, 206, 222, 238, 254, 270, 286, 302, 318, 334, 350, 366, 382, 398, 414, 430, 446, 462, 478, 494, 510, 526, 542, 558, or 574).

Exemplary variants of the heavy chain of Ab1 include the sequence of any of the Ab1 heavy chains shown (i.e., any of SEQ ID NO: 3, 18, 19, 652, 656, 657, 658, 661, 664, 665, 704, or 708) wherein the entire CDR1 sequence is replaced or wherein at least one residues in the CDR1 sequence is substituted by the residue in the corresponding position of any of the other heavy chain CDR1 sequences set forth (i.e., any of SEQ ID NO: 26, 42, 58, 74, 90, 106, 127, 143, 159, 175, 191, 207, 223, 239, 255, 271, 287, 303, 319, 335, 351, 367, 383, 399, 415, 431, 447, 463, 479, 495, 511, 527, 543, 559, or 575); and/or wherein the entire CDR2 sequence is replaced or wherein at least one residues in the CDR2 sequence is substituted by the residue in the corresponding position of an Ab1 heavy chain CDR2, such as those set forth in Table 1 (i.e., any of SEQ ID NO: 8, or 120) or any of the other heavy chain CDR2 sequences set forth (i.e., any of SEQ ID NO: 27, 43, 59, 75, 91, 107, 121, 128, 144, 160, 176, 192, 208, 224, 240, 256, 272, 288, 304, 320, 336, 352, 368, 384, 400, 416, 432, 448, 464, 480, 496, 512, 528, 544, 560, or 576); and/or wherein the entire CDR3 sequence is replaced or wherein at least one residues in the CDR3 sequence is substituted by the residue in the corresponding position of any of the other heavy chain CDR3 sequences set forth (i.e., any of SEQ ID NO: 28, 44, 60, 76, 92, 108, 129, 145, 161, 177, 193, 209, 225, 241, 257, 273, 289, 305, 321, 337, 353, 369, 385, 401, 417, 433, 449, 465, 481, 497, 513, 529, 545, 561, or 577).

In another embodiment, the subject technology contemplates other antibodies, such as for example chimeric or humanized antibodies, comprising at least one of the polypeptide sequences of SEQ ID NO: 4; SEQ ID NO: 5; and SEQ ID NO: 6 which correspond to the complementarity-determining regions (CDRs, or hypervariable regions) of the variable light chain sequence of SEQ ID NO: 2, and/or at least one of the polypeptide sequences of SEQ ID NO: 7 (CDR1); SEQ ID NO: 8 (CDR2); SEQ ID NO: 120 (CDR2); and SEQ ID NO: 9 (CDR3) which correspond to the complementarity-determining regions (CDRs, or hypervariable regions) of the variable heavy chain sequence of SEQ ID NO: 3 or SEQ ID NO: 19, or combinations of these polypeptide sequences. In another embodiment of the subject technology, the antibodies of the subject technology include combinations of the CDRs and the variable heavy and light chain sequences set forth above including those set forth in FIGS. 1 and 8-11, and those identified in Table 1.

In another embodiment the anti-IL-6 antibody of the subject technology is one comprising at least one of the following: a CDR1 light chain encoded by the sequence in SEQ ID NO: 12 or SEQ ID NO: 694; a light chain CDR2 encoded by the sequence in SEQ ID NO: 13; a light chain CDR3 encoded by the sequence in SEQ ID NO: 14 or SEQ ID NO: 695; a heavy chain CDR1 encoded by the sequence in SEQ ID NO: 15, a heavy chain CDR2 encoded by SEQ ID NO: 16 or SEQ ID NO: 696 and a heavy chain CDR3 encoded by SEQ ID NO: 17 or SEQ ID NO: 697. In addition the subject technology embraces such nucleic acid sequences and variants thereof.

In another embodiment the subject technology is directed to amino acid sequences corresponding to the CDRs of said anti-IL-6 antibody which are selected from SEQ ID NO: 4 (CDR1), SEQ ID NO: 5 (CDR2), SEQ ID NO: 6 (CDR3), SEQ ID NO: 7, SEQ ID NO: 120 and SEQ ID NO: 9.

In another embodiment the anti-IL-6 antibody of the subject technology comprises a light chain nucleic acid sequence of SEQ ID NO: 10, 662, 698, 701, 705, 720, 721, 722, or 723; and/or a heavy chain nucleic acid sequence of SEQ ID NO: 11, 663, 700, 703, 707, 724, or 725. In addition the subject technology is directed to the corresponding polypeptides encoded by any of the foregoing nucleic acid sequences and combinations thereof.

In a specific embodiment of the subject technology the anti-IL-6 antibodies or a portion thereof will be encoded by a nucleic acid sequence selected from those comprised in SEQ ID NO: 10, 12, 13, 14, 662, 694, 695, 698, 701, 705, 720, 721, 722, 723, 11, 15, 16, 17, 663, 696, 697, 700, 703, 707, 724, and 725. For example the CDR1 in the light chain may be encoded by SEQ ID NO: 12 or 694, the CDR2 in the light chain may be encoded by SEQ ID NO: 13, the CDR3 in the light chain may be encoded by SEQ ID NO: 14 or 695; the CDR1 in the heavy chain may be encoded by SEQ ID NO: 15, the CDR2 in the heavy chain may be encoded by SEQ ID NO: 16 or 696, the CDR3 in the heavy chain may be encoded by SEQ ID NO: 17 or 697. As discussed infra antibodies containing these CDRs may be constructed using appropriate human frameworks based on the humanization methods disclosed herein.

In another specific embodiment of the subject technology the variable light chain will be encoded by SEQ ID NO: 10, 662, 698, 701, 705, 720, 721, 722, or 723 and the variable heavy chain of the anti-IL-6 antibodies will be encoded by SEQ ID NO: 11, 663, 700, 703, 707, 724, or 725.

In a more specific embodiment variable light and heavy chains of the anti-IL-6 antibody respectively will be encoded by SEQ ID NO: 10 and 11, or SEQ ID NO: 698 and SEQ ID NO: 700 or SEQ ID NO: 701 and SEQ ID NO: 703 or SEQ ID NO: 705 and SEQ ID NO: 707.

In another specific embodiment the subject technology covers nucleic acid constructs containing any of the foregoing nucleic acid sequences and combinations thereof as well as recombinant cells containing these nucleic acid sequences and constructs containing wherein these nucleic acid sequences or constructs may be extrachromosomal or integrated into the host cell genome.

In another specific embodiment the subject technology covers polypeptides containing any of the CDRs or combinations thereof recited in SEQ ID NO: 4, SEQ ID NO: 5, SEQ ID NO: 6, SEQ ID NO: 7, SEQ ID NO: 8, SEQ ID NO: 120, SEQ ID NO: 9 or polypeptides comprising any of the variable light polypeptides comprised in SEQ ID NO: 2, 20, 647, 651, 660, 666, 699, 702, 706, or 709 and/or the variable heavy polypeptides comprised in SEQ ID NO: 3, 18, 19, 652, 656, 657, 658, 661, 664, 665, 704, or 708. These polypeptides optionally may be attached directly or indirectly to other immunoglobulin polypeptides or effector moieties such as therapeutic or detectable entities.

In another embodiment the anti-IL-6 antibody is one comprising at least one of the following: a variable light chain encoded by the sequence in SEQ ID NO: 10 or SEQ ID NO: 698 or SEQ ID NO: 701 or SEQ ID NO: 705 and a variable chain encoded by the sequence in SEQ ID NO: 11 or SEQ ID NO: 700 or SEQ ID NO: 703 or SEQ ID NO: 707.

In another embodiment the anti-IL-6 antibody is a variant of the foregoing sequences that includes at least one substitution in the framework and/or CDR sequences and which has at least one of the properties of Ab1 in vitro and/or upon in vivo administration.

These in vitro and in vivo properties are described in more detail in the examples below and include: competing with Ab1 for binding to IL-6 and/or peptides thereof; having a binding affinity (Kd) for IL-6 of less than about 50 picomolar, and/or a rate of dissociation (K_{off}) from IL-6 of less than or equal to 10⁻⁴ S⁻¹; having an in-vivo half-life of at least about 22 days in a healthy human subject; ability to prevent or treat hypoalbunemia; ability to prevent or treat elevated CRP; ability to prevent or treat abnormal coagulation; and/or ability to decrease the risk of thrombosis in an individual having a disease or condition associated with increased risk of thrombosis. Additional non-limiting examples of anti-IL-6 activity are set forth herein, for example, under the heading “Anti-IL-6 Activity.”

In another embodiment the anti-IL-6 antibody includes at least one of the Ab1 light-chain and/or heavy chain CDR sequences (see Table 1) or variant(s) thereof which has at least one of the properties of Ab1 in vitro and/or upon in vivo administration (examples of such properties are discussed in the preceding paragraph). One of skill in the art would understand how to combine these CDR sequences to form an antigen-binding surface, e.g. by linkage to at least one scaffold which may comprise human or other mammalian framework sequences, or their functional orthologs derived from a SMIP, camelbody, nanobody, IgNAR or other immunoglobulin or other engineered antibody. For example, embodiments may specifically bind to human IL-6 and include one, two, three, four, five, six, or more of the following CDR sequences or variants thereof: a polypeptide having at least 72.7% (i.e., 8 out of 11 amino acids) identity to the light chain CDR1 of SEQ ID NO: 4; a polypeptide having at least 81.8% (i.e., 9 out of 11 amino acids) identity to the light chain CDR1 of SEQ ID NO: 4; a polypeptide having at least 90.9% (i.e., 10 out of 11 amino acids) identity to the light chain CDR1 of SEQ ID NO: 4; a polypeptide having 100% (i.e., 11 out of 11 amino acids) identity to the light chain CDR1 of SEQ ID NO: 4; a polypeptide having at least 85.7% (i.e., 6 out of 7 amino acids) identity to the light chain CDR2 of SEQ ID NO: 5; a polypeptide having 100% (i.e., 7 out of 7 amino acids) identity to the light chain CDR2 of SEQ ID NO: 5; a polypeptide having at least 50% (i.e., 6 out of 12 amino acids) identity to the light chain CDR3 of SEQ ID NO: 6; a polypeptide having at least 58.3% (i.e., 7 out of 12 amino acids) identity to the light chain CDR3 of SEQ ID NO: 6;

a polypeptide having at least 66.6% (i.e., 8 out of 12 amino acids) identity to the light chain CDR3 of SEQ ID NO: 6; a polypeptide having at least 75% (i.e., 9 out of 12 amino acids) identity to the light chain CDR3 of SEQ ID NO: 6; a polypeptide having at least 83.3% (i.e., 10 out of 12 amino acids) identity to the light chain CDR3 of SEQ ID NO: 6; a polypeptide having at least 91.6% (i.e., 11 out of 12 amino acids) identity to the light chain CDR3 of SEQ ID NO: 6; a polypeptide having 100% (i.e., 12 out of 12 amino acids) identity to the light chain CDR3 of SEQ ID NO: 6; a polypeptide having at least 80% (i.e., 4 out of 5 amino acids) identity to the heavy chain CDR1 of SEQ ID NO: 7; a polypeptide having 100% (i.e., 5 out of 5 amino acids) identity to the heavy chain CDR1 of SEQ ID NO: 7; a polypeptide having at least 50% (i.e., 8 out of 16 amino acids) identity to the heavy chain CDR2 of SEQ ID NO: 120; a polypeptide having at least 56.2% (i.e., 9 out of 16 amino acids) identity to the heavy chain CDR2 of SEQ ID NO: 120; a polypeptide having at least 62.5% (i.e., 10 out of 16 amino acids) identity to the heavy chain CDR2 of SEQ ID NO: 120; a polypeptide having at least 68.7% (i.e., 11 out of 16 amino acids) identity to the heavy chain CDR2 of SEQ ID NO: 120; a polypeptide having at least 75% (i.e., 12 out of 16 amino acids) identity to the heavy chain CDR2 of SEQ ID NO: 120;

a polypeptide having at least 81.2% (i.e., 13 out of 16 amino acids) identity to the heavy chain CDR2 of SEQ ID NO: 120; a polypeptide having at least 87.5% (i.e., 14 out of 16 amino acids) identity to the heavy chain CDR2 of SEQ ID NO: 120; a polypeptide having at least 93.7% (i.e., 15 out of 16 amino acids) identity to the heavy chain CDR2 of SEQ ID NO: 120; a polypeptide having 100% (i.e., 16 out of 16 amino acids) identity to the heavy chain CDR2 of SEQ ID NO: 120; a polypeptide having at least 33.3% (i.e., 4 out of 12 amino acids) identity to the heavy chain CDR3 of SEQ ID NO: 9; a polypeptide having at least 41.6% (i.e., 5 out of 12 amino acids) identity to the heavy chain CDR3 of SEQ ID NO: 9; a polypeptide having at least 50% (i.e., 6 out of 12 amino acids) identity to the heavy chain CDR3 of SEQ ID NO: 9; a polypeptide having at least 58.3% (i.e., 7 out of 12 amino acids) identity to the heavy chain CDR3 of SEQ ID NO: 9; a polypeptide having at least 66.6% (i.e., 8 out of 12 amino acids) identity to the heavy chain CDR3 of SEQ ID NO: 9; a polypeptide having at least 75% (i.e., 9 out of 12 amino acids) identity to the heavy chain CDR3 of SEQ ID NO: 9; a polypeptide having at least 83.3% (i.e., 10 out of 12 amino acids) identity to the heavy chain CDR3 of SEQ ID NO: 9; a polypeptide having at least 91.6% (i.e., 11 out of 12 amino acids) identity to the heavy chain CDR3 of SEQ ID NO: 9; a polypeptide having 100% (i.e., 12 out of 12 amino acids) identity to the heavy chain CDR3 of SEQ ID NO: 9; a polypeptide having at least 90.9% (i.e., 10 out of 11 amino acids) similarity to the light chain CDR1 of SEQ ID NO: 4; a polypeptide having 100% (i.e., 11 out of 11 amino acids) similarity to the light chain CDR1 of SEQ ID NO: 4; a polypeptide having at least 85.7% (i.e., 6 out of 7 amino acids) similarity to the light chain CDR2 of SEQ ID NO: 5; a polypeptide having 100% (i.e., 7 out of 7 amino acids) similarity to the light chain CDR2 of SEQ ID NO: 5; a polypeptide having at least 66.6% (i.e., 8 out of 12 amino acids) similarity to the light chain CDR3 of SEQ ID NO: 6; a polypeptide having at least 75% (i.e., 9 out of 12 amino acids) similarity to the light chain CDR3 of SEQ ID NO: 6; a polypeptide having at least 83.3% (i.e., 10 out of 12 amino acids) similarity to the light chain CDR3 of SEQ ID NO: 6; a polypeptide having at least 91.6% (i.e., 11 out of 12 amino acids) similarity to the light chain CDR3 of SEQ ID NO: 6; a polypeptide having 100% (i.e., 12 out of 12 amino acids) similarity to the light chain CDR3 of SEQ ID NO: 6; a polypeptide having at least 80% (i.e., 4 out of 5 amino acids) similarity to the heavy chain CDR1 of SEQ ID NO: 7; a polypeptide having 100% (i.e., 5 out of 5 amino acids) similarity to the heavy chain CDR1 of SEQ ID NO: 7; a polypeptide having at least 56.2% (i.e., 9 out of 16 amino acids) similarity to the heavy chain CDR2 of SEQ ID NO: 120; a polypeptide having at least 62.5% (i.e., 10 out of 16 amino acids) similarity to the heavy chain CDR2 of SEQ ID NO: 120; a polypeptide having at least 68.7% (i.e., 11 out of 16 amino acids) similarity to the heavy chain CDR2 of SEQ ID NO: 120; a polypeptide having at least 75% (i.e., 12 out of 16 amino acids) similarity to the heavy chain CDR2 of SEQ ID NO: 120; a polypeptide having at least 81.2% (i.e., 13 out of 16 amino acids) similarity to the heavy chain CDR2 of SEQ ID NO: 120; a polypeptide having at least 87.5% (i.e., 14 out of 16 amino acids) similarity to the heavy chain CDR2 of SEQ ID NO: 120; a polypeptide having at least 93.7% (i.e., 15 out of 16 amino acids) similarity to the heavy chain CDR2 of SEQ ID NO: 120; a polypeptide having 100% (i.e., 16 out of 16 amino acids) similarity to the heavy chain CDR2 of SEQ ID NO: 120; a polypeptide having at least 50% (i.e., 6 out of 12 amino acids) similarity to the heavy chain CDR3 of SEQ ID NO: 9; a polypeptide having at least 58.3% (i.e., 7 out of 12 amino acids) similarity to the heavy chain CDR3 of SEQ ID NO: 9; a polypeptide having at least 66.6% (i.e., 8 out of 12 amino acids) similarity to the heavy chain CDR3 of SEQ ID NO: 9; a polypeptide having at least 75% (i.e., 9 out of 12 amino acids) similarity to the heavy chain CDR3 of SEQ ID NO: 9; a polypeptide having at least 83.3% (i.e., 10 out of 12 amino acids) similarity to the heavy chain CDR3 of SEQ ID NO: 9; a polypeptide having at least 91.6% (i.e., 11 out of 12 amino acids) similarity to the heavy chain CDR3 of SEQ ID NO: 9; or a polypeptide having 100% (i.e., 12 out of 12 amino acids) similarity to the heavy chain CDR3 of SEQ ID NO: 9.

Other exemplary embodiments include at least one polynucleotides encoding any of the foregoing, e.g., a polynucleotide encoding a polypeptide that specifically binds to human IL-6 and includes one, two, three, four, five, six, or more of the following CDRs or variants thereof:

a polynucleotide encoding a polypeptide having at least 72.7% (i.e., 8 out of 11 amino acids) identity to the light chain CDR1 of SEQ ID NO: 4; a polynucleotide encoding a polypeptide having at least 81.8% (i.e., 9 out of 11 amino acids) identity to the light chain CDR1 of SEQ ID NO: 4; a polynucleotide encoding a polypeptide having at least 90.9% (i.e., 10 out of 11 amino acids) identity to the light chain CDR1 of SEQ ID NO: 4; a polynucleotide encoding a polypeptide having 100% (i.e., 11 out of 11 amino acids) identity to the light chain CDR1 of SEQ ID NO: 4; a polynucleotide encoding a polypeptide having at least 85.7% (i.e., 6 out of 7 amino acids) identity to the light chain CDR2 of SEQ ID NO: 5; a polynucleotide encoding a polypeptide having 100% (i.e., 7 out of 7 amino acids) identity to the light chain CDR2 of SEQ ID NO: 5; a polynucleotide encoding a polypeptide having at least 50% (i.e., 6 out of 12 amino acids) identity to the light chain CDR3 of SEQ ID NO: 6; a polynucleotide encoding a polypeptide having at least 58.3% (i.e., 7 out of 12 amino acids) identity to the light chain CDR3 of SEQ ID NO: 6; a polynucleotide encoding a polypeptide having at least 66.6% (i.e., 8 out of 12 amino acids) identity to the light chain CDR3 of SEQ ID NO: 6; a polynucleotide encoding a polypeptide having at least 75% (i.e., 9 out of 12 amino acids) identity to the light chain CDR3 of SEQ ID NO: 6; a polynucleotide encoding a polypeptide having at least 83.3% (i.e., 10 out of 12 amino acids) identity to the light chain CDR3 of SEQ ID NO: 6; a polynucleotide encoding a polypeptide having at least 91.6% (i.e., 11 out of 12 amino acids) identity to the light chain CDR3 of SEQ ID NO: 6; a polynucleotide encoding a polypeptide having 100% (i.e., 12 out of 12 amino acids) identity to the light chain CDR3 of SEQ ID NO: 6; a polynucleotide encoding a polypeptide having at least 80% (i.e., 4 out of 5 amino acids) identity to the heavy chain CDR1 of SEQ ID NO: 7; a polynucleotide encoding a polypeptide having 100% (i.e., 5 out of 5 amino acids) identity to the heavy chain CDR1 of SEQ ID NO: 7; a polynucleotide encoding a polypeptide having at least 50% (i.e., 8 out of 16 amino acids) identity to the heavy chain CDR2 of SEQ ID NO: 120; a polynucleotide encoding a polypeptide having at least 56.2% (i.e., 9 out of 16 amino acids) identity to the heavy chain CDR2 of SEQ ID NO: 120; a polynucleotide encoding a polypeptide having at least 62.5% (i.e., 10 out of 16 amino acids) identity to the heavy chain CDR2 of SEQ ID NO: 120; a polynucleotide encoding a polypeptide having at least 68.7% (i.e., 11 out of 16 amino acids) identity to the heavy chain CDR2 of SEQ ID NO: 120; a polynucleotide encoding a polypeptide having at least 75% (i.e., 12 out of 16 amino acids) identity to the heavy chain CDR2 of SEQ ID NO: 120; a polynucleotide encoding a polypeptide having at least 81.2% (i.e., 13 out of 16 amino acids) identity to the heavy chain CDR2 of SEQ ID NO: 120; a polynucleotide encoding a polypeptide having at least 87.5% (i.e., 14 out of 16 amino acids) identity to the heavy chain CDR2 of SEQ ID NO: 120; a polynucleotide encoding a polypeptide having at least 93.7% (i.e., 15 out of 16 amino acids) identity to the heavy chain CDR2 of SEQ ID NO: 120; a polynucleotide encoding a polypeptide having 100% (i.e., 16 out of 16 amino acids) identity to the heavy chain CDR2 of SEQ ID NO: 120; a polynucleotide encoding a polypeptide having at least 33.3% (i.e., 4 out of 12 amino acids) identity to the heavy chain CDR3 of SEQ ID NO: 9; a polynucleotide encoding a polypeptide having at least 41.6% (i.e., 5 out of 12 amino acids) identity to the heavy chain CDR3 of SEQ ID NO: 9; a polynucleotide encoding a polypeptide having at least 50% (i.e., 6 out of 12 amino acids) identity to the heavy chain CDR3 of SEQ ID NO: 9; a polynucleotide encoding a polypeptide having at least 58.3% (i.e., 7 out of 12 amino acids) identity to the heavy chain CDR3 of SEQ ID NO: 9; a polynucleotide encoding a polypeptide having at least 66.6% (i.e., 8 out of 12 amino acids) identity to the heavy chain CDR3 of SEQ ID NO: 9; a polynucleotide encoding a polypeptide having at least 75% (i.e., 9 out of 12 amino acids) identity to the heavy chain CDR3 of SEQ ID NO: 9; a polynucleotide encoding a polypeptide having at least 83.3% (i.e., 10 out of 12 amino acids) identity to the heavy chain CDR3 of SEQ ID NO: 9; a polynucleotide encoding a polypeptide having at least 91.6% (i.e., 11 out of 12 amino acids) identity to the heavy chain CDR3 of SEQ ID NO: 9; a polynucleotide encoding a polypeptide having 100% (i.e., 12 out of 12 amino acids) identity to the heavy chain CDR3 of SEQ ID NO: 9; a polynucleotide encoding a polypeptide having at least 90.9% (i.e., 10 out of 11 amino acids) similarity to the light chain CDR1 of SEQ ID NO: 4; a polynucleotide encoding a polypeptide having 100% (i.e., 11 out of 11 amino acids) similarity to the light chain CDR1 of SEQ ID NO: 4; a polynucleotide encoding a polypeptide having at least 85.7% (i.e., 6 out of 7 amino acids) similarity to the light chain CDR2 of SEQ ID NO: 5; a polynucleotide encoding a polypeptide having 100% (i.e., 7 out of 7 amino acids) similarity to the light chain CDR2 of SEQ ID NO: 5; a polynucleotide encoding a polypeptide having at least 66.6% (i.e., 8 out of 12 amino acids) similarity to the light chain CDR3 of SEQ ID NO: 6; a polynucleotide encoding a polypeptide having at least 75% (i.e., 9 out of 12 amino acids) similarity to the light chain CDR3 of SEQ ID NO: 6; a polynucleotide encoding a polypeptide having at least 83.3% (i.e., 10 out of 12 amino acids) similarity to the light chain CDR3 of SEQ ID NO: 6; a polynucleotide encoding a polypeptide having at least 91.6% (i.e., 11 out of 12 amino acids) similarity to the light chain CDR3 of SEQ ID NO: 6; a polynucleotide encoding a polypeptide having 100% (i.e., 12 out of 12 amino acids) similarity to the light chain CDR3 of SEQ ID NO: 6; a polynucleotide encoding a polypeptide having at least 80% (i.e., 4 out of 5 amino acids) similarity to the heavy chain CDR1 of SEQ ID NO: 7; a polynucleotide encoding a polypeptide having 100% (i.e., 5 out of 5 amino acids) similarity to the heavy chain CDR1 of SEQ ID NO: 7; a polynucleotide encoding a polypeptide having at least 56.2% (i.e., 9 out of 16 amino acids) similarity to the heavy chain CDR2 of SEQ ID NO: 120; a polynucleotide encoding a polypeptide having at least 62.5% (i.e., 10 out of 16 amino acids) similarity to the heavy chain CDR2 of SEQ ID NO: 120; a polynucleotide encoding a polypeptide having at least 68.7% (i.e., 11 out of 16 amino acids) similarity to the heavy chain CDR2 of SEQ ID NO: 120; a polynucleotide encoding a polypeptide having at least 75% (i.e., 12 out of 16 amino acids) similarity to the heavy chain CDR2 of SEQ ID NO: 120; a polynucleotide encoding a polypeptide having at least 81.2% (i.e., 13 out of 16 amino acids) similarity to the heavy chain CDR2 of SEQ ID NO: 120; a polynucleotide encoding a polypeptide having at least 87.5% (i.e., 14 out of 16 amino acids) similarity to the heavy chain CDR2 of SEQ ID NO: 120; a polynucleotide encoding a polypeptide having at least 93.7% (i.e., 15 out of 16 amino acids) similarity to the heavy chain CDR2 of SEQ ID NO: 120;

a polynucleotide encoding a polypeptide having 100% (i.e., 16 out of 16 amino acids) similarity to the heavy chain CDR2 of SEQ ID NO: 120; a polynucleotide encoding a polypeptide having at least 50% (i.e., 6 out of 12 amino acids) similarity to the heavy chain CDR3 of SEQ ID NO: 9; a polynucleotide encoding a polypeptide having at least 58.3% (i.e., 7 out of 12 amino acids) similarity to the heavy chain CDR3 of SEQ ID NO: 9; a polynucleotide encoding a polypeptide having at least 66.6% (i.e., 8 out of 12 amino acids) similarity to the heavy chain CDR3 of SEQ ID NO: 9; a polynucleotide encoding a polypeptide having at least 75% (i.e., 9 out of 12 amino acids) similarity to the heavy chain CDR3 of SEQ ID NO: 9; a polynucleotide encoding a polypeptide having at least 83.3% (i.e., 10 out of 12 amino acids) similarity to the heavy chain CDR3 of SEQ ID NO: 9; a polynucleotide encoding a polypeptide having at least 91.6% (i.e., 11 out of 12 amino acids) similarity to the heavy chain CDR3 of SEQ ID NO: 9; a polynucleotide encoding a polypeptide having 100% (i.e., 12 out of 12 amino acids) similarity to the heavy chain CDR3 of SEQ ID NO: 9.

TABLE 1
Sequences of exemplary anti-IL-6 antibodies.

| | Antibody chains | | CDR1 | | CDR2 | | CDR3 | |
|---|---|---|---|---|---|---|---|---|
| Antibody | PRT. | Nuc. | PRT. | Nuc. | PRT. | Nuc. | PRT. | Nuc. |
| Ab1 light chains * | 2 | 10 | 4 | 12 | 5 | 13 | 6 | 14 |
| | 20 | 720 | 4 | 12 | 5 | 13 | 6 | 14 |
| | 647 | 721 | 4 | 12 | 5 | 13 | 6 | 14 |
| | 651 | | 4 | 12 | 5 | 13 | 6 | 14 |
| | 660 | 662 | 4 | 12 | 5 | 13 | 6 | 14 |
| | 666 | 722 | 4 | 12 | 5 | 13 | 6 | 14 |
| | 699 | 698 | 4 | 694 | 5 | 13 | 6 | 695 |
| | 702 | 701 | 4 | 694 | 5 | 13 | 6 | 695 |
| | 706 | 705 | 4 | 694 | 5 | 13 | 6 | 695 |
| | 709 | 723 | 4 | 12 | 5 | 13 | 6 | 14 |
| Human light | 648 | | 710 | | 713 | | | |
| chains used in | 649 | | 711 | | 714 | | | |
| Ab1 humanization | 650 | | 712 | | 715 | | | |
| Ab1 heavy chains | 3 | 11 | 7 | 15 | 8 | 16 | 9 | 17 |
| | 18 | | 7 | 15 | 8 | 16 | 9 | 17 |
| | 19 | 724 | 7 | 15 | 120 | 696 | 9 | 17 |
| | 652 | 725 | 7 | 15 | 8 | 16 | 9 | 17 |
| | 656 | | 7 | 15 | 8 | 16 | 9 | 17 |
| | 657 | 700 | 7 | 15 | 659 | 696 | 9 | 697 |
| | 658 | | 7 | 15 | 120 | 696 | 9 | 17 |
| | 661 | 663 | 7 | 15 | 8 | 16 | 9 | 17 |
| | 664 | | 7 | 15 | 8 | 16 | 9 | 17 |
| | 665 | | 7 | 15 | 120 | 696 | 9 | 17 |
| | 704 | 703 | 7 | 15 | 120 | 696 | 9 | 697 |
| | 708 | 707 | 7 | 15 | 120 | 696 | 9 | 697 |
| Human heavy | 653 | | 716 | | 717 | | | |
| chains used in | 654 | | 716 | | 717 | | | |
| Ab1 humanization | 655 | | 74 | 82 | 718 | | | |
| Ab2 light chains | 21 | 29 | 23 | 31 | 24 | 32 | 25 | 33 |
| | 667 | 669 | 23 | 31 | 24 | 32 | 25 | 33 |
| Ab2 heavy chains | 22 | 30 | 26 | 34 | 27 | 35 | 28 | 36 |
| | 668 | 670 | 26 | 34 | 27 | 35 | 28 | 36 |
| Ab3 light chains | 37 | 45 | 39 | 47 | 40 | 48 | 41 | 49 |
| | 671 | 673 | 39 | 47 | 40 | 48 | 41 | 49 |
| Ab3 heavy chains | 38 | 46 | 42 | 50 | 43 | 51 | 44 | 52 |
| | 672 | 674 | 42 | 50 | 43 | 51 | 44 | 52 |
| Ab4 light chains | 53 | 61 | 55 | 63 | 56 | 64 | 57 | 65 |
| | 675 | 677 | 55 | 63 | 56 | 64 | 57 | 65 |
| Ab4 heavy chains | 54 | 62 | 58 | 66 | 59 | 67 | 60 | 68 |
| | 676 | 678 | 58 | 66 | 59 | 67 | 60 | 68 |
| Ab5 light chains | 69 | 77 | 71 | 79 | 72 | 80 | 73 | 81 |
| | 679 | 681 | 71 | 79 | 72 | 80 | 73 | 81 |
| Ab5 heavy chains | 70 | 78 | 74 | 82 | 75 | 83 | 76 | 84 |
| | 680 | 682 | 74 | 82 | 75 | 83 | 76 | 84 |
| Ab6 light chains | 85 | 93 | 87 | 95 | 88 | 96 | 89 | 97 |
| | 683 | 685 | 87 | 95 | 88 | 96 | 89 | 97 |
| Ab6 heavy chains | 86 | 94 | 90 | 98 | 91 | 99 | 92 | 100 |
| | 684 | 686 | 90 | 98 | 91 | 99 | 92 | 100 |
| Ab7 light chains | 101 | 109 | 103 | 111 | 104 | 112 | 105 | 113 |
| | 119 | | 103 | 111 | 104 | 112 | 105 | 113 |
| | 687 | 689 | 103 | 111 | 104 | 112 | 105 | 113 |
| | 693 | | 103 | 111 | 104 | 112 | 105 | 113 |
| Ab7 heavy chains | 102 | 110 | 106 | 114 | 107 | 115 | 108 | 116 |
| | 117 | | 106 | 114 | 107 | 115 | 108 | 116 |
| | 118 | | 106 | 114 | 121 | | 108 | 116 |
| | 688 | 690 | 106 | 114 | 107 | 115 | 108 | 116 |
| | 691 | | 106 | 114 | 107 | 115 | 108 | 116 |
| | 692 | | 106 | 114 | 121 | | 108 | 116 |
| Ab8 light chain | 122 | 130 | 124 | 132 | 125 | 133 | 126 | 134 |
| Ab8 heavy chain | 123 | 131 | 127 | 135 | 128 | 136 | 129 | 137 |
| Ab9 light chain | 138 | 146 | 140 | 148 | 141 | 149 | 142 | 150 |
| Ab9 heavy chain | 139 | 147 | 143 | 151 | 144 | 152 | 145 | 153 |
| Ab10 light chain | 154 | 162 | 156 | 164 | 157 | 165 | 158 | 166 |
| Ab10 heavy chain | 155 | 163 | 159 | 167 | 160 | 168 | 161 | 169 |
| Ab11 light chain | 170 | 178 | 172 | 180 | 173 | 181 | 174 | 182 |
| Ab11 heavy chain | 171 | 179 | 175 | 183 | 176 | 184 | 177 | 185 |
| Ab12 light chain | 186 | 194 | 188 | 196 | 189 | 197 | 190 | 198 |
| Ab12 heavy chain | 187 | 195 | 191 | 199 | 192 | 200 | 193 | 201 |
| Ab13 light chain | 202 | 210 | 204 | 212 | 205 | 213 | 206 | 214 |
| Ab13 heavy chain | 203 | 211 | 207 | 215 | 208 | 216 | 209 | 217 |
| Ab14 light chain | 218 | 226 | 220 | 228 | 221 | 229 | 222 | 230 |
| Ab14 heavy chain | 219 | 227 | 223 | 231 | 224 | 232 | 225 | 233 |
| Ab15 light chain | 234 | 242 | 236 | 244 | 237 | 245 | 238 | 246 |
| Ab15 heavy chain | 235 | 243 | 239 | 247 | 240 | 248 | 241 | 249 |
| Ab16 light chain | 250 | 258 | 252 | 260 | 253 | 261 | 254 | 262 |
| Ab16 heavy chain | 251 | 259 | 255 | 263 | 256 | 264 | 257 | 265 |
| Ab17 light chain | 266 | 274 | 268 | 276 | 269 | 277 | 270 | 278 |
| Ab17 heavy chain | 267 | 275 | 271 | 279 | 272 | 280 | 273 | 281 |
| Ab18 light chain | 282 | 290 | 284 | 292 | 285 | 293 | 286 | 294 |
| Ab18 heavy chain | 283 | 291 | 287 | 295 | 288 | 296 | 289 | 297 |
| Ab19 light chain | 298 | 306 | 300 | 308 | 301 | 309 | 302 | 310 |
| Ab19 heavy chain | 299 | 307 | 303 | 311 | 304 | 312 | 305 | 313 |
| Ab20 light chain | 314 | 322 | 316 | 324 | 317 | 325 | 318 | 326 |
| Ab20 heavy chain | 315 | 323 | 319 | 327 | 320 | 328 | 321 | 329 |
| Ab21 light chain | 330 | 338 | 332 | 340 | 333 | 341 | 334 | 342 |
| Ab21 heavy chain | 331 | 339 | 335 | 343 | 336 | 344 | 337 | 345 |
| Ab22 light chain | 346 | 354 | 348 | 356 | 349 | 357 | 350 | 358 |
| Ab22 heavy chain | 347 | 355 | 351 | 359 | 352 | 360 | 353 | 361 |
| Ab23 light chain | 362 | 370 | 364 | 372 | 365 | 373 | 366 | 374 |
| Ab23 heavy chain | 363 | 371 | 367 | 375 | 368 | 376 | 369 | 377 |
| Ab24 light chain | 378 | 386 | 380 | 388 | 381 | 389 | 382 | 390 |
| Ab24 heavy chain | 379 | 387 | 383 | 391 | 384 | 392 | 385 | 393 |
| Ab25 light chain | 394 | 402 | 396 | 404 | 397 | 405 | 398 | 406 |
| Ab25 heavy chain | 395 | 403 | 399 | 407 | 400 | 408 | 401 | 409 |
| Ab26 light chain | 410 | 418 | 412 | 420 | 413 | 421 | 414 | 422 |
| Ab26 heavy chain | 411 | 419 | 415 | 423 | 416 | 424 | 417 | 425 |
| Ab27 light chain | 426 | 434 | 428 | 436 | 429 | 437 | 430 | 438 |
| Ab27 heavy chain | 427 | 435 | 431 | 439 | 432 | 440 | 433 | 441 |
| Ab28 light chain | 442 | 450 | 444 | 452 | 445 | 453 | 446 | 454 |
| Ab28 heavy chain | 443 | 451 | 447 | 455 | 448 | 456 | 449 | 457 |
| Ab29 light chain | 458 | 466 | 460 | 468 | 461 | 469 | 462 | 470 |
| Ab29 heavy chain | 459 | 467 | 463 | 471 | 464 | 472 | 465 | 473 |
| Ab30 light chain | 474 | 482 | 476 | 484 | 477 | 485 | 478 | 486 |
| Ab30 heavy chain | 475 | 483 | 479 | 487 | 480 | 488 | 481 | 489 |
| Ab31 light chain | 490 | 498 | 492 | 500 | 493 | 501 | 494 | 502 |
| Ab31 heavy chain | 491 | 499 | 495 | 503 | 496 | 504 | 497 | 505 |
| Ab32 light chain | 506 | 514 | 508 | 516 | 509 | 517 | 510 | 518 |
| Ab32 heavy chain | 507 | 515 | 511 | 519 | 512 | 520 | 513 | 521 |
| Ab33 light chain | 522 | 530 | 524 | 532 | 525 | 533 | 526 | 534 |
| Ab33 heavy chain | 523 | 531 | 527 | 535 | 528 | 536 | 529 | 537 |
| Ab34 light chain | 538 | 546 | 540 | 548 | 541 | 549 | 542 | 550 |
| Ab34 heavy chain | 539 | 547 | 543 | 551 | 544 | 552 | 545 | 553 |
| Ab35 light chain | 554 | 562 | 556 | 564 | 557 | 565 | 558 | 566 |
| Ab35 heavy chain | 555 | 563 | 559 | 567 | 560 | 568 | 561 | 569 |
| Ab36 light chain | 570 | 578 | 572 | 580 | 573 | 581 | 574 | 582 |
| Ab36 heavy chain | 571 | 579 | 575 | 583 | 576 | 584 | 577 | 585 |

* Exemplary sequence variant forms of heavy and light chains are shown on separate lines.
PRT.: Polypeptide sequence.
Nuc.: Exemplary coding sequence.

For reference, sequence identifiers other than those included in Table 1 are summarized in Table 2.

TABLE 2
Summary of sequence identifiers in this application.

| SEQ ID | Description |
|---|---|
| 1 | Human IL-6 |
| 586 | kappa constant light chain polypeptide sequence |
| 587 | kappa constant light chain polynucleotide sequence |
| 588 | gamma-1 constant heavy chain polypeptide sequence |
| 589 | gamma-1 constant heavy chain polynucleotide sequence |
| 590-646 | Human IL-6 peptides (Example 14) |
| 719 | gamma-1 constant heavy chain polypeptide sequence (differs |
| | from SEQ ID NO: 518 at two positions) |
| 726 | C-reactive protein polypeptide sequence |
| 727 | IL-6 receptor alpha |
| 728 | IL-6 receptor beta/gp130 |

Such antibody fragments or variants thereof may be present in at least one of the following non-limiting forms: Fab, Fab′, F(ab′)₂, Fv and single chain Fv antibody forms. In a preferred embodiment, the anti-IL-6 antibodies described herein further comprises the kappa constant light chain sequence comprising the sequence set forth in the polypeptide sequence of SEQ ID NO: 586.

In another preferred embodiment, the anti-IL-6 antibodies described herein further comprises the gamma-1 constant heavy chain polypeptide sequence comprising one of the sequences set forth in the polypeptide sequence of SEQ ID NO: 588 and SEQ ID NO: 719.

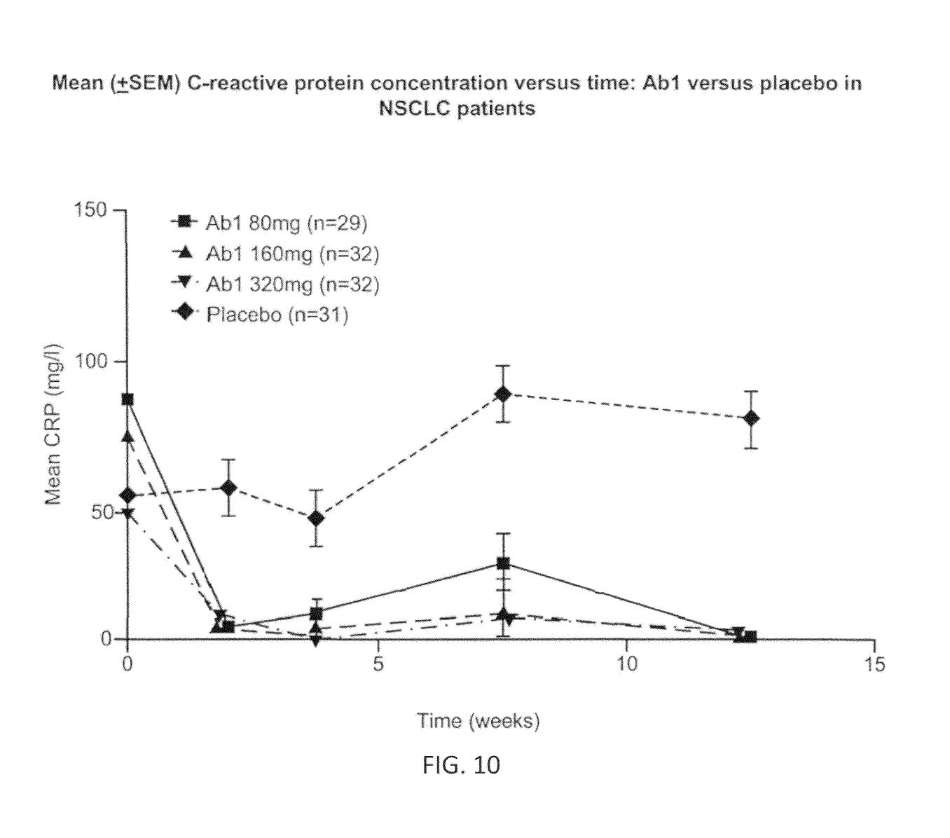

Embodiments of antibodies described herein may include a leader sequence, such as a rabbit Ig leader, albumin pre-peptide, a yeast mating factor pre pro secretion leader sequence (such as P. pastoris or Saccharomyces cerevisiae a or alpha factor), or human HAS leader. Exemplary leader sequences are shown offset from FR1 at the N-terminus of polypeptides shown in FIGS. 10A-B and 11A-B as follows: rabbit Ig leader sequences in SEQ ID NOs: 2 and 660 and SEQ ID NOs: 3 and 661; and an albumin prepeptide in SEQ ID NOs: 706 and 708, which facilitates secretion. Other leader sequences known in the art to confer desired properties, such as secretion, improved stability or half-life, may also be used, either alone or in combinations with one another, on the heavy and/or light chains, which may optionally be cleaved prior to administration to a subject. For example, a polypeptide may be expressed in a cell or cell-free expression system that also expresses or includes (or is modified to express or include) a protease, e.g., a membrane-bound signal peptidase, that cleaves a leader sequence.

In another embodiment, the subject technology contemplates an isolated anti-IL-6 antibody comprising a V_{H} polypeptide sequence comprising: SEQ ID NO: 3, 18, 19, 22, 38, 54, 70, 86, 102, 117, 118, 123, 139, 155, 171, 187, 203, 219, 235, 251, 267, 283, 299, 315, 331, 347, 363, 379, 395, 411, 427, 443, 459, 475, 491, 507, 523, 539, 555, 571, 652, 656, 657, 658, 661, 664, 665, 668, 672, 676, 680, 684, 688, 691, 692, 704, or 708; and further comprising a V_{L} polypeptide sequence comprising: SEQ ID NO: 2, 20, 21, 37, 53, 69, 85, 101, 119, 122, 138, 154, 170, 186, 202, 218, 234, 250, 266, 282, 298, 314, 330, 346, 362, 378, 394, 410, 426, 442, 458, 474, 490, 506, 522, 538, 554, 570, 647, 651, 660, 666, 667, 671, 675, 679, 683, 687, 693, 699, 702, 706, or 709 or a variant thereof wherein at least one of the framework residues (FR residues) or CDR residues in said V_{H or} V_{L} polypeptide has been substituted with another amino acid residue resulting in an anti-IL-6 antibody that specifically binds IL-6. The subject technology contemplates humanized and chimeric forms of these antibodies wherein preferably the FR will comprise human FRs highly homologous to the parent antibody. The chimeric antibodies may include an Fc derived from IgG1, IgG2, IgG3, IgG4, IgG5, IgG6, IgG7, IgG8, IgG9, IgG10, IgG11, IgG12, IgG13, IgG14, IgG15, IgG16, IgG17, IgG18 or IgG19 constant regions and in particular a variable heavy and light chain constant region as set forth in SEQ ID NO: 588 and SEQ ID NO: 586.

In one embodiment of the subject technology, the antibodies or V_{H} or V_{L} polypeptides originate or are selected from at least one rabbit B cell populations prior to initiation of the humanization process referenced herein.

In another embodiment of the subject technology, the anti-IL-6 antibodies and fragments and variants thereof have binding specificity for primate homologs of the human IL-6 protein. Non-limiting examples of primate homologs of the human IL-6 protein are IL-6 obtained from Macaca fascicularis (cynomolgus monkey) and the Rhesus monkey. In another embodiment of the subject technology, the anti-IL-6 antibodies and fragments and variants thereof inhibits the association of IL-6 with IL-6R, and/or the production of IL-6/IL-6R/130 complexes and/or the production of IL-6/IL-6R/gp130 multimers and/or antagonizes the biological effects of at least one of the foregoing.

### Polyclonal Antibody

Polyclonal antibodies are heterogeneous populations of antibody molecules derived from the sera of animals immunized with an antigen. Polyclonal antibodies which selectively bind the IL-6 may be made by methods well-known in the art. See, e.g., Howard & Kaser (2007) Making and Using Antibodies: A Practical Handbook CRC Press.

### Monoclonal Antibody

A monoclonal antibody contains a substantially homogeneous population of antibodies specific to antigens, which population contains substantially similar epitope binding sites. Monoclonal antibodies may be obtained by methods known to those skilled in the art. See, e.g. Kohler and Milstein (1975) Nature 256: 495-497; U.S. Pat. No. 4,376,110; Ausubel, et al. [Eds.] (2011) CURRENT PROTOCOLS IN MOLECULAR BIOLOGY, Greene Publishing Assoc. and Wiley Interscience, NY.; and Harlow & Lane (1998) USING ANTIBODIES: A LABORATORY MANUAL Cold Spring Harbor Laboratory; Colligan, et al. (2005) [Eds.] Current Protocols in Immunology Greene Publishing Assoc. and Wiley Interscience, NY. Such antibodies may be of any immunoglobulin class including IgG, IgM, IgE, IgA, GILD and any subclass thereof. A hybridoma producing an antibody of the present subject technology may be cultivated in vitro, in situ, or in vivo.

### Chimeric Antibody

Chimeric antibodies are molecules different portions of which are derived from different animal species, such as those having variable region derived from a murine antibody and a human immunoglobulin constant region, which are primarily used to reduce immunogenicity in application and to increase yields in production, for example, where murine monoclonal antibodies have higher yields from hybridomas but higher immunogenicity in humans, such that human murine chimeric monoclonal antibodies are used. Chimeric antibodies and methods for their production are known in the art. See Cabilly, et al. (1984) Proc. Natl. Acad. Sci. USA 81: 3273-3277; Morrison, et al. (1994) Proc. Natl. Acad. Sci. USA 81: 6851-6855, Boulianne, et al. (1984) Nature 312: 643-646; Neuberger, et al. (1985) Nature 314: 268-270; European Patent Application 173494 (1986); WO 86/01533 (1986); European Patent Application 184187 (1986); European Patent Application 73494 (1986); Sahagan, et al. (1986) J Immunol. 137: 1066-1074; Liu, et al. (1987) Proc. Natl. Acad. Sci. USA 84: 3439-3443; Sun, et al. (1987) Proc. Natl. Acad. Sci. USA 84: 214-218; Better, et al. (1988) Science 240: 1041-1043; and Harlow & Lane (1998) USING ANTIBODIES: A LABORATORY MANUAL Cold Spring Harbor Laboratory; and U.S. Pat. No. 5,624,659.

### Humanized Antibody

Humanized antibodies are engineered to contain even more human-like immunoglobulin domains, and incorporate only the complementarity-determining regions of the animal-derived antibody. This may be accomplished by examining the sequence of the hyper-variable loops of the variable regions of the monoclonal antibody, and fitting them to the structure of the human antibody chains. See, e.g., U.S. Pat. No. 6,187,287. Likewise, other methods of producing humanized antibodies are now well known in the art. See, e.g., U.S. Pat. Nos. 5,225,539; 5,530,101; 5,585,089; 5,693,762; 6,054,297; 6,180,370; 6,407,213; 6,548,640; 6,632,927; and 6,639,055; Jones, et al. (1986) Nature 321: 522-525; Reichmann, et al. (1988) Nature 332: 323-327; Verhoeyen, et al. (1988) Science 239: 1534-36; and Zhiqiang An (2009) [Ed.] Therapeutic Monoclonal Antibodies: From Bench to Clinic John Wiley & Sons, Inc.

### Antibody Fragments (Antigen-Binding Fragments)

In addition to entire immunoglobulins (or their recombinant counterparts), immunoglobulin fragments comprising the epitope binding site (e.g., Fab′, F(ab′)₂, or other fragments) may be synthesized. “Fragment,” or minimal immunoglobulins may be designed utilizing recombinant immunoglobulin techniques. For instance “Fv” immunoglobulins for use in the present subject technology may be produced by synthesizing a fused variable light chain region and a variable heavy chain region. Combinations of antibodies are also of interest, e.g. diabodies, which comprise two distinct Fv specificities. Antigen-binding fragments of immunoglobulins include but are not limited to SMIPs (small molecule immunopharmaceuticals), camelbodies, nanobodies, and IgNAR.

### Anti-Idiotypic Antibody

An anti-idiotypic (anti-Id) antibody is an antibody which recognizes unique determinants generally associated with the antigen-binding site of an antibody. An Id antibody may be prepared by immunizing an animal of the same species and genetic type (e.g., mouse strain) as the source of the antibody with the antibody to which an anti-Id is being prepared. The immunized animal will recognize and respond to the idiotypic determinants of the immunizing antibody by producing an antibody to these idiotypic determinants (the anti-ld antibody). See e.g., U.S. Pat. No. 4,699,880. The anti-Id antibody may also be used as an “immunogen” to induce an immune response in yet another animal, producing a so-called anti-anti-Id antibody. The anti-anti-Id may be epitopically identical to the original antibody which induced the anti-Id. Thus, by using antibodies to the idiotypic determinants of an antibody it is possible to identify other clones expressing antibodies of identical specificity.

### Engineered and Modified Antibodies

An antibody of the subject technology further may be prepared using an antibody having at least one of the V_{H} and/or V_{L} sequences derived from an antibody starting material to engineer a modified antibody, which modified antibody may have altered properties from the starting antibody. An antibody may be engineered by modifying at least one residues within one or both variable regions (i.e., V_{H} and/or V_{L}), for example within at least one CDR regions and/or within at least one framework regions. Additionally or alternatively, an antibody may be engineered by modifying residues within the constant region(s), for example to alter the effector function(s) of the antibody.

One type of variable region engineering that may be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties. See, e.g., Riechmann, et al. (1998) Nature 332: 323-327; Jones, et al. (1986) Nature 321: 522-525; Queen, et al. (1989) Proc. Natl. Acad. U.S.A. 86: 10029-10033; U.S. Pat. Nos. 5,225,539; 5,530,101; 5,585,089; 5,693,762; and 6,180,370.

Suitable framework sequences may be obtained from public DNA databases or published references that include germline antibody gene sequences. For example, germline DNA sequences for human heavy and light chain variable region genes may be found in the “VBase” human germline sequence database (available on the Internet), as well as in Kabat, E. A., et al. (1991) Sequences of Proteins of Immunological Interest, Fifth Edition, U.S. Department of Health and Human Services, NIH Publication No. 91-3242; Tomlinson, et al. (1992) “The Repertoire of Human Germline V_{H} Sequences Reveals about Fifty Groups of V_{H} Segments with Different Hypervariable Loops” J. Mol. Biol. 227: 776-798; and Cox, et al. (1994) Eur. J Immunol. 24: 827-836.

Another type of variable region modification is to mutate amino acid residues within the V_{H} and/or V_{L} CDR 1, CDR2 and/or CDR3 regions to thereby improve at least one binding properties (e.g., affinity) of the antibody of interest. Site-directed mutagenesis or PCR-mediated mutagenesis may be performed to introduce the mutation(s) and the effect on antibody binding, or other functional property of interest, may be evaluated in appropriate in vitro or in vivo assays. Preferably conservative modifications (as discussed herein) may be introduced. The mutations may be amino acid substitutions, additions or deletions, but are preferably substitutions. Moreover, typically no more than one, two, three, four or five residues within a CDR region are altered.

Engineered antibodies of the subject technology include those in which modifications have been made to framework residues within V_{H} and/or V_{L}, e.g. to improve the properties of the antibody. Typically such framework modifications are made to decrease the immunogenicity of the antibody. For example, one approach is to “backmutate” at least one framework residues to the corresponding germline sequence. More specifically, an antibody that has undergone somatic mutation may contain framework residues that differ from the germline sequence from which the antibody is derived. Such residues may be identified by comparing the antibody framework sequences to the germline sequences from which the antibody is derived.

In addition or alternative to modifications made within the framework or CDR regions, antibodies of the subject technology may be engineered to include modifications within the Fc region, typically to alter at least one functional properties of the antibody, such as serum half-life, complement fixation, Fc receptor binding, and/or antigen-dependent cellular cytotoxicity. Furthermore, an antibody of the subject technology may be chemically modified (e.g., at least one chemical moieties may be attached to the antibody) or be modified to alter its glycosylation, again to alter at least one functional properties of the antibody. Such embodiments are described further below. The numbering of residues in the Fc region is that of the EU index of Kabat.

The hinge region of CH1 may be modified such that the number of cysteine residues in the hinge region is altered, e.g., increased or decreased. See U.S. Pat. No. 5,677,425. The number of cysteine residues in the hinge region of CH1 may be altered to, for example, facilitate assembly of the light and heavy chains or to increase or decrease the stability of the antibody. The Fc hinge region of an antibody may be mutated to decrease the biological half-life of the antibody. More specifically, at least one amino acid mutations may be introduced into the CH2-CH3 domain interface region of the Fc-hinge fragment such that the antibody has impaired Staphylococcyl protein A (SpA) binding relative to native Fc-hinge domain SpA binding. See, e.g., U.S. Pat. No. 6,165,745.

The antibody may be modified to increase its biological half-life. Various approaches are possible. For example, at least one of the following mutations may be introduced: T252L, T254S, T256F. See U.S. Pat. No. 6,277,375. Alternatively, to increase the biological half-life, the antibody may be altered within the CH1 or CL region to contain a salvage receptor binding epitope taken from two loops of a CH2 domain of an Fc region of an IgG. See U.S. Pat. Nos. 5,869,046 and 6,121,022.

The Fc region may be altered by replacing at least one amino acid residue with a different amino acid residue to alter the effector function(s) of the antibody. For example, at least one amino acid selected from amino acid residues 234, 235, 236, 237, 297, 318, 320 and 322 may be replaced with a different amino acid residue such that the antibody has an altered affinity for an effector ligand but retains the antigen-binding ability of the parent antibody. The effector ligand to which affinity may be altered may be, for example, an Fc receptor or the Cl component of complement. See U.S. Pat. Nos. 5,624,821 and 5,648,260.

The Fc region may be modified to increase the affinity of the antibody for an Fcy receptor by modifying at least one amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. See WO 00/42072. Moreover, the binding sites on human IgG1 for FcγRI, FcγRII, FcγRIII and FcRn have been mapped and variants with improved binding. See Shields, et al. (2001) J. Biol. Chem. 276: 6591-6604. Specific mutations at positions 256, 290, 298, 333, 334 and 339 are shown to improve binding to FcγRIII. Additionally, the following combination mutants are shown to improve FcγRIII binding: T256A/S298A, S298A/E333A, S298A/K224A and S298A/E333A/K334A.

The glycosylation of an antibody may be modified. For example, an aglycosylated antibody may be made (i.e., the antibody lacks glycosylation). Glycosylation may be altered to, for example, increase the affinity of the antibody for antigen. Such carbohydrate modifications may be accomplished by, for example, altering at least one sites of glycosylation within the antibody sequence. For example, at least one amino acid substitutions may be made that result in elimination of at least one variable region framework glycosylation sites to thereby eliminate glycosylation at that site. Such aglyclosylation may increase the affinity of the antibody for antigen. See, e.g., U.S. Pat. Nos. 5,714,350 and 6,350,861.

Additionally or alternatively, an antibody may be made that has an altered type of glycosylation, such as a hypofucosylated antibody having reduced amounts of fucosyl residues or an antibody having increased bisecting GlcNac structures. Such carbohydrate modifications may be accomplished by, for example, expressing the antibody in a host cell with altered glycosylation machinery. Cells with altered glycosylation machinery have been described in the art and may be used as host cells in which to express recombinant antibodies of the subject technology to thereby produce an antibody with altered glycosylation. See U.S. Patent Application Publication No. 2004/0110704 and Yamane-Ohnuki, et al. (2004) Biotechnol Bioeng. 87: 614-22; EP 1,176,195; WO 2003/035835; Shields, et al. (2002) J. Biol. Chem. 277: 26733-26740; WO 99/54342; Umana, et al. (1999) Nat. Biotech. 17: 176-180; and Tarentino, et al. (1975) Biochem. 14: 5516-23.

An antibody may be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which at least one PEG groups become attached to the antibody or antibody fragment. Preferably, the pegylation is carried out via an acylation reaction or an alkylation reaction with a reactive PEG molecule (or an analogous reactive water-soluble polymer).

The subject technology also provides variants and equivalents that are substantially homologous to the antibodies, antibody fragments, diabodies, SMIPs, camelbodies, nanobodies, IgNAR, polypeptides, variable regions and CDRs set forth herein. These may contain, e.g., conservative substitution mutations, (i.e., the substitution of at least one amino acids by similar amino acids). For example, conservative substitution refers to the substitution of an amino acid with another within the same general class, e.g., one acidic amino acid with another acidic amino acid, one basic amino acid with another basic amino acid, or one neutral amino acid by another neutral amino acid. In another embodiment, the subject technology further contemplates the above-recited polypeptide homologs of the antibody fragments, variable regions and CDRs set forth herein further having anti-IL-6 activity. Non-limiting examples of anti-IL-6 activity are set forth herein, for example, under the heading “Anti-IL-6 Activity,” infra.

Anti-IL-6 antibodies have also been disclosed in the following published and unpublished patent applications, which are co-owned by the assignee of the present application: WO 2008/144763; U.S. Patent Application Publication Nos. 2009/0028784, 2009/0297513, and 2009/0297436. Other anti-IL-6 antibodies have been disclosed in the following U.S. Patents and Published Patent Application Nos: U.S. Pat. Nos. 7,482,436; 7,291,721; 6,121,423; 2008/0075726; 2007/0178098; 2007/0154481; 2006/0257407; and 2006/0188502.

### Polypeptide Sequence Variants

For any anti-IL-6 antibodies sequence described herein, further characterization or optimization may be achieved by systematically either adding or removing amino acid residues to generate longer or shorter peptides, and testing those and sequences generated by walking a window of the longer or shorter size up or down the antigen from that point. Coupling this approach to generating new candidate targets with testing for effectiveness of antigenic molecules based on those sequences in an immunogenicity assay, as known in the art or as described herein, may lead to further manipulation of the antigen. Further still, such optimized sequences may be adjusted by, e.g., the addition, deletions, or other mutations as known in the art and/or discussed herein to further optimize the anti-IL-6 antibodies (e.g., increasing serum stability or circulating half-life, increasing thermal stability, enhancing delivery, enhance immunogenicity, increasing solubility, targeting to a particular in vivo location or cell type).

In another embodiment, the subject technology contemplates polypeptide sequences having at least about 90% sequence homology to any at least one of the polypeptide sequences of antibody fragments, variable regions and CDRs set forth herein. More preferably, the subject technology contemplates polypeptide sequences having at least about 95% sequence homology, even more preferably at least about 98% sequence homology, and still more preferably at least about 99% sequence homology to any at least one of the polypeptide sequences of antibody fragments, variable regions and CDRs set forth herein. Methods for determining homology between nucleic acid and amino acid sequences are well known to those of ordinary skill in the art.

The anti-IL-6 antibodies polypeptides described herein may comprise conservative substitution mutations, (i.e., the substitution of at least one amino acids by similar amino acids). For example, conservative substitution refers to the substitution of an amino acid with another within the same general class, e.g., one acidic amino acid with another acidic amino acid, one basic amino acid with another basic amino acid, or one neutral amino acid by another neutral amino acid.

Anti-IL-6 antibodies polypeptide sequences may have at least about 60, 65, 70, 75, 80, 81, 82, 83, 84, 85, 86, 87, 88, 89, 90, 91, 92, 93, 94, 95, 96, 97, 98, 98.5, 99, 99.5, 99.8, 99.9, or 100% sequence homology to any at least one of the polypeptide sequences set forth herein. More preferably, the subject technology contemplates polypeptide sequences having at least about 95% sequence homology, even more preferably at least about 98% sequence homology, and still more preferably at least about 99% sequence homology to any at least one of the polypeptide sequences of Anti-IL-6 antibodies polypeptide sequences set forth herein. Methods for determining homology between amino acid sequences, as well as nucleic acid sequences, are well known to those of ordinary skill in the art. See, e.g., Nedelkov & Nelson (2006) New and Emerging Proteomic Techniques Humana Press. Thus, an anti-IL-6 antibodies polypeptide may have at least about 60, 65, 70, 75, 80, 81, 82, 83, 84, 85, 86, 87, 88, 89, 90, 91, 92, 93, 94, 95, 96, 97, 98, 98.5, 99, 99.5, 99.8, 99.9, or 100% sequence homology with a polypeptide sequence.

The term homology, or identity, is understood as meaning the number of agreeing amino acids (identity) with other proteins, expressed in percent. The identity is preferably determined by comparing a given sequence with other proteins with the aid of computer programs. If sequences which are compared with each other are different in length, the identity is to be determined in such a way that the number of amino acids which the short sequence shares with the longer sequence determines the percentage identity. The identity can be determined routinely by means of known computer programs which are publicly available such as, for example, ClustalW. Thompson, et al. (1994) Nucleic Acids Research 22: 4673-4680. ClustalW is publicly available from the European Molecular Biology Laboratory and may be downloaded from various internet pages, inter alia the IGBMC (Institut de Génétique et de Biologie Moléculaire et Cellulaire) and the EBI and all mirrored EBI internet pages (European Bioinformatics Institute). If the ClustalW computer program Version 1.8 is used to determine the identity between, for example, the reference protein of the present application and other proteins, the following parameters are to be set: KTUPLE=1, TOPDIAG=5, WINDOW=5, PAIRGAP=3, GAPOPEN=10, GAPEXTEND=0.05, GAPDIST=8, MAXDIV=40, MATRIX=GONNET, ENDGAPS(OFF), NOPGAP, NOHGAP. See also European Bioinformatics Institute (EBI) toolbox available on-line and Smith (2002) Protein Sequencing Protocols [2¹¹″ Ed.] Humana Press.

One possibility of finding similar sequences is to carry out sequence database researches. Here, at least one sequences may be entered as what is known as a query. This query sequence is then compared with sequences present in the selected databases using statistical computer programs. Such database queries (blast searches) are known to the skilled worker and may be carried out at different suppliers. If, for example, such a database query is carried out at the NCBI (National Center for Biotechnology Information), the standard settings for the respective comparison query should be used. For protein sequence comparisons (blastp), these settings are: Limit entrez=not activated; Filter=low complexity activated; Expect value=10; word size=3; Matrix=BLOSUM62; Gap costs: Existence=11, Extension=1. The result of such a query is, among other parameters, the degree of identity between the query sequence and the similar sequences found in the databases. Methods and materials for making fragments of Anti-IL-6 antibodies polypeptides are well known in the art. See, e.g., Maniatis, et al. (2001) Molecular Cloning: A Laboratory Manual[3^{rd} Ed.] Cold Spring Harbor Laboratory Press.

Variant anti-IL-6 antibodies polypeptides may retain their antigenic specificity to bind IL-6. Fully specific variants may contain only conservative variations or variations in non-critical residues or in non-critical regions. Variants may also contain substitution of similar amino acids that result in no change or an insignificant change in their specificity. Alternatively, such substitutions may positively or negatively affect specificity to some degree. Non-specific variants typically contain at least one non-conservative amino acid substitutions, deletions, insertions, inversions, or truncation or a substitution, insertion, inversion, or deletion in a critical residue or critical region of an epitope. Molecular biology and biochemistry techniques for modifying anti-IL-6 antibodies polypeptides while preserving specificity are well known in the art. See, e.g., Ho, et al. (1989) Gene 77(1): 51-59; Landt, et al. (1990) Gene 96(1): 125-128; Hopp & Woods (1991) Proc. Natl. Acad. Sci. USA 78(6): 3824-3828; Kolaskar & Tongaonkar (1990) FEBS Letters 276(1-2): 172-174; and Welling, et al. (1985) FEBS Letters 188(2): 215-218

Amino acids that are essential for function may be identified by methods known in the art, such as site-directed mutagenesis or alanine-scanning mutagenesis. Cunningham, et al. (1989) Sci. 244: 1081-85. The latter procedure introduces single alanine mutations at every residue in the molecule. The resulting mutant molecules are then tested for biological activity such as epitope binding. Sites that are critical for ligand-receptor binding may also be determined by structural analysis such as crystallography, nuclear magnetic resonance, or photoaffinity labeling. Smith, et al. (1992) J. Mol. Biol. 224: 899-904; de Vos, et al. (1992) Sci. 255: 306-12.

For example, one class of substitutions is conserved amino acid substitutions. Such substitutions are those that substitute a given amino acid in an anti-IL-6 antibody polypeptide with another amino acid of like characteristics. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg, replacements among the aromatic residues Phe, Tyr. Guidance concerning which amino acid changes are likely to be phenotypically silent is found in, for example, Bowie, et al. (1990) Sci. 247: 1306-10. Hence, one of ordinary skill in the art appreciates that the inventors possess peptide variants without delineation of all the specific variants. As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the subject technology. See, e.g., Creighton (1992) Proteins: Structures and Molecular Properties [2^{nd} Ed.] W.H. Freeman.

Moreover, polypeptides often contain amino acids other than the twenty “naturally occurring” amino acids. Further, many amino acids, including the terminal amino acids, may be modified by natural processes, such as processing and other post-translational modifications, or by chemical modification techniques well known in the art. Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, g-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See Creighton (1992) Proteins: Structure and Molecular Properties [2^{nd} Ed.] and Lundblad (1995) Techniques in Protein Modification[1^{st} Ed.] Many detailed reviews are available on this subject. See, e.g., Wold (1983) Posttranslational Covalent Modification of Proteins Acad. Press, NY; Seifter, et al. (1990) Meth. Enzymol. 182: 626-46; and Rattan, et al. (1992) Ann. NY Acad. Sci. 663: 48-62.

In another embodiment, the subject technology further contemplates the generation and use of anti-idiotypic antibodies that bind any of the foregoing sequences. In an exemplary embodiment, such an anti-idiotypic antibody could be administered to a subject who has received an anti-IL-6 antibody to modulate, reduce, or neutralize, the effect of the anti-IL-6 antibody. A further exemplary use of such anti-idiotypic antibodies is for detection of the anti-IL-6 antibodies of the present subject technology, for example to monitor the levels of the anti-IL-6 antibodies present in a subject's blood or other bodily fluids.

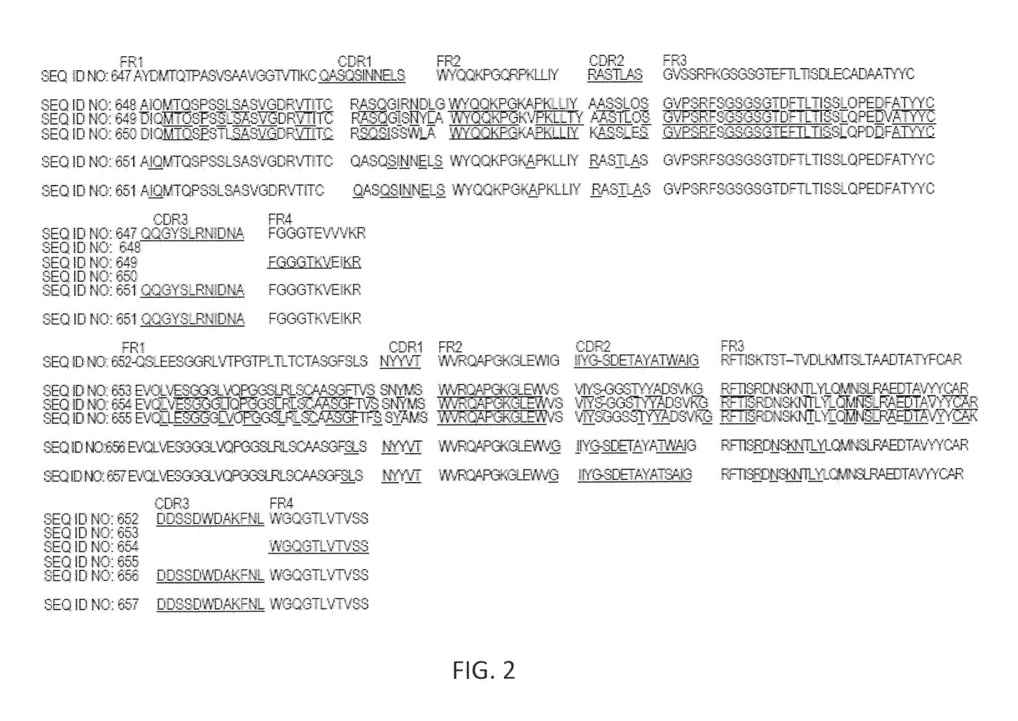

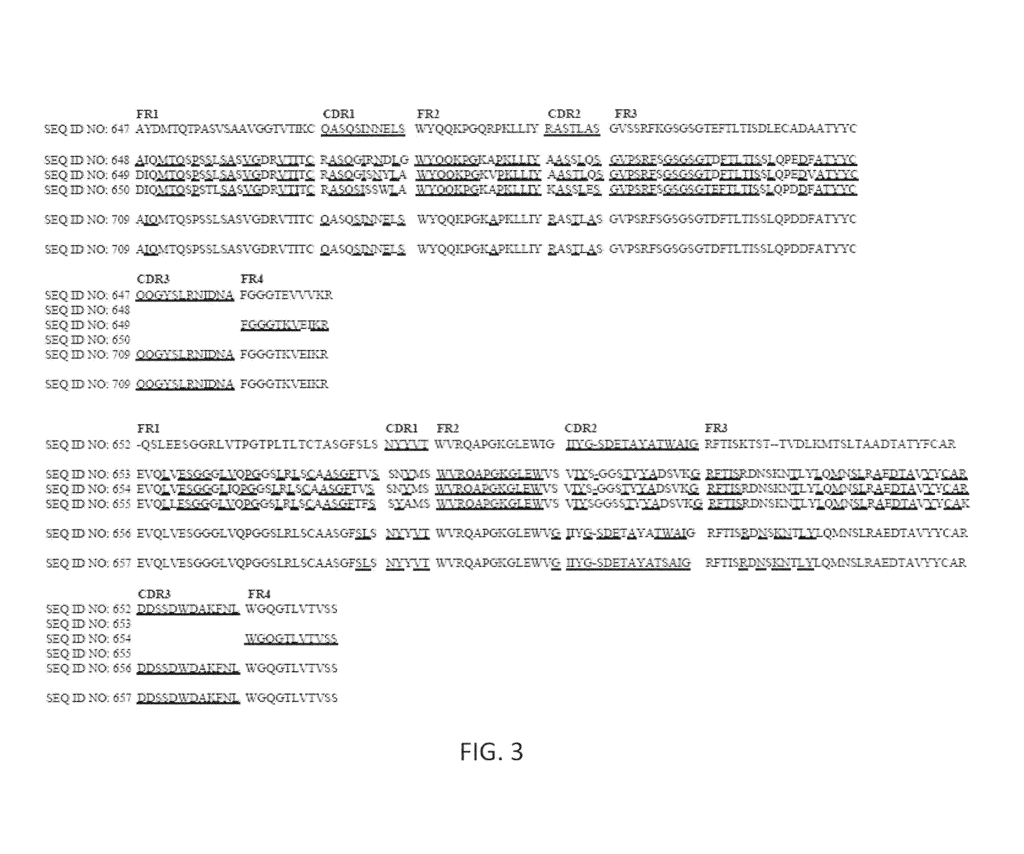

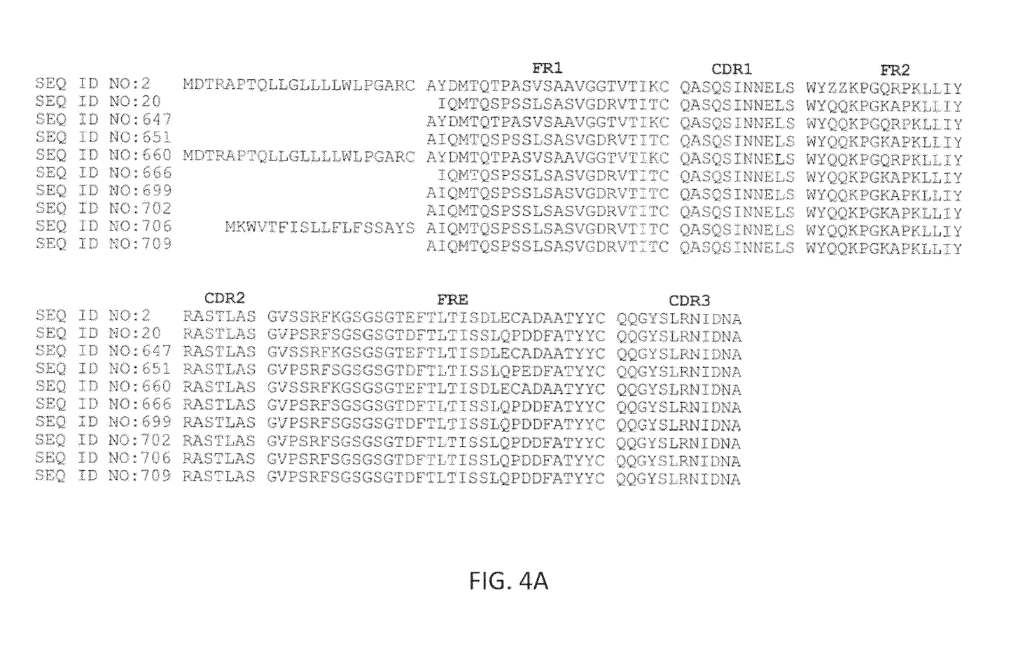

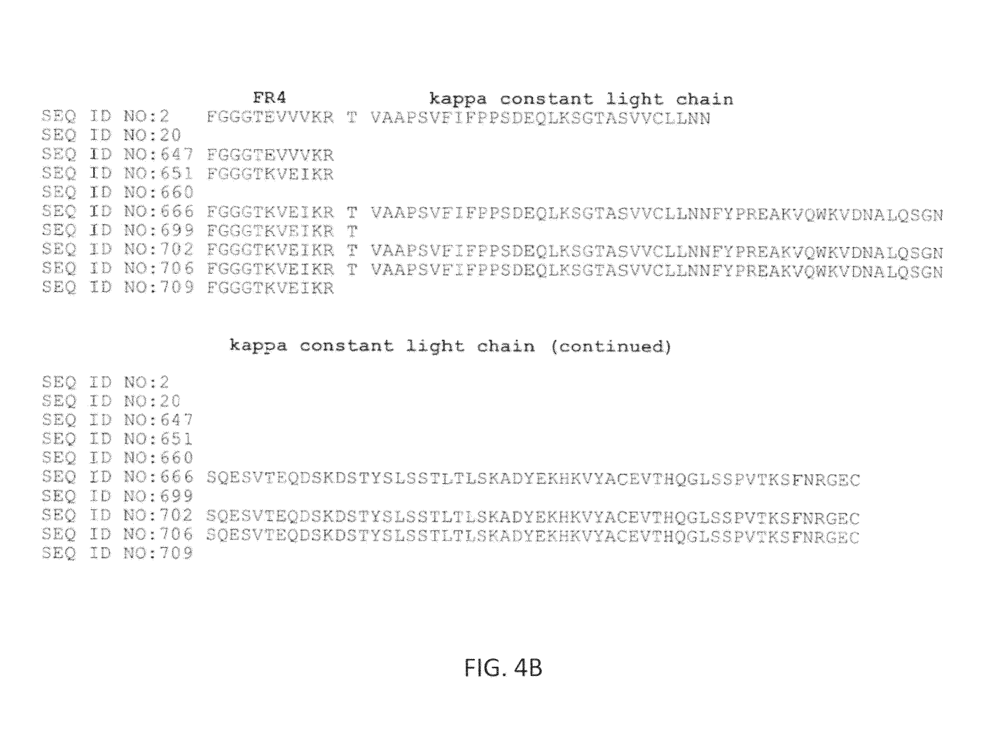

The present subject technology also contemplates anti-IL-6 antibodies comprising any of the polypeptide or polynucleotide sequences described herein substituted for any of the other polynucleotide sequences described herein. For example, without limitation thereto, the present subject technology contemplates antibodies comprising the combination of any of the variable light chain and variable heavy chain sequences described herein, and further contemplates antibodies resulting from substitution of any of the CDR sequences described herein for any of the other CDR sequences described herein. As noted preferred anti-IL-6 antibodies or fragments or variants thereof may contain a variable heavy and/or light sequence as shown in FIG. 2-5, such as SEQ ID NO: 651,657,709 or variants thereof wherein at least one CDR or FR residues are modified without adversely affecting antibody binding to IL-6 or other desired functional activity.

### Fusion Proteins

Fusions comprising the anti-IL-6 antibodies polypeptides are also within the scope of the present subject technology. For example, the fusion protein may be linked to a GST fusion protein in which the anti-IL-6 antibodies polypeptide sequences are fused to the C-terminus of the GST sequences. Such fusion proteins may facilitate the purification of the recombinant Anti-IL-6 antibodies polypeptides. Alternatively, anti-IL-6 antibodies polypeptides may be fused with a protein that binds B-cell follicles, thus initiating both a humoral immune response and activation of T cells. Berney, et al. (1999) J. Exp. Med. 190: 851-60. Alternatively, for example, the Anti-IL-6 antibodies polypeptides may be genetically coupled with and anti-dendritic cell antibody to deliver the antigen to the immune system and stimulate a cellular immune response. He, et al. (2004) Clin. Cancer Res. 10: 1920-27. A chimeric or fusion protein of the subject technology may be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. The fusion gene may be synthesized by conventional techniques including automated DNA synthesizers.

Fusion proteins may include C-terminal or N-terminal translocation sequences. Further, fusion proteins can comprise additional elements, e.g., for protein detection, purification, or other applications. Detection and purification facilitating domains including but not limited to metal chelating peptides such as polyhistidine tracts, histidine-tryptophan modules, or other domains that allow purification on immobilized metals; maltose binding protein; protein A domains that allow purification on immobilized immunoglobulin; or the domain utilized in the FLAG extension/affinity purification system (Immunex Corp, Seattle Wash.)

A fusion protein may be prepared from a protein of the subject technology by fusion with a portion of an immunoglobulin comprising a constant region of an immunoglobulin. More preferably, the portion of the immunoglobulin comprises a heavy chain constant region which is optionally and more preferably a human heavy chain constant region. The heavy chain constant region is most preferably an IgG heavy chain constant region, and optionally and most preferably is an Fc chain, most preferably an IgG Fc fragment that comprises CH2 and CH3 domains. Although any IgG subtype may optionally be used, the IgG1 subtype is preferred. The Fc chain may optionally be a known or “wild type” Fc chain, or alternatively may be mutated. See, e.g., U.S. Patent Application Publication No. 2006/0034852. The term “Fc chain” also optionally comprises any type of Fc fragment. Several of the specific amino acid residues that are involved in antibody constant region-mediated activity in the IgG subclass have been identified. Inclusion, substitution or exclusion of these specific amino acids therefore allows for inclusion or exclusion of specific immunoglobulin constant region-mediated activity. Furthermore, specific changes may result in aglycosylation for example and/or other desired changes to the Fc chain. At least some changes may optionally be made to block a function of Fc which is considered to be undesirable, such as an undesirable immune system effect. See McCafferty, et al. (2002) Antibody Engineering: A Practical Approach (Eds.) Oxford University Press.

The inclusion of a cleavable linker sequences such as Factor Xa (see, e.g., Ottavi, (1998) Biochimie 80: 289-93), subtilisin protease recognition motif (see, e.g., Polyak (1997) Protein Eng. 10: 615-19); enterokinase (Invitrogen, San Diego, Calif.), between the translocation domain (for efficient plasma membrane expression) and the rest of the newly translated polypeptide may be useful to facilitate purification. For example, one construct can include a polypeptide encoding a nucleic acid sequence linked to six histidine residues followed by a thioredoxin, an enterokinase cleavage site (see, e.g., Williams (1995) Biochemistry 34: 1787-97), and an C-terminal translocation domain. The histidine residues facilitate detection and purification while the enterokinase cleavage site provides a means for purifying the desired protein(s) from the remainder of the fusion protein. Technology pertaining to vectors encoding fusion proteins and application of fusion proteins are well described in the scientific and patent literature. See, e.g., Kroll (1993) DNA Cell. Biol. 12: 441-53.

### Conjugates

The anti-IL-6 antibodies, antibodies that bind the Anti-IL-6 antibodies and fragments thereof, may be conjugated to other moieties. Such conjugates are often used in the preparation of vaccines. The anti-IL-6 antibodies polypeptide may be conjugated to a carbohydrate (e.g., mannose, fucose, glucose, GlcNAs, maltose), which is recognized by the mannose receptor present on dendritic cells and macrophages. The ensuing binding, aggregation, and receptor-mediated endocytosis and phagocytosis functions provide enhanced innate and adaptive immunity. See Mahnke, et al. (2000) J. Cell Biol. 151: 673-84; Dong, et al. (1999) J. Immunol. 163: 5427-34. Other moieties suitable for conjugation to elicit an immune response includes but not limited to Keyhole Limpet Hemocyanin (KLH), diphtheria toxoid, cholera toxoid, Pseudomonas exoprotein A, and microbial outer membrane proteins (OMPS).

### Polynucleotides Encoding Anti-IL-6 Antibody Polypeptides

The subject technology is further directed to polynucleotides encoding polypeptides of the antibodies having binding specificity to IL-6. In one embodiment of the subject technology, polynucleotides of the subject technology comprise, or alternatively consist of, the following polynucleotide sequence encoding the variable light chain polypeptide sequence of SEQ ID NO: 2 which is encoded by the polynucleotide sequence of SEQ ID NO: 10 or the polynucleotide sequence of SEQ ID NO: 662,698,701, or 705.

In another embodiment of the subject technology, polynucleotides of the subject technology comprise, or alternatively consist of, the following polynucleotide sequence encoding the variable heavy chain polypeptide sequence of SEQ ID NO: 3 which is encoded by the polynucleotide sequence of SEQ ID NO: 11 or the polynucleotide sequence of SEQ ID NO: 663, 700, 703, or 707.

In a further embodiment of the subject technology, polynucleotides encoding fragments or variants of the antibody having binding specificity to IL-6 comprise, or alternatively consist of, at least one of the polynucleotide sequences of SEQ ID NO: 12 or 694; SEQ ID NO: 13; and SEQ ID NO: 14 or 695 which correspond to polynucleotides encoding the complementarity-determining regions (CDRs, or hypervariable regions) of the light chain variable sequence of SEQ ID NO: 2.

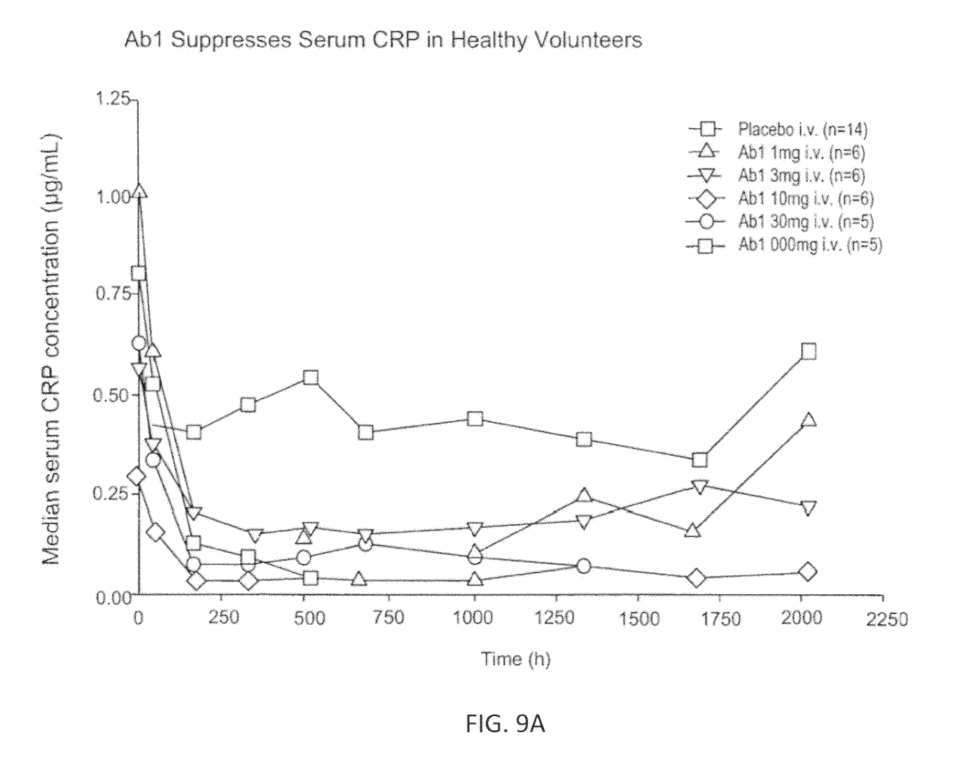

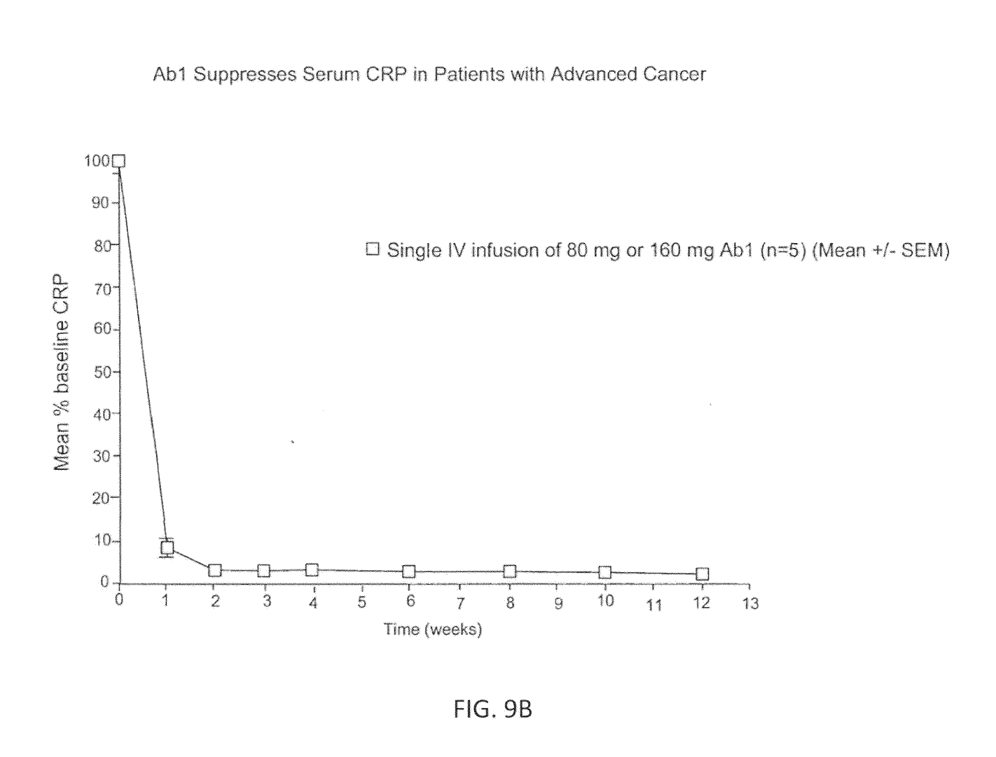

In a further embodiment of the subject technology, polynucleotides encoding fragments or variants of the antibody having binding specificity to IL-6 comprise, or alternatively consist of, at least one of the polynucleotide sequences of SEQ ID NO: 15; SEQ ID NO: 16 or 696; and SEQ ID NO: 17 or 697 which correspond to polynucleotides encoding the complementarity-determining regions (CDRs, or hypervariable regions) of the heavy chain variable sequence of SEQ ID NO: 3 or SEQ ID NO: 661 or SEQ ID NO: 657 or others depicted in FIG. 8 or 9.

The subject technology also contemplates polynucleotide sequences including at least one of the polynucleotide sequences encoding antibody fragments or variants described herein. In one embodiment of the subject technology, polynucleotides encoding fragments or variants of the antibody having binding specificity to IL-6 comprise, or alternatively consist of, one, two, three or more, including all of the following polynucleotides encoding antibody fragments: the polynucleotide SEQ ID NO: 10 encoding the light chain variable region of SEQ ID NO: 2; the polynucleotide SEQ ID NO: 11 encoding the heavy chain variable region of SEQ ID NO: 3; the polynucleotide SEQ ID NO: 720 encoding the light chain polypeptide of SEQ ID NO: 20; the polynucleotide SEQ ID NO: 721 encoding the light chain polypeptide of SEQ ID NO: 647; the polynucleotide SEQ ID NO: 662 encoding the light chain polypeptide of SEQ ID NO: 660; the polynucleotide SEQ ID NO: 722 encoding the light chain polypeptide of SEQ ID NO: 666; the polynucleotide SEQ ID NO: 698 encoding the light chain polypeptide of SEQ ID NO: 699; the polynucleotide SEQ ID NO: 701 encoding the light chain polypeptide of SEQ ID NO: 702; the polynucleotide SEQ ID NO: 705 encoding the light chain polypeptide of SEQ ID NO: 706; the polynucleotide SEQ ID NO: 723 encoding the light chain polypeptide of SEQ ID NO: 709; the polynucleotide SEQ ID NO: 724 encoding the heavy chain polypeptide of SEQ ID NO: 19; the polynucleotide SEQ ID NO: 725 encoding the heavy chain polypeptide of SEQ ID NO: 652; the polynucleotide SEQ ID NO: 700 encoding the heavy chain polypeptide of SEQ ID NO: 657; the polynucleotide SEQ ID NO: 663 encoding the heavy chain polypeptide of SEQ ID NO: 661; the polynucleotide SEQ ID NO: 703 encoding the heavy chain polypeptide of SEQ ID NO: 704; the polynucleotide SEQ ID NO: 707 encoding the heavy chain polypeptide of SEQ ID NO: 708; the polynucleotides of SEQ ID NO: 12, 13, 14, 694 and 695 encoding the complementarity-determining regions of the aforementioned light chain polypeptides; and the polynucleotides of SEQ ID NO: 15, 16, 17, 696 and 697 encoding the complementarity-determining regions of the aforementioned heavy chain polypeptides, and polynucleotides encoding the variable heavy and light chain sequences in SEQ ID NO: 657 and SEQ ID NO: 709 respectively, e.g., the nucleic acid sequences in SEQ ID NO: 700 and SEQ ID NO: 723 and fragments or variants thereof, e.g., based on codon degeneracy. These nucleic acid sequences encoding variable heavy and light chain sequences may be expressed alone or in combination and these sequences preferably are fused to suitable variable constant sequences, e.g., those in SEQ ID NO: 589 and SEQ ID NO: 587.

Exemplary nucleotide sequences encoding anti-IL-6 antibodies of the present subject technology are identified in Table 1. The polynucleotide sequences shown are to be understood to be illustrative, rather than limiting. One of skill in the art can readily determine the polynucleotide sequences that would encode a given polypeptide and can readily generate coding sequences suitable for expression in a given expression system, such as by adapting the polynucleotide sequences provided and/or by generating them de novo, and can readily produce codon-optimized expression sequences, for example as described in published U.S. Patent Application No. 2008/0120732 or using other methods known in the art.

In another embodiment of the subject technology, polynucleotides of the subject technology further comprise, the following polynucleotide sequence encoding the kappa constant light chain sequence of SEQ ID NO: 586 which is encoded by the polynucleotide sequence of SEQ ID NO: 587.

In another embodiment of the subject technology, polynucleotides of the subject technology further comprise, the following polynucleotide sequence encoding the gamma-1 constant heavy chain polypeptide sequence of SEQ ID NO: 588 which is encoded by the polynucleotide sequence of SEQ ID NO: 589.

In one embodiment, the subject technology is directed to an isolated polynucleotide comprising a polynucleotide encoding an anti-IL-6 V_{H} antibody amino acid sequence selected from SEQ ID NO: 3, 18, 19, 652, 656, 657, 658, 661, 664, 665, 704, and 708 or encoding a variant thereof wherein at least one framework residue (FR residue) has been substituted with an amino acid present at the corresponding position in a rabbit anti-IL-6 antibody V_{H} polypeptide or a conservative amino acid substitution. In addition, the subject technology specifically encompasses humanized anti-IL-6 antibodies or humanized antibody binding fragments or variants thereof and nucleic acid sequences encoding the foregoing comprising the humanized variable heavy chain and/or light chain polypeptides depicted in the sequences contained in FIG. 1-5, or those identified in Table 1, or variants thereof wherein at least one framework or CDR residues may be modified. Preferably, if any modifications are introduced they will not affect adversely the binding affinity of the resulting anti-IL-6 antibody or fragment or variant thereof.

In another embodiment, the subject technology is directed to an isolated polynucleotide comprising the polynucleotide sequence encoding an anti-IL-6 V_{L} antibody amino acid sequence selected from SEQ ID NO: 2, 20, 647, 651, 660, 666, 699, 702, 706, and 709 or encoding a variant thereof wherein at least one framework residue (FR residue) has been substituted with an amino acid present at the corresponding position in a rabbit anti-IL-6 antibody V_{L} polypeptide or a conservative amino acid substitution.

In yet another embodiment, the subject technology is directed to at least one heterologous polynucleotides comprising a sequence encoding the polypeptides set forth in SEQ ID NO: 2 and SEQ ID NO: 3; SEQ ID NO: 2 and SEQ ID NO: 18; SEQ ID NO: 2 and SEQ ID NO: 19; SEQ ID NO: 20 and SEQ ID NO: 3; SEQ ID NO: 20 and SEQ ID NO: 18; or SEQ ID NO: 20 and SEQ ID NO: 19.

In another embodiment, the subject technology is directed to an isolated polynucleotide that expresses a polypeptide containing at least one CDR polypeptide derived from an anti-IL-6 antibody wherein said expressed polypeptide alone specifically binds IL-6 or specifically binds IL-6 when expressed in association with another polynucleotide sequence that expresses a polypeptide containing at least one CDR polypeptide derived from an anti-IL-6 antibody wherein said at least one CDR is selected from those contained in the V_{L} or V_{H} polypeptides set forth in SEQ ID NO: 3, 18, 19, 652, 656, 657, 658, 661, 664, 665, 704, 708, 2, 20, 647, 651, 660, 666, 699, 702, 706, or 709.

Host cells and vectors comprising said polynucleotides are also contemplated.

In another specific embodiment the subject technology covers nucleic acid constructs containing any of the foregoing nucleic acid sequences and combinations thereof as well as recombinant cells containing these nucleic acid sequences and constructs containing wherein these nucleic acid sequences or constructs may be extrachromosomal or integrated into the host cell genome.

The subject technology further contemplates vectors comprising the polynucleotide sequences encoding the variable heavy and light chain polypeptide sequences, as well as the individual complementarity determining regions (CDRs, or hypervariable regions) set forth herein, as well as host cells comprising said sequences. In one embodiment of the subject technology, the host cell is a yeast cell. In another embodiment of the subject technology, the yeast host cell belongs to the genus Pichia.

In some instances, more than one exemplary polynucleotide encoding a given polypeptide sequence is provided, as summarized in Table 3.

TABLE 3
Multiple exemplary polynucleotides encoding particular polypeptides.

| Polypeptide SEQ ID NO | Exemplary coding SEQ ID NOs |
|---|---|
| 4 | 12, 111, 694 |
| 5 | 13, 112, 389, 501 |
| 6 | 14, 113, 695 |
| 9 | 17, 116, 697 |
| 39 | 47, 260 |
| 40 | 48, 261 |
| 60 | 68, 265 |
| 72 | 80, 325, 565, 581 |
| 89 | 97, 134, 166 |
| 103 | 12, 111, 694 |
| 104 | 13, 112, 389, 501 |
| 105 | 14, 113, 695 |
| 108 | 17, 116, 697 |
| 126 | 97, 134, 166 |
| 158 | 97, 134, 166 |
| 190 | 198, 214 |
| 191 | 199, 215 |
| 205 | 213, 469, 485 |
| 206 | 198, 214 |
| 207 | 199, 215 |
| 252 | 47, 260 |
| 253 | 48, 261 |
| 257 | 68, 265 |
| 317 | 80, 325, 565, 581 |
| 333 | 341, 533 |
| 381 | 13, 112, 389, 501 |
| 415 | 423, 439 |
| 431 | 423, 439 |
| 461 | 213, 469, 485 |
| 475 | 483, 499 |
| 476 | 484, 500 |
| 477 | 213, 469, 485 |
| 478 | 486, 502 |
| 479 | 487, 503 |
| 480 | 488, 504 |
| 481 | 489, 505 |
| 491 | 483, 499 |
| 492 | 484, 500 |
| 493 | 13, 112, 389, 501 |
| 494 | 486, 502 |
| 495 | 487, 503 |
| 496 | 488, 504 |
| 497 | 489, 505 |
| 525 | 341, 533 |
| 545 | 553, 585 |
| 554 | 562, 578 |
| 556 | 564, 580 |
| 557 | 80, 325, 565, 581 |
| 558 | 566, 582 |
| 570 | 562, 578 |
| 572 | 564, 580 |
| 573 | 80, 325, 565, 581 |
| 574 | 566, 582 |
| 577 | 553, 585 |

In some instances, multiple sequence identifiers refer to the same polypeptide or polynucleotide sequence, as summarized in Table 4. References to these sequence identifiers are understood to be interchangeable, except where context indicates otherwise.

TABLE 4
Repeated sequences. Each cell lists a group of repeated sequences
included in the sequence listing.
SEQ ID NOs of repeated sequences

4, 103
5, 104, 381, 493
6, 105
9, 108
12, 111
13, 112
14, 113
17, 116
39, 252
40, 253
48, 261
60, 257
68, 265
72, 317, 557, 573
80, 325, 565, 581
89, 126, 158
97, 134, 166
120, 659
190, 206
191, 207
198, 214
199, 215
205, 461, 477
213, 469
333, 525
415, 431
423, 439
475, 491
476, 492
478, 494
479, 495
480, 496
481, 497
483, 499
484, 500
486, 502
487, 503
488, 504
489, 505
545, 577
554, 570
556, 572
558, 574
562, 578
564, 580
566, 582

Certain exemplary embodiments include polynucleotides that hybridize under moderately or highly stringent hybridization conditions to a polynucleotide having one of the exemplary coding sequences recited in Table 1, and also include polynucleotides that hybridize under moderately or highly stringent hybridization conditions to a polynucleotide encoding the same polypeptide as a polynucleotide having one of the exemplary coding sequences recited in Table 1, or polypeptide encoded by any of the foregoing polynucleotides.

The phrase “high stringency hybridization conditions” refers to conditions under which a probe will hybridize to its target subsequence, typically in a complex mixture of nucleic acid, but to no other sequences. High stringency conditions are sequence dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, high stringency conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). High stringency conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). High stringency conditions may also be achieved with the addition of destabilizing agents such as formamide. For selective or specific hybridization, a positive signal is at least two times background, optionally 10 times background hybridization. Exemplary high stringency hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. Such hybridizations and wash steps can be carried out for, e.g., 1, 2, 5, 10, 15, 30, 60; or more minutes.

Nucleic acids that do not hybridize to each other under high stringency conditions are still substantially related if the polypeptides that they encode are substantially related. This occurs, for example, when a copy of a nucleic acid is created using the maximum codon degeneracy permitted by the genetic code. In such cases, the nucleic acids typically hybridize under moderate stringency hybridization conditions. Exemplary “moderate stringency hybridization conditions” include a hybridization in a buffer of 40% formamide, 1 M NaCl, 1% SDS at 37° C., and a wash in 1×SSC at 45° C. Such hybridizations and wash steps can be carried out for, e.g., 1, 2, 5, 10, 15, 30, 60, or more minutes. A positive hybridization is at least twice background. Those of ordinary skill will readily recognize that alternative hybridization and wash conditions can be utilized to provide conditions of similar stringency.

Expression vectors for use in the methods of the subject technology will further include yeast specific sequences, including a selectable auxotrophic or drug marker for identifying transformed yeast strains. A drug marker may further be used to amplify copy number of the vector in a yeast host cell.

The polypeptide coding sequence of interest is operably linked to transcriptional and translational regulatory sequences that provide for expression of the polypeptide in yeast cells. These vector components may include, but are not limited to, at least one of the following: an enhancer element, a promoter, and a transcription termination sequence. Sequences for the secretion of the polypeptide may also be included, e.g. a signal sequence, and the like. A yeast origin of replication is optional, as expression vectors are often integrated into the yeast genome.

In one embodiment of the subject technology, the polypeptide of interest is operably linked, or fused, to sequences providing for optimized secretion of the polypeptide from yeast diploid cells.

Nucleic acids are “operably linked” when placed into a functional relationship with another nucleic acid sequence. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence. Generally, “operably linked” means that the DNA sequences being linked are contiguous, and, in the case of a secretory leader, contiguous and in reading frame. However, enhancers do not have to be contiguous Linking is accomplished by ligation at convenient restriction sites or alternatively via a PCR/recombination method familiar to those skilled in the art (Gateway® Technology; Invitrogen, Carlsbad Calif.). If such sites do not exist, the synthetic oligonucleotide adapters or linkers are used in accordance with conventional practice.

Promoters are untranslated sequences located upstream (5′) to the start codon of a structural gene (generally within about 100 to 1000 bp) that control the transcription and translation of particular nucleic acid sequences to which they are operably linked. Such promoters fall into several classes: inducible, constitutive, and repressible promoters (that increase levels of transcription in response to absence of a repressor). Inducible promoters may initiate increased levels of transcription from DNA under their control in response to some change in culture conditions, e.g., the presence or absence of a nutrient or a change in temperature.

The yeast promoter fragment may also serve as the site for homologous recombination and integration of the expression vector into the same site in the yeast genome; alternatively a selectable marker is used as the site for homologous recombination. Pichia transformation is described in Cregg, et al. (1985) Mol. Cell. Biol. 5:3376-3385.

Examples of suitable promoters from Pichia include the AOX1 and promoter (Cregg, et al. (1989) Mol. Cell. Biol. 9:1316-1323); ICL1 promoter (Menendez, et al. (2003) Yeast 20(13):1097-108); glyceraldehyde-3-phosphate dehydrogenase promoter (GAP) (Waterham, et al. (1997) Gene 186(1):37-44); and FLD1 promoter (Shen, et al. (1998) Gene 216(1):93-102). The GAP promoter is a strong constitutive promoter and the AOX and FLD1 promoters are inducible.

Other yeast promoters include ADH1, alcohol dehydrogenase II, GAL4, PHO3, PHO5, Pyk, and chimeric promoters derived therefrom. Additionally, non-yeast promoters may be used in the subject technology such as mammalian, insect, plant, reptile, amphibian, viral, and avian promoters. Most typically the promoter will comprise a mammalian promoter (potentially endogenous to the expressed genes) or will comprise a yeast or viral promoter that provides for efficient transcription in yeast systems.

The polypeptides of interest may be produced recombinantly not only directly, but also as a fusion polypeptide with a heterologous polypeptide, e.g. a signal sequence or other polypeptide having a specific cleavage site at the N-terminus of the mature protein or polypeptide. In general, the signal sequence may be a component of the vector, or it may be a part of the polypeptide coding sequence that is inserted into the vector. The heterologous signal sequence selected preferably is one that is recognized and processed through one of the standard pathways available within the host cell. The S. cerevisiae alpha factor pre-pro signal has proven effective in the secretion of a variety of recombinant proteins from P. pastoris. Other yeast signal sequences include the alpha mating factor signal sequence, the invertase signal sequence, and signal sequences derived from other secreted yeast polypeptides. Additionally, these signal peptide sequences may be engineered to provide for enhanced secretion in diploid yeast expression systems. Other secretion signals of interest also include mammalian signal sequences, which may be heterologous to the protein being secreted, or may be a native sequence for the protein being secreted. Signal sequences include pre-peptide sequences, and in some instances may include propeptide sequences. Many such signal sequences are known in the art, including the signal sequences found on immunoglobulin chains, e.g., K28 preprotoxin sequence, PHA-E, FACE, human MCP-1, human serum albumin signal sequences, human Ig heavy chain, human Ig light chain, and the like. See Hashimoto, et al. (1998) Protein Eng 11(2): 75; and Kobayashi, et al. (1998) Therapeutic Apheresis 2(4): 257.

Transcription may be increased by inserting a transcriptional activator sequence into the vector. These activators are cis-acting elements of DNA, usually about from 10 to 300 bp, which act on a promoter to increase its transcription. Transcriptional enhancers are relatively orientation and position independent, having been found 5′ and 3′ to the transcription unit, within an intron, as well as within the coding sequence itself. The enhancer may be spliced into the expression vector at a position 5′ or 3′ to the coding sequence, but is preferably located at a site 5′ from the promoter.

Expression vectors used in eukaryotic host cells may also contain sequences necessary for the termination of transcription and for stabilizing the mRNA. Such sequences are commonly available from 3′ to the translation termination codon, in untranslated regions of eukaryotic or viral DNAs or cDNAs. These regions contain nucleotide segments transcribed as polyadenylated fragments in the untranslated portion of the mRNA.

Construction of suitable vectors containing at least one of the above-listed components employs standard ligation techniques or PCR/recombination methods. Isolated plasmids or DNA fragments are cleaved, tailored, and re-ligated in the form desired to generate the plasmids required or via recombination methods. For analysis to confirm correct sequences in plasmids constructed, the ligation mixtures are used to transform host cells, and successful transformants selected by antibiotic resistance (e.g. ampicillin or Zeocin® (phleomycin)) where appropriate. Plasmids from the transformants are prepared, analyzed by restriction endonuclease digestion and/or sequenced.

As an alternative to restriction and ligation of fragments, recombination methods based on att sites and recombination enzymes may be used to insert DNA sequences into a vector. Such methods are described, for example, by Landy (1989) Ann. Rev. Biochem. 58: 913-949; and are known to those of skill in the art. Such methods utilize intermolecular DNA recombination that is mediated by a mixture of lambda and E. coli-encoded recombination proteins. Recombination occurs between specific attachment (att) sites on the interacting DNA molecules. For a description of att sites see Weisberg and Landy (1983) Site-Specific Recombination in Phage Lambda Cold Spring Harbor, N.Y.:Cold Spring Harbor Press), pages 211-250. The DNA segments flanking the recombination sites are switched, such that after recombination, the att sites are hybrid sequences comprised of sequences donated by each parental vector. The recombination can occur between DNAs of any topology.

Att sites may be introduced into a sequence of interest by ligating the sequence of interest into an appropriate vector; generating a PCR product containing att B sites through the use of specific primers; generating a cDNA library cloned into an appropriate vector containing att sites.

The expression host may be further modified by the introduction of sequences encoding at least one enzymes that enhance folding and disulfide bond formation, i.e. foldases, chaperonins, Such sequences may be constitutively or inducibly expressed in the yeast host cell, using vectors, markers, are known in the art. Preferably the sequences, including transcriptional regulatory elements sufficient for the desired pattern of expression, are stably integrated in the yeast genome through a targeted methodology.

For example, the eukaryotic PDI is not only an efficient catalyst of protein cysteine oxidation and disulfide bond isomerization, but also exhibits chaperone activity. Co-expression of PDI can facilitate the production of active proteins having multiple disulfide bonds. Also of interest is the expression of BIP (immunoglobulin heavy chain binding protein); cyclophilin; and the like. In one embodiment of the subject technology, each of the haploid parental strains expresses a distinct folding enzyme, e.g. one strain may express BIP, and the other strain may express PDI or combinations thereof.

Vectors are used to introduce a foreign substance, such as DNA, RNA or protein, into an organism or host cell. Typical vectors include recombinant viruses (for polynucleotides) and liposomes or other lipid aggregates (for polypeptides and/or polynucleotides). A “DNA vector” is a replicon, such as plasmid, phage or cosmid, to which another polynucleotide segment may be attached so as to bring about the replication of the attached segment. An “expression vector” is a DNA vector which contains regulatory sequences which will direct polypeptide synthesis by an appropriate host cell. This usually means a promoter to bind RNA polymerase and initiate transcription of mRNA, as well as ribosome binding sites and initiation signals to direct translation of the mRNA into a polypeptide(s). Incorporation of a polynucleotide sequence into an expression vector at the proper site and in correct reading frame, followed by transformation of an appropriate host cell by the vector, enables the production of a polypeptide encoded by said polynucleotide sequence. Exemplary expression vectors and techniques for their use are described in the following publications: Old, et al. (1989) Principles of Gene Manipulation: An Introduction to Genetic Engineering, Blackwell Scientific Publications, 4th edition; Sambrook, et al. (1989) Molecular Cloning: A Laboratory Manual, 2nd Edition, Cold Spring Harbor Laboratory Press; Sambrook et al. (2001) Molecular Cloning: A Laboratory Manual, 3rd Edition, Cold Spring Harbor Laboratory Press; Gorman, “High Efficiency Gene Transfer into Mammalian Cells,” in DNA Cloning, Volume II, Glover, D. M., Ed., IRL Press, Washington, D.C., pages 143-190.

For example, a liposomes or other lipid aggregate may comprise a lipid such as phosphatidylcholines (lecithins) (PC), phosphatidylethanolamines (PE), lysolecithins, lysophosphatidylethanolamines, phosphatidylserines (PS), phosphatidylglycerols (PG), phosphatidylinositol (PI), sphingomyelins, cardiolipin, phosphatidic acids (PA), fatty acids, gangliosides, glucolipids, glycolipids, mono-, di or triglycerides, ceramides, cerebrosides and combinations thereof; a cationic lipid (or other cationic amphiphile) such as 1,2-dioleyloxy-3-(trimethylamino) propane (DOTAP); N-cholesteryloxycarbaryl-3,7,12-triazapentadecane-1,15-diamine (CTAP); N-[1-(2,3-ditetradecyloxy)propyl]-N,N-dimethyl-N-hydroxyethylammonium bromide (DMRIE); N-[1-(2,3-dioleyloxy)propyl]-N,N-dimethyl-N-hydroxy ethylammonium bromide (DORIE); N-[1-(2,3-dioleyloxy)propyl]-N,N,N-trimethylammonium chloride (DOTMA); 3 beta [N-(N′,N′-dimethylaminoethane)carbamoly] cholesterol (DC-Choi); and dimethyldioctadecylammonium (DDAB); dioleoylphosphatidyl ethanolamine (DOPE), cholesterol-containing DOPC; and combinations thereof; and/or a hydrophilic polymer such as polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide and combinations thereof. Other suitable cationic lipids are described in Miller (1998) Angewandte Chemie International Edition 37(13-14): 1768-1785 and Cooper, et al. (1998) Chem. Eur. J. 4(1): 137-151. Liposomes can be crosslinked, partially crosslinked, or free from crosslinking Crosslinked liposomes can include crosslinked as well as non-crosslinked components. Suitable cationic liposomes or cytofectins are commercially available and can also be prepared as described in Sipkins et al. (1998) Nature Medicine 4(5): 623-626 or as described in Miller, supra. Exemplary liposomes include a polymerizable zwitterionic or neutral lipid, a polymerizable integrin targeting lipid and a polymerizable cationic lipid suitable for binding a nucleic acid. Liposomes can optionally include peptides that provide increased efficiency, for example as described in U.S. Pat. No. 7,297,759. Additional exemplary liposomes and other lipid aggregates are described in U.S. Pat. No. 7,166,298.

### Additional Exemplary Embodiments of the Subject Technology

In another embodiment, the subject technology contemplates at least one anti-IL-6 antibodies or antibody fragments or variants thereof which may specifically bind to the same linear or conformational epitope(s) and/or compete for binding to the same linear or conformational epitope(s) on an intact human IL-6 polypeptide or fragment thereof as an anti-IL-6 antibody comprising Ab1 and chimeric, humanized, single chain antibodies and fragments thereof (containing at least one CDRs of the afore-identified antibodies) that specifically bind IL-6, which preferably are aglycosylated. In a preferred embodiment, the anti-IL-6 antibody or fragment or variant thereof may specifically bind to the same linear or conformational epitope(s) and/or compete for binding to the same linear or conformational epitope(s) on an intact human IL-6 polypeptide or a fragment thereof as Ab1 and chimeric, humanized, single chain antibodies and fragments thereof (containing at least one CDRs of the afore-mentioned antibody) that specifically bind IL-6, which preferably are aglycosylated.

In another embodiment of the subject technology, the anti-IL-6 antibody which may specifically bind to the same linear or conformational epitopes on an intact IL-6 polypeptide or fragment thereof that is (are) specifically bound by Ab1 may bind to an IL-6 epitope(s) ascertained by epitopic mapping using overlapping linear peptide fragments which span the full length of the native human IL-6 polypeptide. In one embodiment of the subject technology, the IL-6 epitope comprises, or alternatively consists of, at least one residues comprised in IL-6 fragments selected from those respectively encompassing amino acid residues 37-51, amino acid residues 70-84, amino acid residues 169-183, amino acid residues 31-45 and/or amino acid residues 58-72.

The subject technology is also directed to an anti-IL-6 antibody that binds with the same IL-6 epitope and/or competes with an anti-IL-6 antibody for binding to IL-6 as an antibody or antibody fragment disclosed herein, including but not limited to an anti-IL-6 antibody selected from Ab1 and chimeric, humanized, single chain antibodies and fragments thereof (containing at least one CDRs of the afore-mentioned antibody) that specifically bind IL-6, which preferably are aglycosylated.

In another embodiment, the subject technology is also directed to an isolated anti-IL-6 antibody or antibody fragment or variant thereof comprising at least one of the CDRs contained in the V_{H} polypeptide sequences comprising: SEQ ID NO: 3, 18, 19, 22, 38, 54, 70, 86, 102, 117, 118, 123, 139, 155, 171, 187, 203, 219, 235, 251, 267, 283, 299, 315, 331, 347, 363, 379, 395, 411, 427, 443, 459, 475, 491, 507, 523, 539, 555, 571, 652, 656, 657, 658, 661, 664, 665, 668, 672, 676, 680, 684, 688, 691, 692, 704, or 708 and/or at least one of the CDRs contained in the V_{L} polypeptide sequence consisting of: 2, 20, 21, 37, 53, 69, 85, 101, 119, 122, 138, 154, 170, 186, 202, 218, 234, 250, 266, 282, 298, 314, 330, 346, 362, 378, 394, 410, 426, 442, 458, 474, 490, 506, 522, 538, 554, 570, 647, 651, 660, 666, 667, 671, 675, 679, 683, 687, 693, 699, 702, 706, or 709 and the V_{H} and V_{L} sequences depicted in the antibody alignments comprised in FIGS. 8-11 of this application.

In one embodiment of the subject technology, the anti-IL-6 antibody described herein may comprise at least 2 complementarity determining regions (CDRs) in each the variable light and the variable heavy regions which are identical to those contained in an anti-IL-6 antibody comprising Ab1 and chimeric, humanized, single chain antibodies and fragments thereof (containing at least one CDRs of the afore-mentioned antibody) that specifically bind IL-6, which preferably are aglycosylated.

In a preferred embodiment, the anti-IL-6 antibody described herein may comprise at least 2 complementarity determining regions (CDRs) in each the variable light and the variable heavy regions which are identical to those contained in Ab1. In another embodiment, all of the CDRs of the anti-IL-6 antibody discussed above are identical to the CDRs contained in an anti-IL-6 antibody comprising Ab1 and chimeric, humanized, single chain antibodies and fragments thereof (containing at least one CDRs of the afore-mentioned antibody) that specifically bind IL-6, which preferably are aglycosylated. In a preferred embodiment of the subject technology, all of the CDRs of the anti-IL-6 antibody discussed above are identical to the CDRs contained in Ab1, e.g., an antibody comprised of the V_{H} and V_{L} sequences comprised in SEQ ID NO: 657 and SEQ ID NO: 709 respectively.

The subject technology further contemplates that the one or more anti-IL-6 antibodies discussed above are aglycosylated or substantially non-glycosylated (e.g., may contain one or more, e.g., 1-5 mannose residues); that contain an Fc region that has been modified to alter effector function, half-life, proteolysis, and/or glycosylation; are human, humanized, single chain or chimeric; and are a humanized antibody derived from a rabbit (parent) anti-IL-6 antibody. Exemplary constant regions that provide for the production of aglycosylated antibodies in Pichia are comprised in SEQ ID NO: 588 and SEQ ID NO: 586 which respectively are encoded by the nucleic acid sequences in SEQ ID NO: 589 and SEQ ID NO: 587.

The subject technology further contemplates at least one anti-IL-6 antibodies wherein the framework regions (FRs) in the variable light region and the variable heavy regions of said antibody respectively are human FRs which are unmodified or which have been modified by the substitution of at most 2 or 3 human FR residues in the variable light or heavy chain region with the corresponding FR residues of the parent rabbit antibody, and wherein said human FRs have been derived from human variable heavy and light chain antibody sequences which have been selected from a library of human germline antibody sequences based on their high level of homology to the corresponding rabbit variable heavy or light chain regions relative to other human germline antibody sequences contained in the library.

In one embodiment of the subject technology, the anti-IL-6 antibody or fragment or variant thereof may specifically bind to IL-6 expressing human cells and/or to circulating soluble IL-6 molecules in vivo, including IL-6 expressed on or by human cells in a patient with a disease associated with cells that express IL-6.

The subject technology further contemplates anti-IL-6 antibodies or fragments or variants thereof directly or indirectly attached to a detectable label or therapeutic agent.

The subject technology also contemplates at least one nucleic acid sequences which result in the expression of an anti-IL-6 antibody or antibody fragment or variant thereof as set forth above, including those comprising, or alternatively consisting of, yeast or human preferred codons. The subject technology also contemplates vectors (including plasmids or recombinant viral vectors) comprising said nucleic acid sequence(s). The subject technology also contemplates host cells or recombinant host cells expressing at least one of the antibodies set forth above, including a mammalian, yeast, bacterial, and insect cells. In a preferred embodiment, the host cell is a yeast cell. In a further preferred embodiment, the yeast cell is a diploidal yeast cell. In a more preferred embodiment, the yeast cell is a Pichia yeast.

The subject technology also contemplates a method of treatment comprising administering to a patient with a disease or condition associated with psoriatic arthritis a therapeutically effective amount of at least one anti-IL-6 antibody or antigen-binding fragment or variant thereof. The diseases that may be treated are presented in the non-limiting list set forth above. In another embodiment the treatment further includes the administration of another therapeutic agent or regimen selected from chemotherapy, radiotherapy, cytokine administration or gene therapy agent. For example, drugs associated with the treatment of psoriatic arthritis including but not limited to TNF-α inhibitors, glyococordicoids, triamcinolone, dexamethasone, prednisone, may also be administered sequentially or subsequently with at least one anti-IL-6 antibody or antigen-binding fragment or variant thereof described herein. Further examples of drugs associated with the treatment of psoriatic arthritis include but are not limited to ARISTOCORT (triamcinolone), BAYCADROM (dexamethasone), DECADRON (dexamethasone), DELTASONE (prednisone), DEXAMETHASONE INTENSOL (dexamethasone), ENBREL (etancercept), HUMIRA (adalimumab), REMICADE (infliximab), RIDUARA (aruaofin), and SIMPONI® (golimumab).

### Anti-IL-6 Activity

As stated previously, IL-6 is a member of a family of cytokines that promote cellular responses through a receptor complex consisting of at least one subunit of the signal-transducing glycoprotein gp130 and the IL-6 receptor (IL-6R). The IL-6R may also be present in a soluble form (sIL-6R). IL-6 binds to IL-6R, which then dimerizes the signal-transducing receptor gp130.

It is believed that the anti-IL-6 antibodies of the subject technology, or IL-6 binding fragments or variants thereof, are useful by exhibiting anti-IL-6 activity. In one non-limiting embodiment of the subject technology, the anti-IL-6 antibodies of the subject technology, or IL-6 binding fragments or variants thereof, exhibit anti-IL-6 activity by binding to IL-6 which may be soluble IL-6 or cell surface expressed IL-6 and/or may prevent or inhibit the binding of IL-6 to IL-6R and/or activation (dimerization) of the gp130 signal-transducing glycoprotein and the formation of IL-6/IL-6R/gp130 multimers and the biological effects of any of the foregoing. The subject anti-IL-6 antibodies may possess different antagonistic activities based on where (i.e., epitope) the particular antibody binds IL-6 and/or how it affects the formation of the foregoing IL-6 complexes and/or multimers and the biological effects thereof. Consequently, different anti-IL-6 antibodies according to the subject technology e.g., may be better suited for preventing or treating conditions involving the formation and accumulation of substantial soluble IL-6 such as rheumatoid arthritis whereas other antibodies may be favored in treatments wherein the prevention of IL-6/IL-6R/gp130 or IL-6/IL-6R/gp130 multimers is a desired therapeutic outcome. This can be determined in binding and other assays.

The anti-IL-6 activity of the anti-IL-6 antibody of the present subject technology, and fragments and variants thereof having binding specificity to IL-6, may also be described by their strength of binding or their affinity for IL-6. This also may affect their therapeutic properties. In one embodiment of the subject technology, the anti-IL-6 antibodies of the present subject technology, and fragments thereof having binding specificity to IL-6, bind to IL-6 with a dissociation constant (K_{D}) of less than or equal to 5×10⁻⁷, 10⁻⁷, 5×10⁻⁸, 10⁻⁸, 5×10⁻⁹, 10⁻⁹, 5×10⁻¹⁰, 10⁻¹⁰, 5×10⁻¹¹, 10⁻¹¹, 5×10⁻¹², 10⁻¹², 5×10⁻¹³, 10⁻¹³, 5×10⁻¹⁴, 10⁻¹⁴, 5×10⁻¹⁵ or 10⁻¹⁵. Preferably, the anti-IL-6 antibodies and fragments and variants thereof bind IL-6 with a dissociation constant of less than or equal to 5×10⁻¹⁰.

In another embodiment of the subject technology, the anti-IL-6 activity of the anti-IL-6 antibodies of the present subject technology, and fragments and variants thereof having binding specificity to IL-6, bind to IL-6 with an off-rate of less than or equal to 10⁻⁴ S⁻¹, 5×10⁻⁵ S⁻¹, 10⁻⁵ S⁻¹, 5×10⁻⁶ S⁻¹, 10⁻⁶ S⁻¹, 5×10⁻⁷ S⁻¹, or 10⁻⁷ S⁻¹. In one embodiment of the subject technology, the anti-IL-6 antibodies of the subject technology, and fragments and variants thereof having binding specificity to IL-6, bind to a linear or conformational IL-6 epitope.

In a further embodiment of the subject technology, the anti-IL-6 activity of the anti-IL-6 antibodies of the present subject technology, and fragments and variants thereof having binding specificity to IL-6, exhibit anti-IL-6 activity by ameliorating or reducing the symptoms of, or alternatively treating, or preventing, diseases and disorders associated with IL-6. Non-limiting examples of diseases and disorders associated with IL-6 are set forth infra. In another embodiment of the subject technology, the anti-IL-6 antibodies described herein, or IL-6 binding fragments and variants thereof, do not have binding specificity for IL-6R or the gp-130 signal-transducing glycoprotein.

### B-Cell Screening and Isolation

In one embodiment, the present subject technology provides methods of isolating a clonal population of antigen-specific B cells that may be used for isolating at least one antigen-specific cell. As described and exemplified infra, these methods contain a series of culture and selection steps that can be used separately, in combination, sequentially, repetitively, or periodically. Preferably, these methods are used for isolating at least one antigen-specific cell, which can be used to produce a monoclonal antibody, which is specific to a desired antigen, or a nucleic acid sequence corresponding to such an antibody.

In one embodiment, the present subject technology provides a method comprising the steps of:
(a) preparing a cell population comprising at least one antigen-specific B cell;
(b) enriching the cell population, e.g., by chromatography, to form an enriched cell population comprising at least one antigen-specific B cell;
(c) isolating a single B cell from the enriched B cell population; and
(d) determining whether the single B cell produces an antibody specific to the antigen.

In another embodiment, the present subject technology provides an improvement to a method of isolating a single, antibody-producing B cell, the improvement comprising enriching a B cell population obtained from a host that has been immunized or naturally exposed to an antigen, wherein the enriching step precedes any selection steps, comprises at least one culturing step, and results in a clonal population of B cells that produces a single monoclonal antibody specific to said antigen.

Throughout this application, a “clonal population of B cells” refers to a population of B cells that only secrete a single antibody specific to a desired antigen. That is to say that these cells produce only one type of monoclonal antibody specific to the desired antigen.

In the present application, “enriching” a cell population cells means increasing the frequency of desired cells, typically antigen-specific cells, contained in a mixed cell population, e.g., a B cell-containing isolate derived from a host that is immunized against a desired antigen. Thus, an enriched cell population encompasses a cell population having a higher frequency of antigen-specific cells as a result of an enrichment step, but this population of cells may contain and produce different antibodies.

The general term “cell population” encompasses pre- and a post-enrichment cell populations, keeping in mind that when multiple enrichment steps are performed, a cell population can be both pre- and post-enrichment. For example, in one embodiment, the present subject technology provides a method:
(a) harvesting a cell population from an immunized host to obtain a harvested cell population;
(b) creating at least one single cell suspension from the harvested cell population;
(c) enriching at least one single cell suspension to form a first enriched cell population;
(d) enriching the first enriched cell population to form a second enriched cell population;
(e) enriching the second enriched cell population to form a third enriched cell population; and
(f) selecting an antibody produced by an antigen-specific cell of the third enriched cell population.

Each cell population may be used directly in the next step, or it can be partially or wholly frozen for long- or short- term storage or for later steps. Also, cells from a cell population can be individually suspended to yield single cell suspensions. The single cell suspension can be enriched, such that a single cell suspension serves as the pre-enrichment cell population. Then, at least one antigen-specific single cell suspensions together form the enriched cell population; the antigen-specific single cell suspensions can be grouped together, e.g., re-plated for further analysis and/or antibody production.

In one embodiment, the present subject technology provides a method of enriching a cell population to yield an enriched cell population having an antigen-specific cell frequency that is about 50% to about 100%, or increments therein. Preferably, the enriched cell population has an antigen-specific cell frequency at least about 50%, 60%, 70%, 75%, 80%, 90%, 95%, 99%, or 100%.

In another embodiment, the present subject technology provides a method of enriching a cell population whereby the frequency of antigen-specific cells is increased by at least about 2-fold, 5-fold, 10-fold, 20-fold, 50-fold, 100-fold, or increments therein.

Throughout this application, the term “increment” is used to define a numerical value in varying degrees of precision, e.g., to the nearest 10, 1, 0.1, 0.01. The increment can be rounded to any measurable degree of precision, and the increment need not be rounded to the same degree of precision on both sides of a range. For example, the range 1 to 100 or increments therein includes ranges such as 20 to 80, 5 to 50, and 0.4 to 98. When a range is open-ended, e.g., a range of less than 100, increments therein means increments between 100 and the measurable limit. For example, less than 100 or increments therein means 0 to 100 or increments therein unless the feature, e.g., temperature, is not limited by 0.

Antigen-specificity can be measured with respect to any antigen. The antigen can be any substance to which an antibody can bind including, but not limited to, peptides, proteins or fragments thereof; carbohydrates; organic and inorganic molecules; receptors produced by animal cells, bacterial cells, and viruses; enzymes; agonists and antagonists of biological pathways; hormones; and cytokines Exemplary antigens include, but are not limited to, IL-2, IL-4, IL-6, IL-10, IL-12, IL-13, IL-18, IFN-α, IFN-γ, BAFF, CXCL13, IP-10, VEGF, EPO, EGF, HRG, Hepatocyte Growth Factor (HGF) and Hepcidin. Preferred antigens include IL-6, IL-13, TNF-α, VEGF-α, Hepatocyte Growth Factor (HGF) and Hepcidin. In a method utilizing more than one enrichment step, the antigen used in each enrichment step can be the same as or different from one another. Multiple enrichment steps with the same antigen may yield a large and/or diverse population of antigen-specific cells; multiple enrichment steps with different antigens may yield an enriched cell population with cross-specificity to the different antigens.

Enriching a cell population can be performed by any cell-selection means known in the art for isolating antigen-specific cells. For example, a cell population can be enriched by chromatographic techniques, e.g., Miltenyi bead or magnetic bead technology. The beads can be directly or indirectly attached to the antigen of interest. In a preferred embodiment, the method of enriching a cell population includes at least one chromatographic enrichment step.

A cell population can also be enriched by performed by any antigen-specificity assay technique known in the art, e.g., an ELISA assay or a halo assay. ELISA assays include, but are not limited to, selective antigen immobilization (e.g., biotinylated antigen capture by streptavidin, avidin, or neutravidin coated plate), non-specific antigen plate coating, and through an antigen build-up strategy (e.g., selective antigen capture followed by binding partner addition to generate a heteromeric protein-antigen complex). The antigen can be directly or indirectly attached to a solid matrix or support, e.g., a column. A halo assay comprises contacting the cells with antigen-loaded beads and labeled anti-host antibody specific to the host used to harvest the B cells. The label can be, e.g., a fluorophore. In one embodiment, at least one assay enrichment step is performed on at least one single cell suspension. In another embodiment, the method of enriching a cell population includes at least one chromatographic enrichment step and at least one assay enrichment step.

Methods of “enriching” a cell population by size or density are known in the art. See, e.g., U.S. Pat. No. 5,627,052. These steps can be used in the present method in addition to enriching the cell population by antigen-specificity.

The cell populations of the present subject technology contain at least one cell capable of recognizing an antigen. Antigen-recognizing cells include, but are not limited to, B cells, plasma cells, and progeny thereof. In one embodiment, the present subject technology provides a clonal cell population containing a single type of antigen-specific B-cell, i.e., the cell population produces a single monoclonal antibody specific to a desired antigen.

In such embodiment, it is believed that the clonal antigen-specific population of B cells consists predominantly of antigen-specific, antibody-secreting cells, which are obtained by the novel culture and selection protocol provided herein. Accordingly, the present subject technology also provides methods for obtaining an enriched cell population containing at least one antigen-specific, antibody-secreting cell. In one embodiment, the present subject technology provides an enriched cell population containing about 50% to about 100%, or increments therein, at least about 60%, 70%, 80%, 90%, or 100% of antigen-specific, antibody-secreting cells.

In one embodiment, the present subject technology provides a method of isolating a single B cell by enriching a cell population obtained from a host before any selection steps, e.g., selecting a particular B cell from a cell population and/or selecting an antibody produced by a particular cell. The enrichment step can be performed as one, two, three, or more steps. In one embodiment, a single B cell is isolated from an enriched cell population before confirming whether the single B cell secretes an antibody with antigen-specificity and/or a desired property.

In one embodiment, a method of enriching a cell population is used in a method for antibody production and/or selection. Thus, the present subject technology provides a method comprising enriching a cell population before selecting an antibody. The method can include the steps of: preparing a cell population comprising at least one antigen-specific cell, enriching the cell population by isolating at least one antigen-specific cell to form an enriched cell population, and inducing antibody production from at least one antigen-specific cell. In a preferred embodiment, the enriched cell population contains more than one antigen-specific cell. In one embodiment, each antigen-specific cell of the enriched population is cultured under conditions that yield a clonal antigen-specific B cell population before isolating an antibody producing cell therefrom and/or producing an antibody using said B cell, or a nucleic acid sequence corresponding to such an antibody. In contrast to prior techniques where antibodies are produced from a cell population with a low frequency of antigen-specific cells, the present subject technology allows antibody selection from among a high frequency of antigen-specific cells. Because an enrichment step is used prior to antibody selection, the majority of the cells, preferably virtually all of the cells, used for antibody production are antigen-specific. By producing antibodies from a population of cells with an increased frequency of antigen specificity, the quantity and variety of antibodies are increased.

In the antibody selection methods of the present subject technology, an antibody is preferably selected after an enrichment step and a culture step that results in a clonal population of antigen-specific B cells. The methods can further comprise a step of sequencing a selected antibody or portions thereof from at least one isolated, antigen-specific cells. Any method known in the art for sequencing can be employed and can include sequencing the heavy chain, light chain, variable region(s), and/or complementarity determining region(s) (CDR).

In addition to the enrichment step, the method for antibody selection can also include at least one steps of screening a cell population for antigen recognition and/or antibody functionality. For example, the desired antibodies may have specific structural features, such as binding to a particular epitope or mimicry of a particular structure; antagonist or agonist activity; or neutralizing activity, e.g., inhibiting binding between the antigen and a ligand. In one embodiment, the antibody functionality screen is ligand-dependent. Screening for antibody functionality includes, but is not limited to, an in vitro protein-protein interaction assay that recreates the natural interaction of the antigen ligand with recombinant receptor protein; and a cell-based response that is ligand dependent and easily monitored (e.g., proliferation response). In one embodiment, the method for antibody selection includes a step of screening the cell population for antibody functionality by measuring the inhibitory concentration (IC50). In one embodiment, at least one of the isolated, antigen-specific cells produces an antibody having an IC50 of less than about 100, 50, 30, 25, 10 μg/mL, or increments therein.

In addition to the enrichment step, the method for antibody selection can also include at least one steps of screening a cell population for antibody binding strength. Antibody binding strength can be measured by any method known in the art (e.g., Biacore®). In one embodiment, at least one of the isolated, antigen-specific cells produces an antibody having a high antigen affinity, e.g., a dissociation constant (Kd) of less than about 5×10⁻¹⁰ M-1, preferably about 1×10⁻¹³ to 5×10⁻¹⁰, 1×10⁻¹² to 1×10⁻¹⁰, 1×10⁻¹² to 7.5×10⁻¹¹, 1×10⁻¹¹, 2×10⁻¹¹, about 1.5×10⁻¹¹ or less, or increments therein. In this embodiment, the antibodies are said to be affinity mature. In a preferred embodiment, the affinity of the antibodies is comparable to or higher than the affinity of any one of Panorex® (edrecolomab), Rituxan® (rituximab), Herceptin® (traztuzumab), Mylotarg® (gentuzumab), Campath® (alemtuzumab), Zevalin® (ibritumomab), Erbitux® (cetuximab), Avastin® (bevicizumab), Raptiva® (efalizumab), Remicade® (infliximab), Humira® (adalimumab), and Xolair® (omalizumab). Preferably, the affinity of the antibodies is comparable to or higher than the affinity of Humira®. The affinity of an antibody can also be increased by known affinity maturation techniques. In one embodiment, at least one cell population is screened for at least one of, preferably both, antibody functionality and antibody binding strength.

In addition to the enrichment step, the method for antibody selection can also include at least one steps of screening a cell population for antibody sequence homology, especially human homology. In one embodiment, at least one of the isolated, antigen-specific cells produces an antibody that has a homology to a human antibody of at least about 50% to about 100%, or increments therein, or at least about 60%, 70%, 80%, 85%, 90%, or 95% homologous. The antibodies can be humanized to increase the homology to a human sequence by techniques known in the art such as CDR grafting or selectivity determining residue grafting (SDR).

In another embodiment, the present subject technology also provides the antibodies themselves according to any of the embodiments described above in terms of IC50, Kd, and/or homology.

### Methods of Humanizing Antibodies

In another embodiment of the subject technology, there is provided a method for humanizing antibody heavy and light chains. In this embodiment, the following method is followed for the humanization of the heavy and light chains:

Light Chain

1. Identify the amino acid that is the first one following the signal peptide sequence. This is the start of Framework 1. The signal peptide starts at the first initiation methionine and is typically, but not necessarily 22 amino acids in length for rabbit light chain protein sequences. The start of the mature polypeptide can also be determined experimentally by N-terminal protein sequencing, or can be predicted using a prediction algorithm. This is also the start of Framework 1 as classically defined by those in the field.

### Example

### RbtV_{L} Amino acid residue 1 in FIG. 1, starting ‘AYDM . . . ’ (SEQ ID NO: 733)

2. Identify the end of Framework

3. This is typically 86-90 amino acids following the start of Framework 1 and is typically a cysteine residue preceded by two tyrosine residues. This is the end of the Framework 3 as classically defined by those in the field.

### Example

### RbtV_{L} amino acid residue 88 in FIG. 1, ending as ‘TYYC’ (SEQ ID NO: 733)

3. Use the rabbit light chain sequence of the polypeptide starting from the beginning of Framework 1 to the end of Framework 3 as defined above and perform a sequence homology search for the most similar human antibody protein sequences. This will typically be a search against human germline sequences prior to antibody maturation in order to reduce the possibility of immunogenicity, however any human sequences can be used. Typically a program like BLAST can be used to search a database of sequences for the most homologous. Databases of human antibody sequences can be found from various sources such as NCBI (National Center for Biotechnology Information).

### Example

RbtV_{L} amino acid sequence from residues numbered 1 through 88 in FIG. 1 is BLASTed against a human antibody germline database. The top three unique returned sequences are shown in FIG. 1 as L12A (SEQ ID NO: 734), V1 (SEQ ID NO: 735), and Vx02 (SEQ ID NO: 736).

4. Generally the most homologous human germline variable light chain sequence is then used as the basis for humanization. However those skilled in the art may decide to use another sequence that wasn't the highest homology as determined by the homology algorithm, based on other factors including sequence gaps and framework similarities.

### Example

In FIG. 1, L12A (SEQ ID NO: 734) was the most homologous human germline variable light chain sequence and is used as the basis for the humanization of RbtV_{L}.

5. Determine the framework and CDR arrangement (FR1, FR2, FR3, CDR1 & CDR2) for the human homolog being used for the light chain humanization. This is using the traditional layout as described in the field. Align the rabbit variable light chain sequence with the human homolog, while maintaining the layout of the framework and CDR regions.

### Example

In FIG. 1, the RbtV_{L} sequence is aligned with the human homologous sequence L12A, and the framework and CDR domains are indicated.

6. Replace the human homologous light chain sequence CDR1 and CDR2 regions with the CDR1 and CDR2 sequences from the rabbit sequence. If there are differences in length between the rabbit and human CDR sequences then use the entire rabbit CDR sequences and their lengths. It is possible that the specificity, affinity and/or immunogenicity of the resulting humanized antibody may be unaltered if smaller or larger sequence exchanges are performed, or if specific residue(s) are altered, however the exchanges as described have been used successfully, but do not exclude the possibility that other changes may be permitted.

### Example

In FIG. 1, the CDR1 and CDR2 amino acid residues of the human homologous variable light chain L12A are replaced with the CDR1 and CDR2 amino acid sequences from the RbtV_{L} rabbit antibody light chain sequence. The human L12A frameworks 1, 2 and 3 are unaltered. The resulting humanized sequence is shown below as V_{L}h from residues numbered 1 through 88. Note that the only residues that are different from the L12A human sequence are underlined, and are thus rabbit-derived amino acid residues. In this example only 8 of the 88 residues are different than the human sequence.

7. After framework 3 of the new hybrid sequence created in Step 6, attach the entire CDR3 of the rabbit light chain antibody sequence. The CDR3 sequence can be of various lengths, but is typically 9 to 15 amino acid residues in length. The CDR3 region and the beginning of the following framework 4 region are defined classically and identifiable by those skilled in the art. Typically the beginning of Framework 4, and thus after the end of CDR3 consists of the sequence ‘FGGG . . . ’ (SEQ ID NO: 743), however some variation may exist in these residues.

### Example

In FIG. 1, the CDR3 of RbtV_{L} (amino acid residues numbered 89-100) is added after the end of framework 3 in the humanized sequence indicated as V_{L}h.

8. The rabbit light chain framework 4, which is typically the final 11 amino acid residues of the variable light chain and begins as indicated in Step 7 above and typically ends with the amino acid sequence ‘ . . . VVKR’ (SEQ ID NO: 744) is replaced with the nearest human light chain framework 4 homolog, usually from germline sequence. Frequently this human light chain framework 4 is of the sequence ‘FGGGTKVEIKR’ (SEQ ID NO: 745). It is possible that other human light chain framework 4 sequences that are not the most homologous or otherwise different may be used without affecting the specificity, affinity and/or immunogenicity of the resulting humanized antibody. This human light chain framework 4 sequence is added to the end of the variable light chain humanized sequence immediately following the CDR3 sequence from Step 7 above. This is now the end of the variable light chain humanized amino acid sequence.

### Example

In FIG. 1, Framework 4 (FR4) of the RbtV_{L} rabbit light chain sequence is shown above a homologous human FR4 sequence. The human FR4 sequence is added to the humanized variable light chain sequence (V_{L}h) right after the end of the CD3 region added in Step 7 above.

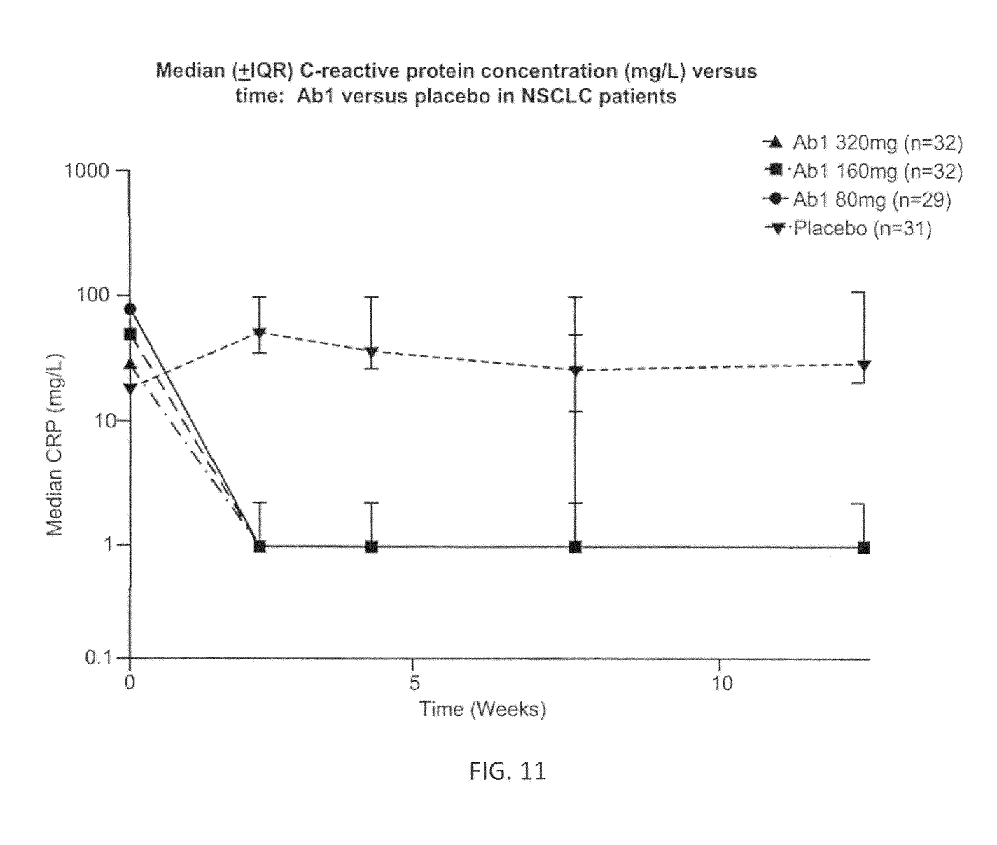

In addition, FIGS. 8 and 9 depict preferred humanized anti-IL-6 variable heavy and variable light chain sequences humanized from the variable heavy and light regions in Ab1 according to the subject technology. These humanized light and heavy chain regions are respectively contained in the polypeptides set forth in SEQ ID NO: 647, or 651 and in SEQ ID NO: 652, 656, 657 or 658. The CDR2 of the humanized variable heavy region in SEQ ID NO: 657 (containing a serine substitution in CDR2) is set forth in SEQ ID NO: 658. Alignments illustrating variants of the light and heavy chains are shown in FIGS. 10 and 11, respectively, with sequence differences within the CDR regions highlighted. Sequence identifiers of CDR sequences and of exemplary coding sequences are summarized in Table 1, above.

Heavy Chain

1. Identify the amino acid that is the first one following the signal peptide sequence. This is the start of Framework 1. The signal peptide starts at the first initiation methionine and is typically 19 amino acids in length for rabbit heavy chain protein sequences. Typically, but not necessarily always, the final 3 amino acid residues of a rabbit heavy chain signal peptide are ‘ . . . VQC’, followed by the start of Framework 1. The start of the mature polypeptide can also be determined experimentally by N-terminal protein sequencing, or can be predicted using a prediction algorithm. This is also the start of Framework 1 as classically defined by those in the field.

### Example

### RbtV_{H} Amino acid residue 1 in FIG. 1, starting ‘QEQL . . . ’ (SEQ ID NO: 738)

2. Identify the end of Framework 3. This is typically 95-100 amino acids following the start of Framework 1 and typically has the final sequence of ‘ . . . CAR’ (although the alanine can also be a valine). This is the end of the Framework 3 as classically defined by those in the field.

### Example

### RbtV_{H} amino acid residue 98 in FIG. 1, ending as ‘ . . . FCVR’ (SEQ ID NO: 738)

3. Use the rabbit heavy chain sequence of the polypeptide starting from the beginning of Framework 1 to the end of Framework 3 as defined above and perform a sequence homology search for the most similar human antibody protein sequences. This will typically be against a database of human germline sequences prior to antibody maturation in order to reduce the possibility of immunogenicity, however any human sequences can be used. Typically a program like BLAST can be used to search a database of sequences for the most homologous. Databases of human antibody sequences can be found from various sources such as NCBI (National Center for Biotechnology Information).

### Example

RbtV_{H} amino acid sequence from residues numbered 1 through 98 in FIG. 1 is BLASTed against a human antibody germline database. The top three unique returned sequences are shown in FIG. 1 as 3-64-04 (SEQ ID NO: 739), 3-66-04 (SEQ ID NO: 740), and 3-53-02 (SEQ ID NO: 741).

4. Generally the most homologous human germline variable heavy chain sequence is then used as the basis for humanization. However those skilled in the art may decide to use another sequence that wasn't the most homologous as determined by the homology algorithm, based on other factors including sequence gaps and framework similarities.

### Example

3-64-04 in FIG. 1 was the most homologous human germline variable heavy chain sequence and is used as the basis for the humanization of RbtV_{H}.

5. Determine the framework and CDR arrangement (FR1, FR2, FR3, CDR1 & CDR2) for the human homolog being used for the heavy chain humanization. This is using the traditional layout as described in the field. Align the rabbit variable heavy chain sequence with the human homolog, while maintaining the layout of the framework and CDR regions.

### Example

In FIG. 1, the RbtV_{H} sequence is aligned with the human homologous sequence 3-64-04, and the framework and CDR domains are indicated.

6. Replace the human homologous heavy chain sequence CDR1 and CDR2 regions with the CDR1 and CDR2 sequences from the rabbit sequence. If there are differences in length between the rabbit and human CDR sequences then use the entire rabbit CDR sequences and their lengths. In addition, it may be necessary to replace the final three amino acids of the human heavy chain Framework 1 region with the final three amino acids of the rabbit heavy chain Framework 1. Typically but not always, in rabbit heavy chain Framework 1 these three residues follow a Glycine residue preceded by a Serine residue. In addition, it may be necessary replace the final amino acid of the human heavy chain Framework 2 region with the final amino acid of the rabbit heavy chain Framework 2. Typically, but not necessarily always, this is a Glycine residue preceded by an Isoleucine residue in the rabbit heavy chain Framework 2. It is possible that the specificity, affinity and/or immunogenicity of the resulting humanized antibody may be unaltered if smaller or larger sequence exchanges are performed, or if specific residue(s) are altered, however the exchanges as described have been used successfully, but do not exclude the possibility that other changes may be permitted. For example, a tryptophan amino acid residue typically occurs four residues prior to the end of the rabbit heavy chain CDR2 region, whereas in human heavy chain CDR2 this residue is typically a Serine residue. Changing this rabbit tryptophan residue to a the human Serine residue at this position has been demonstrated to have minimal to no effect on the humanized antibody's specificity or affinity, and thus further minimizes the content of rabbit sequence-derived amino acid residues in the humanized sequence.

### Example

In FIG. 1, The CDR1 and CDR2 amino acid residues of the human homologous variable heavy chain are replaced with the CDR1 and CDR2 amino acid sequences from the RbtV_{H} rabbit antibody light chain sequence, except for the boxed residue, which is tryptophan in the rabbit sequence (position number 63) and Serine at the same position in the human sequence, and is kept as the human Serine residue. In addition to the CDR1 and CDR2 changes, the final three amino acids of Framework 1 (positions 28-30) as well as the final residue of Framework 2 (position 49) are retained as rabbit amino acid residues instead of human. The resulting humanized sequence is shown below as V_{H}h from residues numbered 1 through 98. Note that the only residues that are different from the 3-64-04 human sequence are underlined, and are thus rabbit-derived amino acid residues. In this example only 15 of the 98 residues are different than the human sequence.

7. After framework 3 of the new hybrid sequence created in Step 6, attach the entire CDR3 of the rabbit heavy chain antibody sequence. The CDR3 sequence can be of various lengths, but is typically 5 to 19 amino acid residues in length. The CDR3 region and the beginning of the following framework 4 region are defined classically and are identifiable by those skilled in the art. Typically the beginning of framework 4, and thus after the end of CDR3 consists of the sequence WGXG . . . (where X is usually Q or P) (SEQ ID NO: 746), however some variation may exist in these residues.

### Example

The CDR3 of RbtV_{H} (amino acid residues numbered 99-110) is added after the end of framework 3 in the humanized sequence indicated as V_{H}h.

8. The rabbit heavy chain framework 4, which is typically the final 11 amino acid residues of the variable heavy chain and begins as indicated in Step 7 above and typically ends with the amino acid sequence ‘ . . . TVSS’ (SEQ ID NO: 747) is replaced with the nearest human heavy chain framework 4 homolog, usually from germline sequence. Frequently this human heavy chain framework 4 is of the sequence ‘WGQGTLVTVSS’ (SEQ ID NO: 748). It is possible that other human heavy chain framework 4 sequences that are not the most homologous or otherwise different may be used without affecting the specificity, affinity and/or immunogenicity of the resulting humanized antibody. This human heavy chain framework 4 sequence is added to the end of the variable heavy chain humanized sequence immediately following the CDR3 sequence from Step 7 above. This is now the end of the variable heavy chain humanized amino acid sequence.

### Example

In FIG. 1, framework 4 (FR4) of the RbtV_{H} rabbit heavy chain sequence is shown above a homologous human heavy FR4 sequence. The human FR4 sequence is added to the humanized variable heavy chain sequence (V_{H}h) right after the end of the CD3 region added in Step 7 above.

### Methods of Producing Antibodies and Fragments Thereof

The subject technology is also directed to the production of the antibodies described herein or fragments thereof. Recombinant polypeptides corresponding to the antibodies described herein or fragments thereof are secreted from polyploidal, preferably diploid or tetraploid strains of mating competent yeast. In an exemplary embodiment, the subject technology is directed to methods for producing these recombinant polypeptides in secreted form for prolonged periods using cultures comprising polyploid yeast, i.e., at least several days to a week, more preferably at least a month or several months, and even more preferably at least 6 months to a year or longer. These polyploid yeast cultures will express at least 10-25 mg/liter of the polypeptide, more preferably at least 50-250 mg/liter, still more preferably at least 500-1000 mg/liter, and most preferably a gram per liter or more of the recombinant polypeptide(s).

In one embodiment of the subject technology a pair of genetically marked yeast haploid cells are transformed with expression vectors comprising subunits of a desired heteromultimeric protein. One haploid cell comprises a first expression vector, and a second haploid cell comprises a second expression vector. In another embodiment diploid yeast cells will be transformed with at least one expression vectors that provide for the expression and secretion of at least one of the recombinant polypeptides. In still another embodiment a single haploid cell may be transformed with at least one vectors and used to produce a polyploidal yeast by fusion or mating strategies. In yet another embodiment a diploid yeast culture may be transformed with at least one vectors providing for the expression and secretion of a desired polypeptide or polypeptides. These vectors may comprise vectors e.g., linearized plasmids or other linear DNA products that integrate into the yeast cell's genome randomly, through homologous recombination, or using a recombinase such as Cre/Lox or Flp/Frt. Optionally, additional expression vectors may be introduced into the haploid or diploid cells; or the first or second expression vectors may comprise additional coding sequences; for the synthesis of heterotrimers; heterotetramers. The expression levels of the non-identical polypeptides may be individually calibrated, and adjusted through appropriate selection, vector copy number, promoter strength and/or induction and the like. The transformed haploid cells are genetically crossed or fused. The resulting diploid or tetraploid strains are utilized to produce and secrete fully assembled and biologically functional proteins, humanized antibodies described herein or fragments thereof.

The use of diploid or tetraploid cells for protein production provides for unexpected benefits. The cells can be grown for production purposes, i.e. scaled up, and for extended periods of time, in conditions that can be deleterious to the growth of haploid cells, which conditions may include high cell density; growth in minimal media; growth at low temperatures; stable growth in the absence of selective pressure; and which may provide for maintenance of heterologous gene sequence integrity and maintenance of high level expression over time. Without wishing to be bound thereby, the inventors theorize that these benefits may arise, at least in part, from the creation of diploid strains from two distinct parental haploid strains. Such haploid strains can comprise numerous minor autotrophic mutations, which mutations are complemented in the diploid or tetraploid, enabling growth and enhanced production under highly selective conditions.

Transformed mating competent haploid yeast cells provide a genetic method that enables subunit pairing of a desired protein. Haploid yeast strains are transformed with each of two expression vectors, a first vector to direct the synthesis of one polypeptide chain and a second vector to direct the synthesis of a second, non-identical polypeptide chain. The two haploid strains are mated to provide a diploid host where optimized target protein production can be obtained.

Optionally, additional non-identical coding sequence(s) are provided. Such sequences may be present on additional expression vectors or in the first or the second expression vectors. As is known in the art, multiple coding sequences may be independently expressed from individual promoters; or may be coordinately expressed through the inclusion of an “internal ribosome entry site” or “IRES”, which is an element that promotes direct internal ribosome entry to the initiation codon, such as ATG, of a cistron (a protein encoding region), thereby leading to the cap-independent translation of the gene. IRES elements functional in yeast are described by Thompson, et al. (2001) PNAS 98: 12866-12868.

In one embodiment of the subject technology, antibody sequences are produced in combination with a secretory J chain, which provides for enhanced stability of IgA. See U.S. Pat. Nos. 5,959,177 and 5,202,422.

In a preferred embodiment the two haploid yeast strains are each auxotrophic, and require supplementation of media for growth of the haploid cells. The pair of auxotrophs are complementary, such that the diploid product will grow in the absence of the supplements required for the haploid cells. Many such genetic markers are known in yeast, including requirements for amino acids (e.g. met, lys, his, arg), nucleosides (e.g. ura3, ade1); and the like. Amino acid markers may be preferred for the methods of the subject technology. Alternatively diploid cells which contain the desired vectors can be selected by other means, e.g., by use of other markers, such as green fluorescent protein, antibiotic resistance genes, various dominant selectable markers, and the like.

Two transformed haploid cells may be genetically crossed and diploid strains arising from this mating event selected by their hybrid nutritional requirements and/or antibiotic resistance spectra. Alternatively, populations of the two transformed haploid strains are spheroplasted and fused, and diploid progeny regenerated and selected. By either method, diploid strains can be identified and selectively grown based on their ability to grow in different media than their parents. For example, the diploid cells may be grown in minimal medium that may include antibiotics. The diploid synthesis strategy has certain advantages. Diploid strains have the potential to produce enhanced levels of heterologous protein through broader complementation to underlying mutations, which may impact the production and/or secretion of recombinant protein. Furthermore, once stable strains have been obtained, any antibiotics used to select those strains do not necessarily need to be continuously present in the growth media.

As noted above, in some embodiments a haploid yeast may be transformed with a single or multiple vectors and mated or fused with a non-transformed cell to produce a diploid cell containing the vector or vectors. In other embodiments, a diploid yeast cell may be transformed with at least one vectors that provide for the expression and secretion of a desired heterologous polypeptide by the diploid yeast cell.

In one embodiment of the subject technology, two haploid strains are transformed with a library of polypeptides, e.g. a library of antibody heavy or light chains. Transformed haploid cells that synthesize the polypeptides are mated with the complementary haploid cells. The resulting diploid cells are screened for functional protein. The diploid cells provide a means of rapidly, conveniently and inexpensively bringing together a large number of combinations of polypeptides for functional testing. This technology is especially applicable for the generation of heterodimeric protein products, where optimized subunit synthesis levels are critical for functional protein expression and secretion.

In another embodiment of the subject technology, the expression level ratio of the two subunits is regulated in order to maximize product generation. Heterodimer subunit protein levels have been shown previously to impact the final product generation. Simmons (2002) J Immunol Methods. 263(1-2): 133-47. Regulation can be achieved prior to the mating step by selection for a marker present on the expression vector. By stably increasing the copy number of the vector, the expression level can be increased. In some cases, it may be desirable to increase the level of one chain relative to the other, so as to reach a balanced proportion between the subunits of the polypeptide. Antibiotic resistance markers are useful for this purpose, e.g. Zeocin® (phleomycin) resistance marker, G418 resistance and provide a means of enrichment for strains that contain multiple integrated copies of an expression vector in a strain by selecting for transformants that are resistant to higher levels of Zeocin® (phleomycin) or G418. The proper ratio (e.g. 1:1; 1:2) of the subunit genes may be important for efficient protein production. Even when the same promoter is used to transcribe both subunits, many other factors contribute to the final level of protein expressed and therefore, it can be useful to increase the number of copies of one encoded gene relative to the other. Alternatively, diploid strains that produce higher levels of a polypeptide, relative to single copy vector strains, are created by mating two haploid strains, both of which have multiple copies of the expression vectors.

Host cells are transformed with the above-described expression vectors, mated to form diploid strains, and cultured in conventional nutrient media modified as appropriate for inducing promoters, selecting transformants or amplifying the genes encoding the desired sequences. A number of minimal media suitable for the growth of yeast are known in the art. Any of these media may be supplemented as necessary with salts (such as sodium chloride, calcium, magnesium, and phosphate), buffers (such as phosphate, HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression, and will be apparent to the ordinarily skilled artisan.

Secreted proteins are recovered from the culture medium. A protease inhibitor, such as phenyl methyl sulfonyl fluoride (PMSF) may be useful to inhibit proteolytic degradation during purification, and antibiotics may be included to prevent the growth of adventitious contaminants. The composition may be concentrated, filtered, dialyzed, using methods known in the art.

The diploid cells of the subject technology are grown for production purposes. Such production purposes desirably include growth in minimal media, which media lacks pre-formed amino acids and other complex biomolecules, e.g., media comprising ammonia as a nitrogen source, and glucose as an energy and carbon source, and salts as a source of phosphate, calcium and the like. Preferably such production media lacks selective agents such as antibiotics, amino acids, purines, pyrimidines The diploid cells can be grown to high cell density, for example at least about 50 g/L; more usually at least about 100 g/L; and may be at least about 300, about 400, about 500 g/L or more.

In one embodiment of the subject technology, the growth of the subject cells for production purposes is performed at low temperatures, which temperatures may be lowered during log phase, during stationary phase, or both. The term “low temperature” refers to temperatures of at least about 15° C., more usually at least about 17° C., and may be about 20° C., and is usually not more than about 25° C., more usually not more than about 22° C. In another embodiment of the subject technology, the low temperature is usually not more than about 28° C. Growth temperature can impact the production of full-length secreted proteins in production cultures, and decreasing the culture growth temperature can strongly enhance the intact product yield. The decreased temperature appears to assist intracellular trafficking through the folding and post-translational processing pathways used by the host to generate the target product, along with reduction of cellular protease degradation.

The methods of the subject technology provide for expression of secreted, active protein, preferably a mammalian protein. In one embodiment, secreted, “active antibodies”, as used herein, refers to a correctly folded multimer of at least two properly paired chains, which accurately binds to its cognate antigen. Expression levels of active protein are usually at least about 10-50 mg/liter culture, more usually at least about 100 mg/liter, preferably at least about 500 mg/liter, and may be 1000 mg/liter or more.

The methods of the subject technology can provide for increased stability of the host and heterologous coding sequences during production. The stability is evidenced, for example, by maintenance of high levels of expression of time, where the starting level of expression is decreased by not more than about 20%, usually not more than 10%, and may be decreased by not more than about 5% over about 20 doublings, 50 doublings, 100 doublings, or more.

The strain stability also provides for maintenance of heterologous gene sequence integrity over time, where the sequence of the active coding sequence and requisite transcriptional regulatory elements are maintained in at least about 99% of the diploid cells, usually in at least about 99.9% of the diploid cells, and preferably in at least about 99.99% of the diploid cells over about 20 doublings, 50 doublings, 100 doublings, or more. Preferably, substantially all of the diploid cells maintain the sequence of the active coding sequence and requisite transcriptional regulatory elements.

Other methods of producing antibodies are well known to those of ordinary skill in the art. For example, methods of producing chimeric antibodies are now well known in the art. See, e.g., U.S. Pat. No. 4,816,567; Morrison, et al. (1984) P.N.A.S. USA 81: 8651-55; Neuberger, et al. (1985) Nature 314: 268-270; Boulianne, et al. (1984) Nature 312: 643-46.

Likewise, other methods of producing humanized antibodies are now well known in the art. See, e.g., U.S. Pat. Nos. 5,225,539; 5,530,101; 5,585,089; 5,693,762; 6,054,297; 6,180,370; 6,407,213; 6,548,640; 6,632,927; and 6,639,055; Jones, et al. (1986) Nature 321: 522-525; Reichmann, et al. (1988) Nature 332: 323-327; Verhoeyen, et al. (1988) Science 239: 1534-36.

Antibody polypeptides of the subject technology having IL-6 binding specificity may also be produced by constructing, using conventional techniques well known to those of ordinary skill in the art, an expression vector containing an operon and a DNA sequence encoding an antibody heavy chain in which the DNA sequence encoding the CDRs required for antibody specificity is derived from a non-human cell source, preferably a rabbit B-cell source, while the DNA sequence encoding the remaining parts of the antibody chain is derived from a human cell source.

A second expression vector is produced using the same conventional means well known to those of ordinary skill in the art, said expression vector containing an operon and a DNA sequence encoding an antibody light chain in which the DNA sequence encoding the CDRs required for antibody specificity is derived from a non-human cell source, preferably a rabbit B-cell source, while the DNA sequence encoding the remaining parts of the antibody chain is derived from a human cell source.

The expression vectors are transfected into a host cell by convention techniques well known to those of ordinary skill in the art to produce a transfected host cell, said transfected host cell cultured by conventional techniques well known to those of ordinary skill in the art to produce said antibody polypeptides.

The host cell may be co-transfected with the two expression vectors described above, the first expression vector containing DNA encoding an operon and a light chain-derived polypeptide and the second vector containing DNA encoding an operon and a heavy chain-derived polypeptide. The two vectors contain different selectable markers, but preferably achieve substantially equal expression of the heavy and light chain polypeptides. Alternatively, a single vector may be used, the vector including DNA encoding both the heavy and light chain polypeptides. The coding sequences for the heavy and light chains may comprise cDNA.

The host cells used to express the antibody polypeptides may be either a bacterial cell such as E. coli, or a eukaryotic cell. In a particularly preferred embodiment of the subject technology, a mammalian cell of a well-defined type for this purpose, such as a myeloma cell or a Chinese hamster ovary (CHO) cell line may be used.

The general methods by which the vectors may be constructed, transfection methods required to produce the host cell and culturing methods required to produce the antibody polypeptides from said host cells all include conventional techniques. Although preferably the cell line used to produce the antibody is a mammalian cell line, any other suitable cell line, such as a bacterial cell line such as an E. coli-derived bacterial strain, or a yeast cell line, may alternatively be used.

Similarly, once produced the antibody polypeptides may be purified according to standard procedures in the art, such as for example cross-flow filtration, ammonium sulphate precipitation, affinity column chromatography and the like.

The antibody polypeptides described herein may also be used for the design and synthesis of either peptide or non-peptide mimetics that would be useful for the same therapeutic applications as the antibody polypeptides of the subject technology. See, for example, Saragobi et al. (1991) Science 253: 792-795.

### Exemplary Embodiments of Heavy and Light Chain Polypeptides and Polynucleotides

This section recites exemplary embodiments of heavy and light chain polypeptides, as well as exemplary polynucleotides encoding such polypeptides. These exemplary polynucleotides are suitable for expression in the disclosed Pichia expression system.

In certain embodiments, the present subject technology encompasses polynucleotides having at least about 70%, 75%, 80%, 85%, 90%, 91%, 92%, 93%, 94%, 95%, 96%, 97%, 98%, 99%, or 100% identity (sequence homology) to the polynucleotides recited in this application or that encode polypeptides recited in this application, or that hybridize to said polynucleotides under conditions of low-stringency, moderate-stringency, or high-stringency conditions, preferably those that encode polypeptides (e.g. an immunoglobulin heavy and light chain, a single-chain antibody, an antibody fragment) that have at least one of the biological activities set forth herein, including without limitation thereto specific binding to an IL-6 polypeptide. In another aspect, the subject technology encompasses a composition comprising such a polynucleotide and/or a polypeptide encoded by such a polynucleotide. In yet another aspect, the subject technology encompasses a method of treatment of a disease or condition associated with IL-6 or that may be prevented, treated, or ameliorated with an IL-6 antagonist such as Ab1 (e.g. psoriatic arthritis) comprising administration of a composition comprising such a polynucleotide and/or polypeptide.

In certain preferred embodiments, a heavy chain polypeptide will comprise at least one of the CDR sequences of the heavy and/or light chain polypeptides recited herein (including those contained in the heavy and light chain polypeptides recited herein) and at least one of the framework region polypeptides recited herein, including those depicted in FIGS. 1 and 8-11 or Table 1, and contained in the heavy and light chain polypeptide sequences recited herein. In certain preferred embodiments, a heavy chain polypeptide will comprise at least one Framework 4 region sequences as depicted in FIGS. 1 and 8-11 or Table 1, or as contained in a heavy or light chain polypeptide recited herein.

In certain preferred embodiments, a light chain polypeptide will comprise at least one of the CDR sequences of the heavy and/or light chain polypeptides recited herein (including those contained in the heavy and light chain polypeptides recited herein) and at least one of the Framework region polypeptides recited herein, including those depicted in FIGS. 1 and 8-11 or Table 1, and contained in the heavy and light chain polypeptide sequences recited herein. In certain preferred embodiments, a light chain polypeptide will comprise at least one Framework 4 region sequences as depicted in FIGS. 1 and 8-11 or Table 1, or as contained in a heavy or light chain polypeptide recited herein.

In any of the embodiments recited herein, certain of the sequences recited may be substituted for each other, unless the context indicates otherwise. The recitation that particular sequences may be substituted for one another, where such recitations are made, are understood to be illustrative rather than limiting, and it is also understood that such substitutions are encompassed even when no illustrative examples of substitutions are recited, For example, wherever at least one of the Ab1 light chain polypeptides is recited, e.g. any of SEQ ID NO: 2, 20, 647, 651, 660, 666, 699, 702, 706, or 709, another Ab1 light chain polypeptide may be substituted unless the context indicates otherwise. Similarly, wherever one of the Ab1 heavy chain polypeptides is recited, e.g. any of SEQ ID NO: 3, 18, 19, 652, 656, 657, 658, 661, 664, 665, 704, or 708, another Ab1 heavy chain polypeptide may be substituted unless the context indicates otherwise. Likewise, wherever one of the Ab1 light chain polynucleotides is recited, e.g. any of SEQ ID NO: 10, 662, 698, 701, or 705, another Ab1 light chain polynucleotide may be substituted unless the context indicates otherwise. Similarly, wherever one of the Ab1 heavy chain polynucleotides is recited, e.g. any of SEQ ID NO: 11, 663, 700, 703, or 707, another Ab1 heavy chain polynucleotide may be substituted unless the context indicates otherwise.

Additionally, recitation of any member of any of the following groups is understood to encompass substitution by any other member of the group, as follows: Ab2 Light chain polypeptides (SEQ ID NO: 21 and 667); Ab2 Light chain polynucleotides (SEQ ID NO: 29 and 669); Ab2 Heavy chain polypeptides (SEQ ID NO: 22 and 668); Ab2 Heavy chain polynucleotides (SEQ ID NO: 30 and 670); Ab3 Light chain polypeptides (SEQ ID NO: 37 and 671); Ab3 Light chain polynucleotides (SEQ ID NO: 45 and 673); Ab3 Heavy chain polypeptides (SEQ ID NO: 38 and 672); Ab3 Heavy chain polynucleotides (SEQ ID NO: 46 and 674); Ab4 Light chain polypeptides (SEQ ID NO: 53 and 675); Ab4 Light chain polynucleotides (SEQ ID NO: 61 and 677); Ab4 Heavy chain polypeptides (SEQ ID NO: 54 and 676); Ab4 Heavy chain polynucleotides (SEQ ID NO: 62 and 678); Ab5 Light chain polypeptides (SEQ ID NO: 69 and 679); Ab5 Light chain polynucleotides (SEQ ID NO: 77 and 681); Ab5 Heavy chain polypeptides (SEQ ID NO: 70 and 680); Ab5 Heavy chain polynucleotides (SEQ ID NO: 78 and 682); Ab6 Light chain polypeptides (SEQ ID NO: 85 and 683); Ab6 Light chain polynucleotides (SEQ ID NO: 93 and 685); Ab6 Heavy chain polypeptides (SEQ ID NO: 86 and 684); Ab6 Heavy chain polynucleotides (SEQ ID NO: 94 and 686); Ab7 Light chain polypeptides (SEQ ID NO: 101, 119, 687, 693); Ab7 Light chain polynucleotides (SEQ ID NO: 109 and 689); Ab7 Heavy chain polypeptides (SEQ ID NO: 102, 117, 118, 688, 691, and 692); Ab7 Heavy chain polynucleotides (SEQ ID NO: 110 and 690); Ab1 Light Chain CDR1 polynucleotides (SEQ ID NO: 12 and 694); Ab1 Light Chain CDR3 polynucleotides (SEQ ID NO: 14 and 695); Ab1 Heavy Chain CDR2 polynucleotides (SEQ ID NO: 16 and 696) and Ab1 Heavy Chain CDR3 polynucleotides (SEQ ID NO: 17 and 697). Exemplary Ab1-encoding polynucleotide sequences include but are not limited to SEQ ID NO: 662, 663, 698, 700, 701, 703, 705, 707, 720, 721, 722, 723, 724, and 725.

### Screening Assays

The subject technology also includes screening assays designed to assist in the identification of diseases and disorders associated with IL-6 in patients exhibiting symptoms of an IL-6 associated disease or disorder, especially psoriatic arthritis.

In one embodiment of the subject technology, the anti-IL-6 antibodies of the subject technology, or IL-6 binding fragments or variants thereof, are used to detect the presence of IL-6 in a biological sample obtained from a patient exhibiting symptoms of a disease or disorder associated with IL-6. The presence of IL-6, or elevated levels thereof when compared to pre-disease levels of IL-6 in a comparable biological sample, may be beneficial in diagnosing a disease or disorder associated with IL-6.

Another embodiment of the subject technology provides a diagnostic or screening assay to assist in diagnosis of diseases or disorders associated with IL-6 in patients exhibiting symptoms of an IL-6 associated disease or disorder identified herein, comprising assaying the level of IL-6 expression in a biological sample from said patient using a post-translationally modified anti-IL-6 antibody or binding fragment or variant thereof. The anti-IL-6 antibody or binding fragment or variant thereof may be post-translationally modified to include a detectable moiety such as set forth previously in the disclosure.

The IL-6 level in the biological sample is determined using a modified anti-IL-6 antibody or binding fragment or variant thereof as set forth herein, and comparing the level of IL-6 in the biological sample against a standard level of IL-6 (e.g., the level in normal biological samples). The skilled clinician would understand that some variability may exist between normal biological samples, and would take that into consideration when evaluating results.

The above-recited assay may also be useful in monitoring a disease or disorder, where the level of IL-6 obtained in a biological sample from a patient believed to have an IL-6 associated disease or disorder is compared with the level of IL-6 in prior biological samples from the same patient, in order to ascertain whether the IL-6 level in said patient has changed with, for example, a treatment regimen. A skilled clinician would understand that a biological sample includes, but is not limited to, sera, plasma, urine, saliva, mucous, pleural fluid, synovial fluid and spinal fluid.

### Labels

As stated above, antibodies and fragments and variants thereof may be modified post-translationally to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

The anti-IL-6 antibodies and antigen-binding fragments thereof described herein may be modified post-translationally to add effector moieties such as chemical linkers, detectable moieties such as for example fluorescent dyes, enzymes, substrates, bioluminescent materials, radioactive materials, chemiluminescent moieties, a cytotoxic agent, radioactive materials, or functional moieties.

A wide variety of entities, e.g., ligands, may be coupled to the oligonucleotides as known in the art. Ligands may include naturally occurring molecules, or recombinant or synthetic molecules. Exemplary ligands include, but are not limited to, avadin, biotin, peptides, peptidomimetics, polylysine (PLL), polyethylene glycol (PEG), mPEG, cationic groups, spermine, spermidine, polyamine, thyrotropin, melanotropin, lectin, glycoprotein, surfactant protein A, mucin, glycosylated polyaminoacids, transferrin, aptamer, immunoglobulins (e.g., antibodies), insulin, transferrin, albumin, sugar, lipophilic molecules (e.g., steroids, bile acids, cholesterol, cholic acid, and fatty acids), vitamin A, vitamin E, vitamin K, vitamin B, folic acid, B12, riboflavin, biotin, pyridoxal, vitamin cofactors, lipopolysaccharide, hormones and hormone receptors, lectins, carbohydrates, multivalent carbohydrates, radiolabeled markers, fluorescent dyes, and derivatives thereof. See, e.g., U.S. Pat. Nos. 6,153,737; 6,172,208; 6,300,319; 6,335,434; 6,335,437; 6,395,437; 6,444,806; 6,486,308; 6,525,031; 6,528,631; and 6,559, 279.

Additionally, moieties may be added to the antigen or epitope to increase half-life in vivo (e.g., by lengthening the time to clearance from the blood stream. Such techniques include, for example, adding PEG moieties (also termed pegylation), and are well-known in the art. See U.S. Patent Application Publication No. 2003/0031671.

An anti-IL-6 antibody or antigen binding fragment thereof, described herein may be “attached” to a substrate when it is associated with the solid label through a non-random chemical or physical interaction. The attachment may be through a covalent bond. However, attachments need not be covalent or permanent. Materials may be attached to a label through a “spacer molecule” or “linker group.” Such spacer molecules are molecules that have a first portion that attaches to the biological material and a second portion that attaches to the label. Thus, when attached to the label, the spacer molecule separates the label and the biological materials, but is attached to both. Methods of attaching biological material (e.g., label) to a label are well known in the art, and include but are not limited to chemical coupling.

### Detectable Labels

The anti-IL-6 antibody or antigen-binding fragments described herein may be modified post-translationally to add effector labels such as chemical linkers, detectable labels such as for example fluorescent dyes, enzymes, substrates, bioluminescent materials, radioactive materials, and chemiluminescent labels, or functional labels such as for example streptavidin, avidin, biotin, a cytotoxin, a cytotoxic agent, and radioactive materials. Further exemplary enzymes include, but are not limited to, horseradish peroxidase, acetylcholinesterase, alkaline phosphatase, β-galactosidase and luciferase. Further exemplary fluorescent materials include, but are not limited to, rhodamine, fluorescein, fluorescein isothiocyanate, umbelliferone, dichlorotriazinylamine, phycoerythrin and dansyl chloride. Further exemplary chemiluminescent labels include, but are not limited to, luminol. Further exemplary bioluminescent materials include, but are not limited to, luciferin and aequorin. Further exemplary radioactive materials include, but are not limited to, bismuth-213 (²¹³Bs), carbon-14 (¹⁴C), carbon-11 (¹¹C), chlorine-18 (Cl¹⁸), chromium-51 (⁵¹Cr), cobalt-57 (⁵⁷Co), cobalt-60 (⁶⁰Co), copper-64 (⁶⁴Cu), copper-67 (⁶⁷Cu), dysprosium-165 (¹⁶⁵Dy), erbium-169 (¹⁶⁹Er), fluorine-18 (¹⁸F), gallium-67 (⁶⁷Ga), gallium-68 (⁶⁸Ga), germanium-68 (⁶⁸Ge), holmium-166 (¹⁶⁶Ho), indium-111 (¹¹¹In), iodine-125 (¹²⁵I), iodine-123 (¹²⁴I), iodine-124 (¹²⁴I), iodine-131 (¹³¹I), iridium-192 (¹⁹²Ir), iron-59 (⁵⁹Fe), krypton-81 (⁸¹Kr), lead-212 (²¹²Pb), lutetium-177 (¹⁷⁷Lu), molybdenum-99 (⁹⁹Mo), nitrogen-13 (¹³N), oxygen-15 (¹⁵O), palladium-103 (¹⁰³Pd), phosphorus-32 (³²P), potassium-42 (⁴²K), rhenium-186 (¹⁸⁶Re), rhenium-188 (¹⁸⁸Re), rubidium-81 (⁸¹Rb), rubidium-82 (⁸²Rb), samarium-153 (¹⁵³Sm), selenium-75 (⁷⁵Se), sodium-24 (²⁴Na), strontium-82 (⁸²Sr), strontium-89 (⁸⁹Sr), sulfur 35 (³⁵S), technetium-99m (⁹⁹Tc), thallium-201 (²⁰¹Tl), tritium (³H), xenon-133 (¹³³Xe), ytterbium-169 (¹⁶⁹Yb), ytterbium-177 (¹⁷⁷Yb), and yttrium-90 (⁹⁰Y).

### Cytotoxic Agents

The anti-IL-6 antibodies and antigen-binding fragments described herein may be conjugated to cytotoxic agents including, but are not limited to, methotrexate, aminopterin, 6-mercaptopurine, 6-thioguanine, cytarabine, 5-fluorouracil dacarbazine; alkylating agents such as mechlorethamine, thiotepa chlorambucil, melphalan, carmustine (BSNU), mitomycin C, lomustine (CCNU), 1-methylnitrosourea, cyclophosphamide, mechlorethamine, busulfan, dibromomannitol, streptozotocin, mitomycin C, cis-dichlorodiamine platinum (II) (DDP) cisplatin and carboplatin (paraplatin); anthracyclines include daunorubicin (formerly daunomycin), doxorubicin (adriamycin), detorubicin, carminomycin, idarubicin, epirubicin, mitoxantrone and bisantrene; antibiotics include dactinomycin (actinomycin D), bleomycin, calicheamicin, mithramycin, and anthramycin (AMC); and antimitotic agents such as the vinca alkaloids, vincristine and vinblastine. Other cytotoxic agents include paclitaxel (TAXOL®), ricin, pseudomonas exotoxin, gemcitabine, cytochalasin B, gramicidin D, ethidium bromide, emetine, etoposide, teniposide, colchicine, dihydroxy anthracin dione, 1-dehydrotestosterone, glucocorticoids, procaine, tetracaine, lidocaine, propranolol, puromycin, procarbazine, hydroxyurea, asparaginase, corticosteroids, mitotane (O,P′-(DDD)), interferons, and mixtures of these cytotoxic agents.

Further cytotoxic agents include, but are not limited to, chemotherapeutic agents such as carboplatin, cisplatin, paclitaxel, gemcitabine, calicheamicin, doxorubicin, 5-fluorouracil, mitomycin C, actinomycin D, cyclophosphamide, vincristine, bleomycin, VEGF antagonists, EGFR antagonists, platins, taxols, irinotecan, 5-fluorouracil, gemcitabine, leucovorin, steroids, cyclophosphamide, melphalan, vinca alkaloids (e.g., vinblastine, vincristine, vindesine and vinorelbine), mustines, tyrosine kinase inhibitors, radiotherapy, sex hormone antagonists, selective androgen receptor modulators, selective estrogen receptor modulators, PDGF antagonists, TNF antagonists, IL-1 antagonists, interleukins (e.g. IL-12 or IL-2), IL-12R antagonists, Erbitux®, Avastin®, Pertuzumab, anti-CD20 antibodies, Rituxan®, ocrelizumab, ofatumumab, DXL625, Herceptin®, or any combination thereof. Toxic enzymes from plants and bacteria such as ricin, diphtheria toxin and Pseudomonas toxin may be conjugated to the humanized antibodies, or binding fragments thereof, to generate cell-type-specific-killing reagents. Youle, et al. (1980) Proc. Nat'l Acad. Sci. USA 77: 5483; Gilliland, et al. (1980) Proc. Nat'l Acad. Sci. USA 77: 4539; Krolick, et al. (1980) Proc. Nat'l Acad. Sci. USA 77: 5419. Other cytotoxic agents include cytotoxic ribonucleases. See U.S. Pat. No. 6,653,104.

The anti-IL-6 antibodies and antigen-binding fragments described herein may be conjugated to a radionuclide that emits alpha or beta particles (e.g., radioimmunoconjuagtes). Such radioactive isotopes include but are not limited to beta-emitters such as phosphorus-32 (³²P), scandium-47 (⁴⁷Sc), copper-67 (⁶⁷Cu), gallium-67 (⁶⁷Ga), yttrium-88 (⁸⁸Y), yttrium-90 (⁹⁰Y), iodine-125 (¹²⁵I), iodine-131 (¹³¹I), samarium-153 (¹⁵³Sm), lutetium-177 (¹⁷⁷Lu), rhenium-186 (¹⁸⁶Re), rhenium-188 (¹⁸⁸Re), and alpha-emitters such as astatine-211 (²¹¹At), lead-212 (²¹²Pb), bismuth-212 (²¹²Bi), bismuth-213 (²¹³Bi) or actinium-225 (²²⁵Ac).

Methods are known in the art for conjugating an anti-IL-6 antibody described herein to a label, such as those methods described by Hunter, et al. (1962) Nature 144: 945; David, et al. (1974) Biochemistry 13: 1014; Pain, et al. (1981) J. Immunol. Meth. 40: 219; and Nygren (1982) Histochem and Cytochem 30: 407.

### Substrates

The anti-IL-6 antibodies and antigen-binding fragments thereof described herein may be attached to a substrate. A number of substrates (e.g., solid supports) known in the art are suitable for use with the anti-IL-6 antibody described herein. The substrate may be modified to contain channels or other configurations. See Fung (2004) [Ed.] Protein Arrays: Methods and Protocols Humana Press and Kambhampati (2004) [Ed.] Protein Microarray Technology John Wiley & Sons.

Substrate materials include, but are not limited to acrylics, agarose, borosilicate glass, carbon (e.g., carbon nanofiber sheets or pellets), cellulose acetate, cellulose, ceramics, gels, glass (e.g., inorganic, controlled-pore, modified, soda-lime, or functionalized glass), latex, magnetic beads, membranes, metal, metalloids, nitrocellulose, NYLON®, optical fiber bundles, organic polymers, paper, plastics, polyacryloylmorpholide, poly(4-methylbutene), poly(ethylene terephthalate), poly(vinyl butyrate), polyacrylamide, polybutylene, polycarbonate, polyethylene, polyethyleneglycol terephthalate, polyformaldehyde, polymethacrylate, polymethylmethacrylate, polypropylene, polysaccharides, polystyrene, polyurethanes, polyvinylacetate, polyvinylchloride, polyvinylidene difluoride (PVDF), polyvinylpyrrolidinone, rayon, resins, rubbers, semiconductor materials, SEPHAROSE®, silica, silicon, styrene copolymers, TEFLON®, and variety of other polymers.

Substrates need not be flat and can include any type of shape including spherical shapes (e.g., beads) or cylindrical shapes (e.g., fibers). Materials attached to solid supports may be attached to any portion of the solid support (e.g., may be attached to an interior portion of a porous solid support material).

The substrate body may be in the form of a bead, box, column, cylinder, disc, dish (e.g., glass dish, PETRI dish), fiber, film, filter, microtiter plate (e.g., 96-well microtiter plate), multi-bladed stick, net, pellet, plate, ring, rod, roll, sheet, slide, stick, tray, tube, or vial. The substrate may be a singular discrete body (e.g., a single tube, a single bead), any number of a plurality of substrate bodies (e.g, a rack of 10 tubes, several beads), or combinations thereof (e.g., a tray comprises a plurality of microtiter plates, a column filled with beads, a microtiter plate filed with beads).

An anti-IL-6 antibody or antigen-binding fragment thereof may be “attached” to a substrate when it is associated with the solid substrate through a non-random chemical or physical interaction. The attachment may be through a covalent bond. However, attachments need not be covalent or permanent. Materials may be attached to a substrate through a “spacer molecule” or “linker group.” Such spacer molecules are molecules that have a first portion that attaches to the biological material and a second portion that attaches to the substrate. Thus, when attached to the substrate, the spacer molecule separates the substrate and the biological materials, but is attached to both. Methods of attaching biological material (e.g., label) to a substrate are well known in the art, and include but are not limited to chemical coupling.

Plates, such as microtiter plates, which support and contain the solid-phase for solid-phase synthetic reactions may be used. Microtiter plates may house beads that are used as the solid-phase. By “particle” or “microparticle” or “nanoparticle” or “bead” or “microbead” or “microsphere” herein is meant microparticulate matter having any of a variety of shapes or sizes. The shape may be generally spherical but need not be spherical, being, for example, cylindrical or polyhedral. As will be appreciated by those in the art, the particles may comprise a wide variety of materials depending on their use, including, but not limited to, cross-linked starch, dextrans, cellulose, proteins, organic polymers including styrene polymers such as polystyrene and methylstyrene as well as other styrene co-polymers, plastics, glass, ceramics, acrylic polymers, magnetically responsive materials, colloids, thoriasol, carbon graphite, titanium dioxide, nylon, latex, and TEFLON®. See e.g., “Microsphere Detection Guide” from Bangs Laboratories, Fishers, Ind.

The anti-IL-6 antibody or antigen-binding fragment may be attached to on any of the forms of substrates described herein (e.g., bead, box, column, cylinder, disc, dish (e.g., glass dish, PETRI dish), fiber, film, filter, microtiter plate (e.g., 96-well microtiter plate), multi-bladed stick, net, pellet, plate, ring, rod, roll, sheet, slide, stick, tray, tube, or vial). In particular, particles or beads may be a component of a gelling material or may be separate components such as latex beads made of a variety of synthetic plastics (e.g., polystyrene). The label (e.g., streptavidin) may be bound to a substrate (e.g., bead).

### Assessment of Inflammatory Markers

Known inflammatory markers (e.g., IL-6) may be measured to assess the risk for psoriatic arthritis or the severity of psoriatic arthritis. These markers may be measured from serum, synovial fluid, or skin biopsies using known methods in the art (e.g., immunoassays).

### IL-6 Serum Levels

Serum IL-6 levels may be measured as a pharmacodynamic marker evaluate the effect of neutralization of IL-6 levels. Serum IL-6 levels may be measured using an immunoassay (e.g., ELISA assay). A decrease of serum IL-6 levels may be indicative of a lessening of inflammation.

### Serum Inflammatory Biomarkers

Serum biomarkers may be measured to determine the expression of pro-inflammatory cytokines and other soluble biomarkers that may correlate with psoriatic arthritis (PsA) disease activity including but not limited to acute phase reactants, serum pro-inflammatory cytokines (e.g., IL-1, TNF-α, IFN-γ, IL-12p40, IL-17), chemokines (e.g., RANTES, MIP-1α, MCP-1), matrix metalloproteinases (e.g., MMP-2, MMP-3, MMP-9) and other biomarkers associated with inflammation and autoimmune pathways that are known in the art. Soluble biomarkers of bone and cartilage metabolism (e.g., osteocalcin and other collagen degradation products) may also be assessed by an immunoassay (e.g., ELISA). A decrease in a serum inflammatory biomarker may be indicative of a lessening of inflammation.

### Immunohistochemistry of Skin Biopsies

Skin biopsies may be collected for biomarker analysis including whole genome array analysis and immunohistochemistry (IHC). Immunohistochemical analysis may include the measurement of epidermal thickness, frequency of resident and inflammatory cell populations (e.g., T cells, macrophages, and keratinocytes) and other inflammatory markers related to the IL-6 pathway known in the art. Specifically, the following specific antigens may be assessed per standard IHC procedure using the formalin-fixed samples: CD3, CD68, keratin 16, FoxP3, IL-6R and MMP-3. A decrease in an inflammatory biomarker in a skin biopsy may be indicative of a lessening of inflammation.

### Administration

In one embodiment of the subject technology, the anti-IL-6 antibodies described herein, or IL-6 binding fragments or variants thereof, as well as combinations of said antibody fragments or variants, are administered to a subject at a concentration of between about 0.1 and 20 mg/kg, such as about 0.4 mg/kg, about 0.8 mg/kg, about 1.6 mg/kg, or about 4 mg/kg, of body weight of recipient subject. For example, compositions comprising the anti-IL-6 antibodies described herein may comprise at least about 0, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mg. In a preferred embodiment of the subject technology, the anti-IL-6 antibodies described herein, or IL-6 binding fragments or variants thereof, as well as combinations of said antibody fragments or variants, are administered to a subject at a concentration of about 0.4 mg/kg of body weight of recipient subject. In a preferred embodiment of the subject technology, the anti-IL-6 antibodies described herein, or IL-6 binding fragments or variants thereof, as well as combinations of said antibody fragments or variants, are administered to a recipient subject with a frequency of once every twenty-six weeks or less, such as once every sixteen weeks or less, once every eight weeks or less, or once every four weeks, or less. In another preferred embodiment of the subject technology, the anti-IL-6 antibodies described herein, or IL-6 binding fragments or variants thereof, as well as combinations thereof, are administered to a recipient subject with a frequency at most once per period of approximately one week, such as at most once per period of approximately two weeks, such as at most once per period of approximately four weeks, such as at most once per period of approximately eight weeks, such as at most once per period of approximately twelve weeks, such as at most once per period of approximately sixteen weeks, such as at most once per period of approximately twenty-four weeks.

The compositions described herein may be administered in any of the following routes: buccal, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The preferred routes of administration are intravenous injection or infusion. The administration can be local, where the composition is administered directly, close to, in the locality, near, at, about, or in the vicinity of, the site(s) of disease, e.g., local (joint) or systemic, wherein the composition is given to the patient and passes through the body widely, thereby reaching the site(s) of disease. Local administration (e.g., subcutaneous injection) may be accomplished by administration to the cell, tissue, organ, and/or organ system, which encompasses and/or is affected by the disease, and/or where the disease signs and/or symptoms are active or are likely to occur (e.g., swollen joint). Administration can be topical with a local effect, composition is applied directly where its action is desired (e.g., joint). Further, administration of a composition comprising an effective amount of an anti-IL-6 antibody selected from the group consisting of Ab1-Ab36 or an antigen-binding fragment thereof, may be subcutaneous.

For each of the recited embodiments, the compounds can be administered by a variety of dosage forms as known in the art. Any biologically-acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds which can be included by admixture are, for example, medically inert ingredients (e.g., solid and liquid diluent), such as lactose, dextrosesaccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

In further embodiments, the present subject technology provides kits including at least one container comprising pharmaceutical dosage units comprising an effective amount of at least one antibody and fragments thereof of the present subject technology. Kits may include instructions, directions, labels, marketing information, warnings, or information pamphlets.

### Dosages

The amount of anti-IL-6 antibodies in a therapeutic composition according to any embodiments of this subject technology may vary according to factors such as the disease state, age, gender, weight, patient history, risk factors, predisposition to disease, administration route, pre-existing treatment regime (e.g., possible interactions with other medications), and weight of the individual. Dosage regimens may be adjusted to provide the optimum therapeutic response. For example, a single bolus may be administered, several divided doses may be administered over time, or the dose may be proportionally reduced or increased as indicated by the exigencies of therapeutic situation.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of antibodies, or antigen-binding fragments thereof, calculated to produce the desired therapeutic effect in association with the required pharmaceutical carrier. The specification for the dosage unit forms of the subject technology are dictated by and directly dependent on the unique characteristics of the antibodies, and fragments thereof, and the particular therapeutic effect to be achieved, and the limitations inherent in the art of compounding such an antibodies, and fragments thereof, for the treatment of sensitivity in individuals. In therapeutic use for treatment of conditions in mammals (e.g., humans) for which the antibodies and fragments thereof of the present subject technology or an appropriate pharmaceutical composition thereof are effective, the antibodies and fragments thereof of the present subject technology may be administered in an effective amount. The dosages as suitable for this subject technology may be a composition, a pharmaceutical composition or any other compositions described herein.

The dosage may be administered as a single dose, a double dose, a triple dose, a quadruple dose, and/or a quintuple dose. The dosages may be administered singularly, simultaneously, and sequentially. For example, two doses may be administered on the same day followed by subsequent two doses four weeks later.

The dosage form may be any form of release known to persons of ordinary skill in the art. The compositions of the present subject technology may be formulated to provide immediate release of the active ingredient or sustained or controlled release of the active ingredient. In a sustained release or controlled release preparation, release of the active ingredient may occur at a rate such that blood levels are maintained within a therapeutic range but below toxic levels over an extended period of time (e.g., 4 to 24 hours). The preferred dosage forms include immediate release, extended release, pulse release, variable release, controlled release, timed release, sustained release, delayed release, long acting, and combinations thereof, and are known in the art.

It will be appreciated that the pharmacological activity of the compositions may be monitored using standard pharmacological models that are known in the art. Furthermore, it will be appreciated that the compositions comprising an anti-IL-6 antibodies or antigen-binding fragments thereof may be incorporated or encapsulated in a suitable polymer matrix or membrane for site-specific delivery, or may be functionalized with specific targeting agents capable of effecting site specific delivery. These techniques, as well as other drug delivery techniques are well known in the art. Determination of optimal dosages for a particular situation is within the capabilities of those skilled in the art. See, e.g., Grennaro (2005) [Ed.] Remington: The Science and Practice of Pharmacy [21^{st} Ed.]

In another embodiment of the subject technology, the anti-IL-6 antibodies described herein, or IL-6 binding fragments or variants thereof, as well as combinations of said antibody fragments or variants, are administered to a subject in a pharmaceutical formulation.

A “pharmaceutical composition” refers to a chemical or biological composition suitable for administration to a mammal. Such compositions may be specifically formulated for administration via at least one of a number of routes, including but not limited to buccal, epicutaneous, epidural, inhalation, intraarterial, intracardial, intracerebroventricular, intradermal, intramuscular, intranasal, intraocular, intraperitoneal, intraspinal, intrathecal, intravenous, oral, parenteral, rectally via an enema or suppository, subcutaneous, subdermal, sublingual, transdermal, and transmucosal. In addition, administration can occur by means of injection, powder, liquid, gel, drops, or other means of administration. Further, a pharmaceutical composition comprising an anti-IL-6 antibody described herein (e.g., ALD518) may be administered subcutaneously.

In one embodiment of the subject technology, the anti-IL-6 antibodies described herein, or IL-6 binding fragments or variants thereof, as well as combinations of said antibody fragments or variants, may be optionally administered in combination with at least one active agent. Such active agents include analgesic, antipyretic, anti-inflammatory, antibiotic, antiviral, and anti-cytokine agents. Active agents include agonists, antagonists, and modulators of TNF-alpha, IL-2, IL-4, IL-6, IL-10, IL-12, IL-13, IL-18, IFN-alpha, IFN-gamma, BAFF, CXCL13, IP-10, VEGF, EPO, EGF, HRG, Hepatocyte Growth Factor (HGF), Hepcidin, including antibodies reactive against any of the foregoing, and antibodies reactive against any of their receptors. Active agents also include 2-Arylpropionic acids, Aceclofenac, Acemetacin, Acetylsalicylic acid (Aspirin), Alclofenac, Alminoprofen, Amoxiprin, Ampyrone, Arylalkanoic acids, Azapropazone, Benorylate/Benorilate, Benoxaprofen, Bromfenac, Carprofen, Celecoxib, Choline magnesium salicylate, Clofezone, COX-2 inhibitors, Dexibuprofen, Dexketoprofen, Diclofenac, Diflunisal, Droxicam, Ethenzamide, Etodolac, Etoricoxib, Faislamine, fenamic acids, Fenbufen, Fenoprofen, Flufenamic acid, Flunoxaprofen, Flurbiprofen, Ibuprofen, Ibuproxam, Indometacin, Indoprofen, Kebuzone, Ketoprofen, Ketorolac, Lornoxicam, Loxoprofen, Lumiracoxib, Magnesium salicylate, Meclofenamic acid, Mefenamic acid, Meloxicam, Metamizole, Methyl salicylate, Mofebutazone, Nabumetone, Naproxen, N-Arylanthranilic acids, Oxametacin, Oxaprozin, Oxicams, Oxyphenbutazone, Parecoxib, Phenazone, Phenylbutazone, Phenylbutazone, Piroxicam, Pirprofen, profens, Proglumetacin, Pyrazolidine derivatives, Rofecoxib, Salicyl salicylate, Salicylamide, Salicylates, Sulfinpyrazone, Sulindac, Suprofen, Tenoxicam, Tiaprofenic acid, Tolfenamic acid, Tolmetin, and Valdecoxib. Antibiotics include Amikacin, Aminoglycosides, Amoxicillin, Ampicillin, Ansamycins, Arsphenamine, Azithromycin, Azlocillin, Aztreonam, Bacitracin, Carbacephem, Carbapenems, Carbenicillin, Cefaclor, Cefadroxil, Cefalexin, Cefalothin, Cefalotin, Cefamandole, Cefazolin, Cefdinir, Cefditoren, Cefepime, Cefixime, Cefoperazone, Cefotaxime, Cefoxitin, Cefpodoxime, Cefprozil, Ceftazidime, Ceftibuten, Ceftizoxime, Ceftobiprole, Ceftriaxone, Cefuroxime, Cephalosporins, Chloramphenicol, Cilastatin, Ciprofloxacin, Clarithromycin, Clindamycin, Cloxacillin, Colistin, Co-trimoxazole, Dalfopristin, Demeclocycline, Dicloxacillin, Dirithromycin, Doripenem, Doxycycline, Enoxacin, Ertapenem, Erythromycin, Ethambutol, Flucloxacillin, Fosfomycin, Furazolidone, Fusidic acid, Gatifloxacin, Geldanamycin, Gentamicin, Glycopeptides, Herbimycin, Imipenem, Isoniazid, Kanamycin, Levofloxacin, Lincomycin, Linezolid, Lomefloxacin, Loracarbef, Macrolides, Mafenide, Meropenem, Meticillin, Metronidazole, Mezlocillin, Minocycline, Monobactams, Moxifloxacin, Mupirocin, Nafcillin, Neomycin, Netilmicin, Nitrofurantoin, Norfloxacin, Ofloxacin, Oxacillin, Oxytetracycline, Paromomycin, Penicillin, Penicillins, Piperacillin, Platensimycin, Polymyxin B, Polypeptides, Prontosil, Pyrazinamide, Quinolones, Quinupristin, Rifampicin, Rifampin, Roxithromycin, Spectinomycin, Streptomycin, Sulfacetamide, Sulfamethizole, Sulfanilimide, Sulfasalazine, Sulfisoxazole, Sulfonamides, Teicoplanin, Telithromycin, Tetracycline, Tetracyclines, Ticarcillin, Tinidazole, Tobramycin, Trimethoprim, Trimethoprim-Sulfamethoxazole, Troleandomycin, Trovafloxacin, and Vancomycin. Active agents also include Aldosterone, Beclometasone, Betamethasone, Corticosteroids, Cortisol, Cortisone acetate, Deoxycorticosterone acetate, Dexamethasone, Fludrocortisone acetate, Glucocorticoids, Hydrocortisone, Methylprednisolone, Prednisolone, Prednisone, Steroids, and Triamcinolone. Antiviral agents include but are not limited to abacavir, aciclovir, acyclovir, adefovir, amantadine, amprenavir, an antiretroviral fixed dose combination, an antiretroviral synergistic enhancer, arbidol, atazanavir, atripla, brivudine, cidofovir, combivir, darunavir, delavirdine, didanosine, docosanol, edoxudine, efavirenz, emtricitabine, enfuvirtide, entecavir, entry inhibitors, famciclovir, fomivirsen, fosamprenavir, foscarnet, fosfonet, fusion inhibitor, ganciclovir, gardasil, ibacitabine, idoxuridine, imiquimod, imunovir, indinavir, inosine, integrase inhibitor, interferon, interferon type I, interferon type II, interferon type III, lamivudine, lopinavir, loviride, maraviroc, MK-0518, moroxydine, nelfinavir, nevirapine, nexavir, nucleoside analogues, oseltamivir, penciclovir, peramivir, pleconaril, podophyllotoxin, protease inhibitor, reverse transcriptase inhibitor, ribavirin, rimantadine, ritonavir, saquinavir, stavudine, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, and zidovudine. Any suitable combination of these active agents is also contemplated.

A “pharmaceutical excipient” or a “pharmaceutically acceptable excipient” is a carrier, usually a liquid, in which an active therapeutic agent is formulated. In one embodiment of the subject technology, the active therapeutic agent is a humanized antibody described herein, or at least one fragments or variants thereof. The excipient generally does not provide any pharmacological activity to the formulation, though it may provide chemical and/or biological stability, and release characteristics. Exemplary formulations can be found, for example, in Grennaro (2005) [Ed.] Remington: The Science and Practice of Pharmacy [21^{st} Ed.]

As used herein “pharmaceutically acceptable carrier” or “excipient” includes any and all solvents, dispersion media, coatings, antibacterial and antifungal agents, isotonic and absorption delaying agents that are physiologically compatible. In one embodiment, the carrier is suitable for parenteral administration. Alternatively, the carrier can be suitable for intravenous, intraperitoneal, intramuscular, or sublingual administration. Pharmaceutically acceptable carriers include sterile aqueous solutions or dispersions and sterile powders for the extemporaneous preparation of sterile injectable solutions or dispersions. The use of such media and agents for pharmaceutically active substances is well known in the art. Except insofar as any conventional media or agent is incompatible with the active compound, use thereof in the pharmaceutical compositions of the subject technology is contemplated. Supplementary active compounds can also be incorporated into the compositions.

Pharmaceutical compositions typically must be sterile and stable under the conditions of manufacture and storage. The subject technology contemplates that the pharmaceutical composition is present in lyophilized form. The composition may be formulated as a solution, microemulsion, liposome, or other ordered structure suitable to high drug concentration. The carrier may be a solvent or dispersion medium containing, for example, water, ethanol, polyol (for example, glycerol, propylene glycol, and liquid polyethylene glycol), and suitable mixtures thereof. The subject technology further contemplates the inclusion of a stabilizer in the pharmaceutical composition.

The antibodies and fragments thereof, of the present subject technology thereof may be formulated into pharmaceutical compositions of various dosage forms. For example, the antibody may be ALD518, a humanized anti-interleukin-6 (anti-IL-6) monoclonal immunoglobulin 1 (IgG1) antibody manufactured in the yeast Pichia pastoris. ALD518 may be supplied as a pH 6.0 frozen injection in single-use vials (80 mg or 160 mg) for intravenous administration. Examplary non-active excipients include but are not limited to histidine (e.g., 25 mM) and sorbitol (e.g., 250 mM). For example, a 160 mg formulation may comprise as non-active excipients, 25 mM histidine, 250 mM sorbitol, and 0.015% polysorbate 80. To prepare the pharmaceutical compositions of the subject technology, at least one anti-IL-6 antibodies or binding fragments thereof, as the active ingredient may be intimately mixed with appropriate carriers and additives according to techniques well known to those skilled in the art of pharmaceutical formulations. See Grennaro (2005) [Ed.] Remington: The Science and Practice of Pharmacy [21^{st} Ed.] For example, the antibodies described herein may be formulated in phosphate buffered saline pH 7.2 and supplied as a 5.0 mg/mL clear colorless liquid solution.

Similarly, compositions for liquid preparations include solutions, emulsions, dispersions, suspensions, syrups, and elixirs, with suitable carriers and additives including but not limited to water, alcohols, oils, glycols, preservatives, flavoring agents, coloring agents, and suspending agents. Typical preparations for parenteral administration comprise the active ingredient with a carrier such as sterile water or parenterally acceptable oil including but not limited to polyethylene glycol, polyvinyl pyrrolidone, lecithin, arachis oil or sesame oil, with other additives for aiding solubility or preservation may also be included. In the case of a solution, it may be lyophilized to a powder and then reconstituted immediately prior to use. For dispersions and suspensions, appropriate carriers and additives include aqueous gums, celluloses, silicates, or oils.

For each of the recited embodiments, the anti-IL-6 antibodies or binding fragments thereof, may be administered by a variety of dosage forms. Any biologically-acceptable dosage form known to persons of ordinary skill in the art, and combinations thereof, are contemplated. Examples of such dosage forms include, without limitation, reconstitutable powders, elixirs, liquids, solutions, suspensions, emulsions, powders, granules, particles, microparticles, dispersible granules, cachets, inhalants, aerosol inhalants, patches, particle inhalants, implants, depot implants, injectables (including subcutaneous, intramuscular, intravenous, and intradermal), infusions, and combinations thereof.

In many cases, it will be preferable to include isotonic agents, e.g., sugars, polyalcohols such as mannitol, sorbitol, or sodium chloride in the composition. Prolonged absorption of the injectable compositions may be brought about by including in the composition an agent which delays absorption, e.g., monostearate salts and gelatin. Moreover, the compounds described herein may be formulated in a time release formulation, e.g. in a composition that includes a slow release polymer. The anti-IL-6 antibodies may be prepared with carriers that will protect the compound against rapid release, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

In one embodiment of the subject technology that may be used to intravenously administer antibodies of the subject technology, including ALD518, for psoriatic arthritis indications, the administration formulation comprises, or alternatively consists of, about 10.5 mg/mL of antibody, 25 mM Histidine base, Phosphoric acid q.s. to pH 6, and 250 mM sorbitol.

In another embodiment of the subject technology that may be used to intravenously administer antibodies of the subject technology, including ALD581, for psoriatic arthritis indications, the administration formulation comprises, or alternatively consists of, about 10.5 mg/mL of antibody, 12.5 mM Histidine base, 12.5 mM Histidine HCl (or 25 mM Histidine base and Hydrochloric acid q.s. to pH 6), 250 mM sorbitol, and 0.015% (w/w) Polysorbate 80.

In one embodiment of the subject technology that may be used to subcutaneously administer antibodies of the subject technology, including ALD518, for psoriatic arthritis indications, the administration formulation comprises, or alternatively consists of, about 50 or 100 mg/mL of antibody, about 5 mM Histidine base, about 5 mM Histidine HCl to make final pH 6, 250 mM sorbitol, and 0.015% (w/w) Polysorbate 80. In another embodiment of the subject technology that may be used to subcutaneously administer antibodies of the subject technology, including Ab1, for psoriatic arthritis indications, the administration formulation comprises, or alternatively consists of, about 20 or 100 mg/mL of antibody, about 5 mM Histidine base, about 5 mM Histidine HCl to make final pH 6, 250 to 280 mM sorbitol (or sorbitol in combination with sucrose), and 0.015% (w/w) Polysorbate 80, said formulation having a nitrogen headspace in the shipping vials.

Pharmaceutical compositions typically must be sterile and stable under the conditions of manufacture and storage. The subject technology contemplates that the pharmaceutical composition is present in lyophilized form. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol), and suitable mixtures thereof. The subject technology further contemplates the inclusion of a stabilizer in the pharmaceutical composition.

In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the alkaline polypeptide can be formulated in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

For each of the recited embodiments, the compounds can be administered by a variety of dosage forms. Any biologically-acceptable dosage form known to persons of ordinary skill in the art, and combinations thereof, are contemplated. Examples of such dosage forms include, without limitation, reconstitutable powders, elixirs, liquids, solutions, suspensions, emulsions, powders, granules, particles, microparticles, dispersible granules, cachets, inhalants, aerosol inhalants, patches, particle inhalants, implants, depot implants, injectables (including subcutaneous, intramuscular, intravenous, and intradermal), infusions, and combinations thereof.

A person of skill in the art would be able to determine an effective dosage and frequency of administration through routine experimentation, for example guided by the disclosure herein and the teachings in Goodman, et al. (2011) Goodman & Gilman's The Pharmacological Basis of Therapeutics [12^{th} Ed.]; Howland, et al. (2005) Lippincott's Illustrated Reviews: Pharmacology [2^{nd} Ed.]; and Golan, (2008) Principles of Pharmacology: The Pathophysiologic Basis of Drug Therapy [2^{nd} Ed.] See, also, Grennaro (2005) [Ed.] Remington: The Science and Practice of Pharmacy [21^{st} Ed.]

The above description of various illustrated embodiments of the subject technology is not intended to be exhaustive or to limit the subject technology to the precise form disclosed. While specific embodiments of, and examples for, the subject technology are described herein for illustrative purposes, various equivalent modifications are possible within the scope of the subject technology, as those skilled in the relevant art will recognize. The teachings provided herein of the subject technology can be applied to other purposes, other than the examples described above.

These and other changes can be made to the subject technology in light of the above detailed description. In general, in the following claims, the terms used should not be construed to limit the subject technology to the specific embodiments disclosed in the specification and the claims. Accordingly, the subject technology is not limited by the disclosure, but instead the scope of the subject technology is to be determined entirely by the following claims.

The subject technology may be practiced in ways other than those particularly described in the foregoing description and examples. Numerous modifications and variations of the subject technology are possible in light of the above teachings and, therefore, are within the scope of the appended claims.

Certain teachings related to methods for obtaining a clonal population of antigen-specific B cells were disclosed in U.S. Patent Application Publication No. 2007/0269868.

Certain teachings related to humanization of rabbit-derived monoclonal antibodies and preferred sequence modifications to maintain antigen binding affinity were disclosed in U.S. Patent Application Publication No. 2009/0104187.

Certain teachings related to producing antibodies or fragments thereof using mating competent yeast and corresponding methods were disclosed in U.S. Patent Application Publication No. 2006/0270045.

Certain teachings related to anti-IL-6 antibodies, methods of producing antibodies or fragments thereof using mating competent yeast and corresponding methods were disclosed in U.S. Patent Application Publication No. 2009/0104187.

Certain teachings related to anti-IL-6 antibodies and methods of using those antibodies or fragments thereof to address certain diseases and/or disorders were disclosed in U.S. Patent Application Publication No. 2010/0150829.

Certain anti-IL-6 antibody polynucleotides and polypeptides are disclosed in the sequence listing accompanying this patent application filing, and the disclosure of said sequence listing is herein incorporated by reference in its entirety.

The following examples are put forth so as to provide those of ordinary skill in the art with a complete disclosure and description of how to make and use the subject technology, and are not intended to limit the scope of what is regarded as the subject technology. Efforts have been made to ensure accuracy with respect to the numbers used (e.g. amounts, temperature, concentrations) but some experimental errors and deviations should be allowed for. Unless otherwise indicated, parts are parts by weight, molecular weight is average molecular weight, temperature is in degrees centigrade; and pressure is at or near atmospheric.

### EXAMPLES

In the following examples, the term “Ab1” refers to an antibody comprising the light chain sequence of SEQ ID NO: 702 and the heavy chain sequence of SEQ ID NO: 704, except where the context indicates otherwise. The laboratory designation “Ab1” also encompasses an anti-IL-6 antibody also known as “clazakizumab,” “ALD518” and “BMS-945429” comprising the light chain sequence of SEQ ID NO: 19 and the heavy chain sequence of SEQ ID NO: 20.

### Example 1

### Production of Enriched Antigen-Specific B Cell Antibody Culture

Panels of antibodies are derived by immunizing traditional antibody host animals to exploit the native immune response to a target antigen of interest. Typically, the host used for immunization is a rabbit or other host that produces antibodies using a similar maturation process and provides for a population of antigen-specific B cells producing antibodies of comparable diversity, e.g., epitopic diversity. The initial antigen immunization can be conducted using complete Freund's adjuvant (CFA), and the subsequent boosts effected with incomplete adjuvant. At about 50-60 days after immunization, preferably at day 55, antibody titers are tested, and the Antibody Selection (ABS) process is initiated if appropriate titers are established. The two key criteria for ABS initiation are potent antigen recognition and function-modifying activity in the polyclonal sera.

At the time positive antibody titers are established, animals are sacrificed and B cell sources isolated. These sources include: the spleen, lymph nodes, bone marrow, and peripheral blood mononuclear cells (PBMCs). Single cell suspensions are generated, and the cell suspensions are washed to make them compatible for low temperature long term storage. The cells are then typically frozen.

To initiate the antibody identification process, a small fraction of the frozen cell suspensions are thawed, washed, and placed in tissue culture media. These suspensions are then mixed with a biotinylated form of the antigen that was used to generate the animal immune response, and antigen-specific cells are recovered using the Miltenyi magnetic bead cell selection methodology. Specific enrichment is conducted using streptavidin beads. The enriched population is recovered and progressed in the next phase of specific B cell isolation.

### Example 2

### Production of Clonal, Antigen-Specific B Cell-Containing Culture

Enriched B cells produced according to Example 1 are then plated at varying cell densities per well in a 96 well microtiter plate. Generally, this is at 50, 100, 250, or 500 cells per well with 10 plates per group. The media is supplemented with 4% activated rabbit T cell conditioned media along with 50K frozen irradiated EL4B feeder cells. These cultures are left undisturbed for 5-7 days at which time supernatant-containing secreted antibody is collected and evaluated for target properties in a separate assay setting. The remaining supernatant is left intact, and the plate is frozen at −70° C. Under these conditions, the culture process typically results in wells containing a mixed cell population that comprises a clonal population of antigen-specific B cells, i.e., a single well will only contain a single monoclonal antibody specific to the desired antigen.

### Example 3

### Screening of Antibody Supernatants for Monoclonal Antibody of Desired Specificity and/or Functional Properties

Antibody-containing supernatants derived from the well containing a clonal antigen-specific B cell population produced according to Example 2 are initially screened for antigen recognition using ELISA methods. This includes selective antigen immobilization (e.g., biotinylated antigen capture by streptavidin coated plate), non-specific antigen plate coating, or alternatively, through an antigen build-up strategy (e.g., selective antigen capture followed by binding partner addition to generate a heteromeric protein-antigen complex). Antigen-positive well supernatants are then optionally tested in a function-modifying assay that is strictly dependant on the ligand. One such example is an in vitro protein-protein interaction assay that recreates the natural interaction of the antigen ligand with recombinant receptor protein. Alternatively, a cell-based response that is ligand dependent and easily monitored (e.g., proliferation response) is utilized. Supernatant that displays significant antigen recognition and potency is deemed a positive well. Cells derived from the original positive well are then transitioned to the antibody recovery phase.

### Example 4

### Recovery of Single, Antibody-Producing B Cell of Desired Antigen Specificity

Cells are isolated from a well that contains a clonal population of antigen-specific B cells (produced according to Example 2 or 3), which secrete a single antibody sequence. The isolated cells are then assayed to isolate a single, antibody-secreting cell. Dynal® (magnetic beads) streptavidin beads are coated with biotinylated target antigen under buffered medium to prepare antigen-containing microbeads compatible with cell viability. Next antigen-loaded beads, antibody-producing cells from the positive well, and a fluorescein isothiocyanate (FITC)-labeled anti-host H&L IgG antibody (as noted, the host can be any mammalian host, e.g., rabbit, mouse, rat) are incubated together at 37° C. This mixture is then re-pipetted in aliquots onto a glass slide such that each aliquot has on average a single, antibody-producing B-cell. The antigen-specific, antibody-secreting cells are then detected through fluorescence microscopy. Secreted antibody is locally concentrated onto the adjacent beads due to the bound antigen and provides localization information based on the strong fluorescent signal. Antibody-secreting cells are identified via FITC detection of antibody-antigen complexes formed adjacent to the secreting cell. The single cell found in the center of this complex is then recovered using a micromanipulator. The cell is snap-frozen in an Eppendorf PCR tube for storage at −80° C. until antibody sequence recovery is initiated.

### Example 5

### Isolation of Antibody Sequences from Antigen-Specific B Cell

Antibody sequences are recovered using a combined RT-PCR based method from a single isolated B-cell produced according to Example 4 or an antigenic specific B cell isolated from the clonal B cell population obtained according to Example 2. Primers are designed to anneal in conserved and constant regions of the target immunoglobulin genes (heavy and light), such as rabbit immunoglobulin sequences, and a two-step nested PCR recovery step is used to obtain the antibody sequence. Amplicons from each well are analyzed for recovery and size integrity. The resulting fragments are then digested with AluI to fingerprint the sequence clonality. Identical sequences display a common fragmentation pattern in their electrophoretic analysis. Significantly, this common fragmentation pattern which proves cell clonality is generally observed even in the wells originally plated up to 1000 cells/well. The original heavy and light chain amplicon fragments are then restriction enzyme digested with HindIII and XhoI or HindIII and BsiWI to prepare the respective pieces of DNA for cloning. The resulting digestions are then ligated into an expression vector and transformed into bacteria for plasmid propagation and production. Colonies are selected for sequence characterization.

### Example 6

### Recombinant Production of Monoclonal Antibody of Desired Antigen Specificity and/or Functional Properties

Correct full-length antibody sequences for each well containing a single monoclonal antibody is established and miniprep DNA is prepared using Qiagen solid-phase methodology. This DNA is then used to transfect mammalian cells to produce recombinant full-length antibody. Crude antibody product is tested for antigen recognition and functional properties to confirm the original characteristics are found in the recombinant antibody protein. Where appropriate, large-scale transient mammalian transfections are completed, and antibody is purified through Protein A affinity chromatography. K_{d} is assessed using standard methods (e.g., Biacore®) as well as IC₅₀ in a potency assay.

### Example 7

### Preparation of Antibodies that Bind Human IL-6

By using the antibody selection protocol described herein, one can generate an extensive panel of antibodies. The antibodies have high affinity towards IL-6 (single to double digit pM Kd) and demonstrate potent antagonism of IL-6 in multiple cell-based screening systems (T1165 and HepG2). Furthermore, the collection of antibodies displays distinct modes of antagonism toward IL-6-driven processes.

### Immunization Strategy

Rabbits were immunized with huIL-6 (R&R). Immunization consisted of a first subcutaneous (sc) injection of 100 μg in complete Freund's adjuvant (CFA) (Sigma) followed by two boosts, two weeks apart, of 50 μg each in incomplete Freund's adjuvant (IFA) (Sigma). Animals were bled on day 55, and serum titers were determined by ELISA (antigen recognition) and by non-radioactive proliferation assay (Promega) using the T1165 cell line.

### Antibody Selection Titer Assessment

Antigen recognition was determined by coating Immulon 4 plates (Thermo) with 1 μg/mL of huIL-6 (50 μL/well) in phosphate buffered saline (PBS, Hyclone) overnight at 4° C. On the day of the assay, plates were washed 3 times with PBS/Tween 20 (PBST tablets, Calbiochem). Plates were then blocked with 200 μL/well of 0.5% fish skin gelatin (FSG, Sigma) in PBS for 30 minutes at 37° C. Blocking solution was removed, and plates were blotted. Serum samples were made (bleeds and pre-bleeds) at a starting dilution of 1:100 (all dilutions were made in FSG 50 μL/well) followed by 1:10 dilutions across the plate (column 12 was left blank for background control). Plates were incubated for 30 minutes at 37° C. Plates were washed 3 times with PBS/Tween 20. Goat anti-rabbit Fc-HRP (Pierce) diluted 1:5000 was added to all wells (50 μL/well), and plates were incubated for 30 minutes at 37° C. Plates were washed as described above. 50 μL/well of TMB-Stable stop (Fitzgerald Industries) was added to plates, and color was allowed to develop, generally for 3 to 5 minutes. The development reaction was stopped with 50 μL/well 0.5 M HCl. Plates were read at 450 nm. Optical density (OD) versus dilution was plotted using Graph Pad Prizm software, and titers were determined.

### Functional Titer Assessment

The functional activity of the samples was determined by a T1165 proliferation assay. T1165 cells were routinely maintained in modified RPMI medium (Hyclone) supplemented with HEPES, sodium pyruvate, sodium bicarbonate, L-glutamine, high glucose, penicillin/streptomycin, 10% heat inactivated fetal bovine serum (FBS) (all supplements from Hyclone), 2-mercaptoethanol (Sigma), and 10 ng/mL of huIL-6 (R&D). On the day of the assay, cell viability was determined by trypan blue (Invitrogen), and cells were seeded at a fixed density of 20,000 cells/well. Prior to seeding, cells were washed twice in the medium described above without human-IL-6 (by centrifuging at 13000 rpm for 5 minutes and discarding the supernatant). After the last wash, cells were resuspended in the same medium used for washing in a volume equivalent to 50 μL/well. Cells were set aside at room temperature.

In a round-bottom, 96-well plate (Costar), serum samples were added starting at 1:100, followed by a 1:10 dilution across the plate (columns 2 to 10) at 30 μL/well in replicates of 5 (rows B to F: dilution made in the medium described above with no huIL-6). Column 11 was medium only for IL-6 control. 30 μL/well of huIL-6 at 4× concentration of the final EC50 (concentration previously determined) were added to all wells (huIL-6 was diluted in the medium described above). Wells were incubated for 1 hour at 37° C. to allow antibody binding to occur. After 1 hour, 50 μL/well of antibody-antigen (Ab-Ag) complex were transferred to a flat-bottom, 96-well plate (Costar) following the plate map format laid out in the round-bottom plate. On Row G, 50 μL/well of medium were added to all wells (columns 2 to 11) for background control. 50 μL/well of the cell suspension set aside were added to all wells (columns 2 to 11, rows B to G). On Columns 1 and 12 and on rows A and H, 200 μL/well of medium was added to prevent evaporation of test wells and to minimize edge effect. Plates were incubated for 72 hours at 37° C. in 4% CO₂. At 72 hours, 20 μL/well of CellTiter96 (Promega) reagents was added to all test wells per manufacturer protocol, and plates were incubated for 2 hours at 37° C. At 2 h, plates were gently mixed on an orbital shaker to disperse cells and to allow homogeneity in the test wells. Plates were read at 490 nm wavelength. Optical density (OD) versus dilution was plotted using Graph Pad Prizm software, and functional titer was determined. A positive assay control plate was conducted as described above using MAB2061 (R&D Systems) at a starting concentration of 1 μg/mL (final concentration) followed by 1:3 dilutions across the plate.

### Tissue Harvesting

Once acceptable titers were established, the rabbit(s) were sacrificed. Spleen, lymph nodes, and whole blood were harvested and processed as follows:

Spleen and lymph nodes were processed into a single cell suspension by disassociating the tissue and pushing through sterile wire mesh at 70 μm (Fisher) with a plunger of a 20 cc syringe. Cells were collected in the modified RPMI medium described above without huIL-6, but with low glucose. Cells were washed twice by centrifugation. After the last wash, cell density was determined by trypan blue. Cells were centrifuged at 1500 rpm for 10 minutes; the supernatant was discarded. Cells were resuspended in the appropriate volume of 10% dimethyl sulfoxide (DMSO, Sigma) in FBS (Hyclone) and dispensed at 1 mL/vial. Vials were then stored at −70° C. for 24 h prior to being placed in a liquid nitrogen (LN2) tank for long-term storage.

Peripheral blood mononuclear cells (PBMCs) were isolated by mixing whole blood with equal parts of the low glucose medium described above without FBS. 35 mL of the whole blood mixture was carefully layered onto 8 mL of Lympholyte Rabbit (Cedarlane) into a 45 mL conical tube (Corning) and centrifuged 30 minutes at 2500 rpm at room temperature without brakes. After centrifugation, the PBMC layers were carefully removed using a glass Pasteur pipette (VWR), combined, and placed into a clean 50 mL vial. Cells were washed twice with the modified medium described above by centrifugation at 1500 rpm for 10 minutes at room temperature, and cell density was determined by trypan blue staining After the last wash, cells were resuspended in an appropriate volume of 10% DMSO/FBS medium and frozen as described herein.

### B Cell Culture

On the day of setting up B cell culture, PBMC, splenocyte, or lymph node vials were thawed for use. Vials were removed from LN2 tank and placed in a 37° C. water bath until thawed. Contents of vials were transferred into 15 mL conical centrifuge tube (Corning) and 10 mL of modified RPMI described above was slowly added to the tube. Cells were centrifuged for 5 minutes at 1.5K rpm, and the supernatant was discarded. Cells were resuspended in 10 mL of fresh media. Cell density and viability was determined by trypan blue. Cells were washed again and resuspended at 1E07 cells/80 μL medium. Biotinylated huIL-6 (B huIL-6) was added to the cell suspension at the final concentration of 3 μg/mL and incubated for 30 minutes at 4° C. Unbound B huIL-6 was removed with two 10 mL washes of phosphate-buffered (PBF): Ca/Mg free PBS (Hyclone), 2 mM ethylenediamine tetraacetic acid (EDTA), 0.5% bovine serum albumin (BSA) (Sigma-biotin free). After the second wash, cells were resuspended at 1E07 cells/80 μL PBF. 20 μL of MACS® streptavidin beads (Milteni)/10E7 cells were added to the cell suspension. Cells were incubated at 4° C. for 15 minutes. Cells were washed once with 2 mL of PBF/10E7 cells. After washing, the cells were resuspended at 1E08 cells/500 μL of PBF and set aside. A MACS® MS column (Milteni) was pre-rinsed with 500 mL of PBF on a magnetic stand (Milteni). Cell suspension was applied to the column through a pre-filter, and unbound fraction was collected. The column was washed with 1.5 mL of PBF buffer. The column was removed from the magnet stand and placed onto a clean, sterile 5 mL Polypropylene Falcon tube. 1 mL of PBF buffer was added to the top of the column, and positive selected cells were collected. The yield and viability of positive and negative cell fraction was determined by trypan blue staining Positive selection yielded an average of 1% of the starting cell concentration.

A pilot cell screen was established to provide information on seeding levels for the culture. Three 10-plate groups (a total of 30 plates) were seeded at 50, 100, and 200 enriched B cells/well. In addition, each well contained 50K cells/well of irradiated EL-4.B5 cells (5,000 Rads) and an appropriate level of T cell supernatant (ranging from 1-5% depending on preparation) in high glucose modified RPMI medium at a final volume of 250 μL/well. Cultures were incubated for 5 to 7 days at 37° C. in 4% CO₂.

### Identification of Selective Antibody Secreting B Cells

Cultures were tested for antigen recognition and functional activity between days 5 and 7.

### Antigen Recognition Screening

The ELISA format used is as described above except 50 μL of supernatant from the B cell cultures (BCC) wells (all 30 plates) was used as the source of the antibody. The conditioned medium was transferred to antigen-coated plates. After positive wells were identified, the supernatant was removed and transferred to a 96-well master plate(s). The original culture plates were then frozen by removing all the supernatant except 40 μL/well and adding 60 μL/well of 16% DMSO in FBS. Plates were wrapped in paper towels to slow freezing and placed at −70° C.

### Functional Activity Screening

Master plates were then screened for functional activity in the T1165 proliferation assay as described before, except row B was media only for background control, row C was media+IL-6 for positive proliferation control, and rows D-G and columns 2-11 were the wells from the BCC (50 μL/well, single points). 40 μL of IL-6 was added to all wells except the media row at 2.5 times the EC50 concentration determined for the assay. After 1 h incubation, the Ab/Ag complex was transferred to a tissue culture (TC) treated, 96-well, flat-bottom plate. 20 μL of cell suspension in modified RPMI medium without huIL-6 (T1165 at 20,000 cells/well) was added to all wells (100 μL final volume per well). Background was subtracted, and observed OD values were transformed into % of inhibition.

### B Cell Recovery

Plates containing wells of interest were removed from −70° C., and the cells from each well were recovered with 5-200 μL washes of medium/well. The washes were pooled in a 1.5 mL sterile centrifuge tube, and cells were pelleted for 2 minutes at 1500 rpm.

The tube was inverted, the spin repeated, and the supernatant carefully removed. Cells were resuspended in 100 μL/tube of medium. 100 μL biotinylated IL-6 coated streptavidin M280 Dynabeads (Invitrogen) and 16 μL of goat anti-rabbit H&L IgG-FITC diluted 1:100 in medium was added to the cell suspension.

20 μL of cell/beads/FITC suspension was removed, and 5 μL droplets were prepared on a glass slide (Corning) previously treated with Sigmacote (Sigma), 35 to 40 droplets/slide. An impermeable barrier of paraffin oil (JT Baker) was added to submerge the droplets, and the slide was incubated for 90 minutes at 37° C., 4% CO₂ in the dark.

Specific B cells that produce antibody can be identified by the fluorescent ring around them due to antibody secretion, recognition of the bead-associated biotinylated antigen, and subsequent detection by the fluorescent-IgG detection reagent. Once a cell of interest was identified, the cell in the center of the fluorescent ring was recovered via a micromanipulator (Eppendorf). The single cell synthesizing and exporting the antibody was transferred into a 250 μL microcentrifuge tube and placed in dry ice. After recovering all cells of interest, these were transferred to −70° C. for long-term storage.

### Example 8

### Yeast Cell Expression

Antibody genes: Genes were cloned and constructed that directed the synthesis of a chimeric humanized rabbit monoclonal antibody.

Expression vector: The vector contains the following functional components: 1) a mutant ColE 1 origin of replication, which facilitates the replication of the plasmid vector in cells of the bacterium Escherichia coli; 2) a bacterial Sh ble gene, which confers resistance to the antibiotic Zeocin® (phleomycin) and serves as the selectable marker for transformations of both E. coli and P. pastoris; 3) an expression cassette composed of the glyceraldehyde dehydrogenase gene (GAP gene) promoter, fused to sequences encoding the Saccharomyces cerevisiae alpha mating factor pre pro secretion leader sequence, followed by sequences encoding a P. pastoris transcriptional termination signal from the P. pastoris alcohol oxidase I gene (AOX1). The Zeocin® (phleomycin) resistance marker gene provides a means of enrichment for strains that contain multiple integrated copies of an expression vector in a strain by selecting for transformants that are resistant to higher levels of Zeocin® (phleomycin).

P. pastoris strains: P. pastoris strains met1, lys3, ura3 and ade1 may be used. Although any two complementing sets of auxotrophic strains could be used for the construction and maintenance of diploid strains, these two strains are especially suited for this method for two reasons. First, they grow more slowly than diploid strains that are the result of their mating or fusion. Thus, if a small number of haploid ade1 or ura3 cells remain present in a culture or arise through meiosis or other mechanism, the diploid strain should outgrow them in culture.

The second is that it is easy to monitor the sexual state of these strains since diploid Ade+ colonies arising from their mating are a normal white or cream color, whereas cells of any strains that are haploid ade1 mutants will form a colony with a distinct pink color. In addition, any strains that are haploid ura3 mutants are resistant to the drug 5-fluoro-orotic acid (FOA) and can be sensitively identified by plating samples of a culture on minimal medium+uracil plates with FOA. On these plates, only uracil-requiring ura3 mutant (presumably haploid) strains can grow and form colonies. Thus, with haploid parent strains marked with ade1 and ura3, one can readily monitor the sexual state of the resulting antibody-producing diploid strains (haploid versus diploid).

### Methods

Construction of pGAPZ-Alpha Expression Vectors for Transcription of Light and Heavy Chain Antibody Genes.

The humanized light and heavy chain fragments were cloned into the pGAPZ expression vectors through a PCR directed process. The recovered humanized constructs were subjected to amplification under standard KOD polymerase (Novagen) kit conditions ((1) 94° C., 2 minutes; (2) 94° C., 30 seconds (3) 55° C., 30 seconds; (4) 72° C., 30 seconds-cycling through steps 2-4 for 35 times; (5) 72° C. 2 minutes) employing the following primers (1) light chain forward AGCGCTTATTCCGCTATCCAGATGACCCAGTC-the AfeI site is single underlined (SEQ ID NO: 729). The end of the HSA signal sequence is double underlined, followed by the sequence for the mature variable light chain (not underlined); the reverse CGTACGTTTGATTTCCACCTTG (SEQ ID NO: 730).

Variable light chain reverse primer. BsiWI site is underlined, followed by the reverse complement for the 3′ end of the variable light chain. Upon restriction enzyme digest with AfeI and BsiWI this enable insertion in-frame with the pGAPZ vector using the human HAS leader sequence in frame with the human kapp light chain constant region for export. (2) A similar strategy is performed for the heavy chain. The forward primer employed is AGCGCTTATTCCGAGGTGCAGCTGGTGGAGTC (SEQ ID NO: 731). The AfeI site is single underlined. The end of the HSA signal sequence is double underlined, followed by the sequence for the mature variable heavy chain (not underlined). The reverse heavy chain primer is CTCGAGACGGTGACGAGGGT (SEQ ID NO: 732). The XhoI site is underlined, followed by the reverse complement for the 3′ end of the variable heavy chain. This enables cloning of the heavy chain in-frame with IgG-γ1 CH1-CH2-CH3 region previous inserted within pGAPZ using a comparable directional cloning strategy.

Transformation of expression vectors into haploid ade1 ura3, met1 and lys3 host strains of P. pastoris. All methods used for transformation of haploid P. pastoris strains and genetic manipulation of the P. pastoris sexual cycle are as described in Higgins, D. R., and Cregg, J. M., Eds. 1998. Pichia Protocols. Methods in Molecular Biology. Humana Press, Totowa, N.J.

Prior to transformation, each expression vector is linearized within the GAP promoter sequences with AvrII to direct the integration of the vectors into the GAP promoter locus of the P. pastoris genome. Samples of each vector are then individually transformed into electrocompetent cultures of the ade1, ura3, met1 and lys3 strains by electroporation and successful transformants are selected on YPD Zeocin® (phleomycin) plates by their resistance to this antibiotic. Resulting colonies are selected, streaked for single colonies on YPD Zeocin® (phleomycin) plates and then examined for the presence of the antibody gene insert by a PCR assay on genomic DNA extracted from each strain for the proper antibody gene insert and/or by the ability of each strain to synthesize an antibody chain by a colony lift/immunoblot method. Wung, et al. (1996) Biotechniques 21: 808-812. Haploid ade1, met1 and lys3 strains expressing one of the three heavy chain constructs are collected for diploid constructions along with haploid ura3 strain expressing light chain gene. The haploid expressing heavy chain genes are mated with the appropriate light chain haploid ura3 to generate diploid secreting protein.

Mating of haploid strains synthesizing a single antibody chain and selection of diploid derivatives synthesizing tetrameric functional antibodies. To mate P. pastoris haploid strains, each ade1 (or met1 or lys3) heavy chain producing strain to be crossed is streaked across a rich YPD plate and the ura3 light chain producing strain is streaked across a second YPD plate (˜10 streaks per plate). After one or two days incubation at 30° C., cells from one plate containing heavy chain strains and one plate containing ura3 light chain strains are transferred to a sterile velvet cloth on a replica-plating block in a cross hatched pattern so that each heavy chain strain contain a patch of cells mixed with each light chain strain. The cross-streaked replica plated cells are then transferred to a mating plate and incubated at 25° C. to stimulate the initiation of mating between strains. After two days, the cells on the mating plates are transferred again to a sterile velvet on a replica-plating block and then transferred to minimal medium plates. These plates are incubated at 30° C. for three days to allow for the selective growth of colonies of prototrophic diploid strains. Colonies that arose are picked and streaked onto a second minimal medium plate to single colony isolate and purify each diploid strain. The resulting diploid cell lines are then examined for antibody production.

Putative diploid strains are tested to demonstrate that they are diploid and contain both expression vectors for antibody production. For diploidy, samples of a strain are spread on mating plates to stimulate them to go through meiosis and form spores. Haploid spore products are collected and tested for phenotype. If a significant percentage of the resulting spore products are single or double auxotrophs it may be concluded that the original strain must have been diploid. Diploid strains are examined for the presence of both antibody genes by extracting genomic DNA from each and utilizing this DNA in PCR reactions specific for each gene.

Fusion of haploid strains synthesizing a single antibody chain and selection of diploid derivatives synthesizing tetrameric functional antibodies. As an alternative to the mating procedure described above, individual cultures of single-chain antibody producing haploid ade1 and ura3 strains are spheroplasted and their resulting spheroplasts fused using polyethylene glycol/CaCl₂. The fused haploid strains are then embedded in agar containing 1 M sorbitol and minimal medium to allow diploid strains to regenerate their cell wall and grow into visible colonies. Resulting colonies are picked from the agar, streaked onto a minimal medium plate, and the plates are incubated for two days at 30° C. to generate colonies from single cells of diploid cell lines. The resulting putative diploid cell lines are then examined for diploidy and antibody production as described above.

Purification and analysis of antibodies. A diploid strain for the production of full length antibody is derived through the mating of met1 light chain and lys3 heavy chain using the methods described above. Culture media from shake-flask or fermenter cultures of diploid P. pastoris expression strains are collected and examined for the presence of antibody protein via SDS-PAGE and immunoblotting using antibodies directed against heavy and light chains of human IgG, or specifically against the heavy chain of IgG.

To purify the yeast secreted antibodies, clarified media from antibody producing cultures are passed through a protein A column and after washing with 20 mM sodium phosphate, pH 7.0, binding buffer, protein A bound protein is eluted using 0.1 M glycine HCl buffer, pH 3.0. Fractions containing the most total protein are examined by Coomassie blue strained SDS-PAGE and immunoblotting for antibody protein. Antibody is characterized using the ELISA described above for IL-6 recognition.

Assay for Antibody Activity.

The recombinant yeast-derived humanized antibody is evaluated for functional activity through the IL-6 driven T1165 cell proliferation assay and IL-6 stimulated HepG2 haptoglobin assay described above.

### Example 9

### Acute Phase Response Neutralization by Intravenous Administration of Anti-IL-6 Antibody Ab1

Human IL-6 can provoke an acute phase response in rats, and one of the major acute phase proteins that is stimulated in the rat is alpha-2 macroglobulin (A2M). A study was designed to assess the dose of antibody Ab1 required to ablate the A2M response to a single subcutaneous injection of 100 μg of human IL-6 given one hour after different doses (0.03, 0.1, 0.3, 1, and 3 mg/kg) of antibody Ab1 administered intravenously (n=10 rats/dose level) or polyclonal human IgG1 as the control (n=10 rats). Plasma was recovered and the A2M was quantitated via a commercial sandwich ELISA kit (ICL Inc., Newberg OR; cat. no.-E-25A2M). The endpoint was the difference in the plasma concentration of A2M at the 24 hour time point (post-Ab1).

The ID50 for antibody Ab1 was 0.1 mg/kg with complete suppression of the A2M response at the 0.3 mg/kg. This demonstrates that the IL-6 may be neutralized in vivo by anti-IL-6 antibodies described herein.

### Example 10

### Multi-Dose Pharmacokinetic Evaluation of Antibody Ab1 in Non-Human Primates

Antibody Ab1 was dosed in a single bolus infusion to a single male and single female cynomolgus monkey in phosphate buffered saline. Plasma samples were removed at fixed time intervals and the level of antibody Ab1 was quantitated through of the use of an antigen capture ELISA assay. Biotinylated IL-6 (50 μl of 3 ng/mL) was captured on Streptavidin coated 96 well microtiter plates. The plates were washed and blocked with 0.5% Fish skin gelatin. Appropriately diluted plasma samples were added and incubated for 1 hour at room temperature. The supernatants removed and an anti-hFc-HRP conjugated secondary antibody applied and left at room temperature.

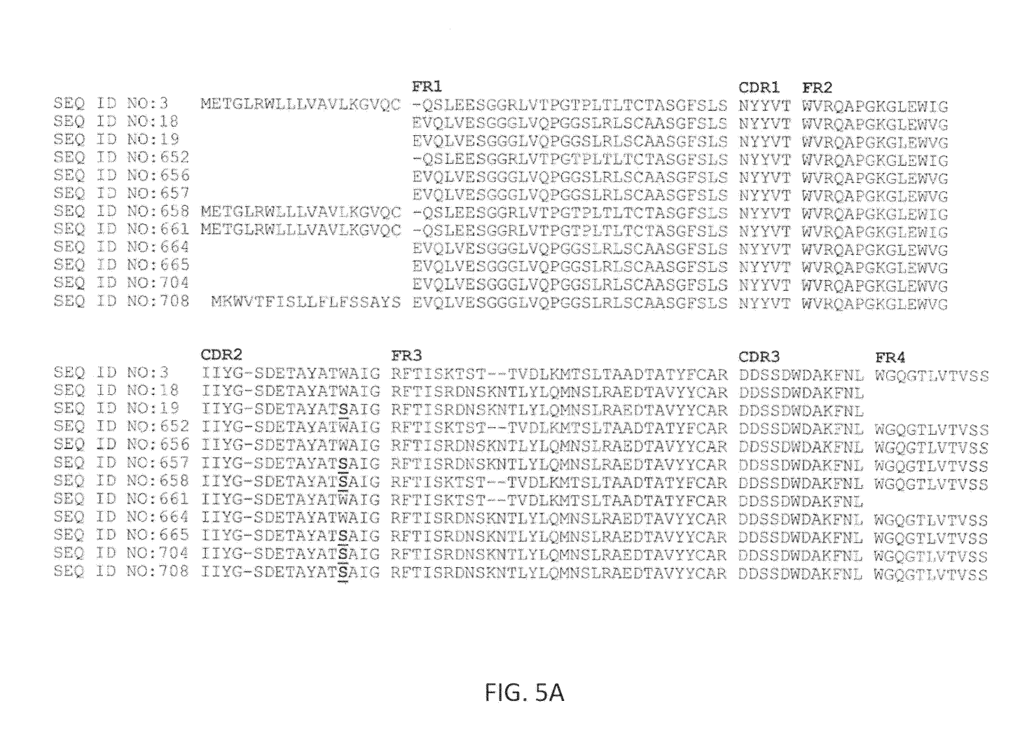

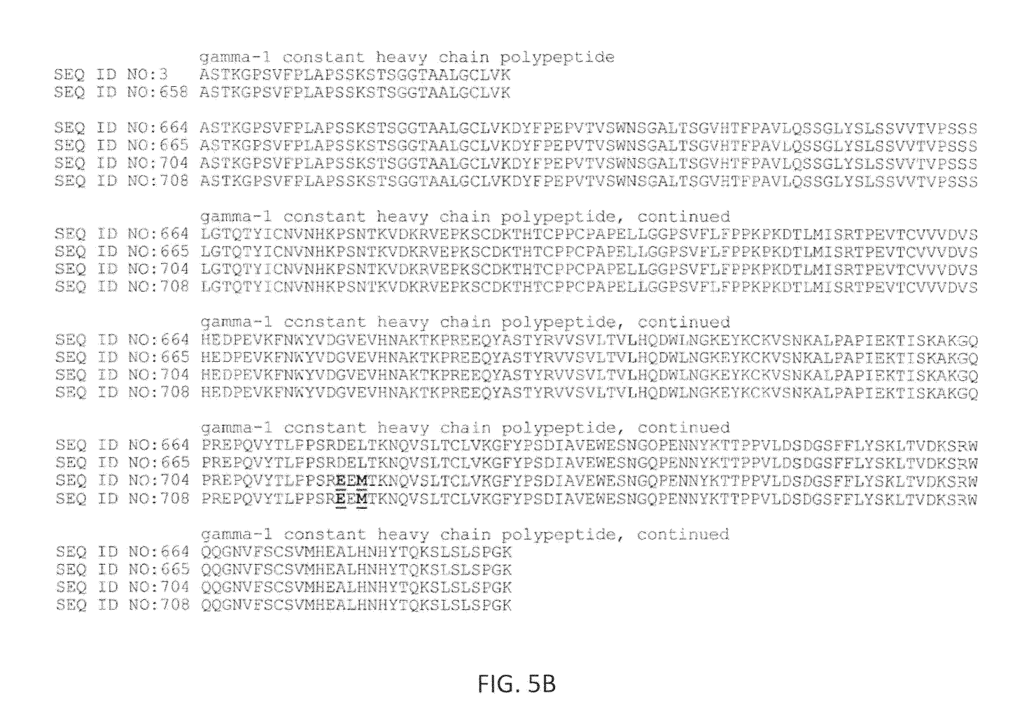

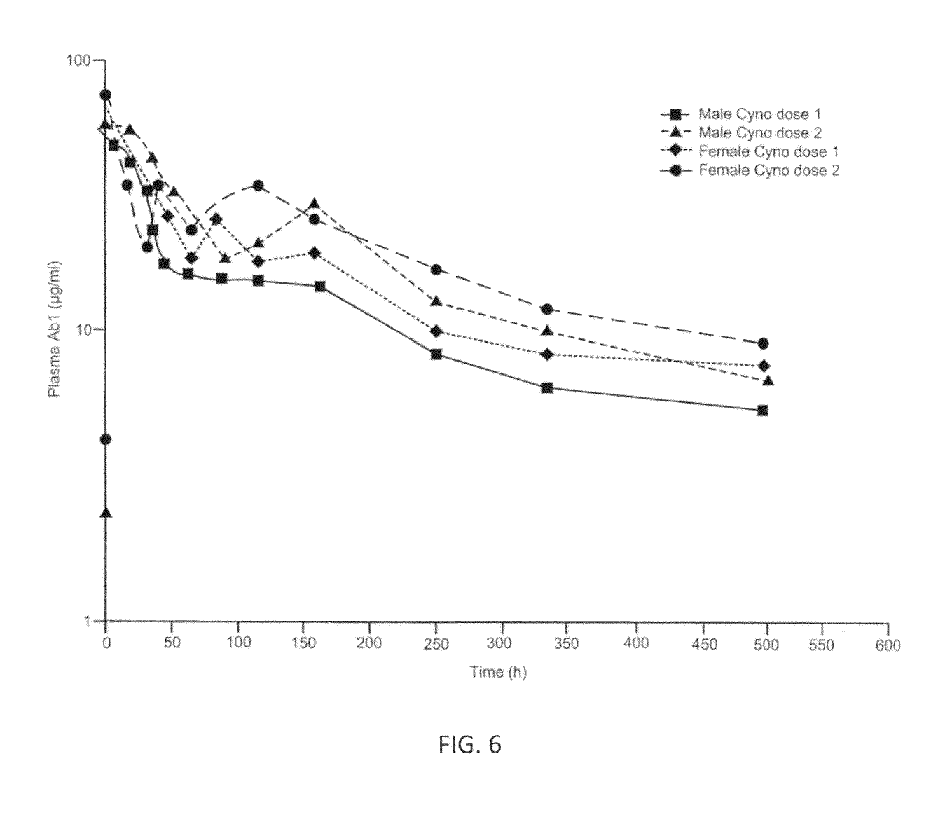

The plates were then aspirated and TMB added to visualize the amount of antibody. The specific levels were then determined through the use of a standard curve. A second dose of antibody Ab1 was administered at day 35 to the same two cynomolgus monkeys and the experiment replicated using an identical sampling plan. The resulting concentrations are then plot vs. time as show in FIG. 6.

This humanized full length aglycosylated antibody expressed and purified Pichia pastoris displays comparable characteristics to mammalian expressed protein. In addition, multiple doses of this product display reproducible half-lives inferring that this production platform does not generate products that display enhanced immunogenicity.

### Example 11

### Octet Mechanistic Characterization of Antibody Proteins

IL-6 signaling is dependent upon interactions between IL-6 and two receptors, IL-6R1 (CD126) and gp130 (IL-6 signal transducer). To determine the antibody mechanism of action, mechanistic studies were performed using bio-layer interferometry with an Octet QK instrument (ForteBio; Menlo Park, Calif.). Studies were performed in two different configurations. In the first orientation, biotinylated IL-6 (R&D systems part number 206-IL-001MG/CF, biotinylated using Pierce EZ-link sulfo-NHS-LC-LC-biotin product number 21338 according to manufacturer's protocols) was initially bound to a streptavidin coated biosensor (ForteBio part number 18-5006). Binding is monitored as an increase in signal.

The IL-6 bound to the sensor was then incubated either with the antibody in question or diluent solution alone. The sensor was then incubated with soluble IL-6R1 (R&D systems product number 227-SR-025/CF) molecule. If the IL-6R1 molecule failed to bind, the antibody was deemed to block IL-6/IL-6R1 interactions. These complexes were incubated with gp130 (R&D systems 228-GP-010/CF) in the presence of IL-6R1 for stability purposes. If gp130 did not bind, it was concluded that the antibody blocked gp130 interactions with IL-6.

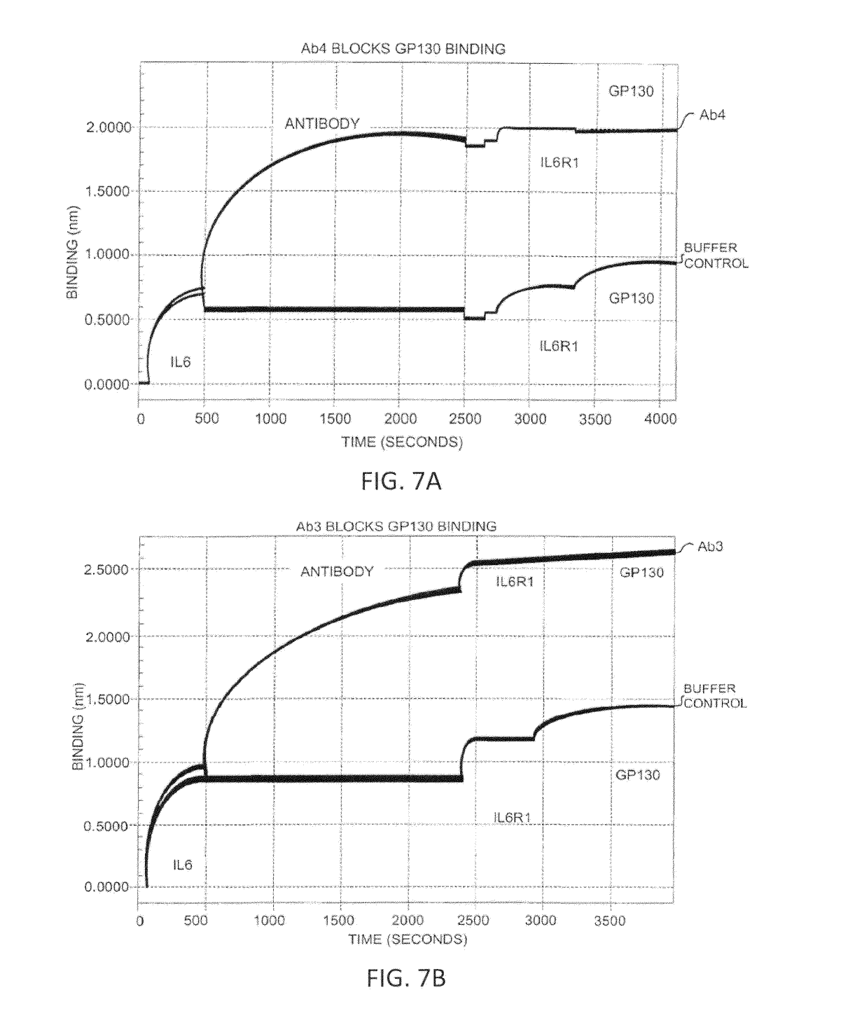

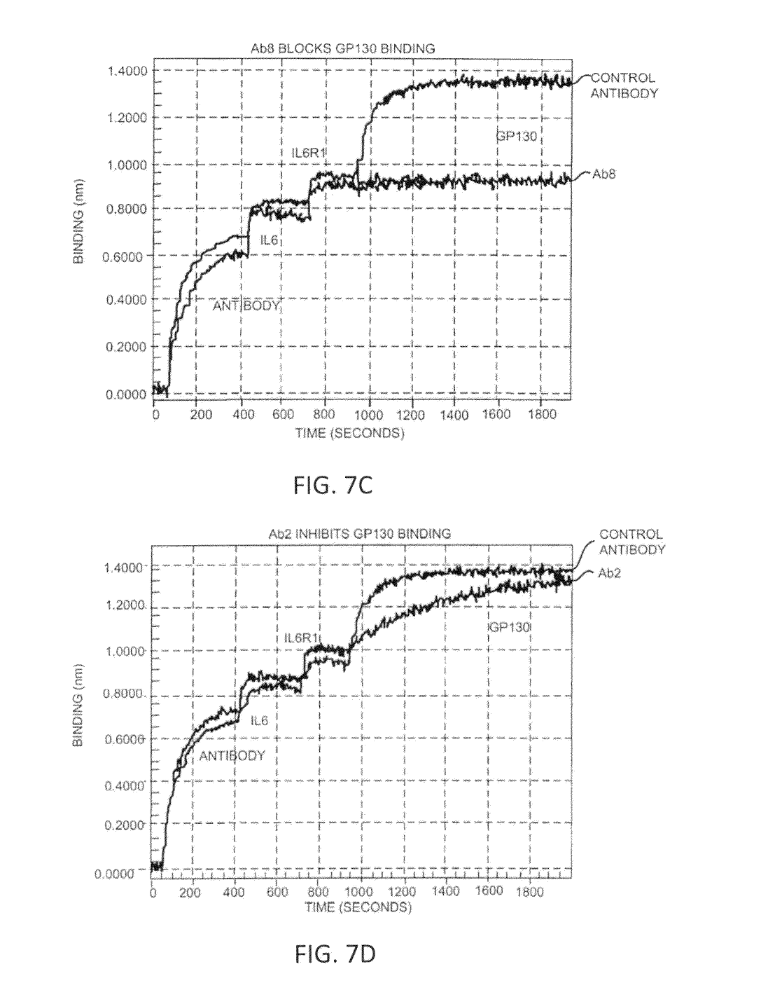

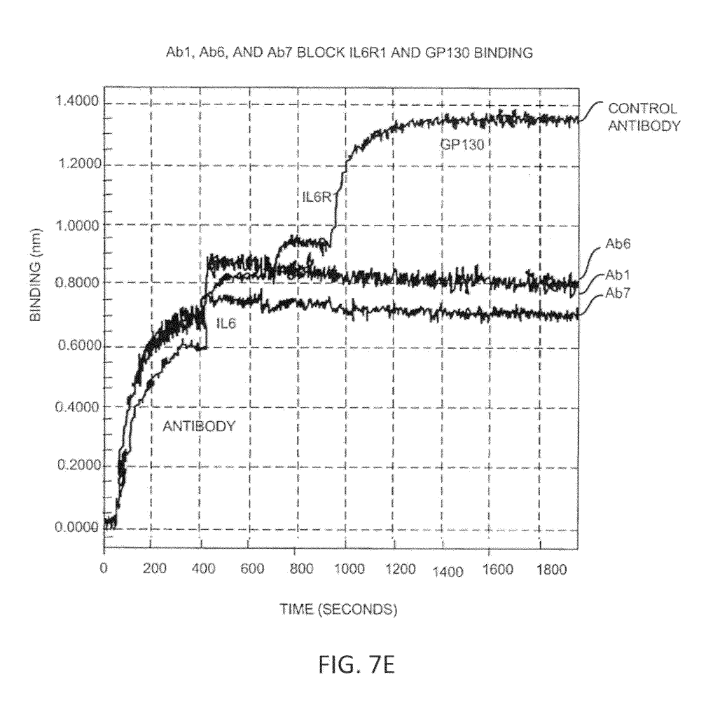

In the second orientation, the antibody was bound to a biosensor coated with an anti-human IgG1 Fc-specific reagent (ForteBio part number 18-5001). The IL-6 was bound to the immobilized antibody and the sensor was incubated with IL-6R1. If the IL-6R1 did not interact with the IL-6, then it was concluded that the IL-6 binding antibody blocked IL-6/IL-6R1 interactions. In those situations where antibody/IL-6/IL-6R1 was observed, the complex was incubated with gp130 in the presence of IL-6R1. If gp130 did not interact, then it was concluded that the antibody blocked IL-6/130 interactions. All studies were performed in a 200 μL final volume, at 30° C. and 1000 rpm. For these studies, all proteins were diluted using ForteBio's sample diluent buffer (part number 18-5028). Results are presented in FIG. 7A-E and TABLE 5.

TABLE 5
Anti-IL6 Antibodies binding to R1 or GP130

| Antibody | Blocks IL6 binding to R1 | Blocks IL6 Binding to GP130 |
|---|---|---|
| Ab1 | Yes | Yes |
| Ab2 | No | Partial |
| Ab3 | No | Yes |
| Ab4 | No | Yes |
| Ab6 | Yes | Yes |
| Ab7 | Yes | Yes |
| Ab8 | No | Yes |

### Example 12

### Peptide Mapping

In order to determine the epitope recognized by Ab1 on human IL-6, the antibody was employed in a western-blot based assay. The form of human IL-6 utilized in this example had a sequence of 183 amino acids in length. A 57-member library of overlapping 15 amino acid peptides encompassing this sequence was commercially synthesized and covalently bound to a PepSpots nitrocellulose membrane (JPT Peptide technologies, Berlin, Germany). The sequences of the overlapping 15 amino acid peptides are in SEQ ID NOs: 590-646. Blots were prepared and probed according to the manufacturer's recommendations.

Briefly, blots were pre-wet in methanol, rinsed in PBS, and blocked for over 2 hours in 10% non-fat milk in PBS/0.05% Tween (Blocking Solution). The Ab1 antibody was used at 1 mg/mL final dilution, and the HRP-conjugated Mouse Anti-Human-Kappa secondary antibody (Southern BioTech #9220-05) was used at a 1:5000 dilution. Antibody dilutions/incubations were performed in blocking solution. Blots were developed using Amersham ECL advance reagents (GE# RPN2135) and chemiluminescent signal documented using a CCD camera (Alphalnnotec). The sequence of the form of human IL-6 utilized to generate peptide library is set forth in SEQ ID NO: 1.

### Example 13

### Ab1 has High Affinity for IL-6

Surface plasmon resonance was used to measure association rate (Kₐ), dissociation rate (K_{d}) and dissociation constant (K_{D}) for Ab1 to IL-6 from rat, mouse, dog, human, and cynomolgus monkey at 25° C. (TABLE 6). The dissociation constant for human IL-6 was 4 pM, indicating very high affinity. As expected, affinity generally decreased with phylogenetic distance from human. The dissociation constants of Ab1 for IL-6 of cynomolgus monkey, rat, and mouse were 31 pM, 1.4 nM, and 0.4 nM, respectively. Ab1 affinity for dog IL-6 below the limit of quantitation of the experiment.

The high affinity of Ab1 for mouse, rat, and cynomolgus monkey IL-6 suggest that Ab1 may be used to inhibit IL-6 of these species. This hypothesis was tested using a cell proliferation assay. In brief, each species' IL-6 was used to stimulate proliferation of T1165 cells, and the concentration at which Ab1 could inhibit 50% of proliferation (IC50) was measured Inhibition was consistent with the measured dissociation constants (TABLE 7). These results demonstrate that Ab1 can inhibit the native IL-6 of these species, and suggest the use of these organisms for in vitro or in vivo modeling of IL-6 inhibition by Ab1.

TABLE 6
Surface Plasmon Resonance: Averaged binding constants determined
at 25° C. for Ab1 to IL-6.

| Species (IL-6) | Kₐ (M⁻¹s⁻¹) | K_{d} (s⁻¹) | Kₚ |
|---|---|---|---|
| Rat | 1.6e⁶ | 2.2e⁻³ | 1.4 nM |
| Mouse | 1.1e⁶ | 4.0e⁻⁴ | 0.4 nM |
| Dog | Below LOQ^{a} | Below LOQ^{a} | Below LOQ^{a} |
| Human | 1.6e⁵ | 5e⁻⁷ | 4 pM |
| Cynomolgus | 9.6e⁴ | 3e⁻⁶ | 31 pM |
| monkey | | | |

^{a}Below Limit of Quantitation

TABLE 7
IC50 values for Ab1 against human, cynomolgus monkey, mouse,
rat and dog IL-6 in the T1165 assay.

| IL-6 Species | IC50 (pM) |
|---|---|
| Human | 13 |
| Cynomolgus monkey | 12 |
| Mouse | 1840 |
| Rat | 2060 |
| Dog | No inhibition of cell proliferation |

### Example 14

### Multi-Close Pharmacokinetic Evaluation of Antibody Ab1 in Healthy Human Volunteers

Antibody Ab1 was dosed in a single bolus infusion in histidine and sorbitol to healthy human volunteers. Dosages of 1 mg, 3 mg, 10 mg, 30 mg or 100 mg were administered to each individual in dosage groups containing five to six individuals. Plasma samples were removed at fixed time intervals for up to twelve weeks. Human plasma was collected via venipuncture into a vacuum collection tube containing EDTA. Plasma was separated and used to assess the circulating levels of Ab1 using a monoclonal antibody specific for Ab1, as follows. A 96 well microtiter plate was coated overnight with the monoclonal antibody specific for Ab1 in 1×PBS overnight at 4° C. The remaining steps were conducted at room temperature. The wells were aspirated and subsequently blocked using 0.5% Fish Skin Gelatin (FSG) (Sigma) in 1×PBS for 60 minutes. Human plasma samples were then added and incubated for 60 minutes, then aspirated, then 50 μL of 1 μg/mL biotinylated IL-6 was then added to each well and incubated for 60 minutes. The wells were aspirated, and 50 μL streptavidin-HRP (Pharmingen), diluted 1:5,000 in 0.5% FSG/PBS, was added and incubated for 45 minutes. Development was conducted using standard methods employing TMB for detection. Levels were then determined via comparison to a standard curve prepared in a comparable format.

Average plasma concentration of Ab1 for each dosage group versus time is shown in TABLE 8. Mean AUC and Cₘₐₓ increased linearly with dosage. For dosages of 30 mg and above, the average Ab1 half-life in each dosage group was between approximately 25 and 30 days.

### Table 8. Summary of Ab1 Pharmacokinetics in Health Human Volunteers

TABLE 8
Summary of Ab1 Pharmacokinetics in Health Human Volunteers

| | T_{1/2} | AUC | Cₘₐₓ | |
|---|---|---|---|---|
| Dose of Ab1 | (days) | (μg · h/mL) | (μg/mL) | Tₘₐₓ |
| 1 mg | 10.3 | 35 | 0.1 | 8 |
| 3 mg | 11.6 | 229 | 0.7 | 4 |
| 10 mg | 22.4 | 1473 | 4.0 | 4 |
| 30 mg | 25.1 | 9076 | 19.7 | 4 |
| 100 mg | 30.3 | 26128 | 48.0 | 12 |
| 300 mg | 26.2 | 92891 | 188.0 | 12 |
| 640 mg | 30.2 | 175684 | 306.0 | 12 |

### Example 15

### Pharmacokinetics of Ab1 in Patients with Advanced Cancer

Antibody Ab1 was dosed in a single bolus infusion in phosphate buffered saline to five individuals with advanced cancer. Each individual received a dosage of 80 mg (n=2) or 160 mg (n=3) of Ab1. Plasma samples were drawn weekly, and the level of antibody Ab1 was quantitated as in Example 16.

Average plasma concentration of Ab1 in these individuals as a function of time is shown in FIG. 8. The average Ab1 half-life was approximately 31 days.

### Example 16

### Half-Life of Ab1

Overall, the average half-life of Ab1 was approximately 31 days in humans (for dosages of 10 mg and above), and approximately 15-21 days in cynomolgus monkey. The Ab1 half-life in humans and cynomolgus monkeys is unprecedented when compared with the half-lives of other anti-IL-6 antibodies (TABLE 9). As described above, Ab1 was derived from humanization of a rabbit antibody, and is produced from Pichia pastoris in an aglycosylated form. These characteristics results in an antibody with very low immunogenicity in humans. Moreover, the lack of glycosylation prevents Ab1 from interacting with the Fc receptor or complement. Without intent to be limited by theory, it is believed that the unexpectedly long half-life of Ab1 is at least partially attributable to the humanization and/or the lack of glycosylation. The particular sequence and/or structure of the antigen binding surfaces may also contribute to Ab1's half-life.

TABLE 9
Elimination Half-life of Ab1

| | Cynomolgus Monkey | Human |
|---|---|---|
| Dose of AB1 | (days) | (days) |
| Ab1 | 15-21 | ~31 |
| Acemra (Tocilizumab) | 7 | 6 |
| Remicade | 5 | 8-9.5 |
| Synagis | 8.6 | 20 |
| Erbitux | 3-7 | 5 |
| Zenapax | 7 | 20 |
| Avastin | 10 | 20 |
| Pertuzumab | 10 | 18-22 |

### Example 17

### Ab1 Suppresses Serum CRP in Healthy Volunteers and in Patients with Advanced Cancer

Introduction—

Serum CRP concentrations have been identified as a strong prognostic indicator in patients with certain forms of cancer. For example, Hashimoto et al. performed univariate and multivariate analysis of preoperative serum CRP concentrations in patients with hepatocellular carcinoma in order to identify factors affecting survival and disease recurrence. Hashimoto, et al. (2005) Cancer 103(9): 1856-1864. Patients were classified into two groups, those with serum CRP levels >1.0 mg/dL (“the CRP positive group”) and those with serum CRP levels <1.0 mg/dL (“the CRP negative group”). The authors identified “a significant correlation between preoperative serum CRP level and tumor size.” Id. Furthermore, the authors found that “[t]he overall survival and recurrence-free survival rates in the CRP-positive group were significantly lower compared with the rates in the CRP-negative group.” Id. The authors concluded that the preoperative CRP level of patients is an independent and significant predictive indicator or poor prognosis and early recurrence in patients with hepatocellular carcinoma.

Similar correlations have been identified by other investigators. For example, Karakiewicz et al. determined that serum CRP was an independent and informative predictor of renal cell carcinoma-specific mortality. Karakiewicz, et al. (2007) Cancer. 110(6):1241-1247. Accordingly, there remains a need in the art for methods and/or treatments that reduce serum C-Reactive Protein (CRP) concentrations in cancer patients, and particularly those with advanced cancers.

Methods—

Healthy volunteers received a single 1-hour intravenous (IV) infusion of either 100 mg (5 patients), 30 mg (5 patients), 10 mg (6 patients), 3 mg (6 patients) or 1 mg (6 patients) of the Ab1 monoclonal antibody, while another 14 healthy volunteers received intravenous placebo. Comparatively, 2 patients with advanced forms of colorectal cancer received a single 1-hour intravenous (IV) infusion of 80 mg of the Ab1 monoclonal antibody. No further dosages of the Ab1 monoclonal antibody were administered to the test population.

Patients were evaluated prior to administration of the dosage, and thereafter on a weekly basis for at least 5 weeks post dose. At the time of each evaluation, patients were screened for serum CRP concentration.

Results—Healthy Volunteers

As noted above, serum CRP levels are a marker of inflammation; accordingly, baseline CRP levels are typically low in healthy individuals. The low baseline CRP levels can make a further reduction in CRP levels difficult to detect. Nonetheless, a substantial reduction in serum CRP concentrations was detectable in healthy volunteers receiving all concentrations of the Ab1 monoclonal antibody, compared to controls (FIG. 9A). The reduction in serum CRP levels was rapid, occurring within one week of antibody administration, and prolonged, continuing at least through the final measurement was taken (8 or 12 weeks from antibody administration).

Results—Cancer Patients

Five advanced cancer patients (colorectal cancer, cholangiocarcinoma, or NSCLC) having elevated serum CRP levels were dosed with 80 mg or 160 mg of Ab1. Serum CRP levels were greatly reduced in these patients (FIG. 9B). The reduction in serum CRP levels was rapid, with 90% of the decrease occurring within one week of Ab1 administration, and prolonged, continuing at least until the final measurement was taken (up to twelve weeks). In two representative individuals, the CRP levels were lowered to below the normal reference range (less than 5-6 mg/1) within one week. Thus, administration of Ab1 to patients can cause a rapid and sustained suppression of serum CRP levels.

### Example 18

### Ab1 Suppresses Serum CRP in Patients with Advanced Cancer

Introduction—

Serum CRP concentrations have been identified as a strong prognostic indicator in patients with certain forms of cancer. For example, Hashimoto et al. performed univariate and multivariate analysis of preoperative serum CRP concentrations in patients with hepatocellular carcinoma in order to identify factors affecting survival and disease recurrence. Hashimoto, et al. (2005) Cancer 103(9): 1856-1864. Patients were classified into two groups, those with serum CRP levels >1.0 mg/dL (“the CRP positive group”) and those with serum CRP levels <1.0 mg/dL (“the CRP negative group”). The authors identified “a significant correlation between preoperative serum CRP level and tumor size.” Id. Furthermore, the authors found that “[t]he overall survival and recurrence-free survival rates in the CRP-positive group were significantly lower compared with the rates in the CRP-negative group.” Id. The authors concluded that the preoperative CRP level of patients is an independent and significant predictive indicator of poor prognosis and early recurrence in patients with hepatocellular carcinoma.

Similar correlations have been identified by other investigators. For example, Karakiewicz et al. determined that serum CRP was an independent and informative predictor of renal cell carcinoma-specific mortality. Karakiewicz, et al. (2007) Cancer 110(6):1241-1247. Accordingly, there remains a need in the art for methods and/or treatments that reduce serum C-Reactive Protein (CRP) concentrations in cancer patients, and particularly those with advanced cancers.

Methods—

One-hundred twenty-four patients with non-small cell lung cancer (NSCLC) were divided into 4 treatment groups. Patients in one group received one 1-hour intravenous (IV) infusion of either placebo (n=31), 80 mg (n=29), 160 mg (n=32), or 320 mg (n=32) of the Ab1 monoclonal antibody every 8 weeks over a 24 week duration for a total of 3 doses. CRP concentration was quantitated by a C-reactive protein particle-enhanced immunoturbidimetric assay using latex-attached anti-CRP antibodies (i.e. Roche CRP Tinaquant®). Briefly, about 1.0 mL of patient sample serum was collected and stored in a plastic collection tube. Sample was placed into appropriate buffer, and anti-CRP antibody coupled to latex microparticles was added to the sample to start the reaction. These anti-CRP antibodies with conjugated latex microparticles react with antigen in the sample to form an antigen/antibody complex. Following agglutination, this was measured turbidimetrically using a Roche/Hitachi Modular P analyzer.

Patients were evaluated prior to administration of the dosage, and thereafter at weeks 2, 4, 8, and 12. At the time of each evaluation, patients were screened for serum CRP concentration.

Results—

The averaged data for each dosage concentrations (placebo, 80 mg, 160 mg, and 320 mg) of the Ab1 monoclonal antibody are plotted in FIG. 10. All dosage levels of Ab1 antibody demonstrated an immediate drop in CRP concentrations relative to placebo over the period of 12 weeks. CRP levels displayed breakthrough at 8 weeks post-dosing. The CRP levels fell below 5 mg/L by week 12. Median values of CRP demonstrated rapid and sustained decreases for all dosage concentrations relative to placebo (FIG. 11). Thus, administration of Ab1 to advanced cancer patients can cause a rapid and sustained suppression of serum CRP levels.

### Example 19

### Ab1 Suppresses Serum CRP in Patients with Advanced Cancers

Introduction—

Serum CRP concentrations have been identified as a strong prognostic indicator in patients with certain forms of cancer. For example, Hashimoto et al. performed univariate and multivariate analysis of preoperative serum CRP concentrations in patients with hepatocellular carcinoma in order to identify factors affecting survival and disease recurrence. Hashimoto, et al. (2005) Cancer 103(9): 1856-1864. Patients were classified into two groups, those with serum CRP levels >1.0 mg/dL (“the CRP positive group”) and those with serum CRP levels <1.0 mg/dL (“the CRP negative group”). The authors identified “a significant correlation between preoperative serum CRP level and tumor size.” Id. Furthermore, the authors found that “[t]he overall survival and recurrence-free survival rates in the CRP-positive group were significantly lower compared with the rates in the CRP-negative group.” Id. The authors concluded that the preoperative CRP level of patients is an independent and significant predictive indicator of poor prognosis and early recurrence in patients with hepatocellular carcinoma.

Similar correlations have been identified by other investigators. For example, Karakiewicz et al. determined that serum CRP was an independent and informative predictor of renal cell carcinoma-specific mortality. Karakiewicz, et al. (2007) Cancer 110(6): 1241-1247. Accordingly, there remains a need in the art for methods and/or treatments that reduce serum C-Reactive Protein (CRP) concentrations in cancer patients, and particularly those with advanced cancers.

Methods—

Eight patients with various forms of advanced cancer (colorectal (3), NSCLC (1), cholangio (1), and mesothelioma (2)) received a single 1-hour intravenous infusion of either 80 mg (2 patients), 160 mg (3 patients) or 320 mg (3 patients) of the Ab1 monoclonal antibody. No further dosages of the Ab1 monoclonal antibody were administered to the test population.

Patients were evaluated prior to administration of the dosage and thereafter on a weekly basis for at least 8 weeks post dose. At the time of each evaluation, patients were screened for serum CRP concentration. CRP concentration was quantitated by a C-reactive protein particle-enhanced immunoturbidimetric assay using latex-attached anti-CRP antibodies (i.e. Roche CRP Tinaquant®). Briefly, about 1.0 mL of patient sample serum was collected and stored in a plastic collection tube. Sample was placed into appropriate buffer, and anti-CRP antibody coupled to latex microparticles was added to the sample to start the reaction. These anti-CRP antibodies with conjugated latex microparticles react with antigen in the sample to form an antigen/antibody complex. Following agglutination, this was measured turbidimetrically using a Roche/Hitachi Modular P analyzer.

Results—

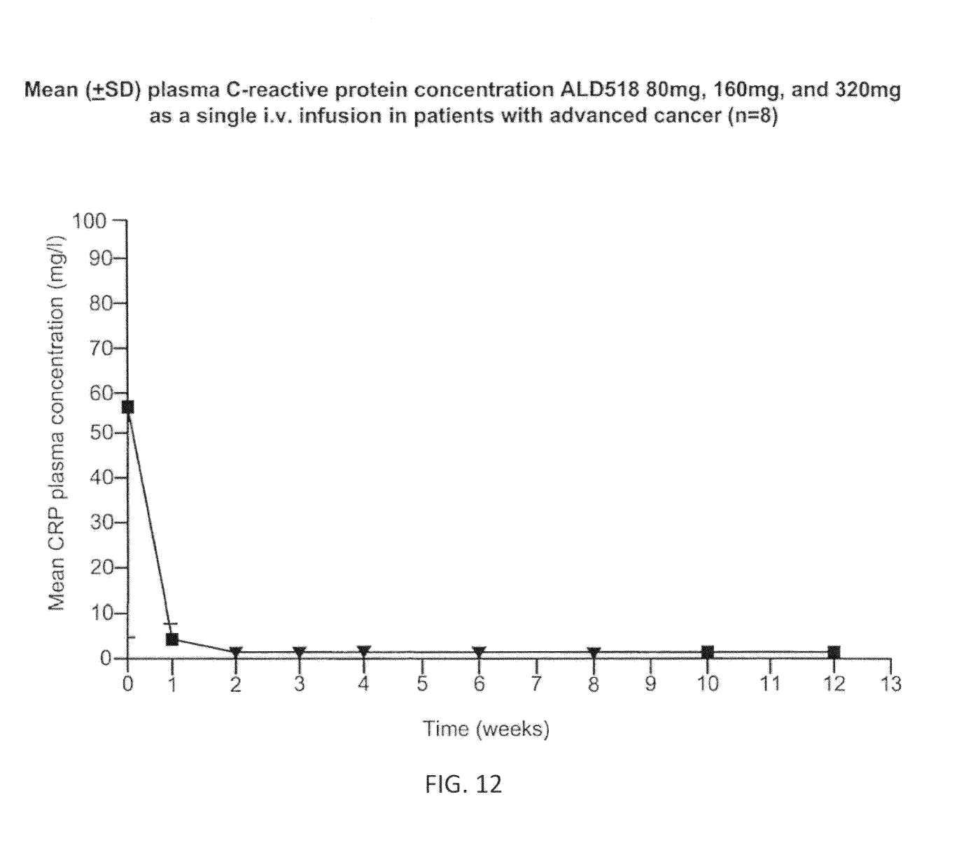

Serum CRP levels were greatly reduced in all patients studied (FIG. 12). The reduction in serum CRP levels was rapid, with approximately 90% of the decrease occurring within one week of Ab1 administration, and prolonged diminished levels continued at least until the final measurement was taken (up to twelve weeks). In all cases except one patient with colorectal cancer, CRP levels fell to at or below the normal reference range (less than 5-6 mg/L) within one week. The colorectal cancer patient achieved similar normal levels by week 4 of the study. Thus, administration of Ab1 to advanced cancer patients can cause a rapid and sustained suppression of serum CRP levels.

### Example 20

### Safety, Pharmacokinetics (PK), and Pharmacodynamics (PD) of Ab1 in Human Subjects

Background—

A humanized antibody derived from Ab1 (humanized Ab1 or ALD518) containing the variable heavy and light sequences in SEQ ID NO: 19 and 20 was administered to rheumatoid arthritis patients. This antibody is a humanized, asialated, IgG1 monoclonal antibody against IL-6 which has been shown to have a half-life (t1/2) of approximately 30 days in humans. In studies in patients with RA, intravenous (IV) with this antibody (humanized Ab1) has demonstrated: efficacy over 16 weeks with rapid American College of Rheumatology (ACR) responses; Complete and durable suppression of C-reactive protein (CRP); Good tolerability, and a safety profile consistent with the biology of IL-6 blockade. This humanized antibody binds to IL-6 with high affinity, preventing interaction and signalling mediated via IL-6R. Rapid and significant treatment responses have been demonstrated with intravenous (IV) administration of humanized Ab1 in patients with RA. In this example we study the safety, pharmacokinetics and pharmacodynamics of subcutaneous (SC) administration of humanized Ab1 in healthy subjects.

The objective of this study was to assess the safety, pharmacokinetics (PK) and pharmacodynamics (PD) of a single SC injection of this humanized antibody in healthy male subjects.

Methods—

In this Phase I, double-blind, placebo-controlled study, 27 subjects were randomized 2:1 to receive a single dose of humanized Ab1 or placebo in the following groups: humanized Ab1 50 mg SC, humanized Ab1100 mg SC or humanized Ab1100 mg IV (n=6 active and n=3 placebo per group). The primary objective was to assess safety of SC humanized Ab1 versus placebo over 12 weeks. Plasma concentrations of humanized Ab1 and serum concentrations of C-reactive protein (CRP) were assessed as secondary objectives. Assessments were performed daily in Week 1 and then on Day 10, Weeks 2, 4, 6 and 8, and then monthly to Week 12. The study was unblinded at Week 12, and humanized Ab1 subjects were monitored to Week 24.

Study Design and Population—

The study included 27 healthy male subjects (aged 18-65 years). Subjects were dosed in three treatment groups of nine subjects each, randomized 2:1 to receive a single dose of humanized Ab1 or placebo on Day 1. Humanized Ab1 treatments per group were: humanized Ab1 IV 100 mg infusion over 60 minutes; humanized Ab1 SC 50 mg injection (1 mL); or humanized Ab1100 mg injection (1 mL). The study was unblinded at Week 12, after which placebo subjects discontinued the trial and ALD518 subjects were monitored to Week 24.

Safety and Immunogenicity Assessments—

The primary objective of the study was to assess the safety of SC humanized Ab1 compared with placebo over 12 weeks. Safety was monitored over 12 weeks for all subjects. The study was unblinded at Week 12, and Humanized AB1 subjects were monitored to Week 24. Laboratory safety tests were performed pre-dose at screening and Day −1, and post dose on Days 2 and 7, Weeks 2, 4, 6, 8 and 12 for all subjects, and Weeks 16, 20 and 24 post-dose for those randomized to Humanized Ab1. Anti-Humanized AB1 antibodies were measured by enzyme-linked immunosorbent assay (ELISA). Blood samples were collected at Day 1 (pre-dose) and Week 12 post-dose for all subjects, and Week 24 post-dose for those randomized to Humanized Ab1.

Pharmacokinetic and Pharmacodynamic Assessments—

Plasma Humanized AB1 and serum CRP concentrations were assessed by ELISA. For all subjects, samples were collected at screening, pre-dose on Day 1, and post-dose on Days 2 and 7 and Weeks 2, 4, 6, 8 and 12. For subjects randomized to Humanized AB1, further samples were collected at Weeks 16, 20 and 24 post-dose.

Statistical Analysis—

All subjects who received a dose of Humanized AB1 or placebo were included in the safety analysis. All subjects who received a dose of Humanized AB1 or placebo were included in PD and immunogenicity analyses. All subjects who received a dose of Humanized AB1 were included in PK analyses (n=18). All PK samples for placebo subjects were confirmed as below quantification. Descriptive statistics were generated for baseline demographics, safety data, plasma Humanized AB1 parameters and serum CRP concentrations. Wilcoxon Rank Sum test was used to compare CRP concentrations for Humanized AB1 treatments versus placebo.

Results—Summary

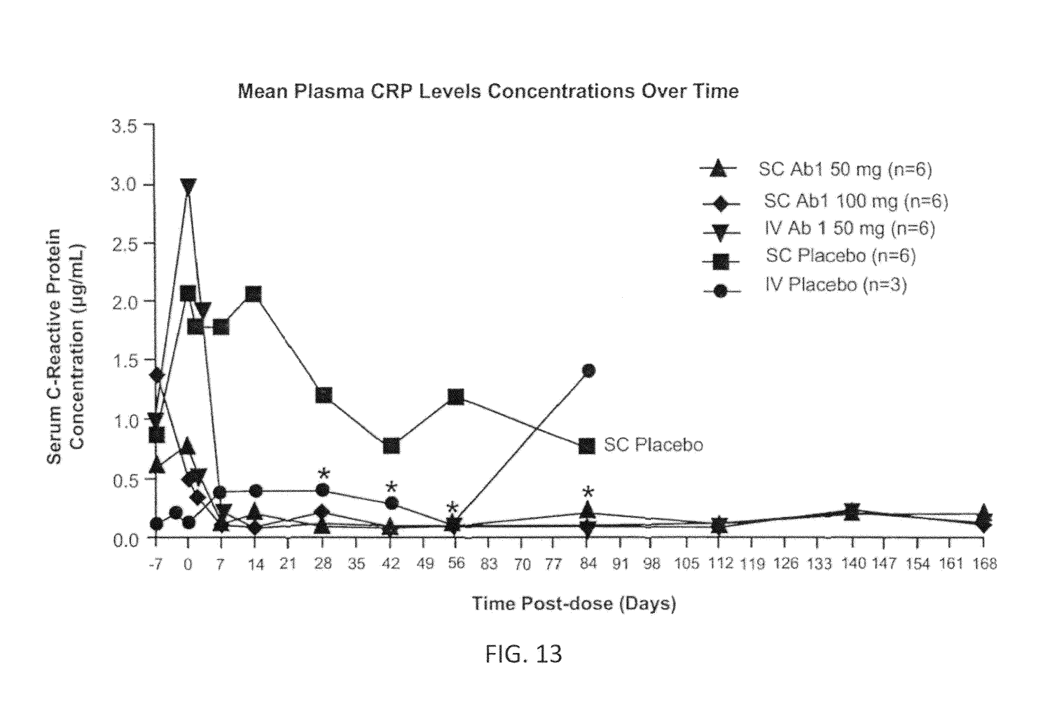

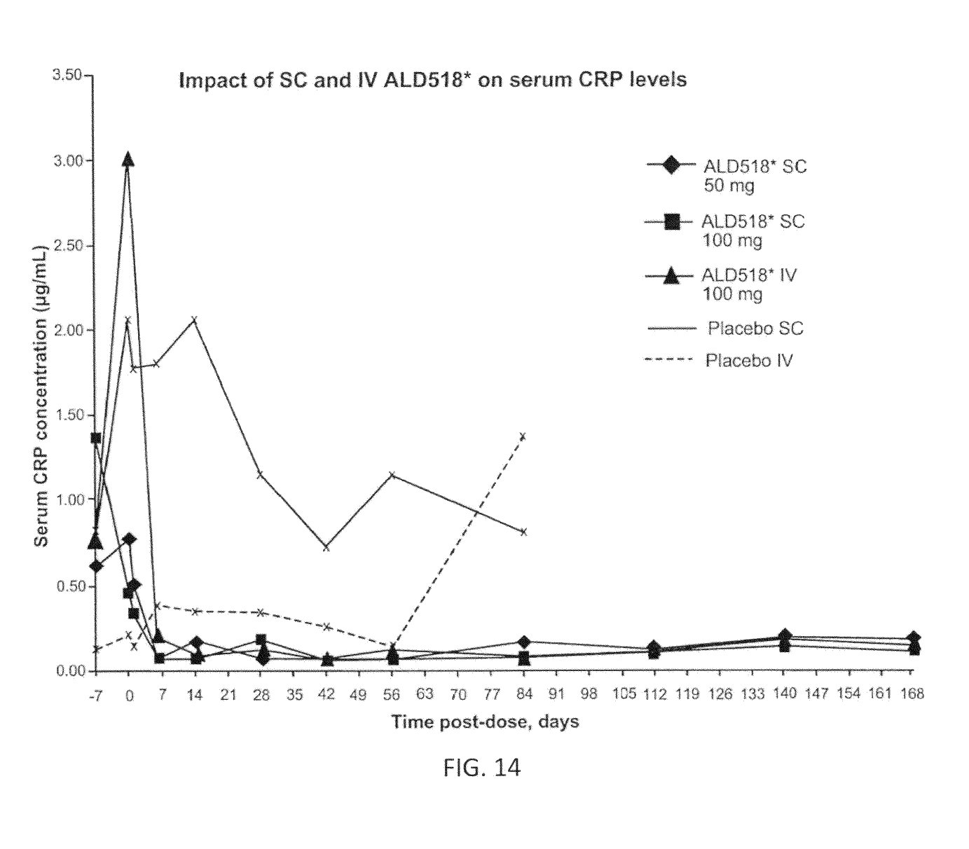

Over 24 weeks, there were no deaths or serious AEs, and no withdrawals due to AEs. Nearly all subjects (89%) experienced AEs, which were mild or moderate except one event of severe gastroenteritis in the Humanized ab1 SC 50 mg group. Injection site reactions occurred in 5/12 Humanized Ab1 SC subjects, 1/6 placebo SC subjects and 1/3 placebo IV subjects (none were reported in Humanized Ab1 IV subjects). These were mild except one case of moderate erythema and pruritis in the Humanized Ab1 100 mg SC group. Increases in direct bilirubin and neutrophil counts below the limit of normal were more common in subjects receiving Humanized Ab1 than placebo; all were CTC Grade 1 or 2. The half-life of Humanized Ab1 was similar across all groups (mean range: 30.7-33.6 days). The median Tmax of Humanized Ab1 was longer after SC (˜1 week) than after IV administration (˜end of infusion). The PK of SC Humanized Ab1 was dose-proportional in terms of AUC and Cmax at doses of 50 mg and 100 mg. Based on AUCO-∞ (day*μg/mL) of 237, 452 and 764 for the Humanized Ab1 50 mg SC, 100 mg SC and 100 mg IV groups, respectively, the bioavailability of Humanized Ab1 was ˜60% for the SC versus IV groups. Subjects receiving Humanized Ab1 experienced rapid and sustained reductions in serum CRP (FIG. 13), similar results were seen when the antibody was administered either intravenous or subcutaneously (FIG. 14).

Subject Disposition and Baseline Demographics—

A total of 27 subjects were enrolled and completed the study (n=18 Humanized Ab1 and n=9 placebo). No subjects were withdrawn for any reason. All subjects were male; 23/27 subjects were Caucasian and 4/27 were Asian. Mean age was 29 (range 20-59) and was similar across the groups. Mean height and weight were also generally comparable across groups, although the IV placebo group were slightly lighter.

Safety and immunogenicity to Week 12 for Humanized AB1 and placebo—A summary of safety is presented in TABLE 10. For the SC Humanized AB1 groups, a total of 11/12 (91%) patients experienced an adverse event (AE) compared with: 6/6 (100%) for the IV Humanized AB1 group; 4/6 (66.6%) for the SC placebo group; and 3/3 (100%) for the IV placebo group.

TABLE 10
Adverse Events

| | Up to Week 12 | | | | | Week 12-Week 24* | | |
|---|---|---|---|---|---|---|---|---|
| MedRA | SC 50 mg | SC 100 mg | IV 100 mg | Placebo SC | Placebo IV | SC 100 mg | SC 100 mg | IV 100 mg |
| Preferred Term | n = 6 | n = 6 | n = 6 | n = 6 | n = 6 | n = 6 | n = 6 | n = 6 |
| Subjects with | 6 | 5 | 6 | 4 | 3 | 3 | 5 | 5 |
| an AE | | | | | | | | |

AE severity

| | | | | | | | | |
|---|---|---|---|---|---|---|---|---|
| Mild | 2 | 2 | 5 | 1 | 2 | 3 | 5 | 7 |
| Moderate | 3 | 3 | 1 | 3 | 1 | 1 | 1 | 0 |
| Severe | 1 | 0 | 0 | 0 | 0 | 0 | 0 | 0 |
| Discontinuations | 0 | 0 | 0 | 0 | 0 | 0 | 0 | 0 |
| Due to AEs | | | | | | | | |
| Deaths | 0 | 0 | 0 | 0 | 0 | 0 | 0 | 0 |

AEs reported in ≧2 subjects in any group

| | | | | | | | | |
|---|---|---|---|---|---|---|---|---|
| Injection site | 1 | 2 | 0 | 0 | 0 | 0 | 0 | 0 |
| erythema | | | | | | | | |
| Injection site | 1 | 2 | 0 | 0 | 1 | 0 | 0 | 0 |
| pruritis | | | | | | | | |
| Gastroenteritis | 1 | 0 | 2 | 0 | 0 | 0 | 0 | 0 |
| URTI | 4 | 4 | 4 | 2 | 2 | 0 | 1 | 2 |
| Skin laceration | 2 | 1 | 2 | 0 | 0 | 0 | 0 | 0 |
| Myalgia | 0 | 0 | 0 | 2 | 0 | 0 | 0 | 0 |
| Headache | 5 | 2 | 1 | 1 | 0 | 0 | 1 | 1 |
| Nasal congestion | 0 | 0 | 2 | 0 | 0 | 0 | 0 | 0 |

*Patients randomized to placebo (IV or SC) discontinued at Week 12 and are not included in Week 24 analyses;
AE = adverse event; SC = subcutaneous; IV = intravenous; URTI = upper respiratory tract infection.

Across groups: No deaths or serious AEs were reported and there were no withdrawals due to AEs. Most AEs were mild or moderate in intensity. One case of gastroenteritis in a SC Humanized AB1 50 mg subject was considered severe, but not serious, and not related to study medication. No anti-Humanized AB1 antibodies were detected in any subject during this period.

Injection Site Reactions—

Injection site reactions were reported in 26% (7/27) of subjects, and all occurred prior to Week 12 (TABLE 11). Injection site reactions occurred in 5/12 SC Humanized AB1 subjects and 1/6 SC placebo subjects. In the IV groups, 0/6 Humanized AB1 subjects and 1/3 placebo subjects experienced injection site reactions. All injection site reactions were mild except in one SC Humanized AB 1 100 mg subject with moderate injection site erythema and pruritis. No injection site reactions occurred after Week 12 in any of the Humanized AB1 groups. Infusion site reactions were reported in 0/6 subjects receiving IV Humanized AB1 and 1/3 IV placebo subjects (infusion site pruritis)

TABLE 11
Ab1 Injection Site Reactions to Week 12*

| | | | | Placebo | Placebo |
|---|---|---|---|---|---|
| | 50 mg | 100 mg | 100 mg | SC | IV |
| | n = 6 | n = 6 | n = 6 | n = 6 | n = 3 |
| Total subjects with | 2 | 3 | 0 | 1 | 1 |
| injection site reaction | | | | | |
| Injection site erythema | 1 | 2 | 0 | 0 | 0 |
| Injection site pain | 1 | 1 | 0 | 1 | 0 |
| Injection site pruritis | 1 | 2 | 0 | 0 | 1 |
| Injection site rash | 1 | 0 | 0 | 0 | 0 |

*All injection site reactions were reported in the first 12 weeks of the study.
SC = subcutaneous;
IV = intravenous

Clinical Laboratory Evaluations—

TABLE 12 shows incidences of increased alanine aminotransferase (ALT) and aspartate aminotransferase (AST) and bilirubin levels across the Humanized AB1 and placebo groups. All ALT and AST levels were Grade 1 by the Common Terminology Criteria for Adverse Events (CTCAE), and no levels were ≧3 times the upper limit of normal (ULN). All increases in total and direct bilirubin were CTCAE Grade 1 or 2 and no subject met criteria for drug-induced liver damage. Only one subject (SC Humanized AB1 100 mg group) had total bilirubin out of range (26 μmol/L, range 0-24 μmol/L), at Week 24.

TABLE 12
Clinical Laboratory Evaluations Over 24 Weeks (Ab1)

| | SC 50 mg | SC 100 mg | IV 100 mg | Placebo* |
|---|---|---|---|---|
| | n = 6 | n = 6 | n = 6 | n = 9 |
| Elevated ALT | 0 | 1 | 3 | 2 |
| Elevated AST | 0 | 1 | 1 | 1 |
| Elevated total bilirubin | 0 | 1 | 1 | 0 |
| Elevated direct | 2 | 4 | 5 | 2 |
| bililrubin | | | | |
| Low neutrophil count^{†} | 4 | 1 | 2 | 3 |
| Low platelet count^{†} | 2 | 0 | 0 | 1 |

*SC and IV groups combined up to Week 12 only, after which placebo-treated patients discontinued;
^{†}Below the lower limit of normal;
SC = subcutaneous;
IV = intravenous;
ALT = alanine aminotransferase;
AST = aspartate aminotransferase

Sporadic decreases in neutrophil and platelet counts were also observed in the Humanized AB1 and placebo groups. Neutrophil counts below the lower limit of normal were more common in subjects receiving Humanized AB 1 than placebo but all decreases were CTCAE Grade 1 or 2. Only one subject (SC Humanized AB1 50 mg group) had consistent mild neutropenia to Week 24 (1.6×10⁹/L at Week 24). Reductions in platelet counts were all CTCAE Grade 1 (lowest level 134×10⁹/L) and no subject had a low platelet count past Week 8.

Pharmacokinetics—

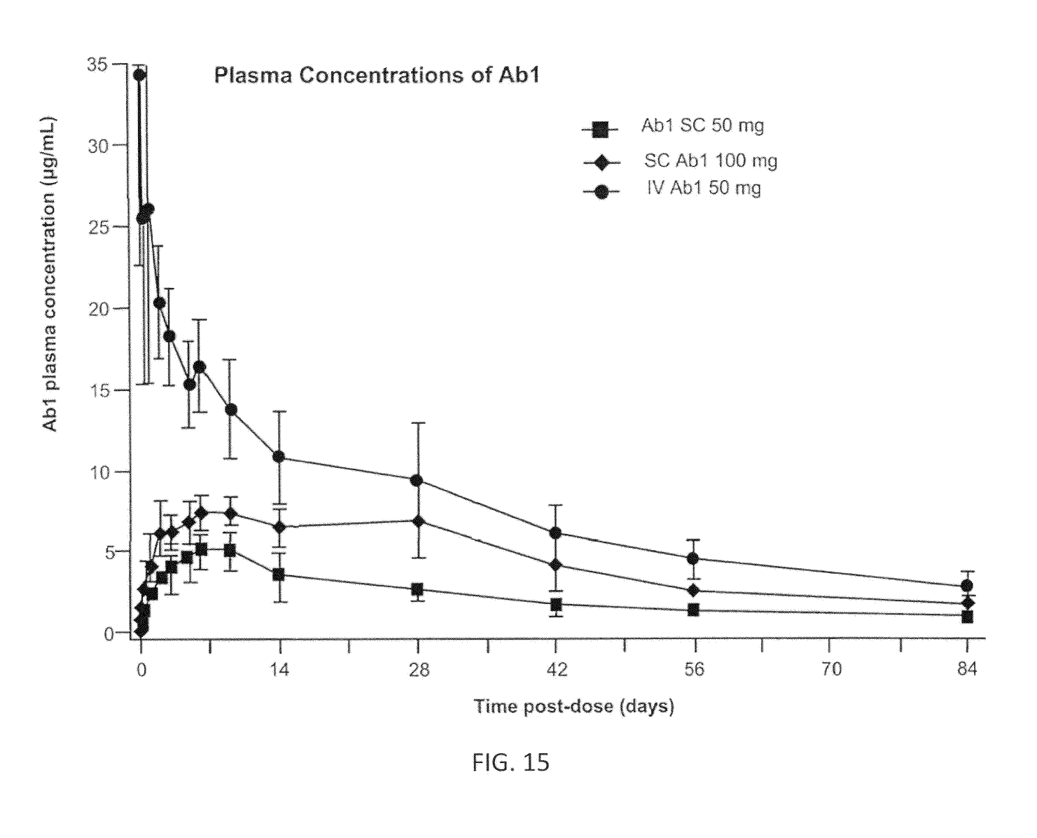

Bioavailability of Humanized AB1 was 60% for SC Humanized AB1 50 and 100 mg versus IV Humanized AB1 100 mg groups based on the mean AUC_{0-∞}. (TABLE 13). The half-life of Humanized AB1 was similar across all groups (mean range: 30.7-33.6 days) (Table G). Peak plasma concentration (Cₘₐₓ) of SC Humanized AB1 was reduced as compared to IV (FIG. 15). Median time to maximum plasma concentration (Tₘₐₓ) of Humanized Ab1 was longer after SC Humanized AB1 (at approximately one week) than after IV Humanized Ab1 administration (at approximately the end of infusion).

TABLE 13
Ab1 Plasma Pharmacokinetic Parameters to Week 24

| | SC 50 mg | | SC 100 mg | | IV 100 mg | |
|---|---|---|---|---|---|---|
| | n = 6 | | n = 6 | | n = 6 | |
| Cₘₐₓ (μg/mL) (CV)* | 5.57 | (24%) | 9.19 | (34%) | 33.6 | (30%) |
| Tₘₐₓ (days) (min, max)^{†} | 6 | (6, 14) | 5.5 | (2, 28) | 0.17 | (0, 17, 0.34) |
| AUC₅₋₂₄ (day · μg/mL) (CV)* | 218 | (34%) | 435 | (19%) | 732 | (22%) |
| AUC_{8-γ} (day · μg/mL) (CV)* | 224 | (39%) | 444 | (20%) | 746 | (22%) |
| t_{1/2} (days ± SD)^{‡} | 33.6 ± 21.7 | | 31.1 ± 9.0 | | 30.7 ± 5.9 | |
| CL (mL/day) (CV)* | 223 | (32%) | 225 | (21%) | 134 | (27%) |

*Data are geometric mean (coefficient of variation %, CV %).
^{†}Data are median (minimum, maximum).
^{‡}Data are mean (±SD).
CV = coefficient of variation;
Cₘₐₓ = maximum plasma concentration;
AUC = area under curve;
SD = standard deviation;
CL = apparent total body clearance for IV and apparent total body clearance divided by bioavailability for SC;
IV = intravenous;
SC = subcutaneous;
Tₘₐₓ = time to maximum plasma concentration;
t_{1/2} = terminal plasma half-life

Pharmacodynamics—

CRP levels were reduced in all subjects who received Humanized AB1 irrespective of dose or administration route. From Weeks 4 to 12, CRP levels were significantly lower in subjects who received Humanized Ab1 compared with placebo (unadjusted p-value <0.05). A high correlation between the IgG produced and antigen specificity for an exemplary IL-6 protocol was observed with 9 of 11 wells showed specific IgG correlation with antigen recognition. In Humanized AB1 subjects, CRP levels were lowered to <20% of pre-dose levels in: 72% (13/18) of subjects at Week 1; 73% (11/15) of subjects at Week 12; and 56% (10/18) of subjects at Week 24.

Conclusions—

In this Phase I study, the anti-IL-6 antibody Humanized Ab1 was generally well tolerated when administered in a single SC dose in healthy male subjects. Injection site reactions were generally mild. No anti-Humanized Ab1 antibodies were detected. Changes in liver enzymes, neutrophil and platelet counts were reversible. The bioavailability of SC Humanized AB1 was approximately 60% of that observed with IV Humanized Ab1. The half-life of Humanized AB1 was approximately 30 days, irrespective of route of administration. These data concur with previous data using IV Humanized Ab12. Subcutaneous Humanized AB1 led to rapid and large reductions in serum CRP. Reductions in CRP observed during the first 12 weeks of the study were sustained over 24 weeks of assessment. These preliminary data support the continued development and evaluation of subcutaneous Humanized Ab1 for the treatment of patients with psoriatic arthritis.

In summary, in this Phase I study, the anti-IL-6 antibody Humanized Ab1 was well tolerated when administered in a single SC dose; injection site reactions were generally mild. The bioavailability of SC Humanized Ab1 was ˜60% of IV Humanized Ab1, and the half-life was ˜30 days. Rapid and significant reductions in CRP were observed, which were sustained over 24 weeks of assessment.

### Example 21

### Randomized, Double-Blind, Placebo-Controlled, Dose Ranging, Multi-Center (Phase IIIB) Study to Evaluate the Efficacy and Safety of BMS-945429 (“Clazakizumab”) Subcutaneous Injection in Adults with Active Psoriatic Arthritis

Background—

Psoriatic arthritis (PsA), a seronegative spondyloarthropathy is a complex disease involving peripheral and axial joints, periarticular structures (e.g., enthesitis, inflammation of other soft tissues, dactylitis) as well as the skin and nails. Without appropriate management, the number of joints affected by PsA and the severity of joint damage increase over time, which can lead to marked restrictions of the daily activities and to substantially compromised quality of life. Evidence has shown that accelerated atherosclerosis, obesity, metabolic syndrome and cardiovascular disease are associated with active PsA. Other co-morbidities such as pulmonary fibrosis, uveitis, and, less commonly, aortic and aortic valve inflammation also contribute to complexity of PsA.

Unlike RA, effective treatment options are limited for PsA. Saad et al. (2008) J Rheumatol. 35:883-90; Kavanaugh et al. (2006) J Rheumatol. 33:1417-21; Lee Gavin (2010) The Hong Kong Medical Diary. 15:26-7; Nash P. (2006) J Rheumatol. 33:1431-4. Responses to the traditional disease-modifying anti-rheumatic drugs (DMARDs) have been suboptimal. Kavanaugh et al. (2006) J Rheumatol. 33:1417-21; Soriano et al. (2006) J Rheumatol. 33:1422-30. Anti-tumor necrosis factor (TNF) therapies are efficacious for both skin and joint diseases but approximately 40% of subjects treated with anti-TNF agents do not reach a minimal improvement in joint responses (ACR20) and a large portion of subjects do not reach higher level responses (i.e. ACR50 or 70). Mease et al. (2000) The Lancet. 356:385-90; Mease et al. (2004) Arthritis and Rheum. 50(7):2264-72; Genovese et al. (2007) J Rheumatol. 34:1040-50; Mease et al. (2005) Arthritis and Rheum. 52:3279-89; Mease P. (2007) Therapeutics and Clinical Risk Management. 3:133-48; Antoni et al. (2005) Arthritis and Rheum. 52:1227-36; Antoni et al. (2008) J Rheumatol. 35:869-876; Antoni et al. (2005) Ann Rheum Dis. 64:1150-7. Several effective RA therapies have not provided the desired response in PsA and left the anti-TNF agents the major class of approved biologic therapy for PsA. Mease et al. (2011) Arthritis and Rheum. 63:939-948. Several agents under development for PsA had good efficacy for psoriatic skin lesions but with less optimal joint efficacy. Mease P. (2006) Bulletin of the NYU Hospital for Joint Diseases. 64: 25-31; Weger W. (2010) British Journal of Pharmacology. 160:810-20; Gottlieb et al. (2009) The Lancet. 373:633-40.

Therefore, there is still a significant unmet need in PsA for therapies that provide higher levels of efficacy in the joints in a greater proportion of subjects especially with the additional attributes of durability of effect over time, low immunogenicity, a subcutaneous dosing regimen that may allow for less frequent administration, and a risk benefit profile that remains acceptable.

Introduction:

This example presents results from the 24-week double-blind period for clinical study report (CSR) IM133004, a Phase 2b, randomized, double-blind, placebo-controlled, dose-ranging, multicenter study, followed by a long-term extension (LTE) in subjects with psoriatic arthritis (PsA) as diagnosed by the Classification Criteria for Psoriatic Arthritis (CASPAR) with active disease, who had an inadequate response to nonsteroidal anti-inflammatory drug (NSAIDs) and naive to or with inadequate response to non-biologic disease-modifying anti-rheumatic drugs (DMARDs).

Clazakizumab (BMS-945429) is a genetically engineered humanized IgG1 anti-interleukin-6 monoclonal antibody (anti-IL-6 mAb) that is being developed by Bristol-Myers Squibb Co. (BMS) for the treatment of rheumatoid arthritis (RA) and other non-oncology related indications. Clazakizumab is also known as ALD-518 and is being developed by Alder BioPharmaceuticals, Inc. (Alder) for use in cancer patients.

Interleukin-6 is a soluble pleiotropic cytokine that plays a critical role in the pathogenesis of many inflammatory conditions, including RA.1 Interleukin-6 mediates various functions of multiple cell types, both immune and non-immune. These functions include cell proliferation and differentiation, cell activation, B-cell secretion of antibodies, hepatocyte production of acute phase proteins, and hematopoiesis. The clinical relevance of IL-6 in RA is demonstrated by the approval of tocilizumab (Actemra®), a monoclonal antibody that binds to the IL-6 receptor and blocks the biologic activity of IL-6. Monthly intravenous (IV) administrations of tocilizumab significantly improve signs and symptoms and suppress joint damage in RA patients.

In contrast, clazakizumab binds to the soluble human IL-6 cytokine, rather than to the IL-6 receptor. Together with other properties such as high potency and long half-life (˜30 days), the characteristics of clazakizumab may result in a differentiated clinical profile compared to tocilizumab or other biologics.

In this study, clazakizumab was administered subcutaneously (SC) in subjects with moderate to severe active rheumatoid arthritis with inadequate response to methotrexate (MTX). This dose-ranging, placebo/active-controlled study was designed to compare the efficacy and safety of clazakizumab with MTX or clazakizumab monotherapy to placebo on background MTX over 24 weeks. Given the significant unmet needs in PsA, this study in PSA (IM133004) was designed to evaluate the safety and efficacy of SC injection of clazakizumab in PsA.

Objectives—

The primary objective during the double-blind period was to compare the efficacy of three doses of clazakizumab subcutaneous (SC) versus placebo (PBO) as assessed by American College of Rheumatology 20% improvement (ACR20) response rates at 16 weeks.

The secondary objectives of the double-blind period were to: (i) assess additional efficacy outcomes of clazakizumab SC at 16 weeks as measured by psoriasis area and severity index (PASI; specifically PASI75), ACR50 and ACR70 response rates, physical function and health related quality of life outcomes; (ii) assess efficacy outcomes of clazakizumab SC at 24 weeks as measured by PASI75, ACR20, ACR50 and ACR70 response rates, physical function and health related quality of life outcomes; and (iii) assess safety, tolerability and immunogenicity of clazakizumab SC injections.

Methodology—

This clinical study report presents results from the 24-week double-blind period for Study IM133004 (incorporated herein by reference), a Phase 2b, randomized, double-blind, placebo-controlled, dose-ranging, multicenter study, followed by an long-term extension (LTE) in subjects with psoriatic arthritis (PsA) by the Classification Criteria for Psoriatic Arthritis (CASPAR) with active disease, who had an inadequate response to nonsteroidal anti-inflammatory drug (NSAIDs) and/or non-biologic disease-modifying anti-rheumatic drugs (DMARDs).

Period I (Randomization/Day 1 to Week 16):

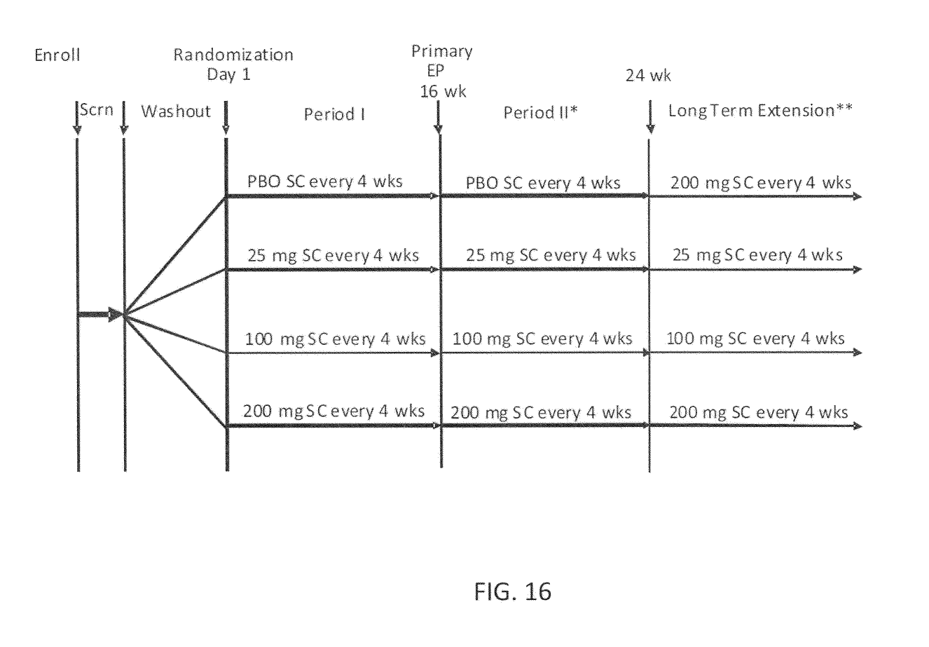

Upon meeting the inclusion/exclusion criteria, subjects were randomized to 1 of the 4 treatment arms (placebo or clazakizumab 25 mg SC every 4 weeks, 100 mg SC every 4 weeks, or 200 mg SC every 4 weeks) with a 1:1:1:1 ratio as shown in FIG. 16.

After the study began, that FDA mandated that all subjects be on active treatment to avoid the potential for radiographic progression over 24 weeks in subjects randomized to placebo. The protocol was amended to attempt to put all subjects on methotrexate (MTX) and to standardize the MTX treatment by placing such subjects on MTX at Week 16. Subjects who were not on MTX between Weeks 0 and 16 were allowed to continue without MTX until Week 16 when they were all placed on MTX.

Period II (Week 16 to Week 24):

All subjects who completed Period I continued to receive the same treatment assignments during Weeks 16 to 24. Subjects who had been enrolled in this study but were not yet randomized, and who were not on a background of MTX, were placed on oral MTX 15 mg/week (but no less than 10 mg/wk) at Week 16; this allowed subjects randomized to placebo to be placed on active therapy. Those subjects (who enrolled early in the study) who were between Weeks 0 and 16 and who were not on MTX were allowed to continue without receiving MTX until Week 16 when they were all placed on MTX. Subjects who were after Week 16 by the time of the FDA mandate (for active treatment) and were not already on MTX were allowed to go on without MTX to Week 24 when they started receiving clazakizumab 200 mg as per protocol.

During Period II, doses of oral glucocorticosteroids could be changed but the total dose remained ≦10 mg/day of prednisone or prednisone equivalent. NSAID dose changes were also allowed according to investigator's clinical judgment for appropriate disease management.

In addition, subjects that did not achieve at least a 20% reduction compared to baseline in the swollen and tender joint count during Weeks 16 to 24, and in the LTE until the switch to the final dose of 25 mg clazakizumab, were eligible for rescue therapy.

Number of Subjects (Planned and Analyzed):

168 subjects were planned to be included (42 subjects per arm); 165 subjects were analyzed (41 each in the placebo, 25 mg clazakizumab, and 200 mg clazakizumab groups and 42 subjects in the 100 mg clazakizumab group).

Diagnosis and Main Criteria for Inclusion:

Subjects must have had a diagnosis of PsA by CASPAR criteria and had active disease for at least 12 weeks prior to screening. Subjects must have had inadequate responses to NSAIDs and/or non-biologic DMARD therapy. Subjects must have had a minimum of >3 swollen and >3 tender joints (66/68 joint counts); active psoriatic skin lesions of >3% Body Surface Area (BSA); and a high sensitivity CRP (hsCRP) of >ULN (by central laboratory values) at screening. Subjects who were on MTX were allowed if they had been taking MTX for at least 3 months at a dose >15 mg/week to a maximum weekly dose of ˜25 mg, and were at a stable dose of MTX for 4 weeks prior to randomization (Day 1). Other non-biologic DMARDs had to be washed out according to the protocol.

Subjects are excluded if they had previously received or were currently receiving an approved biologic therapy for PsA or psoriasis. Subjects were excluded if they had active systemic inflammatory condition other than PsA which might have interfered with the results of clinical or laboratory tests planned in the study (eg, systemic lupus erythematosus or any other systemic rheumatic disease other than PsA).

Criteria for Evaluation: Efficacy:

Clinical joint, skin, dactylitis, and enthesitis assessments were conducted. The primary efficacy assessment was the proportion of subjects meeting the ACR criteria for improvement (ACR20) at Week 16. The secondary efficacy assessments comprised: individual components of the ACR core data set, ACR50, ACR70, PASI75, Health Assessment Questionnaire—Disability Index (HAQ-DI), Short Form (36) (SF-36). Safety: The evaluation of drug safety was based on clinical AEs, vital signs, ECGs and laboratory abnormalities reported during the double-blind study period. Immunogenicity: Serum samples were assayed for the presence of anti-clazakizumab antibodies.

Statistical Considerations:

For the primary endpoint of ACR20 at Week 16, assuming the placebo response rate of 15% with an a=0.017 (two-sided), approximately 42 subjects per arm (total 168) would provide around 85% power to detect a difference in treatment response rate of 37%.

The primary testing procedure involved 3 comparisons of clazakizumab (25 mg SC every 4 weeks, 100 mg SC every 4 weeks, or 200 mg SC every 4 weeks) versus placebo and Dunnett-Tamhane step-up procedure was performed to control the overall type I error rate at 0.05 for multiple comparisons.

Efficacy Results:

Overall Efficacy Summary:

The study met its primary objective of at least one dose of clazakizumab being statistically superior to placebo on the primary endpoint of ACR20 at Week 16.

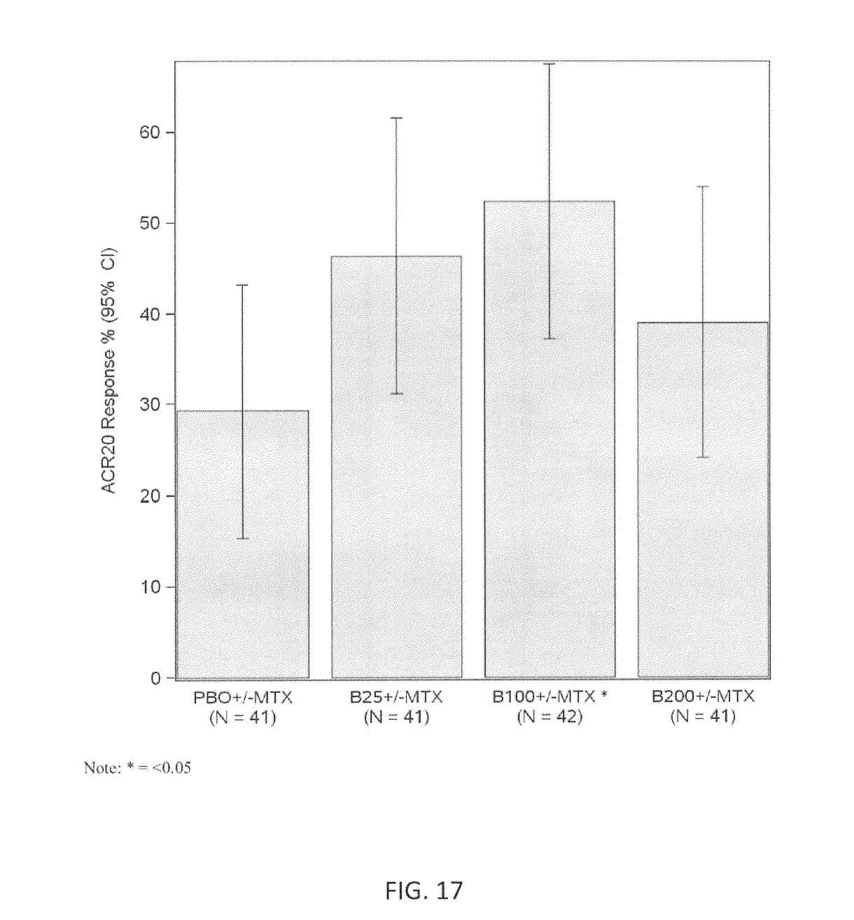

The proportion of subjects with an ACR20 response rate at Day 113 (Week 16) was numerically higher in the 3 clazakizumab groups compared with the placebo group (Table 14 and FIG. 17). In the 100 mg clazakizumab group, this improvement (difference of 23.1%) was statistically superior to placebo (adjusted p-value=0.039; Table 14).

TABLE 14
Proportion of Subjects Achieving ACR20 at Day 113 (Week 16) (All Randomized and Treated Subjects)

| | PBO +/− MTX | B25 +/− MTX | B100 +/− MTX | B200 +/− MTX |
|---|---|---|---|---|
| | N = 41 | N = 41 | N = 42 | N = 41 |
| # OF SUBJECTS <Y/X> (%) | 12/41 (29.3) | 19/41 (46.3) | 22/42 (52.4) | 16/41 (39.0) |
| 95% CI | (15.3, 43.2) | (31.1, 61.6) | (37.3, 67.5) | (24.1, 54.0) |
| ESTIMATE OF DIFFERENCE (%) | | 17.1 | 23.1 | 9.8 |
| 95% CI | | (−3.6, 37.7) | (2.6, 43.7) | (−10.7, 30.2) |
| P-VALUE (VS PLACEBO) | | 0.101 | 0.039 | 0.178 |

Y = Number of subjects with measure/event of interest, X = Number of subjects in the analysis.
For CI within each group, normal approximation is used if Y >= 5 and X − Y >= 5. Otherwise exact method is used.
For CI of difference, normal approximation is used if Y >= 5 and X − Y >= 5 in both arms. Otherwise exact method is used.
P-value is based on Dunnett-Tamhane step up procedure.
ACR20 = 20% ACR response;
PBO = Placebo;
MTX = Methotrexate;
CI = Confidence interval.

No dose response for efficacy was noted in this study; the 25 mg and 100 mg clazakizumab doses tended to show better efficacy results for most parameters compared to the 200 mg clazakizumab dose group and placebo.

When examining efficacy result for the 5 efficacy domains of PsA, the benefits of clazakizumab treatment were shown as follows:

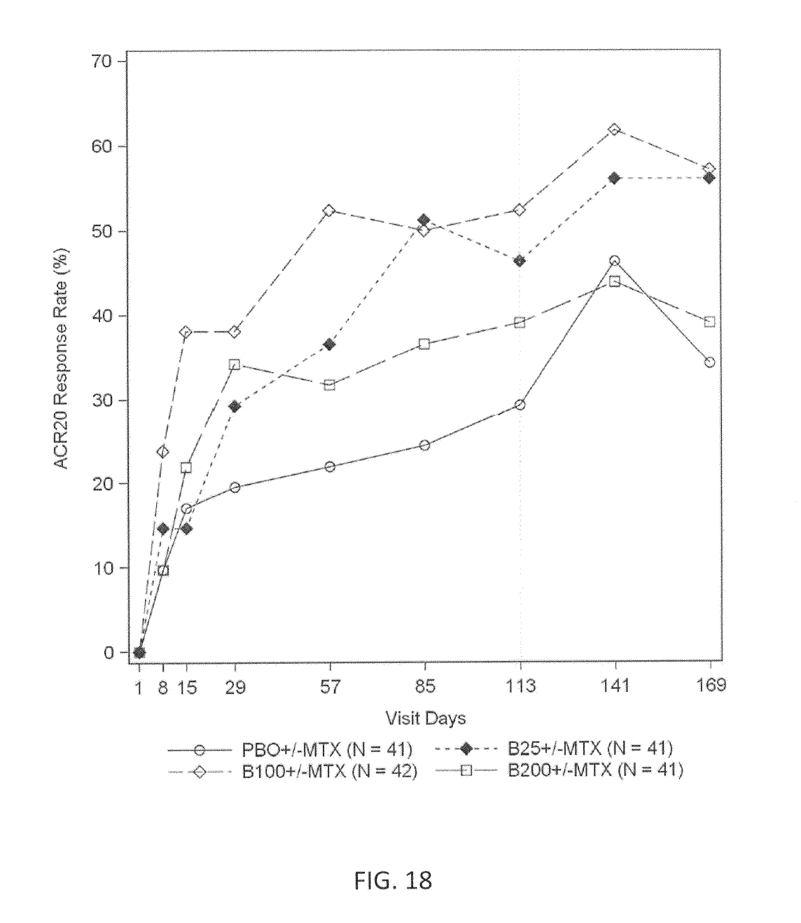

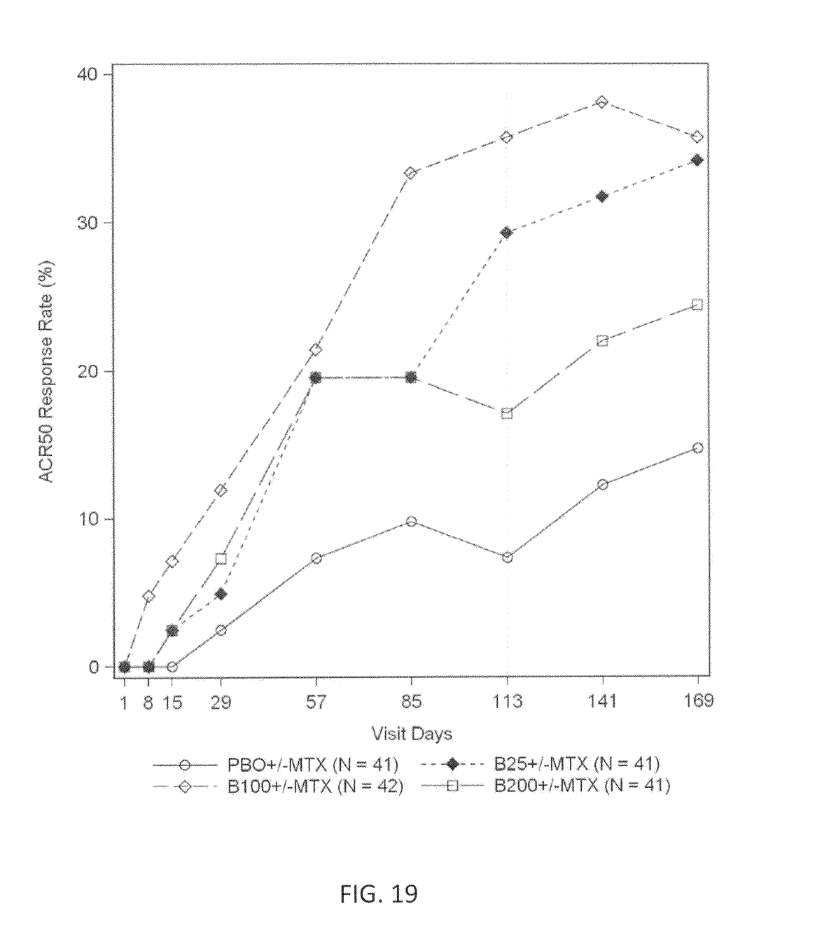

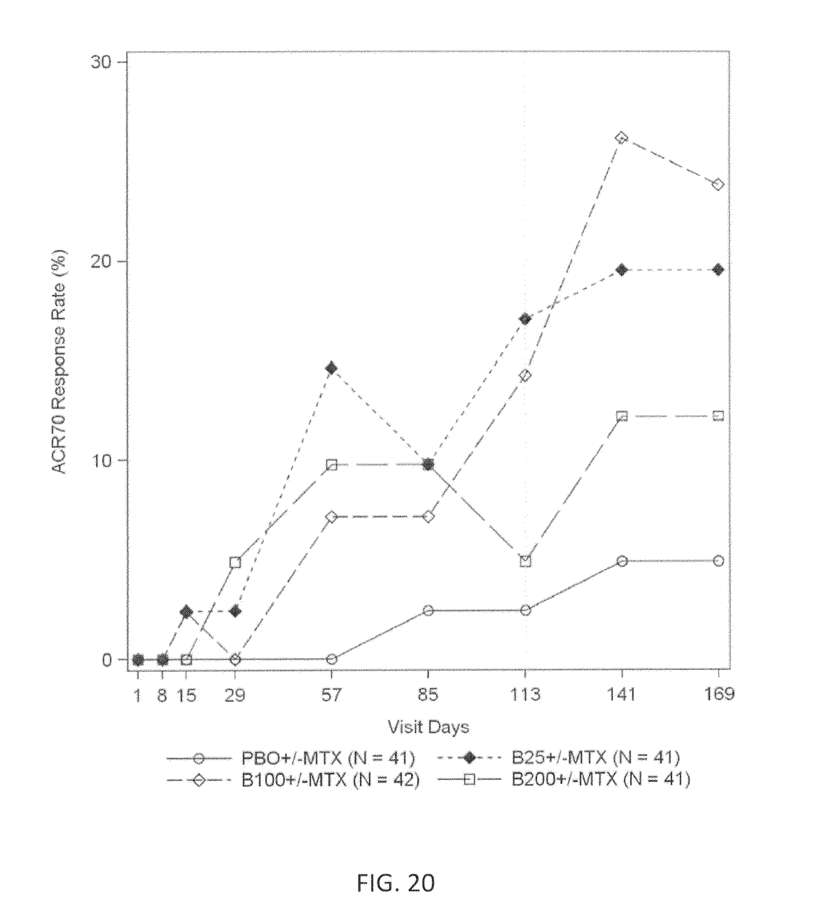

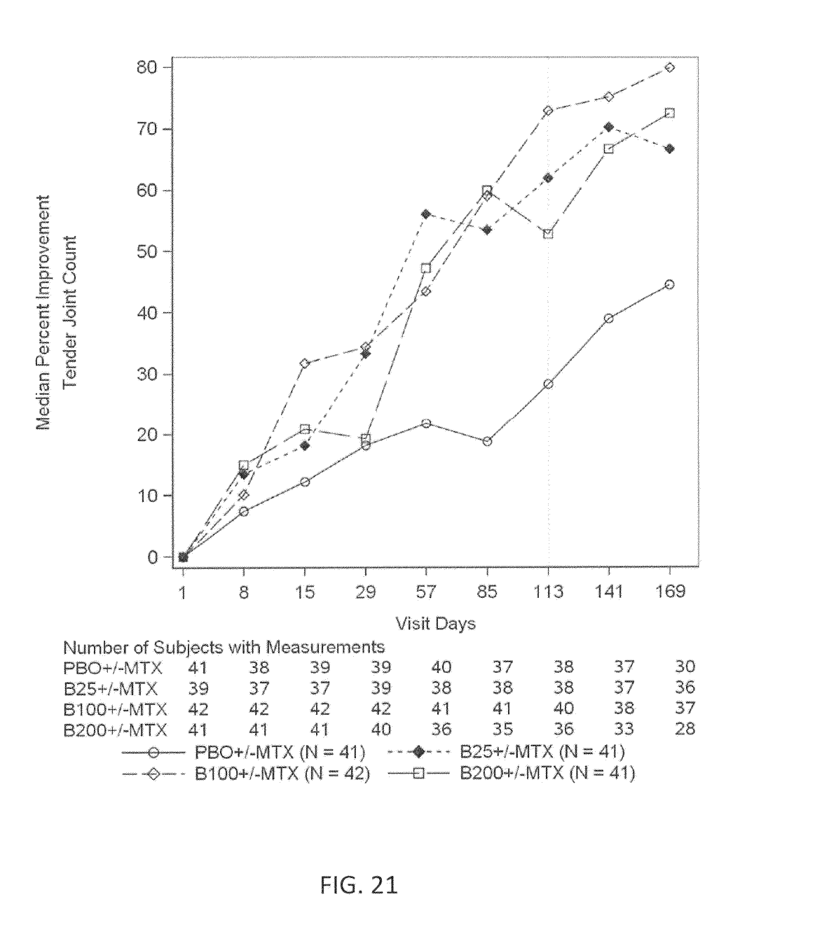

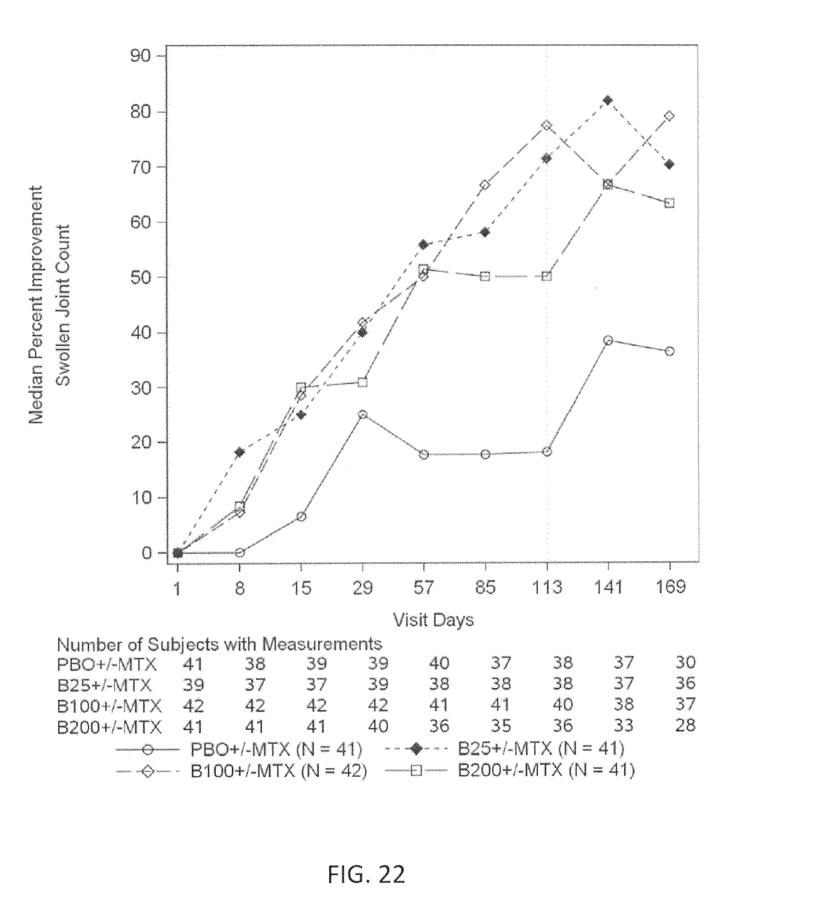

Joints—improvement was noted in tender and swollen joints as shown by positive results of the ACR20, ACR50, ACR70 (FIGS. 18-20, respectively), individual components of the American College of Rheumatology (ACR) (FIG. 21 for tender joint count and FIG. 22 for swollen joint count), DAS28-CRP (Disease Associated Score, C-reactive protein, data not shown), DAS28-CRP <2.6 (data not shown), HAQ-DI (Health Assessment Questionnaire, Disability Index, data not shown), and the PsARC (composite index that is predominantly associated with outcomes related to changes in tender and swollen joints, data not shown). Clazakizumab showed early (onset of action by 4 weeks) and consistent improvement over placebo in these joint measures with greater improvement more often being seen in the 25 mg and 100 mg clazakizumab doses compared to the 200 mg dose at Day 113 (Week 16) and Day 169 (Week 24).

Skin—there was no difference in the PASI75 (Psoriasis Area Severity Index) between any of the clazakizumab doses and placebo at Day 113 (Week 16); however, there appeared to be a marginal improvement over placebo in both the 25 and 100 mg clazakizumab dose groups on the PASI75 and PASI50 response at Day 169 (Week 24). No consistent results were noted when comparing mean change from baseline in PASI over time (data not shown).

Enthesitis—there was apparent improvement in enthesitis with clazakizumab treatment compared with placebo. The SPARCC Enthesitis Index showed that the proportion of subjects with enthesitis at baseline decreased over time in the 3 clazakizumab dose groups compared with placebo at both Day 113 (Week 16) and Day 169 (Week 24). There was no meaningful difference in the mean change from baseline LEI (Leeds Enthesitis Index) score among the 3 clazakizumab groups and the placebo group (data not shown).

Dactylitis—the results for this parameter based on the LDI (Leeds Dactylitis Index) were not able to be analyzed for this study due to unreliable data collection for this measure. However, in the clazakizumab groups there was an apparent decrease over time in tender and swollen digits among those subjects with at least 1 tender and swollen digit (data not shown).

Spine—In this study, the BASDAI (Bath Ankylosing Spondylitis Index) score was used as a surrogate measure of spinal involvement; in the 25 and 100 mg clazakizumab groups showed a small numerical improvement over placebo on this scale (data not shown).

Safety Results:

Overall Safety Summary:

Clazakizumab was tolerated at all doses studied. Few subjects discontinued therapy over the first 24 weeks of the study. Nevertheless, the study data did reveal dose-related safety findings. Overall, the 25 mg clazakizumab dose group had the lowest frequency of AEs, discontinuation due to AEs, transaminase abnormalities and injection site reactions whereas a higher incidence of these safety measures was noted in the 200 mg group. Although the safety profile of the 100 mg clazakizumab dose group was similar in many respects to the 25 mg dose group, the benefit-risk profile of clazakizumab appeared most favorable at 25 mg. No new safety signals were observed compared to the RA patients treated with clazakizumab. No subject died during the study and no subject had laboratory abnormalities that met the criteria for Hy's Law.

Safety data were available for 165 subjects in the As-Treated Analysis Population including those subjects who received at least 1 dose of double-blind treatment. This dataset included the 149 subjects who completed through Week 16 of the study (Period I) and the 140 subjects who completed Weeks 16 to 24 (Period II). Safety observations for the double-blind treatment period include results from Period I (through Week 16; data not shown) and results from both Periods I and II (through Week 24; data not shown). Safety observations for Periods I and II (cumulative through Week 24) are summarized as follows:

Deaths:

No subject died during the double-blind treatment period of the study.

Serious Adverse Events (SAEs):

Serious adverse events were reported for 10 (6.1%) subjects (including 7 [4.2%] subjects in Period I): 2 subjects (4.9%) in the placebo group, 2 subjects (4.9%) in the 25 mg clazakizumab group, 2 subjects (4.8%) in the 100 mg clazakizumab group, and 4 subjects (9.8%) in the 200 mg clazakizumab group. With the exception of transient hypoesthesia and muscular weakness in Subject IM133004-70-103 in the 200 mg clazakizumab group, none of these SAEs was reported by the investigator to be related to study drug. In the placebo group, SAE PTs were bladder neoplasm and transitional cell carcinoma in 1 subject and prostate cancer in 1 subject. In the 25 mg clazakizumab group, SAE was bradycardia and chest pain each in 1 subject. In the 100 mg clazakizumab group, SAE was psoriasis and dystonia each in 1 subject. In the 200 mg clazakizumab group, SAEs were hypoesthesia and muscular weakness in 1 subject, and intervertebral disc disorder, acute myocardial infarction, and hemiparesis each in 1 subject.

Discontinuations Due to Adverse Events (AEs):

Thirteen (7.9%) subjects discontinued due to AEs including 2 subjects in the placebo group who discontinued due to SAEs of transitional cell carcinoma and prostate cancer, and 1 subject in the 200 mg clazakizumab group who discontinued due to SAEs of hypoesthesia and muscular weakness. Eight (4.8%) of these subjects discontinued during Period I.

Adverse Events of Special Interest:

Infections and infestations (including nasopharyngitis, pharyngitis, urinary tract infection [UTI], upper respiratory tract infection [URTI], and bronchitis) were noted in a higher proportion of subjects in the placebo group (48.8%) compared to the 3 clazakizumab groups (36.6%, 35.7%, and 24.4%, in the 25 mg, 100 mg and 200 mg groups, respectively). There were no cases of tuberculosis or reports of opportunistic infections. Hepatic disorders (including increased ALT and AST) were only reported in the 3 clazakizumab groups (22.0%, 16.7%, and 26.8%, in the 25 mg, 100 mg and 200 mg groups, respectively). There was a low incidence (<10% of subjects) of local injection site events in the 25 mg (9.8%) and 100 mg (9.5%) clazakizumab groups and in the placebo group (4.9%); local injection site events were reported with higher frequency in the 200 mg clazakizumab group (17.1%). No subject experienced autoimmune disorders, systemic injection events, demyelinating disorders or gastrointestinal perforations during the double-blind treatment period. There were two malignancies reported in the placebo group (transitional cell carcinoma and prostate carcinoma) but no malignancies were reported in the 3 clazakizumab groups.

Overall AEs:

AEs were reported for 123 (74.5%) subjects (including 115 [60.7%] subjects during Period I) and AEs were assessed as drug-related for 71 (43.0%) subjects (including 64 [38.8%] subjects during Period I). The highest incidence of AEs (82.9%) and related AEs (63.4%) were noted in the 200 mg clazakizumab group. Adverse events reported for >5% of subjects overall included increased ALT (13.9%), increased AST (8.5%), nasopharyngitis (7.9%), hypercholesterolemia (7.3%), pharyngitis (6.1%), URTI (6.1%), headache (6.1%), and hypertension (6.1%).

Laboratory Abnormalities:

With a few exceptions, changes from baseline in most laboratory values were similar across the treatment groups. Changes from baseline that were more prevalent in the clazakizumab groups compared with the placebo group included decreases in mean absolute neutrophil count, decrease in mean platelet count, increases in total cholesterol levels, increases in mean ALT (Alanine aminotransferase), AST (Alkaline phosphatase), and total bilirubin levels, and decreases in mean alkaline phosphatase levels.

Vital Signs and ECG:

No safety concerns were identified based on evaluation of laboratory and vital sign data.

Pharmacokinetic Results:

The following is a brief summary of the key pharmacokinetic findings: The degree of fluctuation between the maximal and minimal (pre-dose) concentration was minimal (less than 2 fold) across dose range following SC administration of clazakizumab. Based on observed trough (Cmin) concentrations, steady-state was reached by the time of Week 20 which is consistent with the half-life of clazakizumab. Following the SC dose of clazakizumab in the range of 25 mg to 200 mg, Cmax and AUC(TAU) (Area under the serum concentration-time curve over a dosing interval, TAU=4 weeks) increased slightly less than proportionally to dose, which may be attributed to limitations in sample size and variability in sample collection times. Median Tmax values were 3 to 7 days (ranged 2 to 21 days) across the treatment groups (data not shown).

Pharmacodynamic Results:

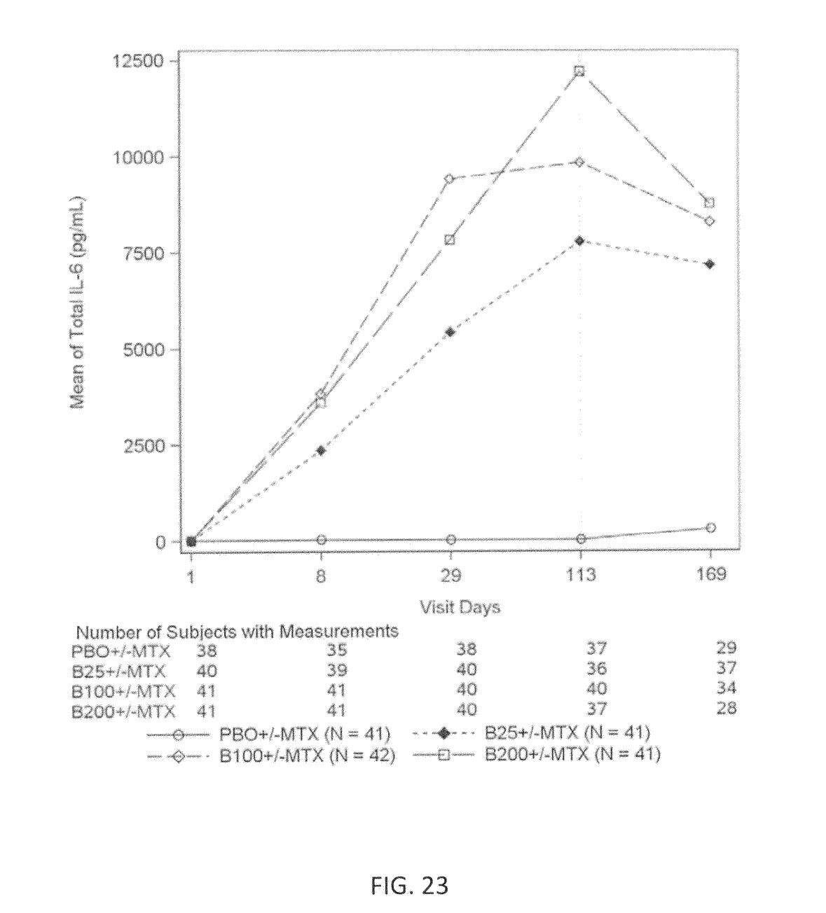

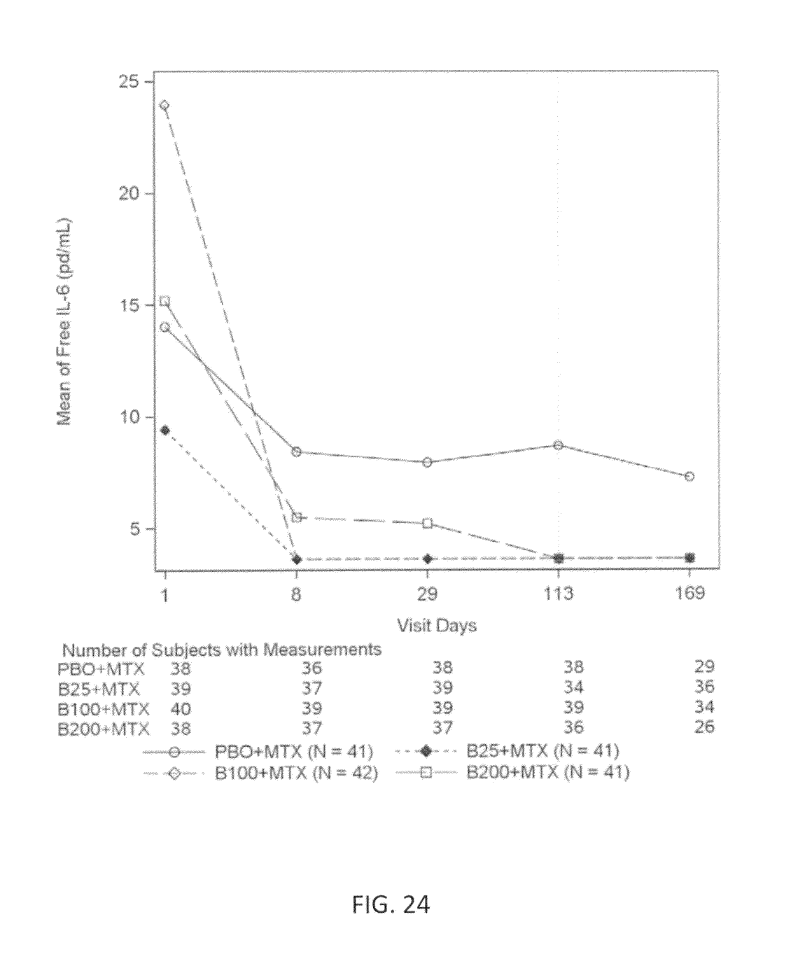

As an indicator of target engagement, total (FIG. 23) and free (FIG. 24) levels of serum IL-6 were measured using validated immunoassays that discriminate between clazakizumab bound IL-6 (total IL-6) vs. non-clazakizumab bound ligand (free IL-6). At baseline, levels of IL-6 ranged between 10 to 25 pg/mL (Table S.9.3). In the placebo arm, there was little change in total or free IL-6 throughout the course of the study. In the clazakizumab treated subjects, free IL-6 decreased below the level of detection (3.6 pg/mL) immediately postdosing and across all dose groups. These changes were observed at the earliest time point (Day 8) and were sustained throughout the study (Day 169 [Week 24]). For the 200 mg clazakizumab dose group, mean levels of free IL-6 demonstrated detectable levels of free IL-6 at Days 8 and 29, however, this result was due to 1 outlier that had delayed suppression of IL-6 levels. Upon further treatment, this subject showed undetectable levels of free IL-6 starting at Day 57 that were maintained through Day 169. Postdosing with clazakizumab, there was a rapid increase in total IL-6 levels reflective of the increase in drug bound ligand. Total IL-6 levels increased approximately 200-fold above baseline at Day 8 (mean range 2355 to 3832 pg/mL) and appeared to plateau at later time points to approximately 500-fold above baseline (mean range 7175 to 12209 pg/mL at Days 113 and 169). An observed increase in the total IL-6 levels in the 200 mg dose group at Day 113 was driven by a few outliers and was not representative of the entire group. There was a modest dose response that was observed across the dose groups with a lower increase in total IL-6 levels in the 25 mg clazakizumab dose group compared to the two higher clazakizumab dose groups (100 and 200 mg). Overall, these results demonstrate target engagement of clazakizumab treatment at all dose groups and throughout the course of the study.

### Example 22

### Phase III, Randomized, Multi-Center, Double-Dummy, Double-Blind, Placebo and Active-Comparator Study to Evaluate the Efficacy and Safety of Clazakizumab Compared to Placebo and Adalimumab in Subjects with Active Rheumatoid Arthritis Who are Inadequate Responders to Oral Conventional Synthetic Disease Modifying Anti-Rheumatic Drugs (DMARDs)

Herein we provide clinical regimens using low dosing regimens which further validate the improved clinical benefit of the subject anti-IL-6 antibody comprising the heavy and light polypeptides of SEQ ID NO:709 and 657 (clazakizumab) relative to established RA therapies in achieving a high level of disease control using a stringent measure of signs and symptoms (i.e. DAS 28 CRP <2.6), along with clinically meaningful benefits in physical function and inhibition of structural damage, and a favorable overall safety profile in patients who are inadequate responders to conventional synthetic DMARDs or naive to MTX.

Particularly, these studies will confirm the efficacy and safety of SC clazakizumab compared to placebo and to adalimumab in combination with MTX, in subjects with moderate to severe RA who have IR to at least one conventional synthetic DMARD including MTX. Data from this study will demonstrate that clazakizumab is superior to adalimumab in the proportion of patients who achieve a high level of disease control, as measured by DAS 28 CRP <2.6.

### Efficacy

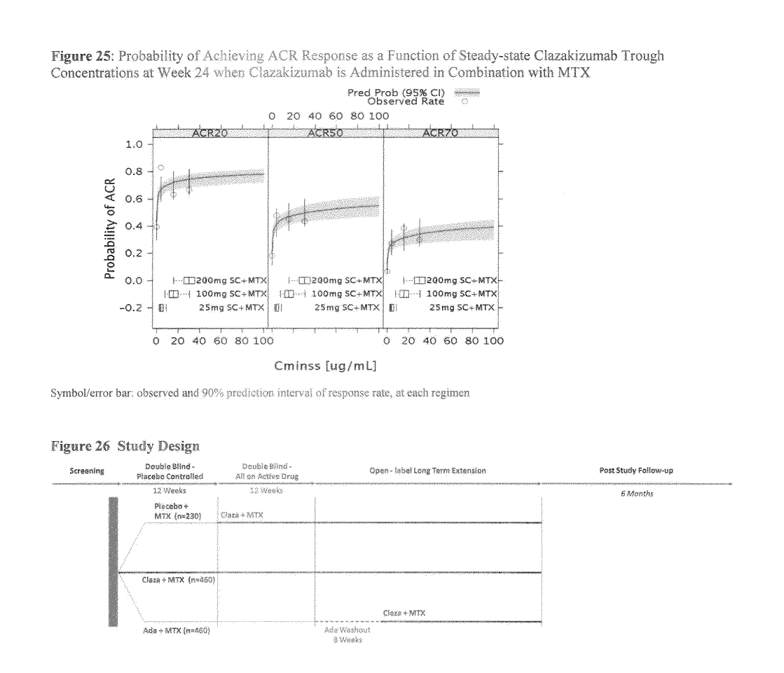

The subject anti-IL-6 antibody comprising the variable heavy and light polypeptides of SEQ ID NO:709 and 657 (clazakizumab) when used at doses of 25 mg, 100 mg, and 200 mg/month with background MTX, 100 mg and 200 mg monotherapy) demonstrated efficacy over placebo. In addition, each of the clazakizumab+MTX doses was associated with more patients achieving stringent measures of response than with adalimumab+MTX. Overall, there was not a strong dose response relationship at the doses tested on ACR20, ACR50, ACR70, DAS28-CRP<2.6, CDAI<2.8, or SDAI<3.3. These findings were corroborated by exposure response analyses, which examined the relationship between steady-state Cₘᵢₙ concentrations (Cmins), and ACR response rates. Within the range of Cmins achieved with the 25 mg and higher, the relationship between Cmins and the probability of achieving an ACR response was relatively flat (FIG. 25). While the overall magnitude of response was lower for monotherapy regimens compared to the MTX combination therapies, the relationship between Cₘᵢₙₛ and the probability of achieving ACR response was again flat across the exposures associated with the 100 and 200 mg monotherapy doses. Exposure response analyses for alternate efficacy endpoints, including DAS 28 CRP reduction and CDAI reduction (which excludes CRP) were similar.

In these clinical trials RA patients are treated with low doses (1 mg, 5 mg, and 25 mg/month of clazakizumab with background MTX) to confirm the optimal benefit/risk of the 25 mg clazakizumab dose in RA patients with similar disease activity and is expected to have a more favorable benefit-risk profile than the lower doses. Therefore, clazakizumab 25 mg SC q 4 weeks is planned to be the dose used in this study.

### Safety

Clazakizumab was well-tolerated and efficacious (resulted in remission of disease symptoms) at all doses studied. Nevertheless, Phase 2b data did reveal some dose-related safety findings. However, it was observed that the clazakizumab 25 mg+MTX group had the lowest frequency of AEs, discontinuation due to AEs, LFT abnormalities and injection site reactions among the 3 MTX combination treatment groups studied and yet was still effective in treating disease symptoms. There were no substantive differences in the safety profiles of the 100 and 200 mg monotherapy treatment groups. In addition, with the exception of LFT abnormalities, there was no meaningful difference in the safety profile of clazakizumab administered as combination vs as monotherapy. These results suggest that the safety profile of clazakizumab is acceptable at all doses studied, but is most safe and effective at 25 mg or lower monthly dosages.

### a) Study Rationale

Based thereon the current study aims to demonstrate clinical benefits of clazakizumab, when used in combination with MTX, in RA patients with moderately to severely active disease, who are inadequately responding to conventional synthetic DMARDs. Many elements of the study are based on prior study designs for studying new drugs in RA for registrational purposes. The chosen patient population is appropriate to study the efficacy and safety of clazakizumab in keeping with the treat-to-target strategy that early biologic intervention use in appropriate patients may prevent the long-term structural damage observed with inadequate early control. The confirmatory Phase 3 study is also intended to demonstrate that clazakizumab has clinically beneficial effects on other measures of signs and symptoms and physical function in patients with moderate to severe RA. The study contains placebo and active (adalimumab) comparators. Inclusion of the placebo arm will allow for ensuring assay sensitivity of effect size and adalimumab is chosen as an active comparator as it is the biologic standard of care agent in this population. Placebo duration of 12 weeks allows for determination of the effect size of clazakizumab at an appropriate time interval while permitting adequate management of symptoms in patients that are not meeting therapeutic goals. Comparison of clazakizumab to adalimumab will occur at 24 weeks, a time point by which it is expected that both clazakizumab and adalimumab will have achieved maximal efficacy to allow for assessment of the higher efficacy measures (i.e. DAS CRP <2.6).

The long term extension of the study is intended to evaluate the long term safety of treatment with clazakizumab as well as the durability of the clinical response to clazakizumab. During the long term extension, subjects on adalimumab will be switched to clazakizumab to determine if there is a further improvement in signs and symptoms of RA in those subjects.

### b) Dose Selection Rationale

RA subjects who are inadequate responders to synthetic DMARDs (including MTX) and have moderate to severely active disease, who were clazakizumab 25 mg demonstrated the best benefit-risk profile compared to other tested doses. Based thereon clazakizumab is administered 25 mg SC q 4 weeks.

### Pharmacodynamic Data

As an indicator of target engagement, inhibition of the soluble IL-6 and soluble IL-6 receptor complex formation was derived using a validated method that incorporates the direct measurements of clazakizumab concentration, total IL-6 concentrations, and binding affinity of clazakizumab to IL-6. When assessing IL-6/IL-6 soluble receptor complex inhibition across clazakizumab doses, there were differences across dose arms, such that higher doses exhibited higher distributions of IL-6/IL-6 soluble receptor complex inhibition. Examination of the clazakizumab IL-6/IL-6 soluble receptor complex inhibition data by ACR20 response, suggests that ACR20 responders had higher levels of inhibition at Week 12 than non-responders with the 25 mg dose in combination with MTX (FIG. 26). Similar trends were observed with ACR50 and ACR70. A validated enzyme linked immunosorbent assay (ELISA) method is used to measure concentrations of clazakizumab in serum.

Trough concentrations (Cmin) of clazakizumab will be summarized by visit day. Collected PK data will be combined with data from other studies for population PK analysis. This analysis will examine the potential effects of covariates such as age, body weight, ethnicity etc. on PK. Exposure-Response relationship between measures of exposure and selected efficacy (e.g., ACR, DAS28-CRP) and safety (eg, liver abnormality tests) endpoints will be characterized. Results from these analyses will be reported separately.

### Immunogenicity and Biomarkers

Predose serum samples will be collected at baseline (predose Day 1), at specified time points during the double-blind period and long term extension period, as well as at specified times after the subjects discontinues from the study.

Samples are assayed for the presence of anti-clazakizumab antibodies using a validated ECL assay method. The incidence of the formation of anti-clazakizumab antibodies will be summarized by treatment. The effect of anti-clazakizumab antibodies on the systemic exposure, safety, and efficacy of clazakizumab will be evaluated.

### Objectives:

These clinical studies will compare the efficacy of Clazakizumab versus PBO, both on background MTX, in terms of reducing signs and symptoms of RA as assessed by proportion of subjects achieving ACR20 at 12 weeks of treatment.

Also, these clinical studies will evaluate the following:
1) the efficacy of Clazakizumab versus PBO, both on background MTX, in improving physical function in RA as assessed by change in HAQ-DI over baseline at 12 weeks of treatment.
2) the efficacy of Clazakizumab versus PBO, both on background MTX, in achieving low disease activity as assessed by proportion of subjects with DAS28-CRP<2.6 at 12 weeks of treatment;
3) the efficacy of Clazakizumab versus Ada, both on background MTX, in achieving low disease activity as assessed by proportion of subjects with DAS28-CRP<2.6 at 24 weeks of treatment; and
4) the efficacy of Clazakizumab versus Ada, both on background MTX, in reducing RA signs and symptoms as assessed by proportion of subjects with ACR70 at 24 weeks of treatment.

Further, these clinical studies will evaluate the following:
1) efficacy responses (ACR20/50/70; HAQ-DI; SDAI≦3.3; CDAI≦2.8; DAS28-CRP<2.6; DAS28-ESR<2.6; change from baseline of DAS28-CRP; change from baseline of DAS28-ESR) over 24 weeks;
2) safety of Clazakizumab on background MTX through assessments of adverse events (AEs) and laboratory parameters; and
3) systemic exposure, immunogenicity and pharmacodynamics (PD) of Clazakizumab on background MTX.

Still further, these clinical studies will evaluate the following:
1) the long-term maintenance of efficacy responses (ACR20/50/70; HAQ-DI; SDAI≦3.3; CDAI≦2.8; DAS28-CRP<2.6; DAS28-ESR<2.6; change from baseline of DAS28-CRP; change from baseline of DAS28-ESR) of Clazakizumab on background MTX beyond 24 weeks;
2) the median time to onset of efficacy measures of Clazakizumab on background MTX;
3) the efficacy of Clazakizumab on background MTX in OLE in subjects who had received Ada in the double-blind period;
4) the efficacy of Ada versus PBO both on background MTX in achieving DAS28-CRP<2.6 over 12 weeks of treatment.
5) the efficacy of Clazakizumab on background MTX on Quality of Life measure (SF-36). 6) work productivity (WPAI-RA) with Clazakizumab versus PBO over 12 weeks and versus Ada over 24 weeks, all on background MTX;
7) fatigue (FACIT) with Clazakizumab versus PBO over 12 weeks and versus Ada over 24 weeks, all on background MTX;
8) the effects of covariates on the PK of Clazakizumab on background MTX and evaluate the exposure-response relationship for efficacy, safety, and PD markers (e.g. CRP, total IL-6, and free IL-6); and
9) biomarkers (including soluble, intracellular, and genomic) which may be used to predict and monitor treatment response and safety associated with treatment with Clazakizumab or Ada.

### Study Design and Duration:

Subjects with moderate to severe rheumatoid arthritis, who are inadequate clinical responders to conventional synthetic DMARDs (upon meeting the requirements based on inclusion and exclusion criteria), are randomized into 1 of the 3 treatment arms as shown in FIG. 26.

### Screening

Upon obtaining the informed consent, a subject's eligibility will be determined. Subjects must have experienced an inadequate clinical response to one or more conventional synthetic DMARDs (which must include MTX) as documented by a treating physician or investigator (see Section 3.3.1 for definition of inadequate response).

All subjects must have been receiving treatment with a minimum dose of 15 mg per week (maximum 25 mg per week) of methotrexate for at least 12 weeks and at a stable dose for 6 weeks prior to randomization. A lower dose of methotrexate is permitted in some circumstances Also, to minimize potential methotrexate toxicity all subjects should receive folic acid, folinic acid, or leucovorin according to the manufacturer recommendations and the local medical standard of care guidelines.

Oral prednisone or equivalent is permitted, if the dose is 10 mg/day and if it has been stable for 4 weeks before screening. Non-steroidal anti-inflammatory drugs (NSAIDs) must be stable for 4 weeks before screening and consistent with labeling recommendations. IA, IV and IM corticosteroid injections may not be administered within 4 weeks of screening.

### Period 1: Double-Blind/Placebo Controlled Period; Randomization to Week 12 (Primary Endpoint)

Following the screening period, eligible subjects will be randomized to 1 of 3 parallel arms on Day 1 at a 2:2:1 ratio as follows:

### Period 1 Treatment Arms:

1. Clazakizumab 25 mg SC every 4 weeks
PLUS Placebo for adalimumab SC every 2 weeks with background methotrexate (n=460)
   2. Adalimumab 40 mg SC every 2 weeks
PLUS Placebo for clazakizumab SC every 4 weeks with background methotrexate (n=460)

### 3. No Active Treatment

Placebo for adalimumab SC every 2 weeks
PLUS Placebo for clazakizumb SC every 4 weeks with background methotrexate (n=230)

Subjects and caregivers are trained during the first two study visits of this period in self administration of the study medication using pre-filled syringes. All subsequent injections will be self administered or administered by a caregiver, not by the physician or medical staff at the study site. During this period, the dose of methotrexate, NSAIDs, and oral prednisone (or its equivalent) should remain stable. Intra-articular corticosteroid injections and intramuscular corticosteroid injections are not permitted. Analgesics are permitted with certain restrictions (See Restricted and Prohibited medications)

### Period 2: Double-Blind/Active Drug Period; Week 12 to Week 24

During this period, subjects assigned to Treatment Arm #3 (no active treatment) will switch to the active study drug regimen described in Treatment Arm #1 (clazakizumab 25 mg SC every 4 weeks). For these subjects, concomitant medication requirements will not change. In all treatment arms, subjects will receive their final dose of study drug for this period at week 24.

### Period 3: Long Term Extension (LTE)

Subjects who continue to demonstrate clinical benefit at the end of Period 2, may elect to enter the LTE period. Subjects assigned to the Treatment Arm #2 will receive clazakizumab placebo at the LTE Day 1 and LTE Wk 4 visits. Following this washout period, subjects assigned to this arm will receive active clazakizumab 25 mg every 4 weeks. Subjects assigned to Treatment Arms #1 and #3 will receive active clazakizumab 25 mg every 4 weeks beginning with the LTE Day 1 visit.

Methotrexate (up to 25 mg), oral prednisone (≦10 mg/day) or its equivalent, NSAIDs and analgesics may be adjusted at the investigator's discretion during the LTE period. A single course of high dose oral, IM, or IA corticosteroid injection is permitted every six months.

The LTE will continue as an open-label study up to 12 months after the approval of study drug by the responsible health authority or until it becomes commercially available within the country, whichever occurs sooner. During the long term extension all subjects who continue to demonstrate clinical benefit as determined by their study physician will be eligible to continue to receive study drug. It is possible that the study drug dosing regimen will change during the long term extension or that subjects may be offered enrollment in another study or drug access program. If this occurs, details will be provided in the form of an amendment to the protocol.

### Post-Study Drug Follow-Up Period (6 Months)

Subjects who discontinue treatment of study drug or complete the study will have follow-up visits for up to six months, to perform safety and laboratory assessments. During this period, when subjects are no longer receiving study drug, it is recommended that they not be treated with other biologic therapy, due to clazakizumab's half-life of around 30 days. However, this decision remains at the investigator's discretion. If the study drug becomes commercially available, and if the subject chooses to receive treatment with the commercial product, then this period is not required.

### Key Inclusion/Exclusion Criteria:

Adult patients with RA for at least 16 weeks who have had a clinically inadequate response to conventional synthetic DMARDs including MTX but who have not used biologic therapy. Subjects must have clinical and laboratory evidence of active, moderate to severe RA. Subjects are excluded for high risk of infection, liver dysfunction, GI inflammation, and the presence of other non-RA rheumatologic disease.

### Inclusion Criterion:

a) documented diagnosis of active RA by standard criteria (ACR/EULAR [2010]) at least 16 weeks prior to screening;
b) ACR global functional status class of 1 to 3;
c) Documented evidence of inadequate clinical response to one or more conventional synthetic DMARDs (which must include MTX);
d) Methotrexate and conventional synthetic DMARDs: All subjects must have been receiving treatment with a minimum dose of 15 mg per week (maximum dose of 25 mg per week) of methotrexate for at least 16 weeks and at a stable dose for 6 weeks prior to randomization. A lower dose of methotrexate dose is permitted if there is verifiable documentation in the medical record prior to entry into the study that the subject could not receive or reach a dose of 15 mg due to toxicity and the dose is at least 10 mg methotrexate at the time of screening. In countries where the standard of care requires use of a lower doses of methotrexate (e.g. Japan), a minimum dose of 7.5 mg per week is permitted (to minimize potential methotrexate toxicity, all subjects must receive folic acid, folinic acid, or leucovorin according to manufacturer recommendations and local medical standard of care guidelines;
e) minimum of 6 swollen and 6 tender joints on a 66/68 joint count at screening and at baseline (Day 1);
f) subjects must have at least one of the following values at the screening:
   i. hsCRP of 0.8 mg/dL (8 mg/L) as measured by the Central Laboratory
   ii. Erythrocyte Sedimentation Rate (ESR) 28 mm/h ExclusionCriterion: 1. Target Disease Exceptions
      a) Subjects with documented juvenile rheumatoid arthritis.

### 2. Medical History and Concurrent Diseases

a) Subjects at risk for tuberculosis (TB). Specifically, subjects with:
   i. Current clinical, radiographic or laboratory evidence of active TB. Chest x-rays (PA and lateral) obtained within the 3 months prior to randomization will be permitted but the images must be available and reviewed by the investigator. TB testing (IFNg release assay or PPD) performed in the past month prior to randomization will be accepted, however a copy of the report must be placed in the subject binder.
   ii. A history of active TB unless there is documentation that the prior anti-TB treatment was appropriate in duration and type.
   iii. Treatment for latent TB per local guidelines or at minimum one of the following antimicrobial regimens (whichever is longer) which has not yet been completed.
      INH 300 mg daily for 6 months
      Rifampicin 600 mg daily for 4 months
      INH 300 mg daily+Rifampicin 600 mg daily for 3 months
      Directly-observed rifapentine 900 mg plus INH 900 mg weekly for 3 months
b) Subjects with any acute infection within the last 60 days prior to screening that required hospitalization or treatment with parenteral antibiotics. Subjects with any acute infection within the last 30 days prior to randomization that required oral antimicrobial therapy. Subjects with active infection at the time of randomization will be excluded.
c) Subjects with history of chronic or recurrent bacterial infection (such as chronic pyelonephritis, osteomyelitis and bronchiectasis etc.)
d) Subjects who have a history of systemic fungal infections (such as histoplasmosis, blastomycosis, or coccidiomycosis etc.).
e) Subjects with history of recurrent herpes zoster (more than one episode) or recurrent herpes simplex (more than 2 episodes per year) outbreaks or disseminated (more than one dermatome) herpes zoster or disseminated herpes simplex will be excluded. Symptoms of herpes zoster or herpes simplex must have resolved more than 60 days prior to randomization.
f) Subjects with history of Human Immunodeficiency Virus (HIV) infection or who test positive for HIV at screening.
g) Subjects with history of primary or secondary immunodeficiency or a family history of a primary immune deficiency in a first degree relative.
h) Subjects with autoimmune disease other than RA (eg, SLE, multiple sclerosis [MS], vasculitis, seronegative spondyloarthritis, Inflammatory Bowel Disease etc.). However, patients with secondary Sjogren's syndrome will be allowed. Subjects with active fibomyalgia will be excluded.
i) Prior history of or current inflammatory joint disease other than RA (eg, gout, reactive arthritis, Lyme's disease etc.).
j) Subjects who are not appropriate candidates for treatment with adalimumab based on approved local label.
k) Current symptoms of severe, progressive, or uncontrolled renal, hepatic, hematological, gastrointestinal, pulmonary, cardiovascular, neurological, endocrine, metabolic, cutaneous, or psychiatric disease, or any medical conditions that, in the opinion of the investigator, might place the subject at unacceptable risk for participation in this study.
l) Subjects who have a history of known diverticulitis, perforated diverticular diseases, or small bowel and/or upper GI perforation.
m) Subjects who have class 3 or 4 congestive heart failure.
n) Subjects who have a history of any demyelinating disease
o) Subjects who have received a vaccination with a live vaccine in the 6 weeks before randomization. Subjects who are in close contact with other people who have received a live vaccine may be enrolled at the investigator's discretion.
p) Have present or previous malignancies, except documented history of cured non metastatic squamous or basal skin cell carcinoma, or cervical carcinoma in situ, with no recurrence in the 5 years prior to randomization
q) Subjects who have undergone a major surgical procedure within the 60 days prior to randomization.
r) Subjects with history of surgery on more than 5 joints.
s) Subjects with a history of (within 12 months of signing the consent), or known current problems with drug or alcohol abuse or known cirrhosis including alcoholic cirrhosis. For all subjects, alcohol should not be consumed within 72 hours prior to study related lab testing, and subjects should limit their alcohol intake to 4 drink s per week.
t) Subjects with a history or suspicion of unreliability, poor cooperation, or non compliance with medical treatment.

### 3. Physical and Laboratory Test Findings

a) Subjects with positive Hepatitis B surface antigen (HBsAg). If required by local health authorities or medical society guidelines, subjects at high risk for HBV infection (including subjects with known family history of HBV infection, latent HBV or HBV carrier, Hepatitis B surface antibody (HBsAb), personal medical history of hepatitis or blood transfusion history) must also be tested for quantitative HBV DNA. Subjects with a positive Hepatitis B core antibody (HBcAb) must also be tested for quantitative HBV DNA. Subjects with positive HBsAg or HBV DNA are excluded from the study.
b) Hepatitis C antibody-positive subjects who are HCV positive by confirmatory testing, such as by PCR.
c) Have any clinically significant laboratory abnormalities including but not limited to:
   i. Hepatic
      ALT 1.5×upper limit of normal (ULN)
      AST 1.5×ULN
      Total bilirubin 1.5×ULN.
   ii. Hematologic
      Hemoglobin <9 g/dL
      Absolute neutrophil count <1,000/mm3 (1.0×109/L)
      Platelets <100,000/mm3 (100×109/L)
d) (1 to 3)—β-D glucan positive subjects (only required when, locally, considered the standard of care)

### 4. Allergies and Adverse Drug Reaction

Subjects who have a known clinically significant allergy or hypersensitivity to any biologic therapy.

### 5. Prohibited Therapies

a) Subjects who have used the following conventional synthetic DMARDs less than 4 weeks prior to randomization
   i. chloroquine
   ii. hydroxychloroquine
   iii. quinacrine
   iv. d-penicillamine
   v. azathioprine
   vi. cyclosporine
   vii. cyclophosphamide
   viii. nimesulide
   ix. tofacitinib
   x. Immunoabsorption (ie, Prosorba) column or cholestyramine
b) Subjects who have used the following conventional synthetic DMARDs less than 8 weeks prior to randomization
   i. Oral or parenteral gold
   ii. leflunimide
c) Subjects who are undergoing physical therapy should be on a stable regimen of treatments for 4 weeks prior to screening.
d) Subjects treated with any biologic DMARD including, but not limited to: TNF inhibitors, abatacept, tocilizumab, rituxumab, and investigational biologic therapy.
e) Subjects treated with IM, IV, or IA corticosteroids less than 28 days prior to signing informed consent.
f) Subjects treated with a non-biologic investigational drug within 28 days of signing informed consent, or less than 5 terminal half lives of its elimination (whichever is longer).
g) Subjects who are receiving calcineurin inhibitors at the time of signing informed consent.
h) Subjects who are receiving nimesulide at the time of signing informed consent. 6. Other Exclusion Criteria
   a) Prisoners or subjects who are involuntarily incarcerated
b) Subjects who are compulsorily detained for treatment of either a psychiatric or physical (eg, infectious disease) illness

### Corticosteroids for Treatment of RA Symptoms

All subjects must continue to receive oral prednisone (<10 mg/day), or its equivalent, at the dose being administered at the time of signing the informed consent. Intra-vascular (IV), intra-articular (IA), and intramuscular (IM) corticosteroid injections are not permitted during the double-blind period.

### Analgesics and NSAIDs

NSAIDs and analgesics (including topical NSAIDs) are not permitted within 12 hours before a joint evaluation.
NSAIDS doses should remain stable with the exception of decreases being permitted due to related adverse events, such as gastric toxicity.
Analgesics
   Acetaminophen (paracetamol) maximal dose 2 g/day with no daily dose exceeding 2.5 g.
   NOTE: combination products including acetaminophen and narcotic analgesics (eg, acetaminophen with codeine phosphate, acetaminophen with propoxyphene napsylate, acetaminophen with oxycodone HCl, acetaminophen with hydrocodone bitartrate, etc.) are allowed provided the acetaminophen component dosage is accounted for in the maximum of 2 g/day.
   Narcotic analgesics must not exceed 30 mg/day of morphine or its equivalent and are not permitted within 12 hours before a joint evaluation.
   Tramadol, gabapentin, and pregabalin are allowed but doses must be stable throughout the double-blind period.
   Acetylsalicylic acid is allowed in low doses (eg, 100 mg/day) for cardiovascular prophylaxis
      Herbal medications and Dietary Supplements

The subject anti-IL-6 antibody optionally may be used in combination with specific herbal medications or supplements. However, the following herbal medications and dietary supplements that have potential hepatotoxic effects and ideally but not necessarily should be avoided:
Ba Jiao Lian
Cascara
Chaparral
Chi R Yun
Comfrey
Ephedra
Flavocoxid
Germander
Greater Celandine
Green Tea extracts
Jin Bu Huan
Kava Kava
Margosa Oil
Ma Huang
Pennyroyal Oil
Senna (high dose or long term use)
Sho Saiko To and Dai Saiko To
Shou Wu Pian
Usnic Acid

### Prohibited Treatments

### Prohibited Treatments During Double-Blind Period

Conventional synthetic DMARDs other than methotrexate (including but not limited to sulfasalazine, hydroxychloroquine, chloroquine)
adrenal corticotropic hormone (ACTH)
chloroquine
hydroxychloroquine
oral or parenteral gold
quinacrine
d-penicillamine
leflunomide
azathioprine
cyclosporine
nimesulide
All investigational and approved biologic RA therapies other than clazakizumab (including but not limited to abatacept, tocilizumab, etanercept, anakinra, infliximab, rituximab, etc)
Use of any investigational drug other than study medication
Intra-articular injections of hyaluronic acid
Immunoabsorption (ie, Prosorba) column or cholestyramine

### Prohibited Treatments During Long Term Extension (LTE)

Prohibited treatments during the LTE are the same as during Double-Blind Period except for the following:
Sulfasalazine at labeled doses for rheumatoid arthritis is permitted during this period
Intra-articular injections of hyaluronic acid may be given during this period however must be limited to 1 or 2 joints, and are permitted one time every six months. Intra-articular steroid injections and intra-articular hyaluronic acid injections must not occur within the same 6 month period.

### Other Restrictions and Precautions

The prescribing label of all concomitant medications used as subject's background therapy should be evaluated by the investigator for continued administration during the subject's participation in this study (eg, known toxicities, drug-drug interactions).

Discontinuation of Subjects from Treatment:

Subjects MUST discontinue investigational product (and non-investigational product at the discretion of the investigator) for any of the following reasons:
Use of prohibited medication
Pregnancy
Missed Doses
   Missed more than one dose of investigational product for any reason during the first 12 weeks of the double-blind period
   Missed more than two consecutive doses of investigational product for any reason after the first 12 weeks of the double-blind period
Any clinical adverse event (AE), laboratory abnormality or intercurrent illness which, in the opinion of the investigator, indicates that continued participation in the study is not in the best interest of the subject (eg, significant LFT abnormalities, GI perforation etc.)
Severe liver enzyme elevations.
Positive testing for TB.
Subject's request to stop study treatment or withdrawal of informed consent
Unblinding a subject for any reason (emergency or non-emergency)
Loss of ability to freely provide consent through imprisonment or involuntarily incarceration for treatment of either a psychiatric or physical (eg, infectious disease) illness

If study treatment is discontinued prior to the subject's completion of the study, the reason for the discontinuation must be documented in the subject's medical records and entered on the appropriate case report form (CRF) page.

### Withdrawal of Consent

Subjects who request to discontinue study treatment will remain in the study and must continue to be followed for protocol specified follow-up procedures. The only exception to this is when a subject specifically withdraws consent for any further contact with him/her or persons previously authorized by subject to provide this information. Subjects should notify the investigator of the decision to withdraw consent from future follow-up in writing, whenever possible. The withdrawal of consent should be explained in detail in the medical records by the investigator, as to whether the withdrawal is from further treatment with study drug only or also from study procedures and/or post treatment study follow-up, and entered on the appropriate CRF page. In the event that vital status (whether the subject is alive or dead) is being measured, publicly available information should be used to determine vital status only as appropriately directed in accordance with local law.

### Treatments:

A pharmaceutical form of an active substance or placebo is used as a reference in a clinical study, including products already with a marketing authorization but used or assembled (formulated or packaged) in a way different from the authorized form, or used for an unauthorized indication, or when used to gain further information about the authorized form.

In this protocol, investigational product(s) is/are:
Clazakizumab SC injection, 25 mg/syringe (25 mg/ml)
Clazakizumab Placebo, SC injection, 0.9% Sodium chloride
Adalimumab (Humira®) SC injection, 40 mg/pre-filled syringe (40 mg/0.8 ml)
Adalimumab Placebo, SC injection pre-filled syringe, 0.9% Sodium chloride, 0.8 ml/syringe
Methotrexate, 2.5 mg tablet

A “double-dummy” design is used to protect the blind during the double-blind period. Depending on randomization assignment, subjects will receive the following regimens: Weeks 0-11:
Treatment Arm 1: one SC injection of clazakizumab every 4 weeks PLUS one SC injection of adalimumab placebo every 2 weeks
Treatment Arm 2: one SC injection of clazakizumab placebo (D5W) every 4 weeks PLUS one SC injection of adalimumab every 2 weeks
Treatment Arm 3: one SC injection of clazakizumab placebo (D5W) every 4 weeks PLUS one SC injection of adalimumab placebo every 2 weeks
   Weeks 12-24 (including week 24 visit):
Treatment Arm 1: one SC injection of clazakizumab every 4 weeks PLUS one SC injection of adalimumab placebo every 2 weeks
Treatment Arm 2: one SC injection of clazakizumab every 4 weeks PLUS one SC injection of adalimumab every 2 weeks
Treatment Arm 3: one SC injection of clazakizumab placebo (D5W) every 4 weeks PLUS one SC injection of adalimumab placebo every 2 weeks

### Week 28 (LTE Week 4):

Treatment Arm 1: one SC injection of clazakizumab
Treatment Arm 2: one SC injection of clazakizumab
Treatment Arm 3: one SC injection of clazakizumab placebo (D5W)
   Week 32 (LTE Week 8) and duration of LTE (open-label clazakizumab):
Treatment Arm 1: one SC injection of clazakizumab
Treatment Arm 2: one SC injection of clazakizumab
Treatment Arm 3: one SC injection of clazakizumab

Injections sites may include the upper arms, thigh, or abdomen. It is recommended that no more than one injection should occur per injection site at a given visit. At each visit, corresponding sites on both sides should be used. When possible, injection sites should be rotated between visits. MTX is maintained at the same dose as at randomization (rounded to the nearest 2.5 mg increment).

### Outcomes Research Assessments

During and after treatment pain physical functioning, disease, fatigue assessments will be performed. Based on the results obtained in RA and PsA patients to date these clinical trials will provide further evidence that the subject anti-IL-6 antibodies are safe and effective at low dosages for treating rheumatoid arthritis as well as psoriatic arthritis and moreover provide for greater patient remission and at lower dosages administered less frequently than current biologics used to treat RA and that these low dosages are further effective in RA patients resistant to DMARDS such as e.g., MTX.

Also, these clinical trials will provide further evidence that the subject anti-IL-6 antibodies are safe and effective at low dosages and that they may be self-administered such as by the use of an auto-injector pen thus simplifying patient compliance and better allowing rheumatoid or psoriatic arthritis patients to manage their disease.

Although the subject technology has been described in some detail by way of illustration and example for purposes of clarity of understanding, it will be obvious that certain changes and modifications will practiced within the scope of the appended claims. Modifications of the above-described modes for carrying out the subject technology that are obvious to persons of skill in medicine, pharmacology, microbiology, and/or related fields are intended to be within the scope of the following claims.

All publications (e.g., Non-Patent Literature), patent application publications, and patent applications mentioned in this specification are indicative of the level of skill of those skilled in the art to which this subject technology pertains. All such publications (e.g., Non-Patent Literature), patent application publications, and patent applications are herein incorporated by reference to the same extent as if each individual publication, patent, patent application publication, or patent application is specifically and individually indicated to be incorporated by reference.

1. A method for treating or preventing rheumatoid arthritis, or managing one or more of the symptoms of rheumatoid arthritis comprising administration of a composition comprising an effective amount of an anti-IL-6 antibody or antibody fragment thereof to a subject in need thereof, wherein the anti-IL-6 antibody or antibody fragment thereof comprises a variable light (V_{L}) chain polypeptide comprising a CDR1 sequence of SEQ ID NO:4, a CDR2 sequence of SEQ ID NO:5, and a CDR3 sequence of SEQ ID NO:6, and a variable heavy (V_{H}) chain polypeptide comprising a CDR1 sequence of SEQ ID NO:7, a CDR2 sequence of SEQ ID NOs:8 or 120, and a CDR3 sequence of SEQ ID NO:9, and wherein said anti-IL-6 antibody or antibody fragment thereof is administered every 4 weeks or monthly at a dosage of at most 1, 5, 10, 15, 20 or 25 mg.

2. The method for treating or preventing rheumatoid arthritis or managing one or more of the symptoms of rheumatoid arthritis of claim 15, comprising administration of a composition comprising an effective amount of an anti-IL-6 antibody or antibody fragment thereof to a subject in need thereof, wherein the anti-IL-6 antibody or antibody fragment thereof comprises a variable light (V_{L}) chain polypeptide comprising the amino acid sequence in SEQ ID NO:20, 702 or 709, and a variable heavy (V_{H}) chain polypeptide comprising the amino acid sequence in SEQ ID NO:18, 19, 657 or 704 and wherein said anti-IL-6 antibody or antibody fragment thereof is administered every 4 weeks or monthly at a dosage of at most 1, 5, 10, 15, 20 or 25 mg.

3. The method for treating or preventing rheumatoid arthritis or managing one or more of the symptoms of rheumatoid arthritis of claim 1, comprising administration of a composition comprising an effective amount of an anti-IL-6 antibody or antibody fragment thereof to a subject in need thereof, wherein the anti-IL-6 antibody or antibody fragment thereof comprises a variable light (V_{L}) chain polypeptide comprising the amino acid sequence in SEQ ID NO:20 or 709, and a variable heavy (V_{H}) chain polypeptide comprising the amino acid sequence in SEQ ID NO:18, 19, or 657, and wherein said anti-IL-6 antibody or antibody fragment thereof is administered every 4 weeks or monthly at a dosage of at most 1, 5, 10, 15, 20 or 25 mg.

4. The method for treating or preventing rheumatoid arthritis or managing one or more of the symptoms of rheumatoid arthritis of claim 1, comprising administration of a composition comprising an effective amount of an anti-IL-6 antibody or antibody fragment thereof to a subject in need thereof, wherein the anti-IL-6 antibody or antibody fragment thereof comprises a light chain polypeptide comprising the polypeptide having the amino acid sequence in SEQ ID NO:702 and a heavy chain comprising the polypeptide having the amino acid sequence of SEQ ID NO:704, and wherein said anti-IL-6 antibody or antibody fragment thereof is administered every 4 weeks or monthly at a dosage of at most 1, 5, 10, 15, 20 or 25 mg.

5-35. (canceled)

36. The method of claim 1, wherein said subject has had an inadequate response to non-steroidal anti-inflammatory drugs (NSAIDs).

37. The method of claim 1, wherein said subject has had an inadequate response to non-biologic Disease Modifying Anti-Rheumatic Drugs (DMARDs).

42. A dosage composition, or syringe or injector pen containing a single dosage of an anti-IL-6 antibody or antibody fragment which is for use in treating or preventing rheumatoid arthritis according to any of the foregoing claims, and wherein said anti-IL-6 antibody or antibody fragment comprises or consists of CDRs, variable heavy or light polypeptides or light and heavy polypeptides having the amino acid sequences as set forth in claim 1, wherein the single dosage of said anti-IL-6 antibody or antibody fragment contained in said composition or syringe or injector pen containing same comprises at most or consists of 1, 5, 10, 15, 20, or 25 mg of said anti-IL-6 antibody or antibody fragment.

44-47. (canceled)

48. A therapeutic regimen for treating or preventing psoriatic and/or rheumatoid arthritis or managing the side effects of psoriatic and/or rheumatoid arthritis in a subject in need thereof, wherein the therapeutic regimen comprises or consists of administering a single dosage of an anti-IL-6 antibody or antibody fragment every 4 weeks or monthly using a syringe or injector pen which single dosage comprises at most or consists of 1, 5, 10, 15, 20, or 25 mg of an anti-IL-6 antibody or antibody fragment according to claim 1.

49. The regimen of claim 48, wherein the anti-IL-6 antibody or antibody fragment comprises the V_{L} polypeptide of SEQ ID NO: 20 or 709 and V_{H} polypeptides having the amino acid sequence of SEQ ID NO: 18, 19 or 657.

49-53. (canceled)

54. The method or regimen of claim 1 or 48, which further includes the administration of methotrexate.

55. The method or regimen of claim 1 or 48 wherein the treated subject has developed a resistance or tolerance to methotrexate.

56. The method or regimen of claim 1 or 48, wherein the treated subject has previously received methotrexate.

57. The method or regimen of any of the foregoing claims, claim 1 or 48, wherein the treated subject has previously received another anti-IL-6 antagonist or an anti-TNF biologic.

58. The method or regimen of any of the foregoing claims, wherein the treated subject has previously received Humira®, Remicade®, or Actemra®.

59-65. (canceled)

67. An improved therapeutic regimen for treating rheumatoid arthritis (“RA”) using an anti-IL-6 antibody, wherein the anti-IL-6 antibody comprises the light chain polypeptide of SEQ ID NO:702 and the heavy chain polypeptide of SEQ ID NO:704, and the anti-IL-6 antibody is administered weekly, every 4 weeks or monthly at a dosage of at most 1-5 mg or is administered weekly, every 4 weeks or or monthly at a dosage of at most at most 5-25 mg, and wherein such regimen provides for greater patient remission at lower dosages and/or less frequent dosing than current biologics used to treat RA.

68. The therapeutic regimen of claim 67, which is used to treat an RA in a patient resistant to methotrexate (“MTX”) or to another biologic.

69. The improved therapeutic regimen of claim 67, wherein the other biologic is Humira®, Remicade® or Actemera®.

70. (canceled)


SEQUENCE LISTING (748 sequences)

SEQ ID NO: 1 - protein, 183 aa, Artificial
  Synthetic Polypeptide
VPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 2 - protein, 163 aa, Artificial
  Synthetic Polypeptide
MDTRAPTQLLGLLLLWLPGARCAYDMTQTPASVSAAVGGTVTIKCQASQSINNELSWYQQKPGQRPKLLIYRASTLASGVSSRFKGSGSGTEFTLTISDLECADAATYYCQQGYSLRNIDNAFGGGTEVVVKRTVAAPSVFIFPPSDEQLKSGTASVVCLLNN

SEQ ID NO: 3 - protein, 166 aa, Artificial
  Synthetic Polypeptide
METGLRWLLLVAVLKGVQCQSLEESGGRLVTPGTPLTLTCTASGFSLSNYYVTWVRQAPGKGLEWIGIIYGSDETAYATWAIGRFTISKTSTTVDLKMTSLTAADTATYFCARDDSSDWDAKFNLWGQGTLVTVSSASTKGPSVFPLAPSSKSTSGGTAALGCLVK

SEQ ID NO: 4 - protein, 11 aa, Artificial
  Synthetic Peptide
QASQSINNELS

SEQ ID NO: 5 - protein, 7 aa, Artificial
  Synthetic Peptide
RASTLAS

SEQ ID NO: 6 - protein, 12 aa, Artificial
  Synthetic Peptide
QQGYSLRNIDNA

SEQ ID NO: 7 - protein, 5 aa, Artificial
  Synthetic Peptide
NYYVT

SEQ ID NO: 8 - protein, 16 aa, Artificial
  Synthetic Peptide
IIYGSDETAYATWAIG

SEQ ID NO: 9 - protein, 12 aa, Artificial
  Synthetic Peptide
DDSSDWDAKFNL

SEQ ID NO: 10 - DNA, 491 bp, Artificial
  Synthetic Sequence
atggacacgagggcccccactcagctgctggggctcctgctgctctggctcccaggtgccagatgtgcctatgatatgacccagactccagcctcggtgtctgcagctgtgggaggcacagtcaccatcaagtgccaggccagtcagagcattaacaatgaattatcctggtatcagcagaaaccagggcagcgtcccaagctcctgatctatagggcatccactctggcatctggggtctcatcgcggttcaaaggcagtggatctgggacagagttcactctcaccatcagcgacctggagtgtgccgatgctgccacttactactgtcaacagggttatagtctgaggaatattgataatgctttcggcggagggaccgaggtggtggtcaaacgtacggtagcggccccatctgtcttcatcttcccgccatctgatgagcagttgaaatctggaactgcctctgttgtgtgcctgctgaataactt

SEQ ID NO: 11 - DNA, 499 bp, Artificial
  Synthetic Sequence
atggagactgggctgcgctggcttctcctggtcgctgtgctcaaaggtgtccagtgtcagtcgctggaggagtccgggggtcgcctggtcacgcctgggacacccctgacactcacctgcacagcctctggattctccctcagtaactactacgtgacctgggtccgccaggctccagggaaggggctggaatggatcggaatcatttatggtagtgatgaaacggcctacgcgacctgggcgataggccgattcaccatctccaaaacctcgaccacggtggatctgaaaatgaccagtctgacagccgcggacacggccacctatttctgtgccagagatgatagtagtgactgggatgcaaaatttaacttgtggggccaaggcaccctggtcaccgtctcgagcgcctccaccaagggcccatcggtcttccccctggcaccctcctccaagagcacctctgggggcacagcggccctgggctgcctggtcaagg

SEQ ID NO: 12 - DNA, 33 bp, Artificial
  Synthetic Sequence
caggccagtcagagcattaacaatgaattatcc

SEQ ID NO: 13 - DNA, 21 bp, Artificial
  Synthetic Sequence
agggcatccactctggcatct

SEQ ID NO: 14 - DNA, 36 bp, Artificial
  Synthetic Sequence
caacagggttatagtctgaggaatattgataatgct

SEQ ID NO: 15 - DNA, 15 bp, Artificial
  Synthetic Sequence
aactactacgtgacc

SEQ ID NO: 16 - DNA, 48 bp, Artificial
  Synthetic Sequence
atcatttatggtagtgatgaaacggcctacgcgacctgggcgataggc

SEQ ID NO: 17 - DNA, 36 bp, Artificial
  Synthetic Sequence
gatgatagtagtgactgggatgcaaaatttaacttg

SEQ ID NO: 18 - protein, 109 aa, Artificial
  Synthetic Polypeptide
EVQLVESGGGLVQPGGSLRLSCAASGFSLSNYYVTWVRQAPGKGLEWVGIIYGSDETAYATWAIGRFTISRDNSKNTLYLQMNSLRAEDTAVYYCARDDSSDWDAKFNL

SEQ ID NO: 19 - protein, 109 aa, Artificial
  Synthetic Polypeptide
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNL

SEQ ID NO: 20 - protein, 99 aa, Artificial
  Synthetic Polypeptide
IQMTQSPSSLSASVGDRVTITCQASQSINNELSWYQQKPGKAPKLLIYRASTLASGVPSRFSGSGSGTDFTLTISSLQPDDFATYYCQQGYSLRNIDNA

SEQ ID NO: 21 - protein, 170 aa, Artificial
  Synthetic Polypeptide
MDTRAPTQLLGLLLLWLPGARCAYDMTQTPASVEVAVGGTVTINCQASETIYSWLSWYQQKPGQPPKLLIYQASDLASGVPSRFSGSGAGTEYTLTISGVQCDDAATYYCQQGYSGSNVDNVFGGGTEVVVKRTVAAPSVFIFPPSDEQLKSGTASVVCLLNNFYPREAK

SEQ ID NO: 22 - protein, 167 aa, Artificial
  Synthetic Polypeptide
METGLRWLLLVAVLKGVQCQEQLKESGGRLVTPGTPLTLTCTASGFSLNDHAMGWVRQAPGKGLEYIGFINSGGSARYASWAEGRFTISRTSTTVDLKMTSLTTEDTATYFCVRGGAVWSIHSFDPWGPGTLVTVSSASTKGPSVFPLAPSSKSTSGGTAALGCLVK

SEQ ID NO: 23 - protein, 11 aa, Artificial
  Synthetic Peptide
QASETIYSWLS

SEQ ID NO: 24 - protein, 7 aa, Artificial
  Synthetic Peptide
QASDLAS

SEQ ID NO: 25 - protein, 12 aa, Artificial
  Synthetic Peptide
QQGYSGSNVDNV

SEQ ID NO: 26 - protein, 5 aa, Artificial
  Synthetic Peptide
DHAMG

SEQ ID NO: 27 - protein, 16 aa, Artificial
  Synthetic Peptide
FINSGGSARYASWAEG

SEQ ID NO: 28 - protein, 12 aa, Artificial
  Synthetic Peptide
GGAVWSIHSFDP

SEQ ID NO: 29 - DNA, 511 bp, Artificial
  Synthetic Sequence
atggacacgagggcccccactcagctgctggggctcctgctgctctggctcccaggtgccagatgtgcctatgatatgacccagactccagcctctgtggaggtagctgtgggaggcacagtcaccatcaattgccaggccagtgagaccatttacagttggttatcctggtatcagcagaagccagggcagcctcccaagctcctgatctaccaggcatccgatctggcatctggggtcccatcgcgattcagcggcagtggggctgggacagagtacactctcaccatcagcggcgtgcagtgtgacgatgctgccacttactactgtcaacagggttatagtggtagtaatgttgataatgttttcggcggagggaccgaggtggtggtcaaacgtacggtagcggccccatctgtcttcatcttcccgccatctgatgagcagttgaaatctggaactgcctctgttgtgtgcctgctgaataacttctatcccagagaggccaaag

SEQ ID NO: 30 - DNA, 501 bp, Artificial
  Synthetic Sequence
atggagactgggctgcgctggcttctcctggtcgctgtgctcaaaggtgtccagtgtcaggagcagctgaaggagtccgggggtcgcctggtcacgcctgggacacccctgacacttacctgcacagcctctggattctccctcaatgaccatgcaatgggctgggtccgccaggctccagggaaggggctggaatacatcggattcattaatagtggtggtagcgcacgctacgcgagctgggcagaaggccgattcaccatctccagaacctcgaccacggtggatctgaaaatgaccagtctgacaaccgaggacacggccacctatttctgtgtcagagggggtgctgtttggagtattcatagttttgatccctggggcccagggaccctggtcaccgtctcgagcgcctccaccaagggcccatcggtcttccccctggcaccctcctccaagagcacctctgggggcacagcggccctgggctgcctggtcaag

SEQ ID NO: 31 - DNA, 33 bp, Artificial
  Synthetic Sequence
caggccagtgagaccatttacagttggttatcc

SEQ ID NO: 32 - DNA, 21 bp, Artificial
  Synthetic Sequence
caggcatccgatctggcatct

SEQ ID NO: 33 - DNA, 36 bp, Artificial
  Synthetic Sequence
caacagggttatagtggtagtaatgttgataatgtt

SEQ ID NO: 34 - DNA, 15 bp, Artificial
  Synthetic Sequence
gaccatgcaatgggc

SEQ ID NO: 35 - DNA, 48 bp, Artificial
  Synthetic Sequence
ttcattaatagtggtggtagcgcacgctacgcgagctgggcagaaggc

SEQ ID NO: 36 - DNA, 36 bp, Artificial
  Synthetic Sequence
gggggtgctgtttggagtattcatagttttgatccc

SEQ ID NO: 37 - protein, 165 aa, Artificial
  Synthetic Polypeptide
MDTRAPTQLLGLLLLWLPGATFAAVLTQTPSPVSAAVGGTVSISCQASQSVYDNNYLSWFQQKPGQPPKLLIYGASTLASGVPSRFVGSGSGTQFTLTITDVQCDDAATYYCAGVYDDDSDNAFGGGTEVVVKRTVAAPSVFIFPPSDEQLKSGTASVVCLLNNF

SEQ ID NO: 38 - protein, 166 aa, Artificial
  Synthetic Polypeptide
METGLRWLLLVAVLKGVQCQSLEESGGRLVTPGTPLTLTCTASGFSLSVYYMNWVRQAPGKGLEWIGFITMSDNINYASWAKGRFTISKTSTTVDLKMTSPTTEDTATYFCARSRGWGTMGRLDLWGPGTLVTVSSASTKGPSVFPLAPSSKSTSGGTAALGCLVK

SEQ ID NO: 39 - protein, 13 aa, Artificial
  Synthetic Peptide
QASQSVYDNNYLS

SEQ ID NO: 40 - protein, 7 aa, Artificial
  Synthetic Peptide
GASTLAS

SEQ ID NO: 41 - protein, 11 aa, Artificial
  Synthetic Peptide
AGVYDDDSDNA

SEQ ID NO: 42 - protein, 5 aa, Artificial
  Synthetic Peptide
VYYMN

SEQ ID NO: 43 - protein, 16 aa, Artificial
  Synthetic Peptide
FITMSDNINYASWAKG

SEQ ID NO: 44 - protein, 12 aa, Artificial
  Synthetic Peptide
SRGWGTMGRLDL

SEQ ID NO: 45 - DNA, 496 bp, Artificial
  Synthetic Sequence
atggacacgagggcccccactcagctgctggggctcctgctgctctggctcccaggtgccacatttgccgccgtgctgacccagactccatctcccgtgtctgcagctgtgggaggcacagtcagcatcagttgccaggccagtcagagtgtttatgacaacaactacttatcctggtttcagcagaaaccagggcagcctcccaagctcctgatctatggtgcatccactctggcatctggggtcccatcgcggttcgtgggcagtggatctgggacacagttcactctcaccatcacagacgtgcagtgtgacgatgctgccacttactattgtgcaggcgtttatgatgatgatagtgataatgccttcggcggagggaccgaggtggtggtcaaacgtacggtagcggccccatctgtcttcatcttcccgccatctgatgagcagttgaaatctggaactgcctctgttgtgtgcctgctgaataacttct

SEQ ID NO: 46 - DNA, 499 bp, Artificial
  Synthetic Sequence
atggagactgggctgcgctggcttctcctggtggctgtgctcaaaggtgtccagtgtcagtcgctggaggagtccgggggtcgcctggtcacccctgggacacccctgacactcacctgcacagcctctggattctccctcagtgtctactacatgaactgggtccgccaggctccagggaaggggctggaatggatcggattcattacaatgagtgataatataaattacgcgagctgggcgaaaggccgattcaccatctccaaaacctcgaccacggtggatctgaaaatgaccagtccgacaaccgaggacacggccacctatttctgtgccaggagtcgtggctggggtacaatgggtcggttggatctctggggcccaggcaccctcgtcaccgtctcgagcgcctccaccaagggcccatcggtcttccccctggcaccctcctccaagagcacctctgggggcacagcggccctgggctgcctggtcaagg

SEQ ID NO: 47 - DNA, 39 bp, Artificial
  Synthetic Sequence
caggccagtcagagtgtttatgacaacaactacttatcc

SEQ ID NO: 48 - DNA, 21 bp, Artificial
  Synthetic Sequence
ggtgcatccactctggcatct

SEQ ID NO: 49 - DNA, 33 bp, Artificial
  Synthetic Sequence
gcaggcgtttatgatgatgatagtgataatgcc

SEQ ID NO: 50 - DNA, 15 bp, Artificial
  Synthetic Sequence
gtctactacatgaac

SEQ ID NO: 51 - DNA, 48 bp, Artificial
  Synthetic Sequence
ttcattacaatgagtgataatataaattacgcgagctgggcgaaaggc

SEQ ID NO: 52 - DNA, 36 bp, Artificial
  Synthetic Sequence
agtcgtggctggggtacaatgggtcggttggatctc

SEQ ID NO: 53 - protein, 164 aa, Artificial
  Synthetic Polypeptide
MDTRAPTQLLGLLLLWLPGAICDPVLTQTPSPVSAPVGGTVSISCQASQSVYENNYLSWFQQKPGQPPKLLIYGASTLDSGVPSRFKGSGSGTQFTLTITDVQCDDAATYYCAGVYDDDSDDAFGGGTEVVVKRTVAAPSVFIFPPSDEQLKSGTASVVCLLNN

SEQ ID NO: 54 - protein, 167 aa, Artificial
  Synthetic Polypeptide
METGLRWLLLVAVLKGVQCQEQLKESGGGLVTPGGTLTLTCTASGFSLNAYYMNWVRQAPGKGLEWIGFITLNNNVAYANWAKGRFTFSKTSTTVDLKMTSPTPEDTATYFCARSRGWGAMGRLDLWGHGTLVTVSSASTKGPSVFPLAPSSKSTSGGTAALGCLVK

SEQ ID NO: 55 - protein, 13 aa, Artificial
  Synthetic Peptide
QASQSVYENNYLS

SEQ ID NO: 56 - protein, 7 aa, Artificial
  Synthetic Peptide
GASTLDS

SEQ ID NO: 57 - protein, 11 aa, Artificial
  Synthetic Peptide
AGVYDDDSDDA

SEQ ID NO: 58 - protein, 5 aa, Artificial
  Synthetic Peptide
AYYMN

SEQ ID NO: 59 - protein, 16 aa, Artificial
  Synthetic Peptide
FITLNNNVAYANWAKG

SEQ ID NO: 60 - protein, 12 aa, Artificial
  Synthetic Peptide
SRGWGAMGRLDL

SEQ ID NO: 61 - DNA, 494 bp, Artificial
  Synthetic Sequence
atggacacgagggcccccactcagctgctggggctcctgctgctctggctcccaggtgccatatgtgaccctgtgctgacccagactccatctcccgtatctgcacctgtgggaggcacagtcagcatcagttgccaggccagtcagagtgtttatgagaacaactatttatcctggtttcagcagaaaccagggcagcctcccaagctcctgatctatggtgcatccactctggattctggggtcccatcgcggttcaaaggcagtggatctgggacacagttcactctcaccattacagacgtgcagtgtgacgatgctgccacttactattgtgcaggcgtttatgatgatgatagtgatgatgccttcggcggagggaccgaggtggtggtcaaacgtacggtagcggccccatctgtcttcatcttcccgccatctgatgagcagttgaaatctggaactgcctctgttgtgtgcctgctgaataactt

SEQ ID NO: 62 - DNA, 502 bp, Artificial
  Synthetic Sequence
atggagactgggctgcgctggcttctcctggtggctgtgctcaaaggtgtccagtgtcaggagcagctgaaggagtccggaggaggcctggtaacgcctggaggaaccctgacactcacctgcacagcctctggattctccctcaatgcctactacatgaactgggtccgccaggctccagggaaggggctggaatggatcggattcattactctgaataataatgtagcttacgcgaactgggcgaaaggccgattcaccttctccaaaacctcgaccacggtggatctgaaaatgaccagtccgacacccgaggacacggccacctatttctgtgccaggagtcgtggctggggtgcaatgggtcggttggatctctggggccatggcaccctggtcaccgtctcgagcgcctccaccaagggcccatcggtcttccccctggcaccctcctccaagagcacctctgggggcacagcggccctgggctgcctggtcaagg

SEQ ID NO: 63 - DNA, 39 bp, Artificial
  Synthetic Sequence
caggccagtcagagtgtttatgagaacaactatttatcc

SEQ ID NO: 64 - DNA, 21 bp, Artificial
  Synthetic Sequence
ggtgcatccactctggattct

SEQ ID NO: 65 - DNA, 33 bp, Artificial
  Synthetic Sequence
gcaggcgtttatgatgatgatagtgatgatgcc

SEQ ID NO: 66 - DNA, 15 bp, Artificial
  Synthetic Sequence
gcctactacatgaac

SEQ ID NO: 67 - DNA, 48 bp, Artificial
  Synthetic Sequence
ttcattactctgaataataatgtagcttacgcgaactgggcgaaaggc

SEQ ID NO: 68 - DNA, 36 bp, Artificial
  Synthetic Sequence
agtcgtggctggggtgcaatgggtcggttggatctc

SEQ ID NO: 69 - protein, 164 aa, Artificial
  Synthetic Polypeptide
MDTRAPTQLLGLLLLWLPGATFAQVLTQTPSPVSAAVGGTVTINCQASQSVDDNNWLGWYQQKRGQPPKYLIYSASTLASGVPSRFKGSGSGTQFTLTISDLECDDAATYYCAGGFSGNIFAFGGGTEVVVKRTVAAPSVFIFPPSDEQLKSGTASVVCLLNNF

SEQ ID NO: 70 - protein, 164 aa, Artificial
  Synthetic Polypeptide
METGLRWLLLVAVLKGVQCQSVEESGGRLVTPGTPLTLTCTVSGFSLSSYAMSWVRQAPGKGLEWIGIIGGFGTTYYATWAKGRFTISKTSTTVDLRITSPTTEDTATYFCARGGPGNGGDIWGQGTLVTVSSASTKGPSVFPLAPSSKSTSGGTAALGCLVKD

SEQ ID NO: 71 - protein, 13 aa, Artificial
  Synthetic Peptide
QASQSVDDNNWLG

SEQ ID NO: 72 - protein, 7 aa, Artificial
  Synthetic Peptide
SASTLAS

SEQ ID NO: 73 - protein, 10 aa, Artificial
  Synthetic Peptide
AGGFSGNIFA

SEQ ID NO: 74 - protein, 5 aa, Artificial
  Synthetic Peptide
SYAMS

SEQ ID NO: 75 - protein, 16 aa, Artificial
  Synthetic Peptide
IIGGFGTTYYATWAKG

SEQ ID NO: 76 - protein, 9 aa, Artificial
  Synthetic Peptide
GGPGNGGDI

SEQ ID NO: 77 - DNA, 493 bp, Artificial
  Synthetic Sequence
atggacacgagggcccccactcagctgctggggctcctgctgctctggctcccaggtgccacatttgcccaagtgctgacccagactccatcgcctgtgtctgcagctgtgggaggcacagtcaccatcaactgccaggccagtcagagtgttgatgataacaactggttaggctggtatcagcagaaacgagggcagcctcccaagtacctgatctattctgcatccactctggcatctggggtcccatcgcggttcaaaggcagtggatctgggacacagttcactctcaccatcagcgacctggagtgtgacgatgctgccacttactactgtgcaggcggttttagtggtaatatctttgctttcggcggagggaccgaggtggtggtcaaacgtacggtagcggccccatctgtcttcatcttcccgccatctgatgagcagttgaaatctggaactgcctctgttgtgtgcctgctgaataacttct

SEQ ID NO: 78 - DNA, 493 bp, Artificial
  Synthetic Sequence
atggagactgggctgcgctggcttctcctggtcgctgtgctcaaaggtgtccagtgtcagtcggtggaggagtccgggggtcgcctggtcacgcctgggacacccctgacactcacctgcacagtctctggcttctccctcagtagctatgcaatgagctgggtccgccaggctccaggaaaggggctggagtggatcggaatcattggtggttttggtaccacatactacgcgacctgggcgaaaggccgattcaccatctccaaaacctcgaccacggtggatctgagaatcaccagtccgacaaccgaggacacggccacctatttctgtgccagaggtggtcctggtaatggtggtgacatctggggccaagggaccctggtcaccgtctcgagcgcctccaccaagggcccatcggtcttccccctggcaccctcctccaagagcacctctgggggcacagcggccctgggctgcctggtcaaggact

SEQ ID NO: 79 - DNA, 39 bp, Artificial
  Synthetic Sequence
caggccagtcagagtgttgatgataacaactggttaggc

SEQ ID NO: 80 - DNA, 21 bp, Artificial
  Synthetic Sequence
tctgcatccactctggcatct

SEQ ID NO: 81 - DNA, 30 bp, Artificial
  Synthetic Sequence
gcaggcggttttagtggtaatatctttgct

SEQ ID NO: 82 - DNA, 15 bp, Artificial
  Synthetic Sequence
agctatgcaatgagc

SEQ ID NO: 83 - DNA, 48 bp, Artificial
  Synthetic Sequence
atcattggtggttttggtaccacatactacgcgacctgggcgaaaggc

SEQ ID NO: 84 - DNA, 27 bp, Artificial
  Synthetic Sequence
ggtggtcctggtaatggtggtgacatc

SEQ ID NO: 85 - protein, 164 aa, Artificial
  Synthetic Polypeptide
MDTRAPTQLLGLLLLWLPGATFAAVLTQTPSPVSVPVGGTVTIKCQSSQSVYNNFLSWYQQKPGQPPKLLIYQASKLASGVPDRFSGSGSGTQFTLTISGVQCDDAATYYCLGGYDDDADNAFGGGTEVVVKRTVAAPSVFIFPPSDEQLKSGTASVVCLLNNF

SEQ ID NO: 86 - protein, 170 aa, Artificial
  Synthetic Polypeptide
METGLRWLLLVAVLKGVQCQSVEESGGRLVTPGTPLTLTCTVSGIDLSDYAMSWVRQAPGKGLEWIGIIYAGSGSTWYASWAKGRFTISKTSTTVDLKITSPTTEDTATYFCARDGYDDYGDFDRLDLWGPGTLVTVSSASTKGPSVFPLAPSSKSTSGGTAALGCLVKD

SEQ ID NO: 87 - protein, 12 aa, Artificial
  Synthetic Peptide
QSSQSVYNNFLS

SEQ ID NO: 88 - protein, 7 aa, Artificial
  Synthetic Peptide
QASKLAS

SEQ ID NO: 89 - protein, 11 aa, Artificial
  Synthetic Peptide
LGGYDDDADNA

SEQ ID NO: 90 - protein, 5 aa, Artificial
  Synthetic Peptide
DYAMS

SEQ ID NO: 91 - protein, 17 aa, Artificial
  Synthetic Peptide
IIYAGSGSTWYASWAKG

SEQ ID NO: 92 - protein, 14 aa, Artificial
  Synthetic Peptide
DGYDDYGDFDRLDL

SEQ ID NO: 93 - DNA, 492 bp, Artificial
  Synthetic Sequence
atggacacgagggcccccactcagctgctggggctcctgctgctctggctcccaggtgccacatttgcagccgtgctgacccagacaccatcgcccgtgtctgtacctgtgggaggcacagtcaccatcaagtgccagtccagtcagagtgtttataataatttcttatcgtggtatcagcagaaaccagggcagcctcccaagctcctgatctaccaggcatccaaactggcatctggggtcccagataggttcagcggcagtggatctgggacacagttcactctcaccatcagcggcgtgcagtgtgacgatgctgccacttactactgtctaggcggttatgatgatgatgctgataatgctttcggcggagggaccgaggtggtggtcaaacgtacggtagcggccccatctgtcttcatcttcccgccatctgatgagcagttgaaatctggaactgcctctgttgtgtgcctgctgaataacttc

SEQ ID NO: 94 - DNA, 511 bp, Artificial
  Synthetic Sequence
atggagactgggctgcgctggcttctcctggtcgctgtgctcaaaggtgtccagtgtcagtcggtggaggagtccgggggtcgcctggtcacgcctgggacacccctgacgctcacctgcacagtctctggaatcgacctcagtgactatgcaatgagctgggtccgccaggctccagggaaggggctggaatggatcggaatcatttatgctggtagtggtagcacatggtacgcgagctgggcgaaaggccgattcaccatctccaaaacctcgaccacggtggatctgaaaatcaccagtccgacaaccgaggacacggccacctatttctgtgccagagatggatacgatgactatggtgatttcgatcgattggatctctggggcccaggcaccctcgtcaccgtctcgagcgcctccaccaagggcccatcggtcttccccctggcaccctcctccaagagcacctctgggggcacagcggccctgggctgcctggtcaaggact

SEQ ID NO: 95 - DNA, 36 bp, Artificial
  Synthetic Sequence
cagtccagtcagagtgtttataataatttcttatcg

SEQ ID NO: 96 - DNA, 21 bp, Artificial
  Synthetic Sequence
caggcatccaaactggcatct

SEQ ID NO: 97 - DNA, 33 bp, Artificial
  Artificial Sequence
ctaggcggttatgatgatgatgctgataatgct

SEQ ID NO: 98 - DNA, 15 bp, Artificial
  Synthetic Sequence
gactatgcaatgagc

SEQ ID NO: 99 - DNA, 51 bp, Artificial
  Synthetic Sequence
atcatttatgctggtagtggtagcacatggtacgcgagctgggcgaaaggc

SEQ ID NO: 100 - DNA, 42 bp, Artificial
  Synthetic Sequence
gatggatacgatgactatggtgatttcgatcgattggatctc

SEQ ID NO: 101 - protein, 164 aa, Artificial
  Synthetic Polypeptide
MDTRAPTQLLGLLLLWLPGARCAYDMTQTPASVSAAVGGTVTIKCQASQSINNELSWYQQKSGQRPKLLIYRASTLASGVSSRFKGSGSGTEFTLTISDLECADAATYYCQQGYSLRNIDNAFGGGTEVVVKRTVAAPSVFIFPPSDEQLKSGTASVVCLLNNF

SEQ ID NO: 102 - protein, 166 aa, Artificial
  Synthetic Polypeptide
METGLRWLLLVAVLSGVQCQSLEESGGRLVTPGTPLTLTCTASGFSLSNYYMTWVRQAPGKGLEWIGMIYGSDETAYANWAIGRFTISKTSTTVDLKMTSLTAADTATYFCARDDSSDWDAKFNLWGQGTLVTVSSASTKGPSVFPLAPSSKSTSGGTAALGCLVK

SEQ ID NO: 103 - protein, 11 aa, Artificial
  Synthetic Peptide
QASQSINNELS

SEQ ID NO: 104 - protein, 7 aa, Artificial
  Synthetic Peptide
RASTLAS

SEQ ID NO: 105 - protein, 12 aa, Artificial
  Synthetic Peptide
QQGYSLRNIDNA

SEQ ID NO: 106 - protein, 5 aa, Artificial
  Synthetic Peptide
NYYMT

SEQ ID NO: 107 - protein, 16 aa, Artificial
  Synthetic Peptide
MIYGSDETAYANWAIG

SEQ ID NO: 108 - protein, 12 aa, Artificial
  Synthetic Peptide
DDSSDWDAKFNL

SEQ ID NO: 109 - DNA, 492 bp, Artificial
  Synthetic Sequence
atggacacgagggcccccactcagctgctggggctcctgctgctctggctcccaggtgccagatgtgcctatgatatgacccagactccagcctcggtgtctgcagctgtgggaggcacagtcaccatcaaatgccaggccagtcagagcattaacaatgaattatcctggtatcagcagaaatcagggcagcgtcccaagctcctgatctatagggcatccactctggcatctggggtctcatcgcggttcaaaggcagtggatctgggacagagttcactctcaccatcagcgacctggagtgtgccgatgctgccacttactactgtcaacagggttatagtctgaggaatattgataatgctttcggcggagggaccgaggtggtggtcaaacgtacggtagcggccccatctgtcttcatcttcccgccatctgatgagcagttgaaatctggaactgcctctgttgtgtgcctgctgaataacttc

SEQ ID NO: 110 - DNA, 499 bp, Artificial
  Synthetic Sequence
atggagactgggctgcgctggcttctcctggtcgctgtgctctcaggtgtccagtgtcagtcgctggaggagtccgggggtcgcctggtcacgcctgggacacccctgacactcacctgcacagcctctggattctccctcagtaactactacatgacctgggtccgccaggctccagggaaggggctggaatggatcggaatgatttatggtagtgatgaaacagcctacgcgaactgggcgataggccgattcaccatctccaaaacctcgaccacggtggatctgaaaatgaccagtctgacagccgcggacacggccacctatttctgtgccagagatgatagtagtgactgggatgcaaaatttaacttgtggggccaagggaccctcgtcaccgtctcgagcgcctccaccaagggcccatcggtcttccccctggcaccctcctccaagagcacctctgggggcacagcggccctgggctgcctggtcaagg

SEQ ID NO: 111 - DNA, 33 bp, Artificial; identical to SEQ ID NO: 12
  Synthetic Sequence

SEQ ID NO: 112 - DNA, 21 bp, Artificial
  Synthetic Sequence
agggcatccactctggcatct

SEQ ID NO: 113 - DNA, 36 bp, Artificial; identical to SEQ ID NO: 14
  Synthetic Sequence

SEQ ID NO: 114 - DNA, 15 bp, Artificial
  Synthetic Sequence
aactactacatgacc

SEQ ID NO: 115 - DNA, 48 bp, Artificial
  Synthetic Sequence
atgatttatggtagtgatgaaacagcctacgcgaactgggcgataggc

SEQ ID NO: 116 - DNA, 36 bp, Artificial; identical to SEQ ID NO: 17
  Synthetic Sequence

SEQ ID NO: 117 - protein, 109 aa, Artificial
  Synthetic Polypeptide
EVQLVESGGGLVQPGGSLRLSCAASGFSLSNYYMTWVRQAPGKGLEWVGMIYGSDETAYANWAIGRFTISRDNSKNTLYLQMNSLRAEDTAVYYCARDDSSDWDAKFNL

SEQ ID NO: 118 - protein, 109 aa, Artificial
  Synthetic Polypeptide
EVQLVESGGGLVQPGGSLRLSCAASGFSLSNYYMTWVRQAPGKGLEWVGMIYGSDETAYANSAIGRFTISRDNSKNTLYLQMNSLRAEDTAVYYCARDDSSDWDAKFNL

SEQ ID NO: 119 - protein, 100 aa, Artificial
  Synthetic Polypeptide
DIQMTQSPSTLSASVGDRVTITCQASQSINNELSWYQQKPGKAPKLLIYRASTLASGVPSRFSGSGSGTEFTLTISSLQPDDFATYYCQQGYSLRNIDNA

SEQ ID NO: 120 - protein, 16 aa, Artificial
  Synthetic Peptide
IIYGSDETAYATSAIG

SEQ ID NO: 121 - protein, 16 aa, Artificial
  Synthetic Peptide
MIYGSDETAYANSAIG

SEQ ID NO: 122 - protein, 123 aa, Artificial
  Synthetic Polypeptide
MDTRAPTQLLGLLLLWLPGATFAAVLTQTPSPVSAAVGGTVTISCQSSQSVGNNQDLSWFQQRPGQPPKLLIYEISKLESGVPSRFSGSGSGTHFTLTISGVQCDDAATYYCLGGYDDDADNA

SEQ ID NO: 123 - protein, 128 aa, Artificial
  Synthetic Polypeptide
METGLRWLLLVAVLKGVQCHSVEESGGRLVTPGTPLTLTCTVSGFSLSSRTMSWVRQAPGKGLEWIGYIWSGGSTYYATWAKGRFTISKTSTTVDLKITSPTTEDTATYFCARLGDTGGHAYATRLNL

SEQ ID NO: 124 - protein, 13 aa, Artificial
  Synthetic Peptide
QSSQSVGNNQDLS

SEQ ID NO: 125 - protein, 7 aa, Artificial
  Synthetic Peptide
EISKLES

SEQ ID NO: 126 - protein, 11 aa, Artificial
  Synthetic Peptide
LGGYDDDADNA

SEQ ID NO: 127 - protein, 5 aa, Artificial
  Synthetic Peptide
SRTMS

SEQ ID NO: 128 - protein, 16 aa, Artificial
  Synthetic Peptide
YIWSGGSTYYATWAKG

SEQ ID NO: 129 - protein, 15 aa, Artificial
  Synthetic Peptide
LGDTGGHAYATRLNL

SEQ ID NO: 130 - DNA, 369 bp, Artificial
  Synthetic Sequence
atggacacgagggcccccactcagctgctggggctcctgctgctctggctcccaggtgccacatttgcagccgtgctgacccagacaccatcacccgtgtctgcagctgtgggaggcacagtcaccatcagttgccagtccagtcagagtgttggtaataaccaggacttatcctggtttcagcagagaccagggcagcctcccaagctcctgatctacgaaatatccaaactggaatctggggtcccatcgcggttcagcggcagtggatctgggacacacttcactctcaccatcagcggcgtacagtgtgacgatgctgccacttactactgtctaggcggttatgatgatgatgctgataatgct

SEQ ID NO: 131 - DNA, 384 bp, Artificial
  Synthetic Sequence
atggagactgggctgcgctggcttctcctggtcgctgtgctcaaaggtgtccagtgtcactcggtggaggagtccgggggtcgcctggtcacgcctgggacacccctgacactcacctgcacagtctctggattctccctcagtagtcgtacaatgtcctgggtccgccaggctccagggaaggggctggagtggatcggatacatttggagtggtggtagcacatactacgcgacctgggcgaaaggccgattcaccatctccaaaacctcgaccacggtggatctgaaaatcaccagtccgacaaccgaggacacggccacctatttctgtgccagattgggcgatactggtggtcacgcttatgctactcgcttaaatctc

SEQ ID NO: 132 - DNA, 39 bp, Artificial
  Synthetic Sequence
cagtccagtcagagtgttggtaataaccaggacttatcc

SEQ ID NO: 133 - DNA, 21 bp, Artificial
  Synthetic Sequence
gaaatatccaaactggaatct

SEQ ID NO: 134 - DNA, 33 bp, Artificial; identical to SEQ ID NO: 97
  Synthetic Sequence

SEQ ID NO: 135 - DNA, 15 bp, Artificial
  Synthetic Sequence
agtcgtacaatgtcc

SEQ ID NO: 136 - DNA, 48 bp, Artificial
  Synthetic Sequence
tacatttggagtggtggtagcacatactacgcgacctgggcgaaaggc

SEQ ID NO: 137 - DNA, 45 bp, Artificial
  Synthetic Sequence
ttgggcgatactggtggtcacgcttatgctactcgcttaaatctc

SEQ ID NO: 138 - protein, 123 aa, Artificial
  Synthetic Polypeptide
MDTRAPTQLLGLLLLWLPGATFAAVLTQTPSSVSAAVGGTVSISCQSSQSVYSNKYLAWYQQKPGQPPKLLIYWTSKLASGAPSRFSGSGSGTQFTLTISGVQCDDAATYYCLGAYDDDADNA

SEQ ID NO: 139 - protein, 126 aa, Artificial
  Synthetic Polypeptide
METGLRWLLLVAVLKGVQCQSVEESGGRLVKPDETLTLTCTASGFSLEGGYMTWVRQAPGKGLEWIGISYDSGSTYYASWAKGRFTISKTSSTTVDLKMTSLTTEDTATYFCVRSLKYPTVTSDDL

SEQ ID NO: 140 - protein, 13 aa, Artificial
  Synthetic Peptide
QSSQSVYSNKYLA

SEQ ID NO: 141 - protein, 7 aa, Artificial
  Synthetic Peptide
WTSKLAS

SEQ ID NO: 142 - protein, 11 aa, Artificial
  Synthetic Peptide
LGAYDDDADNA

SEQ ID NO: 143 - protein, 5 aa, Artificial
  Synthetic Peptide
GGYMT

SEQ ID NO: 144 - protein, 16 aa, Artificial
  Synthetic Peptide
ISYDSGSTYYASWAKG

SEQ ID NO: 145 - protein, 12 aa, Artificial
  Synthetic Peptide
SLKYPTVTSDDL

SEQ ID NO: 146 - DNA, 369 bp, Artificial
  Synthetic Sequence
atggacacgagggcccccactcagctgctggggctcctgctgctctggctcccaggtgccacatttgcagccgtgctgacccagacaccatcgtccgtgtctgcagctgtgggaggcacagtcagcatcagttgccagtccagtcagagtgtttatagtaataagtacctagcctggtatcagcagaaaccagggcagcctcccaagctcctgatctactggacatccaaactggcatctggggccccatcacggttcagcggcagtggatctgggacacaattcactctcaccatcagcggcgtgcagtgtgacgatgctgccacttactactgtctaggcgcttatgatgatgatgctgataatgct

SEQ ID NO: 147 - DNA, 378 bp, Artificial
  Synthetic Sequence
atggagactgggctgcgctggcttctcctggtcgctgtgctcaaaggtgtccagtgtcagtcggtggaagagtccgggggtcgcctggtcaagcctgacgaaaccctgacactcacctgcacagcctctggattctccctggagggcggctacatgacctgggtccgccaggctccagggaaggggctggaatggatcggaatcagttatgatagtggtagcacatactacgcgagctgggcgaaaggccgattcaccatctccaagacctcgtcgaccacggtggatctgaaaatgaccagtctgacaaccgaggacacggccacctatttctgcgtcagatcactaaaatatcctactgttacttctgatgacttg

SEQ ID NO: 148 - DNA, 39 bp, Artificial
  Synthetic Sequence
cagtccagtcagagtgtttatagtaataagtacctagcc

SEQ ID NO: 149 - DNA, 21 bp, Artificial
  Synthetic Sequence
tggacatccaaactggcatct

SEQ ID NO: 150 - DNA, 33 bp, Artificial
  Synthetic Sequence
ctaggcgcttatgatgatgatgctgataatgct

SEQ ID NO: 151 - DNA, 15 bp, Artificial
  Synthetic Sequence
ggcggctacatgacc

SEQ ID NO: 152 - DNA, 48 bp, Artificial
  Synthetic Sequence
atcagttatgatagtggtagcacatactacgcgagctgggcgaaaggc

SEQ ID NO: 153 - DNA, 36 bp, Artificial
  Synthetic Sequence
tcactaaaatatcctactgttacttctgatgacttg

SEQ ID NO: 154 - protein, 123 aa, Artificial
  Synthetic Polypeptide
MDTRAPTQLLGLLLLWLPGATFAAVLTQTPSPVSAAVGGTVTISCQSSQSVYNNNDLAWYQQKPGQPPKLLIYYASTLASGVPSRFKGSGSGTQFTLTISGVQCDDAAAYYCLGGYDDDADNA

SEQ ID NO: 155 - protein, 129 aa, Artificial
  Synthetic Polypeptide
METGLRWLLLVAVLKGVQCQSVEESGGRLVTPGTPLTLTCTVSGLSLSSNTINWVRQAPGKGLEWIGYIWSGGSTYYASWVNGRFTISKTSTTVDLKITSPTTEDTATYFCARGGYASGGYPYATRLDL

SEQ ID NO: 156 - protein, 13 aa, Artificial
  Synthetic Peptide
QSSQSVYNNNDLA

SEQ ID NO: 157 - protein, 7 aa, Artificial
  Synthetic Peptide
YASTLAS

SEQ ID NO: 158 - protein, 11 aa, Artificial
  Synthetic Peptide
LGGYDDDADNA

SEQ ID NO: 159 - protein, 5 aa, Artificial
  Synthetic Peptide
SNTIN

SEQ ID NO: 160 - protein, 16 aa, Artificial
  Synthetic Peptide
YIWSGGSTYYASWVNG

SEQ ID NO: 161 - protein, 16 aa, Artificial
  Synthetic Peptide
GGYASGGYPYATRLDL

SEQ ID NO: 162 - DNA, 369 bp, Artificial
  Synthetic Sequence
atggacacgagggcccccactcagctgctggggctcctgctgctctggctcccaggtgccacatttgcagccgtgctgacccagacaccatcacccgtgtctgcagctgtgggaggcacagtcaccatcagttgccagtccagtcagagtgtttataataataacgacttagcctggtatcagcagaaaccagggcagcctcctaaactcctgatctattatgcatccactctggcatctggggtcccatcgcggttcaaaggcagtggatctgggacacagttcactctcaccatcagcggcgtgcagtgtgacgatgctgccgcttactactgtctaggcggttatgatgatgatgctgataatgct

SEQ ID NO: 163 - DNA, 387 bp, Artificial
  Synthetic Sequence
atggagactgggctgcgctggcttctcctggtcgctgtgctcaaaggtgtccagtgtcagtcggtggaggagtccgggggtcgcctggtcacgcctgggacacccctgacactcacctgcacagtatctggattatccctcagtagcaatacaataaactgggtccgccaggctccagggaaggggctggagtggatcggatacatttggagtggtggtagtacatactacgcgagctgggtgaatggtcgattcaccatctccaaaacctcgaccacggtggatctgaaaatcaccagtccgacaaccgaggacacggccacctatttctgtgccagagggggttacgctagtggtggttatccttatgccactcggttggatctc

SEQ ID NO: 164 - DNA, 39 bp, Artificial
  Synthetic Sequence
cagtccagtcagagtgtttataataataacgacttagcc

SEQ ID NO: 165 - DNA, 21 bp, Artificial
  Synthetic Sequence
tatgcatccactctggcatct

SEQ ID NO: 166 - DNA, 33 bp, Artificial; identical to SEQ ID NO: 97
  Synthetic Sequence

SEQ ID NO: 167 - DNA, 15 bp, Artificial
  Synthetic Sequence
agcaatacaataaac

SEQ ID NO: 168 - DNA, 48 bp, Artificial
  Synthetic Sequence
tacatttggagtggtggtagtacatactacgcgagctgggtgaatggt

SEQ ID NO: 169 - DNA, 48 bp, Artificial
  Synthetic Sequence
gggggttacgctagtggtggttatccttatgccactcggttggatctc

SEQ ID NO: 170 - protein, 123 aa, Artificial
  Synthetic Polypeptide
MDTRAPTQLLGLLLLWLPGATFAAVLTQTPSSVSAAVGGTVTINCQSSQSVYNNDYLSWYQQRPGQRPKLLIYGASKLASGVPSRFKGSGSGKQFTLTISGVQCDDAATYYCLGDYDDDADNT

SEQ ID NO: 171 - protein, 123 aa, Artificial
  Synthetic Polypeptide
METGLRWLLLVAVLKGVQCQSLEESGGRLVTPGTPLTLTCTVSGFTLSTNYYLSWVRQAPGKGLEWIGIIYPSGNTYCAKWAKGRFTISKTSSTTVDLKMTSPTTEDTATYFCARNYGGDESL

SEQ ID NO: 172 - protein, 13 aa, Artificial
  Synthetic Peptide
QSSQSVYNNDYLS

SEQ ID NO: 173 - protein, 7 aa, Artificial
  Synthetic Peptide
GASKLAS

SEQ ID NO: 174 - protein, 11 aa, Artificial
  Synthetic Peptide
LGDYDDDADNT

SEQ ID NO: 175 - protein, 6 aa, Artificial
  Synthetic Peptide
TNYYLS

SEQ ID NO: 176 - protein, 16 aa, Artificial
  Synthetic Peptide
IIYPSGNTYCAKWAKG

SEQ ID NO: 177 - protein, 8 aa, Artificial
  Synthetic Peptide
NYGGDESL

SEQ ID NO: 178 - DNA, 369 bp, Artificial
  Synthetic Sequence
atggacacgagggcccccactcagctgctggggctcctgctgctctggctcccaggtgccacatttgcagccgtgctgacccagacaccatcctccgtgtctgcagctgtgggaggcacagtcaccatcaattgccagtccagtcagagtgtttataataacgactacttatcctggtatcaacagaggccagggcaacgtcccaagctcctaatctatggtgcttccaaactggcatctggggtcccgtcacggttcaaaggcagtggatctgggaaacagtttactctcaccatcagcggcgtgcagtgtgacgatgctgccacttactactgtctgggcgattatgatgatgatgctgataatact

SEQ ID NO: 179 - DNA, 369 bp, Artificial
  Synthetic Sequence
atggagactgggctgcgctggcttctcctggtcgctgtgctcaaaggtgtccagtgtcagtcgctggaggagtccgggggtcgcctggtcacgcctgggacacccctgacactcacttgcacagtctctggattcaccctcagtaccaactactacctgagctgggtccgccaggctccagggaaggggctagaatggatcggaatcatttatcctagtggtaacacatattgcgcgaagtgggcgaaaggccgattcaccatctccaaaacctcgtcgaccacggtggatctgaaaatgaccagtccgacaaccgaggacacagccacgtatttctgtgccagaaattatggtggtgatgaaagtttg

SEQ ID NO: 180 - DNA, 39 bp, Artificial
  Synthetic Sequence
cagtccagtcagagtgtttataataacgactacttatcc

SEQ ID NO: 181 - DNA, 21 bp, Artificial
  Synthetic Sequence
ggtgcttccaaactggcatct

SEQ ID NO: 182 - DNA, 33 bp, Artificial
  Synthetic Sequence
ctgggcgattatgatgatgatgctgataatact

SEQ ID NO: 183 - DNA, 18 bp, Artificial
  Synthetic Sequence
accaactactacctgagc

SEQ ID NO: 184 - DNA, 48 bp, Artificial
  Synthetic Sequence
atcatttatcctagtggtaacacatattgcgcgaagtgggcgaaaggc

SEQ ID NO: 185 - DNA, 24 bp, Artificial
  Synthetic Sequence
aattatggtggtgatgaaagtttg

SEQ ID NO: 186 - protein, 119 aa, Artificial
  Synthetic Polypeptide
MDTRAPTQLLGLLLLWLPGARCDVVMTQTPASVEAAVGGTVTIKCQASETIGNALAWYQQKSGQPPKLLIYKASKLASGVPSRFKGSGSGTEYTLTISDLECADAATYYCQWCYFGDSV

SEQ ID NO: 187 - protein, 128 aa, Artificial
  Synthetic Polypeptide
METGLRWLLLVTVLKGVQCQEQLVESGGGLVQPEGSLTLTCTASGFDFSSGYYMCWVRQAPGKGLEWIACIFTITTNTYYASWAKGRFTISKTSSTTVTLQMTSLTAADTATYLCARGIYSDNNYYAL

SEQ ID NO: 188 - protein, 11 aa, Artificial
  Synthetic Peptide
QASETIGNALA

SEQ ID NO: 189 - protein, 7 aa, Artificial
  Synthetic Peptide
KASKLAS

SEQ ID NO: 190 - protein, 9 aa, Artificial
  Synthetic Peptide
QWCYFGDSV

SEQ ID NO: 191 - protein, 6 aa, Artificial
  Synthetic Peptide
SGYYMC

SEQ ID NO: 192 - protein, 17 aa, Artificial
  Synthetic Peptide
CIFTITTNTYYASWAKG

SEQ ID NO: 193 - protein, 11 aa, Artificial
  Synthetic Peptide
GIYSDNNYYAL

SEQ ID NO: 194 - DNA, 357 bp, Artificial
  Synthetic Sequence
atggacacgagggcccccactcagctgctggggctcctgctgctctggctcccaggtgccagatgtgatgttgtgatgacccagactccagcctccgtggaggcagctgtgggaggcacagtcaccatcaagtgccaggccagtgagaccattggcaatgcattagcctggtatcagcagaaatcagggcagcctcccaagctcctgatctacaaggcatccaaactggcatctggggtcccatcgcggttcaaaggcagtggatctgggacagagtacactctcaccatcagcgacctggagtgtgccgatgctgccacttactactgtcaatggtgttattttggtgatagtgtt

SEQ ID NO: 195 - DNA, 384 bp, Artificial
  Synthetic Sequence
atggagactgggctgcgctggcttctcctggtcactgtgctcaaaggtgtccagtgtcaggagcagctggtggagtccgggggaggcctggtccagcctgagggatccctgacactcacctgcacagcctctggattcgacttcagtagcggctactacatgtgctgggtccgccaggctccagggaaggggctggagtggatcgcgtgtattttcactattactactaacacttactacgcgagctgggcgaaaggccgattcaccatctccaagacctcgtcgaccacggtgactctgcaaatgaccagtctgacagccgcggacacggccacctatctctgtgcgagagggatttattctgataataattattatgccttg

SEQ ID NO: 196 - DNA, 33 bp, Artificial
  Synthetic Sequence
caggccagtgagaccattggcaatgcattagcc

SEQ ID NO: 197 - DNA, 21 bp, Artificial
  Synthetic Sequence
aaggcatccaaactggcatct

SEQ ID NO: 198 - DNA, 27 bp, Artificial
  Synthetic Sequence
caatggtgttattttggtgatagtgtt

SEQ ID NO: 199 - DNA, 18 bp, Artificial
  Synthetic Sequence
agcggctactacatgtgc

SEQ ID NO: 200 - DNA, 51 bp, Artificial
  Synthetic Sequence
tgtattttcactattactactaacacttactacgcgagctgggcgaaaggc

SEQ ID NO: 201 - DNA, 33 bp, Artificial
  Synthetic Sequence
gggatttattctgataataattattatgccttg

SEQ ID NO: 202 - protein, 119 aa, Artificial
  Synthetic Polypeptide
MDTRAPTQLLGLLLLWLPGARCDVVMTQTPASVEAAVGGTVTIKCQASESIGNALAWYQQKPGQPPKLLIYKASTLASGVPSRFSGSGSGTEFTLTISGVQCADAAAYYCQWCYFGDSV

SEQ ID NO: 203 - protein, 128 aa, Artificial
  Synthetic Polypeptide
METGLRWLLLVAVLKGVQCQQQLVESGGGLVKPGASLTLTCKASGFSFSSGYYMCWVRQAPGKGLESIACIFTITDNTYYANWAKGRFTISKPSSPTVTLQMTSLTAADTATYFCARGIYSTDNYYAL

SEQ ID NO: 204 - protein, 11 aa, Artificial
  Synthetic Peptide
QASESIGNALA

SEQ ID NO: 205 - protein, 7 aa, Artificial
  Synthetic Peptide
KASTLAS

SEQ ID NO: 206 - protein, 9 aa, Artificial
  Synthetic Peptide
QWCYFGDSV

SEQ ID NO: 207 - protein, 6 aa, Artificial
  Synthetic Peptide
SGYYMC

SEQ ID NO: 208 - protein, 17 aa, Artificial
  Synthetic Peptide
CIFTITDNTYYANWAKG

SEQ ID NO: 209 - protein, 11 aa, Artificial
  Synthetic Peptide
GIYSTDNYYAL

SEQ ID NO: 210 - DNA, 357 bp, Artificial
  Synthetic Sequence
atggacacgagggcccccactcagctgctggggctcctgctgctctggctcccaggtgccagatgtgatgttgtgatgacccagactccagcctccgtggaggcagctgtgggaggcacagtcaccatcaagtgccaggccagtgagagcattggcaatgcattagcctggtatcagcagaaaccagggcagcctcccaagctcctgatctacaaggcatccactctggcatctggggtcccatcgcggttcagcggcagtggatctgggacagagttcactctcaccatcagcggcgtgcagtgtgccgatgctgccgcttactactgtcaatggtgttattttggtgatagtgtt

SEQ ID NO: 211 - DNA, 384 bp, Artificial
  Synthetic Sequence
atggagactgggctgcgctggcttctcctggtcgctgtgctcaaaggtgtccagtgtcagcagcagctggtggagtccgggggaggcctggtcaagccgggggcatccctgacactcacctgcaaagcctctggattctccttcagtagcggctactacatgtgctgggtccgccaggctccagggaaggggctggagtcgatcgcatgcatttttactattactgataacacttactacgcgaactgggcgaaaggccgattcaccatctccaagccctcgtcgcccacggtgactctgcaaatgaccagtctgacagccgcggacacggccacctatttctgtgcgagggggatttattctactgataattattatgccttg

SEQ ID NO: 212 - DNA, 33 bp, Artificial
  Synthetic Sequence
caggccagtgagagcattggcaatgcattagcc

SEQ ID NO: 213 - DNA, 21 bp, Artificial
  Synthetic Sequence
aaggcatccactctggcatct

SEQ ID NO: 214 - DNA, 27 bp, Artificial
  Synthetic Sequence
caatggtgttattttggtgatagtgtt

SEQ ID NO: 215 - DNA, 18 bp, Artificial
  Synthetic Sequence
agcggctactacatgtgc

SEQ ID NO: 216 - DNA, 51 bp, Artificial
  Synthetic Sequence
tgcatttttactattactgataacacttactacgcgaactgggcgaaaggc

SEQ ID NO: 217 - DNA, 33 bp, Artificial
  Synthetic Sequence
gggatttattctactgataattattatgccttg

SEQ ID NO: 218 - protein, 123 aa, Artificial
  Synthetic Polypeptide
MDTRAPTQLLGLLLLWLPGARCDVVMTQTPASVEAAVGGTVTIKCQASQSVSSYLNWYQQKPGQPPKLLIYRASTLESGVPSRFKGSGSGTEFTLTISDLECADAATYYCQCTYGTSSSYGAA

SEQ ID NO: 219 - protein, 133 aa, Artificial
  Synthetic Polypeptide
METGLRWLLLVAVLKGVQCQSVEESGGRLVTPGTPLTLTCTVSGISLSSNAISWVRQAPGKGLEWIGIISYSGTTYYASWAKGRFTISKTSSTTVDLKITSPTTEDTATYFCARDDPTTVMVMLIPFGAGMDL

SEQ ID NO: 220 - protein, 11 aa, Artificial
  Synthetic Peptide
QASQSVSSYLN

SEQ ID NO: 221 - protein, 7 aa, Artificial
  Synthetic Peptide
RASTLES

SEQ ID NO: 222 - protein, 13 aa, Artificial
  Synthetic Peptide
QCTYGTSSSYGAA

SEQ ID NO: 223 - protein, 5 aa, Artificial
  Synthetic Peptide
SNAIS

SEQ ID NO: 224 - protein, 16 aa, Artificial
  Synthetic Peptide
IISYSGTTYYASWAKG

SEQ ID NO: 225 - protein, 19 aa, Artificial
  Synthetic Peptide
DDPTTVMVMLIPFGAGMDL

SEQ ID NO: 226 - DNA, 369 bp, Artificial
  Synthetic Sequence
atggacacgagggcccccactcagctgctggggctcctgctgctctggctcccaggtgccagatgtgatgttgtgatgacccagactccagcctccgtggaggcagctgtgggaggcacagtcaccatcaagtgccaggccagtcagagcgttagtagctacttaaactggtatcagcagaaaccagggcagcctcccaagctcctgatctacagggcatccactctggaatctggggtcccatcgcggttcaaaggcagtggatctgggacagagttcactctcaccatcagcgacctggagtgtgccgatgctgccacttactactgtcaatgtacttatggtactagtagtagttatggtgctgct

SEQ ID NO: 227 - DNA, 399 bp, Artificial
  Synthetic Sequence
atggagactgggctgcgctggcttctcctggtcgctgtgctcaaaggtgtccagtgtcagtcggtggaggagtccgggggtcgcctggtcacgcctgggacacccctgacactcacctgcaccgtctctggtatctccctcagtagcaatgcaataagctgggtccgccaggctccagggaaggggctggaatggatcggaatcattagttatagtggtaccacatactacgcgagctgggcgaaaggccgattcaccatctccaaaacctcgtcgaccacggtggatctgaaaatcactagtccgacaaccgaggacacggccacctacttctgtgccagagatgaccctacgacagttatggttatgttgataccttttggagccggcatggacctc

SEQ ID NO: 228 - DNA, 33 bp, Artificial
  Synthetic Sequence
caggccagtcagagcgttagtagctacttaaac

SEQ ID NO: 229 - DNA, 21 bp, Artificial
  Synthetic Sequence
agggcatccactctggaatct

SEQ ID NO: 230 - DNA, 39 bp, Artificial
  Synthetic Sequence
caatgtacttatggtactagtagtagttatggtgctgct

SEQ ID NO: 231 - DNA, 15 bp, Artificial
  Synthetic Sequence
agcaatgcaataagc

SEQ ID NO: 232 - DNA, 48 bp, Artificial
  Synthetic Sequence
atcattagttatagtggtaccacatactacgcgagctgggcgaaaggc

SEQ ID NO: 233 - DNA, 57 bp, Artificial
  Synthetic Sequence
gatgaccctacgacagttatggttatgttgataccttttggagccggcatggacctc

SEQ ID NO: 234 - protein, 125 aa, Artificial
  Synthetic Polypeptide
MDTRAPTQLLGLLLLWLPGATFAQVLTQTASPVSAAVGGTVTINCQASQSVYKNNYLSWYQQKPGQPPKGLIYSASTLDSGVPLRFSGSGSGTQFTLTISDVQCDDAATYYCLGSYDCSSGDCYA

SEQ ID NO: 235 - protein, 119 aa, Artificial
  Synthetic Polypeptide
METGLRWLLLVAVLKGVQCQSLEESGGDLVKPEGSLTLTCTASGFSFSSYWMCWVRQAPGKGLEWIACIVTGNGNTYYANWAKGRFTISKTSSTTVTLQMTSLTAADTATYFCAKAYDL

SEQ ID NO: 236 - protein, 13 aa, Artificial
  Synthetic Peptide
QASQSVYKNNYLS

SEQ ID NO: 237 - protein, 7 aa, Artificial
  Synthetic Peptide
SASTLDS

SEQ ID NO: 238 - protein, 13 aa, Artificial
  Synthetic Peptide
LGSYDCSSGDCYA

SEQ ID NO: 239 - protein, 5 aa, Artificial
  Synthetic Peptide
SYWMC

SEQ ID NO: 240 - protein, 17 aa, Artificial
  Synthetic Peptide
CIVTGNGNTYYANWAKG

SEQ ID NO: 241 - protein, 4 aa, Artificial
  Synthetic Peptide
AYDL

SEQ ID NO: 242 - DNA, 375 bp, Artificial
  Synthetic Sequence
atggacacgagggcccccactcagctgctggggctcctgctgctctggctcccaggtgccacatttgcccaagtgctgacccagactgcatcgcccgtgtctgcagctgtgggaggcacagtcaccatcaactgccaggccagtcagagtgtttataagaacaactacttatcctggtatcagcagaaaccagggcagcctcccaaaggcctgatctattctgcatcgactctagattctggggtcccattgcggttcagcggcagtggatctgggacacagttcactctcaccatcagcgacgtgcagtgtgacgatgctgccacttactactgtctaggcagttatgattgtagtagtggtgattgttatgct

SEQ ID NO: 243 - DNA, 357 bp, Artificial
  Synthetic Sequence
atggagactgggctgcgctggcttctcctggtcgctgtgctcaaaggtgtccagtgtcagtcgttggaggagtccgggggagacctggtcaagcctgagggatccctgacactcacctgcacagcctctggattctccttcagtagctactggatgtgctgggtccgccaggctccagggaaggggctggagtggatcgcatgcattgttactggtaatggtaacacttactacgcgaactgggcgaaaggccgattcaccatctccaaaacctcgtcgaccacggtgactctgcaaatgaccagtctgacagccgcggacacggccacctatttttgtgcgaaagcctatgacttg

SEQ ID NO: 244 - DNA, 39 bp, Artificial
  Synthetic Sequence
caggccagtcagagtgtttataagaacaactacttatcc

SEQ ID NO: 245 - DNA, 21 bp, Artificial
  Synthetic Sequence
tctgcatcgactctagattct

SEQ ID NO: 246 - DNA, 39 bp, Artificial
  Synthetic Sequence
ctaggcagttatgattgtagtagtggtgattgttatgct

SEQ ID NO: 247 - DNA, 15 bp, Artificial
  Synthetic Sequence
agctactggatgtgc

SEQ ID NO: 248 - DNA, 51 bp, Artificial
  Synthetic Sequence
tgcattgttactggtaatggtaacacttactacgcgaactgggcgaaaggc

SEQ ID NO: 249 - DNA, 12 bp, Artificial
  Synthetic Sequence
gcctatgacttg

SEQ ID NO: 250 - protein, 123 aa, Artificial
  Synthetic Polypeptide
MDTRAPTQLLGLLLLWLPGSTFAAVLTQTPSPVSAAVGGTVSISCQASQSVYDNNYLSWYQQKPGQPPKLLIYGASTLASGVPSRFKGTGSGTQFTLTITDVQCDDAATYYCAGVFNDDSDDA

SEQ ID NO: 251 - protein, 125 aa, Artificial
  Synthetic Polypeptide
METGLRWLLLVAVPKGVQCQSLEESGGRLVTPGTPLTLTCTLSGFSLSAYYMSWVRQAPGKGLEWIGFITLSDHISYARWAKGRFTISKTSTTVDLKMTSPTTEDTATYFCARSRGWGAMGRLDL

SEQ ID NO: 252 - protein, 13 aa, Artificial
  Synthetic Peptide
QASQSVYDNNYLS

SEQ ID NO: 253 - protein, 7 aa, Artificial
  Synthetic Peptide
GASTLAS

SEQ ID NO: 254 - protein, 11 aa, Artificial
  Synthetic Peptide
AGVFNDDSDDA

SEQ ID NO: 255 - protein, 5 aa, Artificial
  Synthetic Peptide
AYYMS

SEQ ID NO: 256 - protein, 16 aa, Artificial
  Synthetic Peptide
FITLSDHISYARWAKG

SEQ ID NO: 257 - protein, 12 aa, Artificial
  Synthetic Peptide
SRGWGAMGRLDL

SEQ ID NO: 258 - DNA, 369 bp, Artificial
  Synthetic Sequence
atggacacgagggcccccactcagctgctggggctcctgctgctctggctcccaggttccacatttgccgccgtgctgacccagactccatctcccgtgtctgcagctgtgggaggcacagtcagcatcagttgccaggccagtcagagtgtttatgacaacaactatttatcctggtatcagcagaaaccaggacagcctcccaagctcctgatctatggtgcatccactctggcatctggggtcccatcgcggttcaaaggcacgggatctgggacacagttcactctcaccatcacagacgtgcagtgtgacgatgctgccacttactattgtgcaggcgtttttaatgatgatagtgatgatgcc

SEQ ID NO: 259 - DNA, 375 bp, Artificial
  Synthetic Sequence
atggagactgggctgcgctggcttctcctggtcgctgtgcccaaaggtgtccagtgtcagtcgctggaggagtccgggggtcgcctggtcacgcctgggacacccctgacactcacctgcacactctctggattctccctcagtgcatactatatgagctgggtccgccaggctccagggaaggggctggaatggatcggattcattactctgagtgatcatatatcttacgcgaggtgggcgaaaggccgattcaccatctccaaaacctcgaccacggtggatctgaaaatgaccagtccgacaaccgaggacacggccacctatttctgtgccaggagtcgtggctggggtgcaatgggtcggttggatctc

SEQ ID NO: 260 - DNA, 39 bp, Artificial
  Synthetic Sequence
caggccagtcagagtgtttatgacaacaactatttatcc

SEQ ID NO: 261 - DNA, 21 bp, Artificial
  Synthetic Sequence
ggtgcatccactctggcatct

SEQ ID NO: 262 - DNA, 33 bp, Artificial
  Synthetic Sequence
gcaggcgtttttaatgatgatagtgatgatgcc

SEQ ID NO: 263 - DNA, 15 bp, Artificial
  Synthetic Sequence
gcatactatatgagc

SEQ ID NO: 264 - DNA, 48 bp, Artificial
  Synthetic Sequence
ttcattactctgagtgatcatatatcttacgcgaggtgggcgaaaggc

SEQ ID NO: 265 - DNA, 36 bp, Artificial; identical to SEQ ID NO: 68
  Synthetic Sequence

SEQ ID NO: 266 - protein, 123 aa, Artificial
  Synthetic Polypeptide
MDTRAPTQLLGLLLLWLPGATFAAVLTQTPSPVSAAVGGTVTISCQASQSVYNNKNLAWYQQKSGQPPKLLIYWASTLASGVSSRFSGSGSGTQFTLTVSGVQCDDAATYYCLGVFDDDADNA

SEQ ID NO: 267 - protein, 121 aa, Artificial
  Synthetic Polypeptide
METGLRWLLLVAVLKGVQCQSVEESGGRLVTPGTPLTLTCTASGFSLSSYSMTWVRQAPGKGLEYIGVIGTSGSTYYATWAKGRFTISRTSTTVALKITSPTTEDTATYFCVRSLSSITFL

SEQ ID NO: 268 - protein, 13 aa, Artificial
  Synthetic Peptide
QASQSVYNNKNLA

SEQ ID NO: 269 - protein, 7 aa, Artificial
  Synthetic Peptide
WASTLAS

SEQ ID NO: 270 - protein, 11 aa, Artificial
  Synthetic Peptide
LGVFDDDADNA

SEQ ID NO: 271 - protein, 5 aa, Artificial
  Synthetic Peptide
SYSMT

SEQ ID NO: 272 - protein, 16 aa, Artificial
  Synthetic Peptide
VIGTSGSTYYATWAKG

SEQ ID NO: 273 - protein, 8 aa, Artificial
  Synthetic Peptide
SLSSITFL

SEQ ID NO: 274 - DNA, 369 bp, Artificial
  Synthetic Sequence
atggacacgagggcccccactcagctgctggggctcctgctgctctggctcccaggtgccacattcgcagccgtgctgacccagacaccatcgcccgtgtctgcggctgtgggaggcacagtcaccatcagttgccaggccagtcagagtgtttataacaacaaaaatttagcctggtatcagcagaaatcagggcagcctcccaagctcctgatctactgggcatccactctggcatctggggtctcatcgcggttcagcggcagtggatctgggacacagttcactctcaccgtcagcggcgtgcagtgtgacgatgctgccacttactactgtctaggcgtttttgatgatgatgctgataatgct

SEQ ID NO: 275 - DNA, 363 bp, Artificial
  Synthetic Sequence
atggagactgggctgcgctggcttctcctggtcgctgtgctcaaaggtgtccaatgtcagtcggtggaggagtccgggggtcgcctggtcacgcctgggacacccctgacactcacctgcacagcctctggattctccctcagtagctactccatgacctgggtccgccaggctccagggaaggggctggaatatatcggagtcattggtactagtggtagcacatactacgcgacctgggcgaaaggccgattcaccatctccagaacctcgaccacggtggctctgaaaatcaccagtccgacaaccgaggacacggccacctatttctgtgtcaggagtctttcttctattactttcttg

SEQ ID NO: 276 - DNA, 39 bp, Artificial
  Synthetic Sequence
caggccagtcagagtgtttataacaacaaaaatttagcc

SEQ ID NO: 277 - DNA, 21 bp, Artificial
  Synthetic Sequence
tgggcatccactctggcatct

SEQ ID NO: 278 - DNA, 33 bp, Artificial
  Synthetic Sequence
ctaggcgtttttgatgatgatgctgataatgct

SEQ ID NO: 279 - DNA, 15 bp, Artificial
  Synthetic Sequence
agctactccatgacc

SEQ ID NO: 280 - DNA, 48 bp, Artificial
  Synthetic Sequence
gtcattggtactagtggtagcacatactacgcgacctgggcgaaaggc

SEQ ID NO: 281 - DNA, 24 bp, Artificial
  Synthetic Sequence
agtctttcttctattactttcttg

SEQ ID NO: 282 - protein, 120 aa, Artificial
  Synthetic Polypeptide
MDTRAPTQLLGLLLLWLPGARCAFELTQTPASVEAAVGGTVTINCQASQNIYRYLAWYQQKPGQPPKFLIYLASTLASGVPSRFKGSGSGTEFTLTISDLECADAATYYCQSYYSSNSVA

SEQ ID NO: 283 - protein, 128 aa, Artificial
  Synthetic Polypeptide
METGLRWLLLVAVLKGVQCQEQLVESGGDLVQPEGSLTLTCTASELDFSSGYWICWVRQVPGKGLEWIGCIYTGSSGSTFYASWAKGRFTISKTSSTTVTLQMTSLTAADTATYFCARGYSGFGYFKL

SEQ ID NO: 284 - protein, 11 aa, Artificial
  Synthetic Peptide
QASQNIYRYLA

SEQ ID NO: 285 - protein, 7 aa, Artificial
  Synthetic Peptide
LASTLAS

SEQ ID NO: 286 - protein, 10 aa, Artificial
  Synthetic Peptide
QSYYSSNSVA

SEQ ID NO: 287 - protein, 6 aa, Artificial
  Synthetic Peptide
SGYWIC

SEQ ID NO: 288 - protein, 18 aa, Artificial
  Synthetic Peptide
CIYTGSSGSTFYASWAKG

SEQ ID NO: 289 - protein, 10 aa, Artificial
  Synthetic Peptide
GYSGFGYFKL

SEQ ID NO: 290 - DNA, 360 bp, Artificial
  Synthetic Sequence
atggacacgagggcccccactcagctgctggggctcctgctgctctggctcccaggtgccagatgtgcattcgaattgacccagactccagcctccgtggaggcagctgtgggaggcacagtcaccatcaattgccaggccagtcagaacatttatagatacttagcctggtatcagcagaaaccagggcagcctcccaagttcctgatctatctggcatctactctggcatctggggtcccatcgcggtttaaaggcagtggatctgggacagagttcactctcaccatcagcgacctggagtgtgccgatgctgccacttactactgtcaaagttattatagtagtaatagtgtcgct

SEQ ID NO: 291 - DNA, 384 bp, Artificial
  Synthetic Sequence
atggagactgggctgcgctggcttctcctggtcgctgtgctcaaaggtgtccagtgtcaggagcagctggtggagtccgggggagacctggtccagcctgagggatccctgacactcacctgcacagcttctgagttagacttcagtagcggctactggatatgctgggtccgccaggttccagggaaggggctggagtggatcggatgcatttatactggtagtagtggtagcactttttacgcgagttgggcgaaaggccgattcaccatctccaaaacctcgtcgaccacggtgactctgcaaatgaccagtctgacagccgcggacacggccacctatttctgtgcgagaggttatagtggctttggttactttaagttg

SEQ ID NO: 292 - DNA, 33 bp, Artificial
  Synthetic Sequence
caggccagtcagaacatttatagatacttagcc

SEQ ID NO: 293 - DNA, 21 bp, Artificial
  Synthetic Sequence
ctggcatctactctggcatct

SEQ ID NO: 294 - DNA, 30 bp, Artificial
  Synthetic Sequence
caaagttattatagtagtaatagtgtcgct

SEQ ID NO: 295 - DNA, 18 bp, Artificial
  Synthetic Sequence
agcggctactggatatgc

SEQ ID NO: 296 - DNA, 54 bp, Artificial
  Synthetic Sequence
tgcatttatactggtagtagtggtagcactttttacgcgagttgggcgaaaggc

SEQ ID NO: 297 - DNA, 30 bp, Artificial
  Synthetic Sequence
ggttatagtggctttggttactttaagttg

SEQ ID NO: 298 - protein, 122 aa, Artificial
  Synthetic Polypeptide
MDTRAPTQLLGLLLLWLPGARCAYDMTQTPASVEVAVGGTVTIKCQASEDIYRLLAWYQQKPGQPPKLLIYDSSDLASGVPSRFKGSGSGTEFTLAISGVQCDDAATYYCQQAWSYSDIDNA

SEQ ID NO: 299 - protein, 123 aa, Artificial
  Synthetic Polypeptide
METGLRWLLLVAVLKGVQCQSVEESGGRLVTPGTPLTLTCTASGFSLSSYYMSWVRQAPGKGLEWIGIITTSGNTFYASWAKGRLTISRTSTTVDLKITSPTTEDTATYFCARTSDIFYYRNL

SEQ ID NO: 300 - protein, 11 aa, Artificial
  Synthetic Peptide
QASEDIYRLLA

SEQ ID NO: 301 - protein, 7 aa, Artificial
  Synthetic Peptide
DSSDLAS

SEQ ID NO: 302 - protein, 12 aa, Artificial
  Synthetic Peptide
QQAWSYSDIDNA

SEQ ID NO: 303 - protein, 5 aa, Artificial
  Synthetic Peptide
SYYMS

SEQ ID NO: 304 - protein, 16 aa, Artificial
  Synthetic Peptide
IITTSGNTFYASWAKG

SEQ ID NO: 305 - protein, 10 aa, Artificial
  Synthetic Peptide
TSDIFYYRNL

SEQ ID NO: 306 - DNA, 366 bp, Artificial
  Synthetic Sequence
atggacacgagggcccccactcagctgctggggctcctgctgctctggctcccaggtgccagatgtgcctatgatatgacccagactccagcctctgtggaggtagctgtgggaggcacagtcaccatcaagtgccaggccagtgaggacatttataggttattggcctggtatcaacagaaaccagggcagcctcccaagctcctgatctatgattcatccgatctggcatctggggtcccatcgcggttcaaaggcagtggatctgggacagagttcactctcgccatcagcggtgtgcagtgtgacgatgctgccacttactactgtcaacaggcttggagttatagtgatattgataatgct

SEQ ID NO: 307 - DNA, 369 bp, Artificial
  Synthetic Sequence
atggagactgggctgcgctggcttctcctggtcgctgtgctcaaaggtgtccagtgtcagtcggtggaggagtccgggggtcgcctggtcacgccggggacacccctgacactcacctgcacagcctctggattctccctcagtagctactacatgagctgggtccgccaggctccagggaaggggctggaatggatcggaatcattactactagtggtaatacattttacgcgagctgggcgaaaggccggctcaccatctccagaacctcgaccacggtggatctgaaaatcaccagtccgacaaccgaggacacggccacctatttctgtgccagaacttctgatattttttattatcgtaacttg

SEQ ID NO: 308 - DNA, 33 bp, Artificial
  Synthetic Sequence
caggccagtgaggacatttataggttattggcc

SEQ ID NO: 309 - DNA, 21 bp, Artificial
  Synthetic Sequence
gattcatccgatctggcatct

SEQ ID NO: 310 - DNA, 36 bp, Artificial
  Synthetic Sequence
caacaggcttggagttatagtgatattgataatgct

SEQ ID NO: 311 - DNA, 15 bp, Artificial
  Synthetic Sequence
agctactacatgagc

SEQ ID NO: 312 - DNA, 48 bp, Artificial
  Synthetic Sequence
atcattactactagtggtaatacattttacgcgagctgggcgaaaggc

SEQ ID NO: 313 - DNA, 30 bp, Artificial
  Synthetic Sequence
acttctgatattttttattatcgtaacttg

SEQ ID NO: 314 - protein, 123 aa, Artificial
  Synthetic Polypeptide
MDTRAPTQLLGLLLLWLPGATFAAVLTQTASPVSAAVGATVTINCQSSQSVYNDMDLAWFQQKPGQPPKLLIYSASTLASGVPSRFSGSGSGTEFTLTISGVQCDDAATYYCLGAFDDDADNT

SEQ ID NO: 315 - protein, 129 aa, Artificial
  Synthetic Polypeptide
METGLRWLLLVAVLKGVQCQSVEESGGRLVTPGTPLTLTCTVSGFSLTRHAITWVRQAPGKGLEWIGCIWSGGSTYYATWAKGRFTISKTSTTVDLRITSPTTEDTATYFCARVIGDTAGYAYFTGLDL

SEQ ID NO: 316 - protein, 13 aa, Artificial
  Synthetic Peptide
QSSQSVYNDMDLA

SEQ ID NO: 317 - protein, 7 aa, Artificial
  Synthetic Peptide
SASTLAS

SEQ ID NO: 318 - protein, 11 aa, Artificial
  Synthetic Peptide
LGAFDDDADNT

SEQ ID NO: 319 - protein, 5 aa, Artificial
  Synthetic Peptide
RHAIT

SEQ ID NO: 320 - protein, 16 aa, Artificial
  Synthetic Peptide
CIWSGGSTYYATWAKG

SEQ ID NO: 321 - protein, 16 aa, Artificial
  Synthetic Peptide
VIGDTAGYAYFTGLDL

SEQ ID NO: 322 - DNA, 369 bp, Artificial
  Synthetic Sequence
atggacacgagggcccccactcagctgctggggctcctgctgctctggctcccaggtgccacgtttgcagccgtgctgacccagactgcatcacccgtgtctgccgctgtgggagccacagtcaccatcaactgccagtccagtcagagtgtttataatgacatggacttagcctggtttcagcagaaaccagggcagcctcccaagctcctgatctattctgcatccactctggcatctggggtcccatcgcggttcagcggcagtggatctgggacagagttcactctcaccatcagcggcgtgcagtgtgacgatgctgccacttactactgtctaggcgcttttgatgatgatgctgataatact

SEQ ID NO: 323 - DNA, 387 bp, Artificial
  Synthetic Sequence
atggagactgggctgcgctggcttctcctggtcgctgtgctcaaaggtgtccagtgtcagtcggtggaggagtccgggggtcgcctggtcacgcctgggacacccctgacactcacctgcacagtctctggattctccctcactaggcatgcaataacctgggtccgccaggctccagggaaggggctggaatggatcggatgcatttggagtggtggtagcacatactacgcgacctgggcgaaaggccgattcaccatctccaaaacctcgaccacggtggatctcagaatcaccagtccgacaaccgaggacacggccacctacttctgtgccagagtcattggcgatactgctggttatgcttattttacggggcttgacttg

SEQ ID NO: 324 - DNA, 39 bp, Artificial
  Synthetic Sequence
cagtccagtcagagtgtttataatgacatggacttagcc

SEQ ID NO: 325 - DNA, 21 bp, Artificial
  Synthetic Sequence
tctgcatccactctggcatct

SEQ ID NO: 326 - DNA, 33 bp, Artificial
  Synthetic Sequence
ctaggcgcttttgatgatgatgctgataatact

SEQ ID NO: 327 - DNA, 15 bp, Artificial
  Synthetic Sequence
aggcatgcaataacc

SEQ ID NO: 328 - DNA, 48 bp, Artificial
  Synthetic Sequence
tgcatttggagtggtggtagcacatactacgcgacctgggcgaaaggc

SEQ ID NO: 329 - DNA, 48 bp, Artificial
  Synthetic Sequence
gtcattggcgatactgctggttatgcttattttacggggcttgacttg

SEQ ID NO: 330 - protein, 121 aa, Artificial
  Synthetic Polypeptide
MDTRAPTQLLGLLLLWLPGARCAYDMTQTPASVEVAVGGTVTIKCQASQSVYNWLSWYQQKPGQPPKLLIYTASSLASGVPSRFSGSGSGTEFTLTISGVECADAATYYCQQGYTSDVDNV

SEQ ID NO: 331 - protein, 130 aa, Artificial
  Synthetic Polypeptide
METGLRWLLLVAVLKGVQCQSLEEAGGRLVTPGTPLTLTCTVSGIDLSSYAMGWVRQAPGKGLEYIGIISSSGSTYYATWAKGRFTISQASSTTVDLKITSPTTEDSATYFCARGGAGSGGVWLLDGFDP

SEQ ID NO: 332 - protein, 11 aa, Artificial
  Synthetic Peptide
QASQSVYNWLS

SEQ ID NO: 333 - protein, 7 aa, Artificial
  Synthetic Peptide
TASSLAS

SEQ ID NO: 334 - protein, 11 aa, Artificial
  Synthetic Peptide
QQGYTSDVDNV

SEQ ID NO: 335 - protein, 5 aa, Artificial
  Synthetic Peptide
SYAMG

SEQ ID NO: 336 - protein, 16 aa, Artificial
  Synthetic Peptide
IISSSGSTYYATWAKG

SEQ ID NO: 337 - protein, 16 aa, Artificial
  Synthetic Peptide
GGAGSGGVWLLDGFDP

SEQ ID NO: 338 - DNA, 363 bp, Artificial
  Synthetic Sequence
atggacacgagggcccccactcagctgctggggctcctgctgctctggctcccaggtgccagatgtgcctatgatatgacccagactccagcctctgtggaggtagctgtgggaggcacagtcaccatcaagtgccaggccagtcagagtgtttataattggttatcctggtatcagcagaaaccagggcagcctcccaagctcctgatctatactgcatccagtctggcatctggggtcccatcgcggttcagtggcagtggatctgggacagagttcactctcaccatcagcggcgtggagtgtgccgatgctgccacttactactgtcaacagggttatactagtgatgttgataatgtt

SEQ ID NO: 339 - DNA, 390 bp, Artificial
  Synthetic Sequence
atggagactgggctgcgctggcttctcctggtcgctgtgctcaaaggtgtccagtgtcagtcgctggaggaggccgggggtcgcctggtcacgcctgggacacccctgacactcacctgcacagtctctggaatcgacctcagtagctatgcaatgggctgggtccgccaggctccagggaaggggctggaatacatcggaatcattagtagtagtggtagcacatactacgcgacctgggcgaaaggccgattcaccatctcacaagcctcgtcgaccacggtggatctgaaaattaccagtccgacaaccgaggactcggccacatatttctgtgccagagggggtgctggtagtggtggtgtttggctgcttgatggttttgatccc

SEQ ID NO: 340 - DNA, 33 bp, Artificial
  Synthetic Sequence
caggccagtcagagtgtttataattggttatcc

SEQ ID NO: 341 - DNA, 21 bp, Artificial
  Synthetic Sequence
actgcatccagtctggcatct

SEQ ID NO: 342 - DNA, 33 bp, Artificial
  Synthetic Sequence
caacagggttatactagtgatgttgataatgtt

SEQ ID NO: 343 - DNA, 15 bp, Artificial
  Synthetic Sequence
agctatgcaatgggc

SEQ ID NO: 344 - DNA, 48 bp, Artificial
  Synthetic Sequence
atcattagtagtagtggtagcacatactacgcgacctgggcgaaaggc

SEQ ID NO: 345 - DNA, 48 bp, Artificial
  Synthetic Sequence
gggggtgctggtagtggtggtgtttggctgcttgatggttttgatccc

SEQ ID NO: 346 - protein, 123 aa, Artificial
  Synthetic Polypeptide
MDTRAPTQLLGLLLLWLPGAKCADVVMTQTPASVSAAVGGTVTINCQASENIYNWLAWYQQKPGQPPKLLIYTVGDLASGVSSRFKGSGSGTEFTLTISDLECADAATYYCQQGYSSSYVDNV

SEQ ID NO: 347 - protein, 130 aa, Artificial
  Synthetic Polypeptide
METGLRWLLLVAVLKGVQCQEQLKESGGRLVTPGTPLTLTCTVSGFSLNDYAVGWFRQAPGKGLEWIGYIRSSGTTAYATWAKGRFTISATSTTVDLKITSPTTEDTATYFCARGGAGSSGVWILDGFAP

SEQ ID NO: 348 - protein, 11 aa, Artificial
  Synthetic Peptide
QASENIYNWLA

SEQ ID NO: 349 - protein, 7 aa, Artificial
  Synthetic Peptide
TVGDLAS

SEQ ID NO: 350 - protein, 12 aa, Artificial
  Synthetic Peptide
QQGYSSSYVDNV

SEQ ID NO: 351 - protein, 5 aa, Artificial
  Synthetic Peptide
DYAVG

SEQ ID NO: 352 - protein, 16 aa, Artificial
  Synthetic Peptide
YIRSSGTTAYATWAKG

SEQ ID NO: 353 - protein, 16 aa, Artificial
  Synthetic Peptide
GGAGSSGVWILDGFAP

SEQ ID NO: 354 - DNA, 369 bp, Artificial
  Synthetic Sequence
atggacacgagggcccccactcagctgctggggctcctgctgctctggctcccaggtgccaaatgtgccgatgttgtgatgacccagactccagcctccgtgtctgcagctgtgggaggcacagtcaccatcaattgccaggccagtgagaacatttataattggttagcctggtatcagcagaaaccagggcagcctcccaagctcctgatctatactgtaggcgatctggcatctggggtctcatcgcggttcaaaggcagtggatctgggacagagttcactctcaccatcagcgacctggagtgtgccgatgctgccacttactattgtcaacagggttatagtagtagttatgttgataatgtt

SEQ ID NO: 355 - DNA, 390 bp, Artificial
  Synthetic Sequence
atggagactgggctgcgctggcttctcctggtcgctgtgctcaaaggtgtccagtgtcaggagcagctgaaggagtccgggggtcgcctggtcacgcctgggacacccctgacactcacctgcacagtctctggattctccctcaatgactatgcagtgggctggttccgccaggctccagggaaggggctggaatggatcggatacattcgtagtagtggtaccacagcctacgcgacctgggcgaaaggccgattcaccatctccgctacctcgaccacggtggatctgaaaatcaccagtccgacaaccgaggacacggccacctatttctgtgccagagggggtgctggtagtagtggtgtgtggatccttgatggttttgctccc

SEQ ID NO: 356 - DNA, 33 bp, Artificial
  Synthetic Sequence
caggccagtgagaacatttataattggttagcc

SEQ ID NO: 357 - DNA, 21 bp, Artificial
  Synthetic Sequence
actgtaggcgatctggcatct

SEQ ID NO: 358 - DNA, 36 bp, Artificial
  Synthetic Sequence
caacagggttatagtagtagttatgttgataatgtt

SEQ ID NO: 359 - DNA, 15 bp, Artificial
  Synthetic Sequence
gactatgcagtgggc

SEQ ID NO: 360 - DNA, 48 bp, Artificial
  Synthetic Sequence
tacattcgtagtagtggtaccacagcctacgcgacctgggcgaaaggc

SEQ ID NO: 361 - DNA, 48 bp, Artificial
  Synthetic Sequence
gggggtgctggtagtagtggtgtgtggatccttgatggttttgctccc

SEQ ID NO: 362 - protein, 121 aa, Artificial
  Synthetic Polypeptide
MDTRAPTQLLGLLLLWLPGATFAQVLTQTPSSVSAAVGGTVTINCQASQSVYQNNYLSWFQQKPGQPPKLLIYGAATLASGVPSRFKGSGSGTQFTLTISDLECDDAATYYCAGAYRDVDS

SEQ ID NO: 363 - protein, 130 aa, Artificial
  Synthetic Polypeptide
METGLRWLLLVAVLKGVQCQSLEESGGDLVKPGASLTLTCTASGFSFTSTYYIYWVRQAPGKGLEWIACIDAGSSGSTYYATWVNGRFTISKTSSTTVTLQMTSLTAADTATYFCAKWDYGGNVGWGYDL

SEQ ID NO: 364 - protein, 13 aa, Artificial
  Synthetic Peptide
QASQSVYQNNYLS

SEQ ID NO: 365 - protein, 7 aa, Artificial
  Synthetic Peptide
GAATLAS

SEQ ID NO: 366 - protein, 9 aa, Artificial
  Synthetic Peptide
AGAYRDVDS

SEQ ID NO: 367 - protein, 6 aa, Artificial
  Synthetic Peptide
STYYIY

SEQ ID NO: 368 - protein, 18 aa, Artificial
  Synthetic Peptide
CIDAGSSGSTYYATWVNG

SEQ ID NO: 369 - protein, 13 aa, Artificial
  Synthetic Peptide
WDYGGNVGWGYDL

SEQ ID NO: 370 - DNA, 363 bp, Artificial
  Synthetic Sequence
atggacacgagggcccccactcagctgctggggctcctgctgctctggctcccaggtgccacatttgctcaagtgctgacccagactccatcctccgtgtctgcagctgtgggaggcacagtcaccatcaattgccaggccagtcagagtgtttatcagaacaactacttatcctggtttcagcagaaaccagggcagcctcccaagctcctgatctatggtgcggccactctggcatctggggtcccatcgcggttcaaaggcagtggatctgggacacagttcactctcaccatcagcgacctggagtgtgacgatgctgccacttactactgtgcaggcgcttatagggatgtggattct

SEQ ID NO: 371 - DNA, 390 bp, Artificial
  Synthetic Sequence
atggagactgggctgcgctggcttctcctggtcgctgtgctcaaaggtgtccagtgtcagtcgttggaggagtccgggggagacctggtcaagcctggggcatccctgacactcacctgcacagcctctggattctcctttactagtacctactacatctactgggtccgccaggctccagggaaggggctggagtggatcgcatgtattgatgctggtagtagtggtagcacttactacgcgacctgggtgaatggccgattcaccatctccaaaacctcgtcgaccacggtgactctgcaaatgaccagtctgacagccgcggacacggccacctatttctgtgcgaaatgggattatggtggtaatgttggttggggttatgacttg

SEQ ID NO: 372 - DNA, 39 bp, Artificial
  Synthetic Sequence
caggccagtcagagtgtttatcagaacaactacttatcc

SEQ ID NO: 373 - DNA, 21 bp, Artificial
  Synthetic Sequence
ggtgcggccactctggcatct

SEQ ID NO: 374 - DNA, 27 bp, Artificial
  Synthetic Sequence
gcaggcgcttatagggatgtggattct

SEQ ID NO: 375 - DNA, 18 bp, Artificial
  Synthetic Sequence
agtacctactacatctac

SEQ ID NO: 376 - DNA, 54 bp, Artificial
  Synthetic Sequence
tgtattgatgctggtagtagtggtagcacttactacgcgacctgggtgaatggc

SEQ ID NO: 377 - DNA, 39 bp, Artificial
  Synthetic Sequence
tgggattatggtggtaatgttggttggggttatgacttg

SEQ ID NO: 378 - protein, 120 aa, Artificial
  Synthetic Polypeptide
MDTRAPTQLLGLLLLWLPGARCAFELTQTPSSVEAAVGGTVTIKCQASQSISSYLAWYQQKPGQPPKFLIYRASTLASGVPSRFKGSGSGTEFTLTISDLECADAATYYCQSYYDSVSNP

SEQ ID NO: 379 - protein, 127 aa, Artificial
  Synthetic Polypeptide
METGLRWLLLVAVLKGVQCQSLEESGGDLVKPEGSLTLTCKASGLDLGTYWFMCWVRQAPGKGLEWIACIYTGSSGSTFYASWVNGRFTISKTSSTTVTLQMTSLTAADTATYFCARGYSGYGYFKL

SEQ ID NO: 380 - protein, 11 aa, Artificial
  Synthetic Peptide
QASQSISSYLA

SEQ ID NO: 381 - protein, 7 aa, Artificial
  Synthetic Peptide
RASTLAS

SEQ ID NO: 382 - protein, 10 aa, Artificial
  Synthetic Peptide
QSYYDSVSNP

SEQ ID NO: 383 - protein, 6 aa, Artificial
  Synthetic Peptide
TYWFMC

SEQ ID NO: 384 - protein, 18 aa, Artificial
  Synthetic Peptide
CIYTGSSGSTFYASWVNG

SEQ ID NO: 385 - protein, 10 aa, Artificial
  Synthetic Peptide
GYSGYGYFKL

SEQ ID NO: 386 - DNA, 360 bp, Artificial
  Synthetic Sequence
atggacacgagggcccccactcagctgctggggctcctgctgctctggctcccaggtgccagatgtgcattcgaattgacccagactccatcctccgtggaggcagctgtgggaggcacagtcaccatcaagtgccaggccagtcagagcattagtagttacttagcctggtatcagcagaaaccagggcagcctcccaagttcctgatctacagggcgtccactctggcatctggggtcccatcgcgattcaaaggcagtggatctgggacagagttcactctcaccatcagcgacctggagtgtgccgatgctgccacttactactgtcaaagctattatgatagtgtttcaaatcct

SEQ ID NO: 387 - DNA, 381 bp, Artificial
  Synthetic Sequence
atggagactgggctgcgctggcttctcctggtcgctgtgctcaaaggtgtccagtgtcagtcgttggaggagtccgggggagacctggtcaagcctgagggatccctgacactcacctgcaaagcctctggactcgacctcggtacctactggttcatgtgctgggtccgccaggctccagggaaggggctggagtggatcgcttgtatttatactggtagtagtggttccactttctacgcgagctgggtgaatggccgattcaccatctccaaaacctcgtcgaccacggtgactctgcaaatgaccagtctgacagccgcggacacggccacttatttttgtgcgagaggttatagtggttatggttattttaagttg

SEQ ID NO: 388 - DNA, 33 bp, Artificial
  Synthetic Sequence
caggccagtcagagcattagtagttacttagcc

SEQ ID NO: 389 - DNA, 21 bp, Artificial
  Synthetic Sequence
agggcgtccactctggcatct

SEQ ID NO: 390 - DNA, 30 bp, Artificial
  Synthetic Sequence
caaagctattatgatagtgtttcaaatcct

SEQ ID NO: 391 - DNA, 18 bp, Artificial
  Synthetic Sequence
acctactggttcatgtgc

SEQ ID NO: 392 - DNA, 54 bp, Artificial
  Synthetic Sequence
tgtatttatactggtagtagtggttccactttctacgcgagctgggtgaatggc

SEQ ID NO: 393 - DNA, 30 bp, Artificial
  Synthetic Sequence
ggttatagtggttatggttattttaagttg

SEQ ID NO: 394 - protein, 124 aa, Artificial
  Synthetic Polypeptide
MDTRAPTQLLGLLLLWLPGVTFAIEMTQSPFSVSAAVGGTVSISCQASQSVYKNNQLSWYQQKSGQPPKLLIYGASALASGVPSRFKGSGSGTEFTLTISDVQCDDAATYYCAGAITGSIDTDG

SEQ ID NO: 395 - protein, 130 aa, Artificial
  Synthetic Polypeptide
METGLRWLLLVAVLKGVQCQSLEESGGDLVKPGASLTLTCTTSGFSFSSSYFICWVRQAPGKGLEWIACIYGGDGSTYYASWAKGRFTISKTSSTTVTLQMTSLTAADTATYFCAREWAYSQGYFGAFDL

SEQ ID NO: 396 - protein, 13 aa, Artificial
  Synthetic Peptide
QASQSVYKNNQLS

SEQ ID NO: 397 - protein, 7 aa, Artificial
  Synthetic Peptide
GASALAS

SEQ ID NO: 398 - protein, 12 aa, Artificial
  Synthetic Peptide
AGAITGSIDTDG

SEQ ID NO: 399 - protein, 6 aa, Artificial
  Synthetic Peptide
SSYFIC

SEQ ID NO: 400 - protein, 17 aa, Artificial
  Synthetic Peptide
CIYGGDGSTYYASWAKG

SEQ ID NO: 401 - protein, 14 aa, Artificial
  Synthetic Peptide
EWAYSQGYFGAFDL

SEQ ID NO: 402 - DNA, 372 bp, Artificial
  Synthetic Sequence
atggacacgagggcccccactcagctgctggggctcctgctgctctggctcccaggtgtcacatttgccatcgaaatgacccagagtccattctccgtgtctgcagctgtgggaggcacagtcagcatcagttgccaggccagtcagagtgtttataagaacaaccaattatcctggtatcagcagaaatcagggcagcctcccaagctcctgatctatggtgcatcggctctggcatctggggtcccatcgcggttcaaaggcagtggatctgggacagagttcactctcaccatcagcgacgtgcagtgtgacgatgctgccacttactactgtgcaggcgctattactggtagtattgatacggatggt

SEQ ID NO: 403 - DNA, 390 bp, Artificial
  Synthetic Sequence
atggagactgggctgcgctggcttctcctggtcgctgtgctcaaaggtgtccagtgtcagtcgttggaggagtccgggggagacctggtcaagcctggggcatccctgacactcacctgcacaacttctggattctccttcagtagcagctacttcatttgctgggtccgccaggctccagggaaggggctggagtggatcgcatgcatttatggtggtgatggcagcacatactacgcgagctgggcgaaaggccgattcaccatctccaaaacctcgtcgaccacggtgacgctgcaaatgaccagtctgacagccgcggacacggccacctatttctgtgcgagagaatgggcatatagtcaaggttattttggtgcttttgatctc

SEQ ID NO: 404 - DNA, 39 bp, Artificial
  Synthetic Sequence
caggccagtcagagtgtttataagaacaaccaattatcc

SEQ ID NO: 405 - DNA, 21 bp, Artificial
  Synthetic Sequence
ggtgcatcggctctggcatct

SEQ ID NO: 406 - DNA, 36 bp, Artificial
  Synthetic Sequence
gcaggcgctattactggtagtattgatacggatggt

SEQ ID NO: 407 - DNA, 18 bp, Artificial
  Synthetic Sequence
agcagctacttcatttgc

SEQ ID NO: 408 - DNA, 51 bp, Artificial
  Synthetic Sequence
tgcatttatggtggtgatggcagcacatactacgcgagctgggcgaaaggc

SEQ ID NO: 409 - DNA, 42 bp, Artificial
  Synthetic Sequence
gaatgggcatatagtcaaggttattttggtgcttttgatctc

SEQ ID NO: 410 - protein, 124 aa, Artificial
  Synthetic Polypeptide
MDTRAPTQLLGLLLLWLPGARCDVVMTQTPASVEAAVGGTVTIKCQASEDISSYLAWYQQKPGQPPKLLIYAASNLESGVSSRFKGSGSGTEYTLTISDLECADAATYYCQCTYGTISISDGNA

SEQ ID NO: 411 - protein, 124 aa, Artificial
  Synthetic Polypeptide
METGLRWLLLVAVLKGVQCQSVEESGGRLVTPGTPLTLTCTVSGFSLSSYFMTWVRQAPGEGLEYIGFINPGGSAYYASWVKGRFTISKSSTTVDLKITSPTTEDTATYFCARVLIVSYGAFTI

SEQ ID NO: 412 - protein, 11 aa, Artificial
  Synthetic Peptide
QASEDISSYLA

SEQ ID NO: 413 - protein, 7 aa, Artificial
  Synthetic Peptide
AASNLES

SEQ ID NO: 414 - protein, 14 aa, Artificial
  Synthetic Peptide
QCTYGTISISDGNA

SEQ ID NO: 415 - protein, 5 aa, Artificial
  Synthetic Peptide
SYFMT

SEQ ID NO: 416 - protein, 16 aa, Artificial
  Synthetic Peptide
FINPGGSAYYASWVKG

SEQ ID NO: 417 - protein, 11 aa, Artificial
  Synthetic Peptide
VLIVSYGAFTI

SEQ ID NO: 418 - DNA, 372 bp, Artificial
  Synthetic Sequence
atggacacgagggcccccactcagctgctggggctcctgctgctctggctcccaggtgccagatgtgatgttgtgatgacccagactccagcctccgtggaggcagctgtgggaggcacagtcaccatcaagtgccaggccagtgaggatattagtagctacttagcctggtatcagcagaaaccagggcagcctcccaagctcctgatctatgctgcatccaatctggaatctggggtctcatcgcgattcaaaggcagtggatctgggacagagtacactctcaccatcagcgacctggagtgtgccgatgctgccacctattactgtcaatgtacttatggtactatttctattagtgatggtaatgct

SEQ ID NO: 419 - DNA, 372 bp, Artificial
  Synthetic Sequence
atggagactgggctgcgctggcttctcctggtcgctgtgctcaaaggtgtccaatgtcagtcggtggaggagtccgggggtcgcctggtcacgcctgggacacccctgacactcacctgcacagtctctggattctccctcagtagctacttcatgacctgggtccgccaggctccaggggaggggctggaatacatcggattcattaatcctggtggtagcgcttactacgcgagctgggtgaaaggccgattcaccatctccaagtcctcgaccacggtagatctgaaaatcaccagtccgacaaccgaggacacggccacctatttctgtgccagggttctgattgtttcttatggagcctttaccatc

SEQ ID NO: 420 - DNA, 33 bp, Artificial
  Synthetic Sequence
caggccagtgaggatattagtagctacttagcc

SEQ ID NO: 421 - DNA, 21 bp, Artificial
  Synthetic Sequence
gctgcatccaatctggaatct

SEQ ID NO: 422 - DNA, 42 bp, Artificial
  Synthetic Sequence
caatgtacttatggtactatttctattagtgatggtaatgct

SEQ ID NO: 423 - DNA, 15 bp, Artificial
  Synthetic Sequence
agctacttcatgacc

SEQ ID NO: 424 - DNA, 48 bp, Artificial
  Synthetic Sequence
ttcattaatcctggtggtagcgcttactacgcgagctgggtgaaaggc

SEQ ID NO: 425 - DNA, 33 bp, Artificial
  Synthetic Sequence
gttctgattgtttcttatggagcctttaccatc

SEQ ID NO: 426 - protein, 124 aa, Artificial
  Synthetic Polypeptide
MDTRAPTQLLGLLLLWLPGARCDVVMTQTPASVSAAVGGTVTIKCQASEDIESYLAWYQQKPGQPPKLLIYGASNLESGVSSRFKGSGSGTEFTLTISDLECADAATYYCQCTYGIISISDGNA

SEQ ID NO: 427 - protein, 124 aa, Artificial
  Synthetic Polypeptide
METGLRWLLLVAVLKGVQCQSVEESGGRLVTPGTPLTLTCTVSGFSLSSYFMTWVRQAPGEGLEYIGFMNTGDNAYYASWAKGRFTISKTSTTVDLKITSPTTEDTATYFCARVLVVAYGAFNI

SEQ ID NO: 428 - protein, 11 aa, Artificial
  Synthetic Peptide
QASEDIESYLA

SEQ ID NO: 429 - protein, 7 aa, Artificial
  Synthetic Peptide
GASNLES

SEQ ID NO: 430 - protein, 14 aa, Artificial
  Synthetic Peptide
QCTYGIISISDGNA

SEQ ID NO: 431 - protein, 5 aa, Artificial
  Synthetic Peptide
SYFMT

SEQ ID NO: 432 - protein, 16 aa, Artificial
  Synthetic Peptide
FMNTGDNAYYASWAKG

SEQ ID NO: 433 - protein, 11 aa, Artificial
  Synthetic Peptide
VLVVAYGAFNI

SEQ ID NO: 434 - DNA, 372 bp, Artificial
  Synthetic Sequence
atggacacgagggcccccactcagctgctggggctcctgctgctctggctcccaggtgccagatgtgatgttgtgatgacccagactccagcctccgtgtctgcagctgtgggaggcacagtcaccatcaagtgccaggccagtgaggacattgaaagctatctagcctggtatcagcagaaaccagggcagcctcccaagctcctgatctatggtgcatccaatctggaatctggggtctcatcgcggttcaaaggcagtggatctgggacagagttcactctcaccatcagcgacctggagtgtgccgatgctgccacttactattgtcaatgcacttatggtattattagtattagtgatggtaatgct

SEQ ID NO: 435 - DNA, 372 bp, Artificial
  Synthetic Sequence
atggagactgggctgcgctggcttctcctggtcgctgtgctcaaaggtgtccagtgtcagtcggtggaggagtccgggggtcgcctggtcacgcctgggacacccctgacactcacctgcacagtgtctggattctccctcagtagctacttcatgacctgggtccgccaggctccaggggaggggctggaatacatcggattcatgaatactggtgataacgcatactacgcgagctgggcgaaaggccgattcaccatctccaaaacctcgaccacggtggatctgaaaatcaccagtccgacaaccgaggacacggccacctatttctgtgccagggttcttgttgttgcttatggagcctttaacatc

SEQ ID NO: 436 - DNA, 33 bp, Artificial
  Synthetic Sequence
caggccagtgaggacattgaaagctatctagcc

SEQ ID NO: 437 - DNA, 21 bp, Artificial
  Synthetic Sequence
ggtgcatccaatctggaatct

SEQ ID NO: 438 - DNA, 42 bp, Artificial
  Synthetic Sequence
caatgcacttatggtattattagtattagtgatggtaatgct

SEQ ID NO: 439 - DNA, 15 bp, Artificial
  Synthetic Sequence
agctacttcatgacc

SEQ ID NO: 440 - DNA, 48 bp, Artificial
  Synthetic Sequence
ttcatgaatactggtgataacgcatactacgcgagctgggcgaaaggc

SEQ ID NO: 441 - DNA, 33 bp, Artificial
  Synthetic Sequence
gttcttgttgttgcttatggagcctttaacatc

SEQ ID NO: 442 - protein, 124 aa, Artificial
  Synthetic Polypeptide
MDTRAPTQLLGLLLLWLPGATFAAVLTQTPSPVSEPVGGTVSISCQSSKSVMNNNYLAWYQQKPGQPPKLLIYGASNLASGVPSRFSGSGSGTQFTLTISDVQCDDAATYYCQGGYTGYSDHGT

SEQ ID NO: 443 - protein, 127 aa, Artificial
  Synthetic Polypeptide
METGLRWLLLVAVLKGVQCQSVEESGGRLVKPDETLTLTCTVSGIDLSSYPMNWVRQAPGKGLEWIGFINTGGTIVYASWAKGRFTISKTSTTVDLKMTSPTTEDTATYFCARGSYVSSGYAYYFNV

SEQ ID NO: 444 - protein, 13 aa, Artificial
  Synthetic Peptide
QSSKSVMNNNYLA

SEQ ID NO: 445 - protein, 7 aa, Artificial
  Synthetic Peptide
GASNLAS

SEQ ID NO: 446 - protein, 12 aa, Artificial
  Synthetic Peptide
QGGYTGYSDHGT

SEQ ID NO: 447 - protein, 5 aa, Artificial
  Synthetic Peptide
SYPMN

SEQ ID NO: 448 - protein, 16 aa, Artificial
  Synthetic Peptide
FINTGGTIVYASWAKG

SEQ ID NO: 449 - protein, 14 aa, Artificial
  Synthetic Peptide
GSYVSSGYAYYFNV

SEQ ID NO: 450 - DNA, 372 bp, Artificial
  Synthetic Sequence
atggacacgagggcccccactcagctgctggggctcctgctgctctggctcccaggtgccacatttgccgccgtgctgacccagactccatctcccgtgtctgaacctgtgggaggcacagtcagcatcagttgccagtccagtaagagtgttatgaataacaactacttagcctggtatcagcagaaaccagggcagcctcccaagctcctgatctatggtgcatccaatctggcatctggggtcccatcacggttcagcggcagtggatctgggacacagttcactctcaccatcagcgacgtgcagtgtgacgatgctgccacttactactgtcaaggcggttatactggttatagtgatcatgggact

SEQ ID NO: 451 - DNA, 381 bp, Artificial
  Synthetic Sequence
atggagactgggctgcgctggcttctcctggtcgctgtgctcaaaggtgtccagtgtcagtcggtggaggagtccgggggtcgcctggtcaagcctgacgaaaccctgacactcacctgcacagtctctggaatcgacctcagtagctatccaatgaactgggtccgccaggctccagggaaggggctggaatggatcggattcattaatactggtggtaccatagtctacgcgagctgggcaaaaggccgattcaccatctccaaaacctcgaccacggtggatctgaaaatgaccagtccgacaaccgaggacacggccacctatttctgtgccagaggcagttatgtttcatctggttatgcctactattttaatgtc

SEQ ID NO: 452 - DNA, 39 bp, Artificial
  Synthetic Sequence
cagtccagtaagagtgttatgaataacaactacttagcc

SEQ ID NO: 453 - DNA, 21 bp, Artificial
  Synthetic Sequence
ggtgcatccaatctggcatct

SEQ ID NO: 454 - DNA, 36 bp, Artificial
  Synthetic Sequence
caaggcggttatactggttatagtgatcatgggact

SEQ ID NO: 455 - DNA, 15 bp, Artificial
  Synthetic Sequence
agctatccaatgaac

SEQ ID NO: 456 - DNA, 48 bp, Artificial
  Synthetic Sequence
ttcattaatactggtggtaccatagtctacgcgagctgggcaaaaggc

SEQ ID NO: 457 - DNA, 42 bp, Artificial
  Synthetic Sequence
ggcagttatgtttcatctggttatgcctactattttaatgtc

SEQ ID NO: 458 - protein, 121 aa, Artificial
  Synthetic Polypeptide
MDTRAPTQLLGLLLLWLPGATFAAVLTQTPSPVSAAVGGTVSISCQSSQSVYNNNWLSWFQQKPGQPPKLLIYKASTLASGVPSRFKGSGSGTQFTLTISDVQCDDVATYYCAGGYLDSVI

SEQ ID NO: 459 - protein, 126 aa, Artificial
  Synthetic Polypeptide
METGLRWLLLVAVLKGVQCQSVEESGGRLVTPGTPLTLTCTVSGFSLSTYSINWVRQAPGKGLEWIGIIANSGTTFYANWAKGRFTVSKTSTTVDLKITSPTTEDTATYFCARESGMYNEYGKFNI

SEQ ID NO: 460 - protein, 13 aa, Artificial
  Synthetic Peptide
QSSQSVYNNNWLS

SEQ ID NO: 461 - protein, 7 aa, Artificial
  Synthetic Peptide
KASTLAS

SEQ ID NO: 462 - protein, 9 aa, Artificial
  Synthetic Peptide
AGGYLDSVI

SEQ ID NO: 463 - protein, 5 aa, Artificial
  Synthetic Peptide
TYSIN

SEQ ID NO: 464 - protein, 16 aa, Artificial
  Synthetic Peptide
IIANSGTTFYANWAKG

SEQ ID NO: 465 - protein, 13 aa, Artificial
  Synthetic Peptide
ESGMYNEYGKFNI

SEQ ID NO: 466 - DNA, 363 bp, Artificial
  Synthetic Sequence
atggacacgagggcccccactcagctgctggggctcctgctgctctggctcccaggtgccacatttgccgccgtgctgacccagactccatctcccgtgtctgcagctgtgggaggcacagtcagcatcagttgccagtccagtcagagtgtttataataacaactggttatcctggtttcagcagaaaccagggcagcctcccaagctcctgatctacaaggcatccactctggcatctggggtcccatcgcggttcaaaggcagtggatctgggacacagttcactctcaccatcagcgacgtgcagtgtgacgatgttgccacttactactgtgcgggcggttatcttgatagtgttatt

SEQ ID NO: 467 - DNA, 378 bp, Artificial
  Synthetic Sequence
atggagactgggctgcgctggcttctcctggtcgctgtgctcaaaggtgtccagtgtcagtcggtggaggagtccgggggtcgcctggtcacgcctgggacacccctgacactcacctgcacagtctctggattctccctcagtacctattcaataaactgggtccgccaggctccagggaagggcctggaatggatcggaatcattgctaatagtggtaccacattctacgcgaactgggcgaaaggccgattcaccgtctccaaaacctcgaccacggtggatctgaaaatcaccagtccgacaaccgaggacacggccacctatttctgtgccagagagagtggaatgtacaatgaatatggtaaatttaacatc

SEQ ID NO: 468 - DNA, 39 bp, Artificial
  Synthetic Sequence
cagtccagtcagagtgtttataataacaactggttatcc

SEQ ID NO: 469 - DNA, 21 bp, Artificial
  Synthetic Sequence
aaggcatccactctggcatct

SEQ ID NO: 470 - DNA, 27 bp, Artificial
  Synthetic Sequence
gcgggcggttatcttgatagtgttatt

SEQ ID NO: 471 - DNA, 15 bp, Artificial
  Synthetic Sequence
acctattcaataaac

SEQ ID NO: 472 - DNA, 48 bp, Artificial
  Synthetic Sequence
atcattgctaatagtggtaccacattctacgcgaactgggcgaaaggc

SEQ ID NO: 473 - DNA, 39 bp, Artificial
  Synthetic Sequence
gagagtggaatgtacaatgaatatggtaaatttaacatc

SEQ ID NO: 474 - protein, 122 aa, Artificial
  Synthetic Polypeptide
MDTRAPTQLLGLLLLWLPGARCASDMTQTPSSVSAAVGGTVTINCQASENIYSFLAWYQQKPGQPPKLLIFKASTLASGVSSRFKGSGSGTQFTLTISDLECDDAATYYCQQGATVYDIDNN

SEQ ID NO: 475 - protein, 128 aa, Artificial
  Synthetic Polypeptide
METGLRWLLLVAVLKGVQCQSLEESGGRLVTPGTPLTLTCTVSGIDLSAYAMIWVRQAPGEGLEWITIIYPNGITYYANWAKGRFTVSKTSTAMDLKITSPTTEDTATYFCARDAESSKNAYWGYFNV

SEQ ID NO: 476 - protein, 11 aa, Artificial
  Synthetic Peptide
QASENIYSFLA

SEQ ID NO: 477 - protein, 7 aa, Artificial
  Synthetic Peptide
KASTLAS

SEQ ID NO: 478 - protein, 12 aa, Artificial
  Synthetic Peptide
QQGATVYDIDNN

SEQ ID NO: 479 - protein, 5 aa, Artificial
  Synthetic Peptide
AYAMI

SEQ ID NO: 480 - protein, 16 aa, Artificial
  Synthetic Peptide
IIYPNGITYYANWAKG

SEQ ID NO: 481 - protein, 15 aa, Artificial
  Synthetic Peptide
DAESSKNAYWGYFNV

SEQ ID NO: 482 - DNA, 366 bp, Artificial
  Synthetic Sequence
atggacacgagggcccccactcagctgctggggctcctgctgctctggctcccaggtgccagatgtgcctctgatatgacccagactccatcctccgtgtctgcagctgtgggaggcacagtcaccatcaattgccaggccagtgagaacatttatagctttttggcctggtatcagcagaaaccagggcagcctcccaagctcctgatcttcaaggcttccactctggcatctggggtctcatcgcggttcaaaggcagtggatctgggacacagttcactctcaccatcagcgacctggagtgtgacgatgctgccacttactactgtcaacagggtgctactgtgtatgatattgataataat

SEQ ID NO: 483 - DNA, 384 bp, Artificial
  Synthetic Sequence
atggagactgggctgcgctggcttctcctggtcgctgtgctcaaaggtgtccagtgtcagtcgctggaggagtccgggggtcgcctggtcacgcctgggacacccctgacactcacctgcacagtttctggaatcgacctcagtgcctatgcaatgatctgggtccgccaggctccaggggaggggctggaatggatcacaatcatttatcctaatggtatcacatactacgcgaactgggcgaaaggccgattcaccgtctccaaaacctcgaccgcgatggatctgaaaatcaccagtccgacaaccgaggacacggccacctatttctgtgccagagatgcagaaagtagtaagaatgcttattggggctactttaacgtc

SEQ ID NO: 484 - DNA, 33 bp, Artificial
  Synthetic Sequence
caggccagtgagaacatttatagctttttggcc

SEQ ID NO: 485 - DNA, 21 bp, Artificial
  Synthetic Sequence
aaggcttccactctggcatct

SEQ ID NO: 486 - DNA, 36 bp, Artificial
  Synthetic Sequence
caacagggtgctactgtgtatgatattgataataat

SEQ ID NO: 487 - DNA, 15 bp, Artificial
  Synthetic Sequence
gcctatgcaatgatc

SEQ ID NO: 488 - DNA, 48 bp, Artificial
  Synthetic Sequence
atcatttatcctaatggtatcacatactacgcgaactgggcgaaaggc

SEQ ID NO: 489 - DNA, 45 bp, Artificial
  Synthetic Sequence
gatgcagaaagtagtaagaatgcttattggggctactttaacgtc

SEQ ID NO: 490 - protein, 122 aa, Artificial
  Synthetic Polypeptide
MDTRAPTQLLGLLLLWLPGARCASDMTQTPSSVSAAVGGTVTINCQASENIYSFLAWYQQKPGQPPKLLIFRASTLASGVSSRFKGSGSGTQFTLTISDLECDDAATYYCQQGATVYDIDNN

SEQ ID NO: 491 - protein, 128 aa, Artificial; identical to SEQ ID NO: 475
  Synthetic Polypeptide

SEQ ID NO: 492 - protein, 11 aa, Artificial
  Synthetic Peptide
QASENIYSFLA

SEQ ID NO: 493 - protein, 7 aa, Artificial
  Synthetic Peptide
RASTLAS

SEQ ID NO: 494 - protein, 12 aa, Artificial
  Synthetic Peptide
QQGATVYDIDNN

SEQ ID NO: 495 - protein, 5 aa, Artificial
  Synthetic Peptide
AYAMI

SEQ ID NO: 496 - protein, 16 aa, Artificial
  Synthetic Peptide
IIYPNGITYYANWAKG

SEQ ID NO: 497 - protein, 15 aa, Artificial
  Synthetic Peptide
DAESSKNAYWGYFNV

SEQ ID NO: 498 - DNA, 366 bp, Artificial
  Synthetic Sequence
atggacacgagggcccccactcagctgctggggctcctgctgctctggctcccaggtgccagatgtgcctctgatatgacccagactccatcctccgtgtctgcagctgtgggaggcacagtcaccatcaattgccaggccagtgagaacatttatagctttttggcctggtatcagcagaaaccagggcagcctcccaagctcctgatcttcagggcttccactctggcatctggggtctcatcgcggttcaaaggcagtggatctgggacacagttcactctcaccatcagcgacctggagtgtgacgatgctgccacttactactgtcaacagggtgctactgtgtatgatattgataataat

SEQ ID NO: 499 - DNA, 384 bp, Artificial; identical to SEQ ID NO: 483
  Synthetic Sequence

SEQ ID NO: 500 - DNA, 33 bp, Artificial; identical to SEQ ID NO: 484
  Synthetic Sequence

SEQ ID NO: 501 - DNA, 21 bp, Artificial
  Synthetic Sequence
agggcttccactctggcatct

SEQ ID NO: 502 - DNA, 36 bp, Artificial; identical to SEQ ID NO: 486
  Synthetic Sequence

SEQ ID NO: 503 - DNA, 15 bp, Artificial
  Synthetic Sequence
gcctatgcaatgatc

SEQ ID NO: 504 - DNA, 48 bp, Artificial; identical to SEQ ID NO: 488
  Synthetic Sequence

SEQ ID NO: 505 - DNA, 45 bp, Artificial; identical to SEQ ID NO: 489
  Synthetic Sequence

SEQ ID NO: 506 - protein, 124 aa, Artificial
  Synthetic Polypeptide
MDTRAPTQLLGLLLLWLPGATFAIEMTQTPSPVSAAVGGTVTINCQASESVFNNMLSWYQQKPGHSPKLLIYDASDLASGVPSRFKGSGSGTQFTLTISGVECDDAATYYCAGYKSDSNDGDNV

SEQ ID NO: 507 - protein, 123 aa, Artificial
  Synthetic Polypeptide
METGLRWLLLVAVLKGVQCQSLEESGGRLVTPGTPLTLTCTVSGFSLNRNSITWVRQAPGEGLEWIGIITGSGRTYYANWAKGRFTISKTSTTVDLKMTSPTTEDTATYFCARGHPGLGSGNI

SEQ ID NO: 508 - protein, 12 aa, Artificial
  Synthetic Peptide
QASESVFNNMLS

SEQ ID NO: 509 - protein, 7 aa, Artificial
  Synthetic Peptide
DASDLAS

SEQ ID NO: 510 - protein, 13 aa, Artificial
  Synthetic Peptide
AGYKSDSNDGDNV

SEQ ID NO: 511 - protein, 5 aa, Artificial
  Synthetic Peptide
RNSIT

SEQ ID NO: 512 - protein, 16 aa, Artificial
  Synthetic Peptide
IITGSGRTYYANWAKG

SEQ ID NO: 513 - protein, 10 aa, Artificial
  Synthetic Peptide
GHPGLGSGNI

SEQ ID NO: 514 - DNA, 372 bp, Artificial
  Synthetic Sequence
atggacacgagggcccccactcagctgctggggctcctgctgctctggctcccaggtgccacatttgccattgaaatgacccagactccatcccccgtgtctgccgctgtgggaggcacagtcaccatcaattgccaggccagtgagagtgtttttaataatatgttatcctggtatcagcagaaaccagggcactctcctaagctcctgatctatgatgcatccgatctggcatctggggtcccatcgcggttcaaaggcagtggatctgggacacagttcactctcaccatcagtggcgtggagtgtgacgatgctgccacttactattgtgcagggtataaaagtgatagtaatgatggcgataatgtt

SEQ ID NO: 515 - DNA, 369 bp, Artificial
  Synthetic Sequence
atggagactgggctgcgctggcttctcctggtcgctgtgctcaaaggtgtccagtgtcagtcgctggaggagtccgggggtcgcctggtcacgcctgggacacccctgacactcacctgcacagtctctggattctccctcaacaggaattcaataacctgggtccgccaggctccaggggaggggctggaatggatcggaatcattactggtagtggtagaacgtactacgcgaactgggcaaaaggccgattcaccatctccaaaacctcgaccacggtggatctgaaaatgaccagtccgacaaccgaggacacggccacctatttctgtgccagaggccatcctggtcttggtagtggtaacatc

SEQ ID NO: 516 - DNA, 36 bp, Artificial
  Synthetic Sequence
caggccagtgagagtgtttttaataatatgttatcc

SEQ ID NO: 517 - DNA, 21 bp, Artificial
  Synthetic Sequence
gatgcatccgatctggcatct

SEQ ID NO: 518 - DNA, 39 bp, Artificial
  Synthetic Sequence
gcagggtataaaagtgatagtaatgatggcgataatgtt

SEQ ID NO: 519 - DNA, 15 bp, Artificial
  Synthetic Sequence
aggaattcaataacc

SEQ ID NO: 520 - DNA, 48 bp, Artificial
  Synthetic Sequence
atcattactggtagtggtagaacgtactacgcgaactgggcaaaaggc

SEQ ID NO: 521 - DNA, 30 bp, Artificial
  Synthetic Sequence
ggccatcctggtcttggtagtggtaacatc

SEQ ID NO: 522 - protein, 121 aa, Artificial
  Synthetic Polypeptide
MDTRAPTQLLGLLLLWLPGATFAQVLTQTASSVSAAVGGTVTINCQSSQSVYNNYLSWYQQKPGQPPKLLIYTASSLASGVPSRFKGSGSGTQFTLTISEVQCDDAATYYCQGYYSGPIIT

SEQ ID NO: 523 - protein, 122 aa, Artificial
  Synthetic Polypeptide
METGLRWLLLVAVLKGVQCQSLEESGGRLVTPGTPLTLTCTASGFSLNNYYIQWVRQAPGEGLEWIGIIYAGGSAYYATWANGRFTIAKTSSTTVDLKMTSLTTEDTATYFCARGTFDGYEL

SEQ ID NO: 524 - protein, 12 aa, Artificial
  Synthetic Peptide
QSSQSVYNNYLS

SEQ ID NO: 525 - protein, 7 aa, Artificial
  Synthetic Peptide
TASSLAS

SEQ ID NO: 526 - protein, 10 aa, Artificial
  Synthetic Peptide
QGYYSGPIIT

SEQ ID NO: 527 - protein, 5 aa, Artificial
  Synthetic Peptide
NYYIQ

SEQ ID NO: 528 - protein, 16 aa, Artificial
  Synthetic Peptide
IIYAGGSAYYATWANG

SEQ ID NO: 529 - protein, 8 aa, Artificial
  Synthetic Peptide
GTFDGYEL

SEQ ID NO: 530 - DNA, 363 bp, Artificial
  Synthetic Sequence
atggacacgagggcccccactcagctgctggggctcctgctgctctggctcccaggtgccacatttgcgcaagtgctgacccagactgcatcgtccgtgtctgcagctgtgggaggcacagtcaccatcaattgccagtccagtcagagtgtttataataactacttatcctggtatcagcagaaaccagggcagcctcccaagctcctgatctatactgcatccagcctggcatctggggtcccatcgcggttcaaaggcagtggatctgggacacagttcactctcaccatcagcgaagtgcagtgtgacgatgctgccacttactactgtcaaggctattatagtggtcctataattact

SEQ ID NO: 531 - DNA, 366 bp, Artificial
  Synthetic Sequence
atggagactgggctgcgctggcttctcctggtcgctgtgctcaaaggtgtccagtgtcagtcgctggaggagtccgggggtcgcctggtcacgcctgggacacccctgacactcacctgcacagcctctggattctccctcaataactactacatacaatgggtccgccaggctccaggggaggggctggaatggatcgggatcatttatgctggtggtagcgcatactacgcgacctgggcaaacggccgattcaccatcgccaaaacctcgtcgaccacggtggatctgaagatgaccagtctgacaaccgaggacacggccacctatttctgtgccagagggacatttgatggttatgagttg

SEQ ID NO: 532 - DNA, 36 bp, Artificial
  Synthetic Sequence
cagtccagtcagagtgtttataataactacttatcc

SEQ ID NO: 533 - DNA, 21 bp, Artificial
  Synthetic Sequence
actgcatccagcctggcatct

SEQ ID NO: 534 - DNA, 30 bp, Artificial
  Synthetic Sequence
caaggctattatagtggtcctataattact

SEQ ID NO: 535 - DNA, 15 bp, Artificial
  Synthetic Sequence
aactactacatacaa

SEQ ID NO: 536 - DNA, 48 bp, Artificial
  Synthetic Sequence
atcatttatgctggtggtagcgcatactacgcgacctgggcaaacggc

SEQ ID NO: 537 - DNA, 24 bp, Artificial
  Synthetic Sequence
gggacatttgatggttatgagttg

SEQ ID NO: 538 - protein, 122 aa, Artificial
  Synthetic Polypeptide
MDTRAPTQLLGLLLLWLPGATFAQVLTQTPSPVSVPVGDTVTISCQSSESVYSNNLLSWYQQKPGQPPKLLIYRASNLASGVPSRFKGSGSGTQFTLTISGAQCDDAATYYCQGYYSGVINS

SEQ ID NO: 539 - protein, 124 aa, Artificial
  Synthetic Polypeptide
METGLRWLLLVAVLKGVQCQSVEESGGRLVTPGTPLTLTCTVSGFSLSSYFMSWVRQAPGEGLEYIGFINPGGSAYYASWASGRLTISKTSTTVDLKITSPTTEDTATYFCARILIVSYGAFTI

SEQ ID NO: 540 - protein, 13 aa, Artificial
  Synthetic Peptide
QSSESVYSNNLLS

SEQ ID NO: 541 - protein, 7 aa, Artificial
  Synthetic Peptide
RASNLAS

SEQ ID NO: 542 - protein, 10 aa, Artificial
  Synthetic Peptide
QGYYSGVINS

SEQ ID NO: 543 - protein, 5 aa, Artificial
  Synthetic Peptide
SYFMS

SEQ ID NO: 544 - protein, 16 aa, Artificial
  Synthetic Peptide
FINPGGSAYYASWASG

SEQ ID NO: 545 - protein, 11 aa, Artificial
  Synthetic Peptide
ILIVSYGAFTI

SEQ ID NO: 546 - DNA, 366 bp, Artificial
  Synthetic Sequence
atggacacgagggcccccactcagctgctggggctcctgctgctctggctcccaggtgccacatttgcccaagtgctgacccagactccatcccctgtgtctgtccctgtgggagacacagtcaccatcagttgccagtccagtgagagcgtttatagtaataacctcttatcctggtatcagcagaaaccagggcagcctcccaagctcctgatctacagggcatccaatctggcatctggtgtcccatcgcggttcaaaggcagtggatctgggacacagttcactctcaccatcagcggcgcacagtgtgacgatgctgccacttactactgtcaaggctattatagtggtgtcattaatagt

SEQ ID NO: 547 - DNA, 372 bp, Artificial
  Synthetic Sequence
atggagactgggctgcgctggcttctcctggtcgctgtgctcaaaggtgtccagtgtcagtcggtggaggagtccgggggtcgcctggtcacgcctgggacacccctgacactcacctgcacagtgtctggattctccctcagtagctacttcatgagctgggtccgccaggctccaggggaggggctggaatacatcggattcattaatcctggtggtagcgcatactacgcgagctgggcgagtggccgactcaccatctccaaaacctcgaccacggtagatctgaaaatcaccagtccgacaaccgaggacacggccacctatttctgtgccaggattcttattgtttcttatggagcctttaccatc

SEQ ID NO: 548 - DNA, 39 bp, Artificial
  Synthetic Sequence
cagtccagtgagagcgtttatagtaataacctcttatcc

SEQ ID NO: 549 - DNA, 21 bp, Artificial
  Synthetic Sequence
agggcatccaatctggcatct

SEQ ID NO: 550 - DNA, 30 bp, Artificial
  Synthetic Sequence
caaggctattatagtggtgtcattaatagt

SEQ ID NO: 551 - DNA, 15 bp, Artificial
  Synthetic Sequence
agctacttcatgagc

SEQ ID NO: 552 - DNA, 48 bp, Artificial
  Synthetic Sequence
ttcattaatcctggtggtagcgcatactacgcgagctgggcgagtggc

SEQ ID NO: 553 - DNA, 33 bp, Artificial
  Synthetic Sequence
attcttattgtttcttatggagcctttaccatc

SEQ ID NO: 554 - protein, 122 aa, Artificial
  Synthetic Polypeptide
MDTRAPTQLLGLLLLWLPGARCAYDMTQTPASVEVAVGGTVTIKCQATESIGNELSWYQQKPGQAPKLLIYSASTLASGVPSRFKGSGSGTQFTLTITGVECDDAATYYCQQGYSSANIDNA

SEQ ID NO: 555 - protein, 128 aa, Artificial
  Synthetic Polypeptide
METGLRWLLLVAVLKGVQCQSLEESGGRLVTPGTPLTLTCTVSGFSLSKYYMSWVRQAPEKGLKYIGYIDSTTVNTYYATWARGRFTISKTSTTVDLKITSPTSEDTATYFCARGSTYFTDGGHRLDL

SEQ ID NO: 556 - protein, 11 aa, Artificial
  Synthetic Peptide
QATESIGNELS

SEQ ID NO: 557 - protein, 7 aa, Artificial
  Synthetic Peptide
SASTLAS

SEQ ID NO: 558 - protein, 12 aa, Artificial
  Synthetic Peptide
QQGYSSANIDNA

SEQ ID NO: 559 - protein, 5 aa, Artificial
  Synthetic Peptide
KYYMS

SEQ ID NO: 560 - protein, 17 aa, Artificial
  Synthetic Peptide
YIDSTTVNTYYATWARG

SEQ ID NO: 561 - protein, 14 aa, Artificial
  Synthetic Peptide
GSTYFTDGGHRLDL

SEQ ID NO: 562 - DNA, 366 bp, Artificial
  Synthetic Sequence
atggacacgagggcccccactcagctgctggggctcctgctgctctggctcccaggtgccagatgtgcctatgatatgacccagactccagcctctgtggaggtagctgtgggaggcacagtcaccatcaagtgccaggccactgagagcattggcaatgagttatcctggtatcagcagaaaccagggcaggctcccaagctcctgatctattctgcatccactctggcatctggggtcccatcgcggttcaaaggcagtggatctgggacacagttcactctcaccatcaccggcgtggagtgtgatgatgctgccacttactactgtcaacagggttatagtagtgctaatattgataatgct

SEQ ID NO: 563 - DNA, 384 bp, Artificial
  Synthetic Sequence
atggagactgggctgcgctggcttctcctggtcgctgtgctcaaaggtgtccagtgtcagtcgctggaggagtccgggggtcgcctggtcacgcctgggacacccctgacactcacctgcaccgtctctggattctccctcagtaagtactacatgagctgggtccgccaggctccagagaaggggctgaaatacatcggatacattgatagtactactgttaatacatactacgcgacctgggcgagaggccgattcaccatctccaaaacctcgaccacggtggatctgaagatcaccagtccgacaagtgaggacacggccacctatttctgtgccagaggaagtacttattttactgatggaggccatcggttggatctc

SEQ ID NO: 564 - DNA, 33 bp, Artificial
  Synthetic Sequence
caggccactgagagcattggcaatgagttatcc

SEQ ID NO: 565 - DNA, 21 bp, Artificial
  Synthetic Sequence
tctgcatccactctggcatct

SEQ ID NO: 566 - DNA, 36 bp, Artificial
  Synthetic Sequence
caacagggttatagtagtgctaatattgataatgct

SEQ ID NO: 567 - DNA, 15 bp, Artificial
  Synthetic Sequence
aagtactacatgagc

SEQ ID NO: 568 - DNA, 51 bp, Artificial
  Synthetic Sequence
tacattgatagtactactgttaatacatactacgcgacctgggcgagaggc

SEQ ID NO: 569 - DNA, 42 bp, Artificial
  Synthetic Sequence
ggaagtacttattttactgatggaggccatcggttggatctc

SEQ ID NO: 570 - protein, 122 aa, Artificial; identical to SEQ ID NO: 554
  Synthetic Polypeptide

SEQ ID NO: 571 - protein, 124 aa, Artificial
  Synthetic Polypeptide
METGLRWLLLVAVLKGVQCQSLEESGGRLVTPGTPLTLTCTVSGFSLSTYNMGWVRQAPGKGLEWIGSITIDGRTYYASWAKGRFTVSKSSTTVDLKMTSLTTGDTATYFCARILIVSYGAFTI

SEQ ID NO: 572 - protein, 11 aa, Artificial
  Synthetic Peptide
QATESIGNELS

SEQ ID NO: 573 - protein, 7 aa, Artificial
  Synthetic Peptide
SASTLAS

SEQ ID NO: 574 - protein, 12 aa, Artificial
  Synthetic Peptide
QQGYSSANIDNA

SEQ ID NO: 575 - protein, 5 aa, Artificial
  Synthetic Peptide
TYNMG

SEQ ID NO: 576 - protein, 16 aa, Artificial
  Synthetic Peptide
SITIDGRTYYASWAKG

SEQ ID NO: 577 - protein, 11 aa, Artificial
  Synthetic Peptide
ILIVSYGAFTI

SEQ ID NO: 578 - DNA, 366 bp, Artificial; identical to SEQ ID NO: 562
  Synthetic Sequence

SEQ ID NO: 579 - DNA, 372 bp, Artificial
  Synthetic Sequence
atggagactgggctgcgctggcttctcctggtcgctgtgctcaaaggtgtccagtgtcagtcgctggaggagtccgggggtcgcctggtaacgcctgggacacccctgacactcacctgcacagtctctggattctccctcagtacctacaacatgggctgggtccgccaggctccagggaaggggctggaatggatcggaagtattactattgatggtcgcacatactacgcgagctgggcgaaaggccgattcaccgtctccaaaagctcgaccacggtggatctgaaaatgaccagtctgacaaccggggacacggccacctatttctgtgccaggattcttattgtttcttatggggcctttaccatc

SEQ ID NO: 580 - DNA, 33 bp, Artificial; identical to SEQ ID NO: 564
  Synthetic Sequence

SEQ ID NO: 581 - DNA, 21 bp, Artificial
  Synthetic Sequence
tctgcatccactctggcatct

SEQ ID NO: 582 - DNA, 36 bp, Artificial; identical to SEQ ID NO: 566
  Synthetic Sequence

SEQ ID NO: 583 - DNA, 15 bp, Artificial
  Synthetic Sequence
acctacaacatgggc

SEQ ID NO: 584 - DNA, 48 bp, Artificial
  Synthetic Sequence
agtattactattgatggtcgcacatactacgcgagctgggcgaaaggc

SEQ ID NO: 585 - DNA, 33 bp, Artificial
  Synthetic Sequence
attcttattgtttcttatggggcctttaccatc

SEQ ID NO: 586 - protein, 105 aa, Artificial [sequence omitted]

SEQ ID NO: 587 - DNA, 315 bp, Artificial
  Synthetic Sequence
gtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 588 - protein, 330 aa, Artificial
  Synthetic Polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 589 - DNA, 990 bp, Artificial
  Synthetic Sequence
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 590 - protein, 15 aa, Artificial
  Synthetic Peptide
VPPGEDSKDVAAPHR

SEQ ID NO: 591 - protein, 15 aa, Artificial
  Synthetic Peptide
GEDSKDVAAPHRQPL

SEQ ID NO: 592 - protein, 15 aa, Artificial
  Synthetic Peptide
SKDVAAPHRQPLTSS

SEQ ID NO: 593 - protein, 15 aa, Artificial
  Synthetic Peptide
VAAPHRQPLTSSERI

SEQ ID NO: 594 - protein, 15 aa, Artificial
  Synthetic Peptide
PHRQPLTSSERIDKQ

SEQ ID NO: 595 - protein, 15 aa, Artificial
  Synthetic Peptide
QPLTSSERIDKQIRY

SEQ ID NO: 596 - protein, 15 aa, Artificial
  Synthetic Peptide
TSSERIDKQIRYILD

SEQ ID NO: 597 - protein, 15 aa, Artificial
  Synthetic Pepide
ERIDKQIRYILDGIS

SEQ ID NO: 598 - protein, 15 aa, Artificial
  Synthetic Peptide
DKQIRYILDGISALR

SEQ ID NO: 599 - protein, 15 aa, Artificial
  Synthetic Peptide
IRYILDGISALRKET

SEQ ID NO: 600 - protein, 15 aa, Artificial
  Synthetic Peptide
ILDGISALRKETCNK

SEQ ID NO: 601 - protein, 15 aa, Artificial
  Synthetic Peptide
GISALRKETCNKSNM

SEQ ID NO: 602 - protein, 15 aa, Artificial
  Synthetic Peptide
ALRKETCNKSNMCES

SEQ ID NO: 603 - protein, 15 aa, Artificial
  Synthetic Peptide
KETCNKSNMCESSKE

SEQ ID NO: 604 - protein, 15 aa, Artificial
  Synthetic Peptide
CNKSNMCESSKEALA

SEQ ID NO: 605 - protein, 15 aa, Artificial
  Synthetic Peptide
SNMCESSKEALAENN

SEQ ID NO: 606 - protein, 15 aa, Artificial
  Synthetic Peptide
CESSKEALAENNLNL

SEQ ID NO: 607 - protein, 15 aa, Artificial
  Synthetic Peptide
SKEALAENNLNLPKM

SEQ ID NO: 608 - protein, 15 aa, Artificial
  Synthetic Peptide
ALAENNLNLPKMAEK

SEQ ID NO: 609 - protein, 15 aa, Artificial
  Synthetic Peptide
ENNLNLPKMAEKDGC

SEQ ID NO: 610 - protein, 15 aa, Artificial
  Synthetic Peptide
LNLPKMAEKDGCFQS

SEQ ID NO: 611 - protein, 15 aa, Artificial
  Synthetic Peptide
PKMAEKDGCFQSGFN

SEQ ID NO: 612 - protein, 15 aa, Artificial
  Synthetic Peptide
AEKDGCFQSGFNEET

SEQ ID NO: 613 - protein, 15 aa, Artificial
  Synthetic Peptide
DGCFQSGFNEETCLV

SEQ ID NO: 614 - protein, 15 aa, Artificial
  Synthetic Peptide
FQSGFNEETCLVKII

SEQ ID NO: 615 - protein, 15 aa, Artificial
  Synthetic Peptide
GFNEETCLVKIITGL

SEQ ID NO: 616 - protein, 15 aa, Artificial
  Synthetic Peptide
EETCLVKIITGLLEF

SEQ ID NO: 617 - protein, 15 aa, Artificial
  Synthetic Peptide
CLVKIITGLLEFEVY

SEQ ID NO: 618 - protein, 15 aa, Artificial
  Synthetic Peptide
KIITGLLEFEVYLEY

SEQ ID NO: 619 - protein, 15 aa, Artificial
  Synthetic Peptide
TGLLEFEVYLEYLQN

SEQ ID NO: 620 - protein, 15 aa, Artificial
  Synthetic Peptide
LEFEVYLEYLQNRFE

SEQ ID NO: 621 - protein, 15 aa, Artificial
  Synthetic Peptide
EVYLEYLQNRFESSE

SEQ ID NO: 622 - protein, 15 aa, Artificial
  Synthetic Peptide
LEYLQNRFESSEEQA

SEQ ID NO: 623 - protein, 15 aa, Artificial
  Synthetic Peptide
LQNRFESSEEQARAV

SEQ ID NO: 624 - protein, 15 aa, Artificial
  Synthetic Peptide
RFESSEEQARAVQMS

SEQ ID NO: 625 - protein, 15 aa, Artificial
  Synthetic Peptide
SSEEQARAVQMSTKV

SEQ ID NO: 626 - protein, 15 aa, Artificial
  Synthetic Peptide
EQARAVQMSTKVLIQ

SEQ ID NO: 627 - protein, 15 aa, Artificial
  Synthetic Peptide
RAVQMSTKVLIQFLQ

SEQ ID NO: 628 - protein, 15 aa, Artificial
  Synthetic Peptide
QMSTKVLIQFLQKKA

SEQ ID NO: 629 - protein, 15 aa, Artificial
  Synthetic Peptide
TKVLIQFLQKKAKNL

SEQ ID NO: 630 - protein, 15 aa, Artificial
  Synthetic Peptide
LIQFLQKKAKNLDAI

SEQ ID NO: 631 - protein, 15 aa, Artificial
  Synthetic Peptide
FLQKKAKNLDAITTP

SEQ ID NO: 632 - protein, 15 aa, Artificial
  Synthetic Peptide
KKAKNLDAITTPDPT

SEQ ID NO: 633 - protein, 15 aa, Artificial
  Synthetic Peptide
KNLDAITTPDPTTNA

SEQ ID NO: 634 - protein, 15 aa, Artificial
  Synthetic Peptide
DAITTPDPTTNASLL

SEQ ID NO: 635 - protein, 15 aa, Artificial
  Synthetic Peptide
TTPDPTTNASLLTKL

SEQ ID NO: 636 - protein, 15 aa, Artificial
  Synthetic Peptide
DPTTNASLLTKLQAQ

SEQ ID NO: 637 - protein, 15 aa, Artificial
  Synthetic Peptide
TNASLLTKLQAQNQW

SEQ ID NO: 638 - protein, 15 aa, Artificial
  Synthetic Peptide
SLLTKLQAQNQWLQD

SEQ ID NO: 639 - protein, 15 aa, Artificial
  Synthetic Peptide
TKLQAQNQWLQDMTT

SEQ ID NO: 640 - protein, 15 aa, Artificial
  Synthetic Peptide
QAQNQWLQDMTTHLI

SEQ ID NO: 641 - protein, 15 aa, Artificial
  Synthetic Ppeptide
NQWLQDMTTHLILRS

SEQ ID NO: 642 - protein, 15 aa, Artificial
  Synthetic Peptide
LQDMTTHLILRSFKE

SEQ ID NO: 643 - protein, 15 aa, Artificial
  Synthetic Peptide
MTTHLILRSFKEFLQ

SEQ ID NO: 644 - protein, 15 aa, Artificial
  Synthetic Peptide
HLILRSFKEFLQSSL

SEQ ID NO: 645 - protein, 15 aa, Artificial
  Synthetic Peptide
LRSFKEFLQSSLRAL

SEQ ID NO: 646 - protein, 15 aa, Artificial
  Synthetic Peptide
FKEFLQSSLRALRQM

SEQ ID NO: 647 - protein, 111 aa, Artificial
  Synthetic Polypeptide
AYDMTQTPASVSAAVGGTVTIKCQASQSINNELSWYQQKPGQRPKLLIYRASTLASGVSSRFKGSGSGTEFTLTISDLECADAATYYCQQGYSLRNIDNAFGGGTEVVVKR

SEQ ID NO: 648 - protein, 88 aa, Artificial
  Synthetic Polypeptide
AIQMTQSPSSLSASVGDRVTITCRASQGIRNDLGWYQQKPGKAPKLLIYAASSLQSGVPSRFSGSGSGTDFTLTISSLQPEDFATYYC

SEQ ID NO: 649 - protein, 88 aa, Artificial
  Synthetic Polypeptide
DIQMTQSPSSLSASVGDRVTITCRASQGISNYLAWYQQKPGKVPKLLIYAASTLQSGVPSRFSGSGSGTDFTLTISSLQPEDVATYYC

SEQ ID NO: 650 - protein, 88 aa, Artificial
  Synthetic Polypeptide
DIQMTQSPSTLSASVGDRVTITCRASQSISSWLAWYQQKPGKAPKLLIYKASSLESGVPSRFSGSGSGTEFTLTISSLQPDDFATYYC

SEQ ID NO: 651 - protein, 111 aa, Artificial
  Synthetic Polypeptide
AIQMTQSPSSLSASVGDRVTITCQASQSINNELSWYQQKPGKAPKLLIYRASTLASGVPSRFSGSGSGTDFTLTISSLQPEDFATYYCQQGYSLRNIDNAFGGGTKVEIKR

SEQ ID NO: 652 - protein, 117 aa, Artificial
  Synthetic Polypeptide
QSLEESGGRLVTPGTPLTLTCTASGFSLSNYYVTWVRQAPGKGLEWIGIIYGSDETAYATWAIGRFTISKTSTTVDLKMTSLTAADTATYFCARDDSSDWDAKFNLWGQGTLVTVSS

SEQ ID NO: 653 - protein, 97 aa, Artificial
  Synthetic Polypeptide
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 654 - protein, 97 aa, Artificial
  Synthetic Polypeptide
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 655 - protein, 98 aa, Artificial
  Synthetic Polypeptide
EVQLLESGGGLVQPGGSLRLSCAASGFTFSSYAMSWVRQAPGKGLEWVSVIYSGGSSTYYADSVKGRFTISRDNSKNTLYLQMNSLRAEDTAVYYCAK

SEQ ID NO: 656 - protein, 120 aa, Artificial
  Synthetic Polypeptide
EVQLVESGGGLVQPGGSLRLSCAASGFSLSNYYVTWVRQAPGKGLEWVGIIYGSDETAYATWAIGRFTISRDNSKNTLYLQMNSLRAEDTAVYYCARDDSSDWDAKFNLWGQGTLVTVSS

SEQ ID NO: 657 - protein, 120 aa, Artificial
  Synthetic Polypeptide
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSS

SEQ ID NO: 658 - protein, 166 aa, Artificial
  Synthetic Polypeptide
METGLRWLLLVAVLKGVQCQSLEESGGRLVTPGTPLTLTCTASGFSLSNYYVTWVRQAPGKGLEWIGIIYGSDETAYATSAIGRFTISKTSTTVDLKMTSLTAADTATYFCARDDSSDWDAKFNLWGQGTLVTVSSASTKGPSVFPLAPSSKSTSGGTAALGCLVK

SEQ ID NO: 659 - protein, 16 aa, Artificial
  Synthetic Peptide
IIYGSDETAYATSAIG

SEQ ID NO: 660 - protein, 122 aa, Artificial
  Synthetic Polypeptide
MDTRAPTQLLGLLLLWLPGARCAYDMTQTPASVSAAVGGTVTIKCQASQSINNELSWYQQKPGQRPKLLIYRASTLASGVSSRFKGSGSGTEFTLTISDLECADAATYYCQQGYSLRNIDNA

SEQ ID NO: 661 - protein, 125 aa, Artificial
  Synthetic Polypeptide
METGLRWLLLVAVLKGVQCQSLEESGGRLVTPGTPLTLTCTASGFSLSNYYVTWVRQAPGKGLEWIGIIYGSDETAYATWAIGRFTISKTSTTVDLKMTSLTAADTATYFCARDDSSDWDAKFNL

SEQ ID NO: 662 - DNA, 366 bp, Artificial
  Synthetic Sequence
atggacacgagggcccccactcagctgctggggctcctgctgctctggctcccaggtgccagatgtgcctatgatatgacccagactccagcctcggtgtctgcagctgtgggaggcacagtcaccatcaagtgccaggccagtcagagcattaacaatgaattatcctggtatcagcagaaaccagggcagcgtcccaagctcctgatctatagggcatccactctggcatctggggtctcatcgcggttcaaaggcagtggatctgggacagagttcactctcaccatcagcgacctggagtgtgccgatgctgccacttactactgtcaacagggttatagtctgaggaatattgataatgct

SEQ ID NO: 663 - DNA, 375 bp, Artificial
  Synthetic Sequence
atggagactgggctgcgctggcttctcctggtcgctgtgctcaaaggtgtccagtgtcagtcgctggaggagtccgggggtcgcctggtcacgcctgggacacccctgacactcacctgcacagcctctggattctccctcagtaactactacgtgacctgggtccgccaggctccagggaaggggctggaatggatcggaatcatttatggtagtgatgaaacggcctacgcgacctgggcgataggccgattcaccatctccaaaacctcgaccacggtggatctgaaaatgaccagtctgacagccgcggacacggccacctatttctgtgccagagatgatagtagtgactgggatgcaaaatttaacttg

SEQ ID NO: 664 - protein, 450 aa, Artificial
  Synthetic Polypeptide
EVQLVESGGGLVQPGGSLRLSCAASGFSLSNYYVTWVRQAPGKGLEWVGIIYGSDETAYATWAIGRFTISRDNSKNTLYLQMNSLRAEDTAVYYCARDDSSDWDAKFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 665 - protein, 450 aa, Artificial
  Synthetic Polypeptide
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 666 - protein, 216 aa, Artificial
  Synthetic Polypeptide
IQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 667 - protein, 122 aa, Artificial
  Synthetic Polypeptide
MDTRAPTQLLGLLLLWLPGARCAYDMTQTPASVEVAVGGTVTINCQASETIYSWLSWYQQKPGQPPKLLIYQASDLASGVPSRFSGSGAGTEYTLTISGVQCDDAATYYCQQGYSGSNVDNV

SEQ ID NO: 668 - protein, 126 aa, Artificial
  Synthetic Polypeptide
METGLRWLLLVAVLKGVQCQEQLKESGGRLVTPGTPLTLTCTASGFSLNDHAMGWVRQAPGKGLEYIGFINSGGSARYASWAEGRFTISRTSTTVDLKMTSLTTEDTATYFCVRGGAVWSIHSFDP

SEQ ID NO: 669 - DNA, 366 bp, Artificial
  Synthetic Sequence
atggacacgagggcccccactcagctgctggggctcctgctgctctggctcccaggtgccagatgtgcctatgatatgacccagactccagcctctgtggaggtagctgtgggaggcacagtcaccatcaattgccaggccagtgagaccatttacagttggttatcctggtatcagcagaagccagggcagcctcccaagctcctgatctaccaggcatccgatctggcatctggggtcccatcgcgattcagcggcagtggggctgggacagagtacactctcaccatcagcggcgtgcagtgtgacgatgctgccacttactactgtcaacagggttatagtggtagtaatgttgataatgtt

SEQ ID NO: 670 - DNA, 378 bp, Artificial
  Synthetic Sequence
atggagactgggctgcgctggcttctcctggtcgctgtgctcaaaggtgtccagtgtcaggagcagctgaaggagtccgggggtcgcctggtcacgcctgggacacccctgacacttacctgcacagcctctggattctccctcaatgaccatgcaatgggctgggtccgccaggctccagggaaggggctggaatacatcggattcattaatagtggtggtagcgcacgctacgcgagctgggcagaaggccgattcaccatctccagaacctcgaccacggtggatctgaaaatgaccagtctgacaaccgaggacacggccacctatttctgtgtcagagggggtgctgtttggagtattcatagttttgatccc

SEQ ID NO: 671 - protein, 123 aa, Artificial
  Synthetic Polypeptide
MDTRAPTQLLGLLLLWLPGATFAAVLTQTPSPVSAAVGGTVSISCQASQSVYDNNYLSWFQQKPGQPPKLLIYGASTLASGVPSRFVGSGSGTQFTLTITDVQCDDAATYYCAGVYDDDSDNA

SEQ ID NO: 672 - protein, 125 aa, Artificial
  Synthetic Polypeptide
METGLRWLLLVAVLKGVQCQSLEESGGRLVTPGTPLTLTCTASGFSLSVYYMNWVRQAPGKGLEWIGFITMSDNINYASWAKGRFTISKTSTTVDLKMTSPTTEDTATYFCARSRGWGTMGRLDL

SEQ ID NO: 673 - DNA, 369 bp, Artificial
  Synthetic Sequence
atggacacgagggcccccactcagctgctggggctcctgctgctctggctcccaggtgccacatttgccgccgtgctgacccagactccatctcccgtgtctgcagctgtgggaggcacagtcagcatcagttgccaggccagtcagagtgtttatgacaacaactacttatcctggtttcagcagaaaccagggcagcctcccaagctcctgatctatggtgcatccactctggcatctggggtcccatcgcggttcgtgggcagtggatctgggacacagttcactctcaccatcacagacgtgcagtgtgacgatgctgccacttactattgtgcaggcgtttatgatgatgatagtgataatgcc

SEQ ID NO: 674 - DNA, 375 bp, Artificial
  Synthetic Sequence
atggagactgggctgcgctggcttctcctggtggctgtgctcaaaggtgtccagtgtcagtcgctggaggagtccgggggtcgcctggtcacccctgggacacccctgacactcacctgcacagcctctggattctccctcagtgtctactacatgaactgggtccgccaggctccagggaaggggctggaatggatcggattcattacaatgagtgataatataaattacgcgagctgggcgaaaggccgattcaccatctccaaaacctcgaccacggtggatctgaaaatgaccagtccgacaaccgaggacacggccacctatttctgtgccaggagtcgtggctggggtacaatgggtcggttggatctc

SEQ ID NO: 675 - protein, 123 aa, Artificial
  Synthetic Polypeptide
MDTRAPTQLLGLLLLWLPGAICDPVLTQTPSPVSAPVGGTVSISCQASQSVYENNYLSWFQQKPGQPPKLLIYGASTLDSGVPSRFKGSGSGTQFTLTITDVQCDDAATYYCAGVYDDDSDDA

SEQ ID NO: 676 - protein, 126 aa, Artificial
  Synthetic Polypeptide
METGLRWLLLVAVLKGVQCQEQLKESGGGLVTPGGTLTLTCTASGFSLNAYYMNWVRQAPGKGLEWIGFITLNNNVAYANWAKGRFTFSKTSTTVDLKMTSPTPEDTATYFCARSRGWGAMGRLDL

SEQ ID NO: 677 - DNA, 369 bp, Artificial
  Synthetic Sequence
atggacacgagggcccccactcagctgctggggctcctgctgctctggctcccaggtgccatatgtgaccctgtgctgacccagactccatctcccgtatctgcacctgtgggaggcacagtcagcatcagttgccaggccagtcagagtgtttatgagaacaactatttatcctggtttcagcagaaaccagggcagcctcccaagctcctgatctatggtgcatccactctggattctggggtcccatcgcggttcaaaggcagtggatctgggacacagttcactctcaccattacagacgtgcagtgtgacgatgctgccacttactattgtgcaggcgtttatgatgatgatagtgatgatgcc

SEQ ID NO: 678 - DNA, 378 bp, Artificial
  Synthetic Sequence
atggagactgggctgcgctggcttctcctggtggctgtgctcaaaggtgtccagtgtcaggagcagctgaaggagtccggaggaggcctggtaacgcctggaggaaccctgacactcacctgcacagcctctggattctccctcaatgcctactacatgaactgggtccgccaggctccagggaaggggctggaatggatcggattcattactctgaataataatgtagcttacgcgaactgggcgaaaggccgattcaccttctccaaaacctcgaccacggtggatctgaaaatgaccagtccgacacccgaggacacggccacctatttctgtgccaggagtcgtggctggggtgcaatgggtcggttggatctc

SEQ ID NO: 679 - protein, 122 aa, Artificial
  Synthetic Polypeptide
MDTRAPTQLLGLLLLWLPGATFAQVLTQTPSPVSAAVGGTVTINCQASQSVDDNNWLGWYQQKRGQPPKYLIYSASTLASGVPSRFKGSGSGTQFTLTISDLECDDAATYYCAGGFSGNIFA

SEQ ID NO: 680 - protein, 122 aa, Artificial
  Synthetic Polypeptide
METGLRWLLLVAVLKGVQCQSVEESGGRLVTPGTPLTLTCTVSGFSLSSYAMSWVRQAPGKGLEWIGIIGGFGTTYYATWAKGRFTISKTSTTVDLRITSPTTEDTATYFCARGGPGNGGDI

SEQ ID NO: 681 - DNA, 366 bp, Artificial
  Synthetic Sequence
atggacacgagggcccccactcagctgctggggctcctgctgctctggctcccaggtgccacatttgcccaagtgctgacccagactccatcgcctgtgtctgcagctgtgggaggcacagtcaccatcaactgccaggccagtcagagtgttgatgataacaactggttaggctggtatcagcagaaacgagggcagcctcccaagtacctgatctattctgcatccactctggcatctggggtcccatcgcggttcaaaggcagtggatctgggacacagttcactctcaccatcagcgacctggagtgtgacgatgctgccacttactactgtgcaggcggttttagtggtaatatctttgct

SEQ ID NO: 682 - DNA, 366 bp, Artificial
  Synthetic Sequence
atggagactgggctgcgctggcttctcctggtcgctgtgctcaaaggtgtccagtgtcagtcggtggaggagtccgggggtcgcctggtcacgcctgggacacccctgacactcacctgcacagtctctggcttctccctcagtagctatgcaatgagctgggtccgccaggctccaggaaaggggctggagtggatcggaatcattggtggttttggtaccacatactacgcgacctgggcgaaaggccgattcaccatctccaaaacctcgaccacggtggatctgagaatcaccagtccgacaaccgaggacacggccacctatttctgtgccagaggtggtcctggtaatggtggtgacatc

SEQ ID NO: 683 - protein, 122 aa, Artificial
  Synthetic Polypeptide
MDTRAPTQLLGLLLLWLPGATFAAVLTQTPSPVSVPVGGTVTIKCQSSQSVYNNFLSWYQQKPGQPPKLLIYQASKLASGVPDRFSGSGSGTQFTLTISGVQCDDAATYYCLGGYDDDADNA

SEQ ID NO: 684 - protein, 128 aa, Artificial
  Synthetic Polypeptide
METGLRWLLLVAVLKGVQCQSVEESGGRLVTPGTPLTLTCTVSGIDLSDYAMSWVRQAPGKGLEWIGIIYAGSGSTWYASWAKGRFTISKTSTTVDLKITSPTTEDTATYFCARDGYDDYGDFDRLDL

SEQ ID NO: 685 - DNA, 366 bp, Artificial
  Synthetic Sequence
atggacacgagggcccccactcagctgctggggctcctgctgctctggctcccaggtgccacatttgcagccgtgctgacccagacaccatcgcccgtgtctgtacctgtgggaggcacagtcaccatcaagtgccagtccagtcagagtgtttataataatttcttatcgtggtatcagcagaaaccagggcagcctcccaagctcctgatctaccaggcatccaaactggcatctggggtcccagataggttcagcggcagtggatctgggacacagttcactctcaccatcagcggcgtgcagtgtgacgatgctgccacttactactgtctaggcggttatgatgatgatgctgataatgct

SEQ ID NO: 686 - DNA, 384 bp, Artificial
  Synthetic Sequence
atggagactgggctgcgctggcttctcctggtcgctgtgctcaaaggtgtccagtgtcagtcggtggaggagtccgggggtcgcctggtcacgcctgggacacccctgacgctcacctgcacagtctctggaatcgacctcagtgactatgcaatgagctgggtccgccaggctccagggaaggggctggaatggatcggaatcatttatgctggtagtggtagcacatggtacgcgagctgggcgaaaggccgattcaccatctccaaaacctcgaccacggtggatctgaaaatcaccagtccgacaaccgaggacacggccacctatttctgtgccagagatggatacgatgactatggtgatttcgatcgattggatctc

SEQ ID NO: 687 - protein, 122 aa, Artificial
  Synthetic Polypeptide
MDTRAPTQLLGLLLLWLPGARCAYDMTQTPASVSAAVGGTVTIKCQASQSINNELSWYQQKSGQRPKLLIYRASTLASGVSSRFKGSGSGTEFTLTISDLECADAATYYCQQGYSLRNIDNA

SEQ ID NO: 688 - protein, 125 aa, Artificial
  Synthetic Polypeptide
METGLRWLLLVAVLSGVQCQSLEESGGRLVTPGTPLTLTCTASGFSLSNYYMTWVRQAPGKGLEWIGMIYGSDETAYANWAIGRFTISKTSTTVDLKMTSLTAADTATYFCARDDSSDWDAKFNL

SEQ ID NO: 689 - DNA, 366 bp, Artificial
  Synthetic Sequence
atggacacgagggcccccactcagctgctggggctcctgctgctctggctcccaggtgccagatgtgcctatgatatgacccagactccagcctcggtgtctgcagctgtgggaggcacagtcaccatcaaatgccaggccagtcagagcattaacaatgaattatcctggtatcagcagaaatcagggcagcgtcccaagctcctgatctatagggcatccactctggcatctggggtctcatcgcggttcaaaggcagtggatctgggacagagttcactctcaccatcagcgacctggagtgtgccgatgctgccacttactactgtcaacagggttatagtctgaggaatattgataatgct

SEQ ID NO: 690 - DNA, 375 bp, Artificial
  Synthetic Sequence
atggagactgggctgcgctggcttctcctggtcgctgtgctctcaggtgtccagtgtcagtcgctggaggagtccgggggtcgcctggtcacgcctgggacacccctgacactcacctgcacagcctctggattctccctcagtaactactacatgacctgggtccgccaggctccagggaaggggctggaatggatcggaatgatttatggtagtgatgaaacagcctacgcgaactgggcgataggccgattcaccatctccaaaacctcgaccacggtggatctgaaaatgaccagtctgacagccgcggacacggccacctatttctgtgccagagatgatagtagtgactgggatgcaaaatttaacttg

SEQ ID NO: 691 - protein, 450 aa, Artificial
  Synthetic Polypeptide
EVQLVESGGGLVQPGGSLRLSCAASGFSLSNYYMTWVRQAPGKGLEWVGMIYGSDETAYANWAIGRFTISRDNSKNTLYLQMNSLRAEDTAVYYCARDDSSDWDAKFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 692 - protein, 450 aa, Artificial
  Synthetic Polypeptide
EVQLVESGGGLVQPGGSLRLSCAASGFSLSNYYMTWVRQAPGKGLEWVGMIYGSDETAYANSAIGRFTISRDNSKNTLYLQMNSLRAEDTAVYYCARDDSSDWDAKFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 693 - protein, 217 aa, Artificial
  Synthetic Polypeptide
DIQMTQSPSTLSASVGDRVTITCQASQSINNELSWYQQKPGKAPKLLIYRASTLASGVPSRFSGSGSGTEFTLTISSLQPDDFATYYCQQGYSLRNIDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 694 - DNA, 33 bp, Artificial
  Synthetic Sequence
caggccagtcagagcattaacaatgagttatcc

SEQ ID NO: 695 - DNA, 36 bp, Artificial
  Synthetic Sequence
caacagggttatagtctgaggaacattgataatgct

SEQ ID NO: 696 - DNA, 48 bp, Artificial
  Synthetic Sequence
atcatctatggtagtgatgaaaccgcctacgctacctccgctataggc

SEQ ID NO: 697 - DNA, 36 bp, Artificial
  Synthetic Sequence
gatgatagtagtgactgggatgcaaagttcaacttg

SEQ ID NO: 698 - DNA, 336 bp, Artificial
  Synthetic Sequence
gctatccagatgacccagtctccttcctccctgtctgcatctgtaggagacagagtcaccatcacttgccaggccagtcagagcattaacaatgagttatcctggtatcagcagaaaccagggaaagcccctaagctcctgatctatagggcatccactctggcatctggggtcccatcaaggttcagcggcagtggatctgggacagacttcactctcaccatcagcagcctgcagcctgatgattttgcaacttattactgccaacagggttatagtctgaggaacattgataatgctttcggcggagggaccaaggtggaaatcaaacgtacg

SEQ ID NO: 699 - protein, 112 aa, Artificial
  Synthetic Polypeptide
AIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKRT

SEQ ID NO: 700 - DNA, 360 bp, Artificial
  Synthetic Sequence
gaggtgcagctggtggagtctgggggaggcttggtccagcctggggggtccctgagactctcctgtgcagcctctggattctccctcagtaactactacgtgacctgggtccgtcaggctccagggaaggggctggagtgggtcggcatcatctatggtagtgatgaaaccgcctacgctacctccgctataggccgattcaccatctccagagacaattccaagaacaccctgtatcttcaaatgaacagcctgagagctgaggacactgctgtgtattactgtgctagagatgatagtagtgactgggatgcaaagttcaacttgtggggccaagggaccctcgtcaccgtctcgagc

SEQ ID NO: 701 - DNA, 651 bp, Artificial
  Synthetic Sequence
gctatccagatgacccagtctccttcctccctgtctgcatctgtaggagacagagtcaccatcacttgccaggccagtcagagcattaacaatgagttatcctggtatcagcagaaaccagggaaagcccctaagctcctgatctatagggcatccactctggcatctggggtcccatcaaggttcagcggcagtggatctgggacagacttcactctcaccatcagcagcctgcagcctgatgattttgcaacttattactgccaacagggttatagtctgaggaacattgataatgctttcggcggagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 702 - protein, 217 aa, Artificial
  Synthetic Polypeptide
AIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 703 - DNA, 1350 bp, Artificial
  Synthetic Sequence
gaggtgcagctggtggagtctgggggaggcttggtccagcctggggggtccctgagactctcctgtgcagcctctggattctccctcagtaactactacgtgacctgggtccgtcaggctccagggaaggggctggagtgggtcggcatcatctatggtagtgatgaaaccgcctacgctacctccgctataggccgattcaccatctccagagacaattccaagaacaccctgtatcttcaaatgaacagcctgagagctgaggacactgctgtgtattactgtgctagagatgatagtagtgactgggatgcaaagttcaacttgtggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 704 - protein, 450 aa, Artificial
  Synthetic Polypeptide
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 705 - DNA, 705 bp, Artificial
  Synthetic Sequence
atgaagtgggtaacctttatttcccttctgtttctctttagcagcgcttattccgctatccagatgacccagtctccttcctccctgtctgcatctgtaggagacagagtcaccatcacttgccaggccagtcagagcattaacaatgagttatcctggtatcagcagaaaccagggaaagcccctaagctcctgatctatagggcatccactctggcatctggggtcccatcaaggttcagcggcagtggatctgggacagacttcactctcaccatcagcagcctgcagcctgatgattttgcaacttattactgccaacagggttatagtctgaggaacattgataatgctttcggcggagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 706 - protein, 235 aa, Artificial
  Synthetic Polypeptide
MKWVTFISLLFLFSSAYSAIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 707 - DNA, 1404 bp, Artificial
  Synthetic Sequence
atgaagtgggtaacctttatttcccttctgtttctctttagcagcgcttattccgaggtgcagctggtggagtctgggggaggcttggtccagcctggggggtccctgagactctcctgtgcagcctctggattctccctcagtaactactacgtgacctgggtccgtcaggctccagggaaggggctggagtgggtcggcatcatctatggtagtgatgaaaccgcctacgctacctccgctataggccgattcaccatctccagagacaattccaagaacaccctgtatcttcaaatgaacagcctgagagctgaggacactgctgtgtattactgtgctagagatgatagtagtgactgggatgcaaagttcaacttgtggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 708 - protein, 468 aa, Artificial
  Synthetic Polypeptide
MKWVTFISLLFLFSSAYSEVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 709 - protein, 111 aa, Artificial
  Synthetic Polypeptide
AIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKR

SEQ ID NO: 710 - protein, 11 aa, Artificial
  Synthetic Peptide
RASQGIRNDLG

SEQ ID NO: 711 - protein, 11 aa, Artificial
  Synthetic Peptide
RASQGISNYLA

SEQ ID NO: 712 - protein, 11 aa, Artificial
  Synthetic Peptide
RASQSISSWLA

SEQ ID NO: 713 - protein, 7 aa, Artificial
  Synthetic Peptide
AASSLQS

SEQ ID NO: 714 - protein, 7 aa, Artificial
  Synthetic Peptide
AASTLQS

SEQ ID NO: 715 - protein, 7 aa, Artificial
  Synthetic Peptide
KASSLES

SEQ ID NO: 716 - protein, 5 aa, Artificial
  Synthetic Peptide
SNYMS

SEQ ID NO: 717 - protein, 16 aa, Artificial
  Synthetic Peptide
VIYSGGSTYYADSVKG

SEQ ID NO: 718 - protein, 17 aa, Artificial
  Synthetic Peptide
VIYSGGSSTYYADSVKG

SEQ ID NO: 719 - protein, 330 aa, Artificial
  Synthetic Polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 720 - DNA, 297 bp, Artificial
  Synthetic Sequence
atccagatgacccagtctccttcctccctgtctgcatctgtaggagacagagtcaccatcacttgccaggccagtcagagcattaacaatgagttatcctggtatcagcagaaaccagggaaagcccctaagctcctgatctatagggcatccactctggcatctggggtcccatcaaggttcagcggcagtggatctgggacagacttcactctcaccatcagcagcctgcagcctgatgattttgcaacttattactgccaacagggttatagtctgaggaacattgataatgct

SEQ ID NO: 721 - DNA, 333 bp, Artificial
  Synthetic Sequence
gcctatgatatgacccagactccagcctcggtgtctgcagctgtgggaggcacagtcaccatcaagtgccaggccagtcagagcattaacaatgaattatcctggtatcagcagaaaccagggcagcgtcccaagctcctgatctatagggcatccactctggcatctggggtctcatcgcggttcaaaggcagtggatctgggacagagttcactctcaccatcagcgacctggagtgtgccgatgctgccacttactactgtcaacagggttatagtctgaggaatattgataatgctttcggcggagggaccgaggtggtggtcaaacgt

SEQ ID NO: 722 - DNA, 648 bp, Artificial
  Synthetic Sequence
atccagatgacccagtctccttcctccctgtctgcatctgtaggagacagagtcaccatcacttgccaggccagtcagagcattaacaatgagttatcctggtatcagcagaaaccagggaaagcccctaagctcctgatctatagggcatccactctggcatctggggtcccatcaaggttcagcggcagtggatctgggacagacttcactctcaccatcagcagcctgcagcctgatgattttgcaacttattactgccaacagggttatagtctgaggaacattgataatgctttcggcggagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 723 - DNA, 333 bp, Artificial
  Synthetic Sequence
gctatccagatgacccagtctccttcctccctgtctgcatctgtaggagacagagtcaccatcacttgccaggccagtcagagcattaacaatgagttatcctggtatcagcagaaaccagggaaagcccctaagctcctgatctatagggcatccactctggcatctggggtcccatcaaggttcagcggcagtggatctgggacagacttcactctcaccatcagcagcctgcagcctgatgattttgcaacttattactgccaacagggttatagtctgaggaacattgataatgctttcggcggagggaccaaggtggaaatcaaacgt

SEQ ID NO: 724 - DNA, 327 bp, Artificial
  Synthetic Sequence
gaggtgcagctggtggagtctgggggaggcttggtccagcctggggggtccctgagactctcctgtgcagcctctggattctccctcagtaactactacgtgacctgggtccgtcaggctccagggaaggggctggagtgggtcggcatcatctatggtagtgatgaaaccgcctacgctacctccgctataggccgattcaccatctccagagacaattccaagaacaccctgtatcttcaaatgaacagcctgagagctgaggacactgctgtgtattactgtgctagagatgatagtagtgactgggatgcaaagttcaacttg

SEQ ID NO: 725 - DNA, 351 bp, Artificial
  Synthetic Sequence
cagtcgctggaggagtccgggggtcgcctggtcacgcctgggacacccctgacactcacctgcacagcctctggattctccctcagtaactactacgtgacctgggtccgccaggctccagggaaggggctggaatggatcggaatcatttatggtagtgatgaaacggcctacgcgacctgggcgataggccgattcaccatctccaaaacctcgaccacggtggatctgaaaatgaccagtctgacagccgcggacacggccacctatttctgtgccagagatgatagtagtgactgggatgcaaaatttaacttgtggggccaaggcaccctggtcaccgtctcgagc

SEQ ID NO: 726 - protein, 224 aa, Artificial
  Synthetic Polypeptide
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 727 - protein, 468 aa, Artificial
  Synthetic Polypeptide
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 728 - protein, 918 aa, Artificial [sequence omitted]

SEQ ID NO: 729 - DNA, 32 bp, Artificial
  Synthetic Sequence
agcgcttattccgctatccagatgacccagtc

SEQ ID NO: 730 - DNA, 22 bp, Artificial
  Synthetic Sequence
cgtacgtttgatttccaccttg

SEQ ID NO: 731 - DNA, 32 bp, Artificial
  Synthetic Sequence
agcgcttattccgaggtgcagctggtggagtc

SEQ ID NO: 732 - DNA, 20 bp, Artificial
  Synthetic Sequence
ctcgagacggtgacgagggt

SEQ ID NO: 733 - protein, 111 aa, Artificial
  Synthetic Polypeptide
AYDMTQTPASVEVAVGGTVTINCQASETIYSWLSWYQQKPGQPPKLLIYQASDLASGVPSRFSGSGAGTEYTLTISGVQCDDAATYYCQQGYSGSNVDNVFGGGTEVVVKR

SEQ ID NO: 734 - protein, 99 aa, Artificial
  Synthetic Polypeptide
DIQMTQSPSTLSASVGDRVTITCRASQSISSWLAWYQQKPGKAPKLLIYKASSLESGVPSRFSGSGSGTEFTLTISSLQPDDFATYYCFGGGTKVEIKR

SEQ ID NO: 735 - protein, 88 aa, Artificial
  Synthetic Polypeptide
DIQMTQSPSTLSASVGDRVTITCRASQSISSWLAWYQQKPGKAPKLLIYDASSLESGVPSRFSGSGSGTEFTLTISSLQPDDFATYYC

SEQ ID NO: 736 - protein, 88 aa, Artificial
  Synthetic Polypeptide
DIQMTQSPSSLSASVGDRVTITCRASQSISSYLNWYQQKPGKAPKLLIYAASSLQSGVPSRFSGSGSGTDFTLTISSLQPEDFATYYC

SEQ ID NO: 737 - protein, 111 aa, Artificial
  Synthetic Polypeptide
DIQMTQSPSTLSASVGDRVTITCQASETIYSWLSWYQQKPGKAPKLLIYQASDLASGVPSRFSGSGSGTEFTLTISSLQPDDFATYYCQQGYSGSNVDNVFGGGTKVEIKR

SEQ ID NO: 738 - protein, 118 aa, Artificial
  Synthetic Polypeptide
QEQLKESGGRLVTPGTPLTLTCTASGFSLNDHAMGWVRQAPGKGLEYIGFINSGGSARYASWAEGRFTISRTSTTVDLKMTSLTTEDTATYFCVRGGAVWSIHSFDPWGPGTLVTVSS

SEQ ID NO: 739 - protein, 109 aa, Artificial
  Synthetic Polypeptide
QVQLVESGGGLVQPGGSLRLSCSASGFTFSSYAMHWVRQAPGKGLEYVSAISSNGGSTYYADSVKGRFTISRDNSKNTLYLQMNSLRAEDTAVYYCARWGQGTLVTVSS

SEQ ID NO: 740 - protein, 97 aa, Artificial; identical to SEQ ID NO: 653
  Synthetic Polypeptide

SEQ ID NO: 741 - protein, 97 aa, Artificial
  Synthetic Polypeptide
EVQLVETGGGLIQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 742 - protein, 120 aa, Artificial
  Synthetic Polypeptide
QVQLVESGGGLVQPGGSLRLSCSASGFSLNDHAMGWVRQAPGKGLEYVGFINSGGSARYASSAEGRFTISRDNSKNTLYLQMNSLRAEDTAVYYCARGGAVWSIHSFDPWGQGTLVTVSS

SEQ ID NO: 743 - protein, 4 aa, Artificial
  Synthetic Peptide
FGGG

SEQ ID NO: 744 - protein, 4 aa, Artificial
  Synthetic Peptide
VVKR

SEQ ID NO: 745 - protein, 11 aa, Artificial
  Synthetic Peptide
FGGGTKVEIKR

SEQ ID NO: 746 - protein, 4 aa, Artificial
  Synthetic Peptide
WGXG

SEQ ID NO: 747 - protein, 4 aa, Artificial
  Synthetic Peptide
TVSS

SEQ ID NO: 748 - protein, 11 aa, Artificial
  Synthetic Peptide
WGQGTLVTVSS